SEQUENCE LISTING (27 sequences)

SEQ ID NO: 1 - protein, 9 aa, Artificial Sequence
  Synthetic
TSDKAIELY

SEQ ID NO: 2 - protein, 817 aa, Artificial Sequence
  Synthetic
MDLVLNRKEREALCKLLEIAPNCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTDEEYRSSSFTTPKTPPPFSRKRKFGGSRSSASSASSASFTSTPPKPKKNRETPVPTDFPIDLSDYLSHAVYSNKTVSCFAIYTTSDKAIELYDKIEKFKVDFKSRHACELGCILLFITLSKHRVSAIKNFCSTFCTISFLICKGVNKMPEMYNNLCKPPYKLLQENKPLLNYEFQEKEKEASCNWNLVAEFACEYELDDHFIILAHYLDFAKPFPCQKCENRSRLKPHKAHEAHHSNAKLFYESKSQKTICQQAADTVLAKRRLEMLEMTRTEMLCKKFKKHLERLRDLDTIDLLYYMGGVAWYCCLFEEFEKKLQKIIQLLTENIPKYRNIWFKGPINSGKTSFAAALIDLLEGKALNINCPSDKLPFELGCALDKFMVVFEDVKGQNSLNKDLQPGQGINNLDNLRDHLDGAVAVSLEKKHVNKKHQIFPPCIVTANDYFIPKTLIARFSYTLHFSPKANLRDSLDQNMEIRKRRILQSGTTLLLCLIWCLPDTTFKPCLQEEIKNWKQILQSEISYGKFCQMIENVEAGQDPLLNILIEEEGPEETEETQDSGTFSQ

SEQ ID NO: 3 - protein, 9 aa, Artificial Sequence
  Synthetic
  misc_feature (1)..(1): Xaa can be any naturally occurring amino acid
  misc_feature (3)..(3): Xaa can be any naturally occurring amino acid
  X (4)..(4): X is E or D
  misc_feature (5)..(5): Xaa can be any naturally occurring amino acid
  X (6)..(6): X is L, V, or I
  X (7)..(7): X is I, V, or A
  misc_feature (8)..(8): Xaa can be any naturally occurring amino acid
  X (9)..(9): X is L or V
XLXXXXXXX

SEQ ID NO: 4 - protein, 9 aa, Artificial Sequence
  Synthetic
  misc_feature (1)..(1): Xaa can be any naturally occurring amino acid
  misc_feature (3)..(3): Xaa can be any naturally occurring amino acid
  misc_feature (5)..(6): Xaa can be any naturally occurring amino acid
  X (7)..(7): X is L
  misc_feature (8)..(8): Xaa can be any naturally occurring amino acid
  X (9)..(9): X is F or Y
XLXDXXXXX

SEQ ID NO: 5 - protein, 9 aa, Artificial Sequence
  Synthetic
  misc_feature (1)..(1): Xaa can be any naturally occurring amino acid
  X (2)..(2): X is T, S, or L
  X (3)..(3): X is D or E
  X (4)..(4): X is I, V, or A
  misc_feature (5)..(8): Xaa can be any naturally occurring amino acid
XXXXXXXXY

SEQ ID NO: 6 - protein, 9 aa, Artificial Sequence
  Synthetic
  misc_feature (1)..(1): Xaa can be any naturally occurring amino acid
  misc_feature (3)..(5): Xaa can be any naturally occurring amino acid
  X (6)..(6): X is L or V
  misc_feature (7)..(8): Xaa can be any naturally occurring amino acid
  X (9)..(9): X is V or L
XLXXXXXXX

SEQ ID NO: 7 - protein, 9 aa, Artificial Sequence
  Synthetic
  misc_feature (1)..(1): Xaa can be any naturally occurring amino acid
  misc_feature (3)..(3): Xaa can be any naturally occurring amino acid
  X (4)..(4): X is E or D
  misc_feature (5)..(5): Xaa can be any naturally occurring amino acid
  X (6)..(6): X is L or V
  misc_feature (7)..(8): Xaa can be any naturally occurring amino acid
  X (9)..(9): X is V or L
XLXXXXXXX

SEQ ID NO: 8 - protein, 9 aa, Artificial Sequence
  Synthetic
  misc_feature (1)..(1): Xaa can be any naturally occurring amino acid
  misc_feature (4)..(8): Xaa can be any naturally occurring amino acid
  X (9)..(9): X is L or V
XLDXXXXXX

SEQ ID NO: 9 - protein, 9 aa, Artificial Sequence
  Synthetic
  misc_feature (1)..(1): Xaa can be any naturally occurring amino acid
  X (2)..(2): X is L or V
  misc_feature (3)..(6): Xaa can be any naturally occurring amino acid
  misc_feature (8)..(8): Xaa can be any naturally occurring amino acid
  X (9)..(9): X is K, Y, or R
XXXXXXLXX

SEQ ID NO: 10 - protein, 9 aa, Artificial Sequence
  Synthetic
  X (1)..(1): X is R or A
  X (2)..(2): X is V or L
  misc_feature (3)..(8): Xaa can be any naturally occurring amino acid
XXXXXXXXR

SEQ ID NO: 11 - protein, 9 aa, Artificial Sequence
  Synthetic
  X (2)..(2): X is V, T, or A
  misc_feature (3)..(8): Xaa can be any naturally occurring amino acid
  X (9)..(9): X is V or L
EXXXXXXXX

SEQ ID NO: 12 - protein, 9 aa, Artificial Sequence
  Synthetic
  misc_feature (1)..(1): Xaa can be any naturally occurring amino acid
  X (2)..(2): X is P or A
  misc_feature (3)..(3): Xaa can be any naturally occurring amino acid
  misc_feature (5)..(8): Xaa can be any naturally occurring amino acid
  X (9)..(9): X is Y, F, or L
XXXRXXXXX

SEQ ID NO: 13 - protein, 9 aa, Artificial Sequence
  Synthetic
  X (1)..(1): X is A or E
  misc_feature (3)..(4): Xaa can be any naturally occurring amino acid
  misc_feature (6)..(8): Xaa can be any naturally occurring amino acid
  X (9)..(9): X is F, W, or L
XEXXLXXXX

SEQ ID NO: 14 - protein, 9 aa, Artificial Sequence
  Synthetic
  X (1)..(1): X is A or E
  misc_feature (3)..(4): Xaa can be any naturally occurring amino acid
  misc_feature (6)..(6): Xaa can be any naturally occurring amino acid
  misc_feature (8)..(8): Xaa can be any naturally occurring amino acid
  X (9)..(9): X is F, W, or L
XEXXLXLXX

SEQ ID NO: 15 - protein, 9 aa, Artificial Sequence
  Synthetic
  X (2)..(2): X is P or A
  misc_feature (3)..(8): Xaa can be any naturally occurring amino acid
  X (9)..(9): X is I, V, or L
DXXXXXXXX

SEQ ID NO: 16 - protein, 9 aa, Artificial Sequence
  Synthetic
  X (1)..(1): X is M, W, or V
  misc_feature (3)..(8): Xaa can be any naturally occurring amino acid
  X (9)..(9): X is F or L
XYXXXXXXX

SEQ ID NO: 17 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 18 - protein, 9 aa, Artificial Sequence
  Synthetic
IEVMYPPPY

SEQ ID NO: 19 - protein, 107 aa, Artificial Sequence; identical to SEQ ID NO: 17
  Synthetic

SEQ ID NO: 20 - DNA, 26 bp, Artificial Sequence
  Synthetic
gatacaaggctgttagagagataatt

SEQ ID NO: 21 - DNA, 21 bp, Artificial Sequence
  Synthetic
gaccaagaggaagaatcacaa

SEQ ID NO: 22 - DNA, 21 bp, Artificial Sequence
  Synthetic
gctgcgaagaagctcgttaga

SEQ ID NO: 23 - DNA, 21 bp, Artificial Sequence
  Synthetic
ggagcagcttacaccaattga

SEQ ID NO: 24 - DNA, 11 bp, Artificial Sequence
  Synthetic
cgcagcacaag

SEQ ID NO: 25 - DNA, 11 bp, Artificial Sequence
  Synthetic
cgcagcacaag

SEQ ID NO: 26 - DNA, 22 bp, Artificial Sequence
  Synthetic
gccgatgcaggagtcgcacagc

SEQ ID NO: 27 - DNA, 22 bp, Artificial Sequence
  Synthetic
gccgatgcaggagtcgcacagc

(19) United States
(12) Patent Application Publication
(10) Pub. No.: US 2023/0248814 A1
(43) Pub. Date: Aug. 10, 2023

(54) COMPOSITIONS AND METHODS FOR TREATING MERKEL CELL CARCINOMA (MCC) USING HLA CLASS I SPECIFIC EPITOPES

(71) Applicants: The Broad Institute, Inc., Cambridge, MA (US); Dana-Farber Cancer Institute, Inc., Boston, MA (US)

(72) Inventors: Catherine Wu, Boston, MA (US); Steven Carr, Cambridge, MA (US); Susan Klaeger, Cambridge, MA (US); Derin Keskin, Boston, MA (US)

(21) Appl. No.: 17/928,649

(22) PCT Filed: Jun. 1, 2021
(86) PCT No.: PCT/US2021/035196

§ 371 Date: Nov. 30, 2022

Related U.S. Application Data
(60) Provisional application No. 63/033,191, filed on Jun. 1, 2020.

(51) Int. Cl. A61K 39/12; C07K 16/08; C07K 14/725

(52) U.S. Cl. CPC A61K 39/12; C07K 14/7051; C07K 16/084; A61K 2039/5154

(57) ABSTRACT

The subject matter disclosed herein is generally directed to epitopes that specifically bind to subject specific HLA class I molecules in MCC. The epitope identified is specific for MCC and is encoded for in the Merkel Cell Polyomavirus (MCPyV) large T antigen (MCPyV LT).

### CROSS-REFERENCE TO RELATED APPLICATIONS

This application claims the benefit of U.S. Provisional Application No. 63/033,191, filed Jun. 1, 2020. The entire contents of the above-identified application are hereby fully incorporated herein by reference.

### SEQUENCE LISTING

This application contains a sequence listing filed in electronic form as an ASCII.txt file entitled BROD-5170WP_ST25.txt, created on May 31, 2021 and having a size of 19,051 bytes (20 KB on disk). The content of the sequence listing is incorporated herein in its entirety.

### STATEMENT REGARDING FEDERALLY SPONSORED RESEARCH

This invention was made with government support under Grant Nos. CA216772, CA155010, CA224331, HL131768, CA101942, CA006516, CA210986 and CA214125 awarded by the National Institutes of Health. The government has certain rights in the invention.

### TECHNICAL FIELD

The subject matter disclosed herein is generally directed to epitopes that specifically bind to subject specific HLA class I molecules in MCC.

### BACKGROUND

The therapeutic landscape of cancer treatment has been transformed by potent immunotherapeutic agents such as checkpoint blockade inhibitors. Despite their promise, the majority of cancer patients demonstrate an inadequate response, and a more precise understanding of immune evasion is paramount to advancing immunotherapy. One important mechanism is loss of human leukocyte antigen class I (HLA-I). The frequency of partial or complete surface HLA-I loss can reach 80% in many cancers (1) and occurs through genomic or transcriptional alterations to class I antigen presentation machinery (APM) genes (2-4). HLA-I loss correlates with worse prognosis and has been identified as a common mechanism of resistance to immunotherapy (4-8). The restoration of HLA-I expression in HLA-I-low cancers, specifically in the case of transcriptional loss, represents an unmet therapeutic need and may synergize with existing immunotherapeutic agents. While IFN-γ is a known inducer of HLA-I, endogenous intratumoral IFN-γ is largely produced by tumor infiltrating lymphocytes (9, 10) and thus closely linked to tumor HLA-I expression. Moreover, exogenous IFN-γ produces systemic side effects and may exert pro-tumorigenic effects as well (11).

Viruses employ an array of mechanisms to evade immune system recognition, allowing for undetected infection and replication. A common target for viral immune evasion is the HLA class I (HLA I or MHC I) antigen presentation pathway, which requires the coordinated function of several steps, including peptide processing (PSMB8/LMP2, PSMB9/LMP7), peptide transport from the cytosol to the ER (TAP1, TAP2), and peptide loading to the B2M-BLA I heavy chain (HLA-A, -B, -C) complex. To perturb this pathway and avoid viral antigen presentation, viruses block HLA I heavy chain insertion into the ER (CMV), resist proteasomal degradation (EBV), interfere with TAP (herpesviruses), or facilitate ubiquitination and degradation of surface-expressed HLA I, among other mechanisms. Continued characterization of these strategies by which viruses circumvent immune recognition can shed light on mechanisms of class I presentation and regulation, with relevance to virology and cancer.

The development of targeted HLA-I-upregulating agents necessitates a better understanding of how cancers transcriptionally suppress class I APM genes. One intriguing model system to study this is Merkel cell carcinoma (MCC).

MCC is a rare, highly aggressive neuroendocrine skin cancer, caused by the Merkel cell polyomavirus (MCPyV) in roughly 80% of cases (12, 13). MCPyV+ MCC is a low tumor mutational burden (TMB) subtype driven by two viral antigens: Large T antigen (LT), which inactivates RB (Hesbacher et al. 2016), and Small T antigen (ST), which has numerous functions, including recruitment of MYCL to chromatin-modifying complexes (Cheng et al. 2017a). MCPyV- MCC exhibits high TMB secondary to ultraviolet (UV) damage and almost invariably contains mutations in TP53 and RB1. Notably, both subtypes of MCC exhibit low HLA-I expression, observed by immunohistochemistry (IHC) in 84% of MCC tumors and confirmed in MCC cell lines (Ritter et al. 2017; K. G. Paulson et al. 2014). However, HLA-I expression in MCC also appears to be highly plastic, as it can be upregulated in vitro by interferons (IFNs) or histone deacetylase (HDAC) inhibitors (Ritter et al. 2017; K. G. Paulson et al. 2014).

Thorough investigation of the class I antigen presentation system in MCPyV+ MCC has the potential to uncover novel mechanisms of HLA I suppression. Such findings could also have implications for MCPyV- MCC, the high mutational burden variant of MCC that intriguingly also displays low HLA I despite lacking viral antigens. However, existing MCC lines are limited in number, and several lines, particularly those that are MCPyV-, are poor representatives of primary tumors (Daily et al. 2015). To address these limitations, Applicants established an approach to consistently generate MCC lines directly from tumor biopsies and patient-derived xenografts. Applicants hypothesized that viral antigen-mediated signaling suppresses HLA-I surface expression in MCPyV+ MCC through regulatory pathways that may also be perturbed in MCPyV- MCC and other cancers. Applicants systematically characterized class I APM genes in 11 newly generated MCC lines through genomic and proteomic analysis. Applicants then interrogated MCC lines through genome-scale gain- and loss-of-function screens for the restoration of HLA-I. These screens identified MYCL and the noncanonical Polycomb repressive complex 1.1 (PRC1.1) as novel regulators of HLA-I. Applicants further demonstrate that pharmacologic inhibition of PRC1.1 component USP7 can restore HLA-I expression.

Citation or identification of any document in this application is not an admission that such a document is available as prior art to the present invention.

### SUMMARY

In one aspect, the present invention provides for an immunogenic composition for the treatment of Merkel Cell Carcinoma (MCC) comprising a peptide or polynucleotide encoding for the peptide derived from the OBD polypeptide of Merkel Cell Polyomavirus (MCPyV) large T antigen (MCPyV LT). In certain embodiments, the peptide corresponds to amino acids 341-349 of MCPyV LT. In certain embodiments, the peptide comprises TSDKAIELY (SEQ ID NO: 1). In certain embodiments, the peptide is an HLA*A01:01-restricted class I epitope. In certain embodiments, the peptide is presented on an antigen presenting cell. In certain embodiments, the antigen presenting cell is a dendritic cell. In certain embodiments, the peptide is presented by an HLA tetramer.

In another aspect, the present invention provides for an ex-vivo immune cell for the treatment of Merkel Cell Carcinoma (MCC) comprising a chimeric antigen receptor (CAR), endogenous T cell receptor (TCR) or exogenous T cell receptor (TCR) specific for a peptide derived from the OBD polypeptide of Merkel Cell Polyomavirus (MCPyV) large T antigen (MCPyV LT). In certain embodiments, the peptide corresponds to amino acids 341-349 of MCPyV LT. In certain embodiments, the peptide comprises TSDKAIELY (SEQ ID NO: 1). In certain embodiments, the peptide is an HLA*A01:01-restricted class I epitope. In certain embodiments, the immune cell is a T cell or NK cell. In certain embodiments, the immune cell is an autologous T cell.

In another aspect, the present invention provides for an antibody for the treatment of Merkel Cell Carcinoma (MCC) specific for a peptide derived from the OBD polypeptide of Merkel Cell Polyomavirus (MCPyV) large T antigen (MCPyV LT). In certain embodiments, the peptide corresponds to amino acids 341-349 of MCPyV LT. In certain embodiments, the peptide comprises TSDKAIELY (SEQ ID NO: 1). In certain embodiments, the peptide is an HLA*A01:01-restricted class I epitope. In certain embodiments, the antibody is a bispecific antibody or antibody drug conjugate. In certain embodiments, the bi-specific antibody is a bi-specific T-cell engager (BiTE).

In another aspect, the present invention provides for a method of treatment comprising administering the immunogenic composition, immune cell or antibody of any embodiment herein to a subject in need thereof. In certain embodiments, the method further comprises administering a treatment that increases HLA class I expression prior or concurrently, wherein the treatment is selected from the group consisting of an interferon gamma therapy and a USP7 inhibitor.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1F - Generation of patient-derived MCC lines that exhibit classic features of MCC and recapitulate the low HLA-I expression of their corresponding tumors. (FIG. 1A) Immunohistochemistry (IHC) of two MCC cell lines with stains for MCC markers SOX2 and CK20. One representative MCPyV+ (MCC-277) and MCPyV- (MCC-350) line are shown. (FIG. 1B) CoMut plot displaying the top 50 most frequently mutated genes across 7 MCC tumor and cell line pairs. (FIG. 1C) Unsupervised hierarchical clustering of RNA-seq samples, comprised of 9 MCC patient tumors and corresponding cell lines. Heatmaps were constructed using a distance matrix on variance-stabilizing transformed expression values. Top track indicates quantification of transcript reads mapping to the MCPyV genome. (FIG. 1D) Flow cytometry results (left y-axis) for HLA-I surface expression across 11 MCC lines, both at baseline (center bars) and in response to IFN-γ (right bars), compared to isotype control (left bars). The overlaid black line plot indicates the percentage of tumor cells that stained positive for HLA-I by IHC of the corresponding original tumor (right y-axis). (FIG. 1E) IHC of MCC tumor archival samples. Left - Summary of the percent of MCC cells that are HLA I-positive within available pre- (n=6) and post-treatment (n=9) tumor samples (see Table 1 for prior treatments). MCC cell lines were derived from post-treatment samples. Right - representative IHC images of two HLA I-low tumors, MCC-301 and MCC-336, stained for HLA class I (brown) with SOX2 co-stain (red) to identify MCC cells. (FIG. 1F) Unsupervised hierarchical clustering of RNA-seq data from 9 MCC patient tumors and corresponding cell lines. Heatmaps were constructed using a distance matrix on variance stabilizing transformed expression values. Bottom track: quantification of viral RNA reads. Unmapped reads from the RNA-seq bam files of 10 of the MCC tumor and cell line pairs were aligned to the reference sequence of the MCC polyomavirus.

FIGS. 2A-2E - Transcriptional repression of multiple class I pathway genes and NLRC5 alterations underlie the loss of HLA-I surface expression in the panel of MCC lines. (FIG. 2A) RNA-seq heatmaps of class I antigen presentation gene expression. Middle heatmap -unsupervised clustering by Euclidean distance of the MCC cell line panel, both at baseline and after IFN-y treatment. Left - reference heatmap of previously established MCC lines MKL-1 and WaGa. Right - reference heatmap of normal epidermal keratinocytes and dermal fibroblasts. (FIG. 2B) Unsupervised clustering by Euclidian distance of proteomic expression values for class I pathway genes in 4 MCC lines, at baseline and after IFN-γ treatment. (FIG. 2C) scRNA-seq data from MCC-336 (MCPyV⁺) and -350 (MCPyV⁻) fresh tumor samples. UMAP (uniform manifold approximation and projection) visualization of all cells are displayed, shaded by cluster (left) and by sample (middle). Right: Expression levels of HLA-A, -B, -C, and B2M across all clusters (clusters 0-5 = MCC cells; cluster 6 = immune cells) (FIG. 2D) NLRC5 copy number loss is common in MCC. Log₂ copy number ratios are displayed for class I antigen presentation genes (left) and for chromosome 16 (right), where NLRC5 is located. Shading signifies copy number gain and loss, respectively (FIG. 2E). Unsupervised clustering of promoter methylation of class I pathway genes in 8 of the MCC lines, generated from whole-genome bisulfite sequencing.

FIGS. 3A-3M - IFN-γ increases and alters the HLA peptidome in MCC. (FIG. 3A) Number of detected peptides presented on HLA-I in MCC lines at baseline (left bar) and after IFN-γ treatment (right bar). CL = cell line. (FIG. 3B) Correlation heatmap of peptide sequences between MCC lines at baseline and after IFN-y treatment in motif space. (FIG. 3C) 9mer motif changes between untreated and IFN-γ-treated samples for MCC-290 (MCPyV⁻) and -301 (MCPyV⁺) cell lines. (FIG. 3D) HLA allele distribution of presented peptides detected in cell lines at baseline and after IFN-γ treatment. Each HLA allele is represented by a different shade. (FIG. 3E) Summary of changes in peptides presented per HLA gene upon IFN-γ treatment across all MCC lines analyzed for HLA-A (left), -B (middle), and -C (right). (FIG. 3F) Mass spectrum of a detected HLA-A-presented peptide derived from the MCPyV Large T antigen in MCC-367. Red, blue and green peaks represent y-, b- and internal ions respectively, confirming the peptide sequence. (FIG. 3G) IFN-γ secretion by peripheral blood mononuclear cells (PBMCs) from patient MCC-367 co-cultured in an ELISpot with DMSO, HIV-GAG negative control peptide, autologous MCC-367 tumor cells, or the Large T antigen-derived peptide identified in the MCC-367 HLA peptidome in panel F. Left - ELISpot conditions conducted in triplicate. Right - summary statistics (mean ± standard deviation). P values determined by one-way ANOVA followed by post hoc Tukey’s multiple comparisons test. (FIG. 3H) IFN-y stimulation of cell lines. (FIG. 3I) Correlation in the immunopeptidomes between the tumors and cell lines at baseline. (FIG. 3J) Inferred frequencies of peptides presented on each class I HLA allele between corresponding tumors and cell lines. (FIG. 3K) Peptide motif landscapes. (FIG. 3L) Frequencies of peptides mapping to each HLA allele. (FIG. 3M) Upregulation of HLA-A, -B, -C.

FIGS. 4A-4H - MYCL identified as novel regulator of HLA-I through genome-scale ORF screen. (FIG. 4A) Workflow and FACS gating strategy for the genome-scale ORF and CRISPR screens. (FIG. 4B) Results for the gain-of-function ORF screen. Genes were ranked according to their log₂-fold-change enrichment in HLA-I-high versus -low populations. Inset: GSEA analysis of ORF positive hits. (FIG. 4C) Flow cytometry for surface HLA-I (W6/32 antibody) in MCC-301 (left) and MCC-277 (right) cells transduced with the indicated individual ORFs. (FIG. 4D) Flow cytometry for surface HLA-I in MKL-1 cells transduced with a dox-inducible control shRNA, MYCL shRNA MYCL, or MYCL shRNA with rescue expression of MYCL. Top panel - representative flow histograms; middle panel - mean MFIs (normalized to corresponding samples not treated with dox) for each condition (n=3); bottom panel - Western blots for MYCL expression levels in each cell line. P values determined by one-way ANOVA followed by post hoc Tukey’s multiple comparisons test. (FIG. 4E) RNA-seq volcano plot showing LFC expression in MKL-1 cells expressing a shRNA against MYCL compared to a scrambled control shRNA. Class I APM genes with p_adj < 0.05 and log₂-fold change (LFC) > 1 are highlighted in red; other notable class I genes are in black. (FIG. 4F) RNA-seq volcano plot showing LFC expression in WaGa cells expressing an shRNA against both ST and LT antigens, compared to a scrambled control shRNA. Class I APM genes with p_adj < 0.05 and LFC > 1 are highlighted in light shading; other notable class I genes in black. (FIG. 4G) Copy number variations in MYC family genes in 4 of the MCPyV- MCC lines. CN gains and losses are shown in red and blue, respectively. Gray indicates no CNV data available. (FIG. 4H) Unsupervised clustering by Euclidian distance of RNA-seq expression values of class I pathway genes and MYC family genes across all available cancer cell lines in the Cancer Cell Line Encyclopedia (44). For each cancer type, the median expression value from all available cell lines of that cancer classification was used.

FIGS. 5A-5R - The PRC1.1 complex implicated as a novel suppressor of HLA-I in genome-wide CRISPR screen. (FIG. 5A) Results for the loss-of-function CRISPR-KO screen. Guide RNA ranks based on log₂-fold-change enrichment in HLA-I-high versus -low populations were input into the STARS algorithm to generate a gene-level ranking of positive (left) and negative (right) hits. Inset: GSEA analysis of CRISPR positive and negative hits. (FIG. 5B) Flow cytometry for surface HLA-I in MCC-301 PRC1.1 KO lines (PCGF1, USP7, and BCORL1). Knockout lines were made using either the highest or second-highest scoring sgRNA for each gene. (FIG. 5C) Western blot for PCGF1 (top) and USP7 (bottom) in WT MCC-301, a control MCC-301 line transduced with a non-targeting sgRNA and Cas9, or the indicated knockout line. (FIG. 5D) RNA-seq volcano plot showing LFC in gene expression in an MCC-301 PCGF1-KO line compared to MCC-301 transduced with a non-targeting sgRNA and Cas9 control. Inset: GSEA plot demonstrating enrichment of PRC2 targets within genes upregulated in the PCGF1-KO line. (FIG. 5E) Western blot showing TAP1 protein levels in non-targeting control and PCGF1-KO lines at varying IFN-y concentrations. (FIG. 5F) RNA-seq analysis of HLA-I genes and notable screen hits across a cohort of 51 MCC tumors. Left: Unsupervised hierarchical clustering heatmap by Euclidian distance. Top track: tumor purity scores for each tumor, generated by ESTIMATE(51). Bottom track: Viral status of tumor (dark brown = positive; light brown = negative). Right: Similarity matrix between class I genes and screen hits across samples. Blue and red indicate negative and positive Pearson correlation coefficients, respectively. Circles represent p-values < 0.05, triangles represent p-values ≥ 0.05. Size of symbol inversely correlates with magnitude of p-value (not corrected for multiple comparisons). (FIG. 5G) UCSC genome browser view of USP7 and PCGF1 with ChIP-seq tracks for MAX, EP-400, MCPyV ST antigen, and activating histone marks H3K4me3 and H3K27Ac. (FIG. 5H) ChIP-qPCR targeting the USP7 and PCGF1 promoters, using MKL-1 chromatin immunoprecipitated with either a MAX (left) or EP400 (right) antibody. Each condition was repeated in triplicate, and p-values were calculated by performing a one-way ANOVA followed by a post hoc Dunnett multiple comparisons test. (FIG. 5I) Schematic of putative interactions between MCPyV viral antigens and screen hits MYCL and PRC1.1. (FIG. 5J) Results for the loss-of-function CRISPR-KO screen. Guide RNA ranks based on log-fold-change enrichment in MHC-I-hi versus -low populations were input into the STARS algorithm (ref) to generate a gene-level ranking of positive (left) and negative (right) hits. Inset: GSEA analysis displaying select gene sets enriched in CRISPR positive and negative hits. Flow cytometry for surface MHC I in MCC-301 ORF lines (FIG. 5K) or MYCL-overexpressing IMR90 fibroblasts. (FIG. 5L) Flow cytometry for surface MHC I in MCC-301 PRC1.1 KO lines. MCC-301 cells were transduced with lentivirus containing Cas9 and either control sgRNA or sgRNAs targeting PRC1.1 components BCORL1, PCGF1, or USP7. Cells were selected with puromycin for 3 days, and knockout was confirmed via Sanger sequencing and Western blot or qRT-PCR. Cells were stained with anti-HLA-ABC (W6/32 clone) and analyzed on a BD LSRFortessa. Each condition was repeated in technical triplicate. (FIG. 5M) Schematic of PRC1.1 components and MYCL, with indication of screen hits and screen hits that have also been reported to interact with MCPyV viral antigens. (FIG. 5N) and pan-T antigen shRNA knockdown versus scrambled control shRNA in MCPyV+ WaGa line (FIG. 5O). Class I genes with p_adj < 0.05 and LFC > 1 are highlighted; other notable class I genes in black. (FIG. 5P) Copy number variations in MYC family genes in 4 of the virus-negative MCC lines for which whole-genome sequencing was performed. CN gains and losses are shown, respectively. Gray indicates no CNV data (FIG. 5Q) Unsupervised clustering of RNA-seq expression values of class I pathway genes and MYC family genes across all available cancer cell lines in the Cancer Cell Line Encyclopedia. For each cancer type, the median expression value from all cell lines of that cancer classification was used. Color scale is row-min to row-max. (FIG. 5R) RNA-seq analysis of HLA class I genes and notable screen hits across a cohort of 52 MCC tumors. Top: Unsupervised hierarchical clustering heatmap using Pearson correlations. Top track: tumor purity scores for each tumor, generated by ESTIMATE (dx.doi.org/10.1038/ncomms3612). Bottom track: Viral status of tumor (orange = positive; green = negative). Bottom: Similarity matrices between class I genes and screen hits in VP and VN samples. Blue and red, indicate negative and positive Pearson correlation coefficient, respectively, and larger circle size corresponds to smaller p value. P-values not corrected for multiple comparisons.

FIGS. 6A-6E - Pharmacologic inhibition of PRC1.1 component USP7 upregulates HLA-I in MCPyV+ MCC. (FIG. 6A) Top: Dependency data from the Cancer Dependency Map (DepMap)(58, 59) was stratified based on TP53 mutation status (TP53-mut (n=532) vs. TP53-wt (n=235)). Left: Pearson correlation coefficients and FDRs of the top genes that are co-dependent with USP7, with PRC1.1 genes highlighted. Right: Graphical comparison of dependency of USP7 versus PRC1.1 genes PCGF1 and RING1 in TP53-WT and TP53-mut cell lines. Bottom: Graphical comparison of dependency of USP7 versus Polycomb genes MGA, PCGF1, and RING1 in TP53-WT and TP53-mut cell lines. (FIG. 6B) Top: Flow cytometry experiments measuring HLA-I surface levels in MCC lines treated with the USP7 inhibitor XL177A or control compound XL177B. Y-axis displays MFI (HLA-ABC) in inhibitor-treated cells, normalized to the mean MFI (HLA-ABC) of DMSO-treated cells. Sample preparation and flow cytometry analysis was performed in technical triplicate for each condition. ** is P < 0.01; * is P < 0.05; n.s. is P ≥ 0.05. Bottom: MCC-301 cells were incubated for 4 days with the indicated concentration of Inhibitor A (active USP7 inhibitor) or inhibitor B (control, inactive compound). Cells were then stained with a HLA-ABC antibody (W6/32) and analyzed on a BD LSRFortessa. Y-axis displays MFI (HLA-ABC) in inhibitor-treated cells, normalized to MFI(HLA-ABC) of DMSO-treated MCC-301 cells. Each concentration contains duplicate or triplicate measurements from independent but technically identical experiments. (FIG. 6C) HLA I flow cytometry to assess the effect of USP7 inhibitors in MKL-1 p53-WT control lines (left) or p53-KO lines (right). Cells were treated with 100 nM XL177A, XL177B, or DMSO. (FIG. 6D) Heatmap of peptide abundances within the HLA-I-presented peptidomes of MCC-301 cells treated with XL177A (red) or XL177B (black), compared to untreated cells (gray) (n=2 replicates). Only peptides that were significantly differentially expressed between any two treatment groups (determined by two-sample t test) are shown. (FIG. 6E) Frequency of peptides presented on each HLA allele in MCC-301 cells treated with XL177A or XL177B, compared to untreated cells.

FIGS. 7A-7I - Related to FIGS. 1. Further characterization of 11 novel MCC cell lines. FIG. 7A) Cell culture media optimization in the MCC-336 cell line. Cells were counted at day 0, 4, and 7 (n=3 replicates derived from original tumor). FIG. 7B) Growth curves of newly generated MCC cell lines. One million cells were seeded in triplicate on Day 0 and counted at Day 2 and Day 4. FIG. 7C) Immunohistochemistry for 8 of the newly generated MCC cell lines, with staining for MCC markers SOX2 and CK20. FIG. 7D) MCPyV genome coverage at the DNA level detected by ViroPanel (top) and at the transcriptional level detected by RNA-seq (bottom). FIG. 7E) Clustering of MCC tumors and cell lines by similarity in mutational profiles. Similarity scores were calculated based on the concordant presence or absence of mutations between tumor and cell line on a 0 to 1 scale, where a score of 1 indicates identical profiles. FIG. 7F) Pairwise Spearman correlations based on RNA-seq data for corresponding tumor-cell line pairs, along with all possible tumor-tumor pairs, cell line-cell line pairs, and all other pairings. Center line, median; box limits, upper and lower quartiles; whiskers, range excluding outliers. FIG. 7G) Immunohistochemistry of 9 of the MCC cell lines, with staining for classical MCC markers SOX2 and CK20. FIG. 7H) Plot of RNA-seq coverages across the Merkel cell polyomavirus reference genome for both tumor and cell line of all virus-positive RNA-seq samples. Lines show smoothed normalized coverage values for virus-mapped reads in each sample, with sT and LT annotated in red. The 2 highly expressed viral sequences across all samples are shown on the bottom, along with the positions and sequence changes of the two single nucleotide variants. FIG. 7I) IHC staining of 4 original MCC tumor biopsies for HLA class I, HLA-DR, CD4, and CD8.

FIGS. 8A-8F - Effects of interferons on HLA-I and -II expression in MCC lines and IHC characterization. (FIG. 8A) Flow cytometry experiments measuring HLA-I surface expression (W6/32 antibody, PE) in two established MCPyV+ lines, MKL-1 and WaGa, alongside MCC-301. (FIG. 8B) Effect of type I and type II IFNs on surface MHC I expression in MCC by flow cytometry. 5 ×10⁵ MCC cells were treated with the indicated doses of IFNα2b, IFNβ, or IFNγ for 24 hours. Representative histogram plots show cells stained with anti-HLA-I (W6/32, APC) or isotype antibodies. The experiment was performed in the MCPyV- line MCC-290 (left) and the MCPyV+ line MCC-301 (right). (FIG. 8C) Flow cytometry assessment of HLA-DR expression in all 11 MCC lines, both at baseline (center) and after IFN-γ treatment (right), compared to isotype control (left). (FIG. 8D) IHC images of parental MCC tumors, stained for HLA class I with SOX2 co-stain to identify MCC cells. (FIG. 8E) Summary of the percent of MCC cells that are HLA II-positive within available pre- (n=6) and post-treatment (n=9) tumor samples (see Table 1 for prior treatments). MCC cell lines were derived from post-treatment samples. (FIG. 8F) Representative multiplex immunofluorescence images of MCC FFPE tumor tissue sections. Probes include DAPI nuclear, CD8, FOXP3, PD-1, PD-L1, and SOX2.

FIGS. 9A-9F - MCC lines exhibit low HLA-I expression at both the bulk and single cell level. (FIG. 9A) Volcano plot of differentially expressed genes with FDR < 0.01 (notable HLA-I genes labeled) between baseline and IFN-γ-treated MCC cell lines. Negative LFC indicates increased expression in +IFN-γ samples. (FIG. 9B) Proteomics heatmap depicting the relative expression of key IFN-γ pathway components in 4 MCC lines, both at baseline and after IFN-γ treatment. Gray shading indicates that the protein was not detected. (FIG. 9C) Targeted analysis of normalized STAT1 peptide counts (left) and STAT-Y701y phosphosite counts (right) between untreated and IFN-γ-treated cell lines. Absence of bar indicates that the peptide/phosphosite was not detected in that particular sample. (FIG. 9D) scRNA-seq expression of MCC markers SOX2, ATOH1, and synaptophysin (SYP), and immune cell marker CD45 within the MCC-336 and -350 tumor samples. (FIG. 9E) scRNA-seq expression of additional HLA-I genes across all clusters (clusters 0-5: MCC; cluster 6: immune cells). (FIG. 9F) CoMut plot demonstrating the minimal mutational burden in interferon signaling genes within 7 of the MCC lines for which WES data was generated.

FIGS. 10A-10F - Additional immunopeptidome data. (FIG. 10A) Schematic representation of immunopeptidome workflow. HLA molecules are immunoprecipitated from tumor and cell line material, peptides are eluted from HLA complex and analyzed by LC-MS/MS. After database searching, peptides are assigned to their most likely allele by prediction in HLAthena. (FIG. 10B) Bar charts showing the number of detected peptides in primary tumor, cell line, and IFN-γ-treated cell lines for select MCC lines. Left: total peptide counts. Right: Peptide counts normalized to IP input. (FIG. 10C) Correlation heatmap of peptide sequences in motif space between MCC tumors, cell lines at baseline, and cell lines after IFN-γ treatment. (FIG. 10D) Pie charts of HLA-I-presented peptides in select MCC cell lines that were also detected in the corresponding tumor sample (black) or were unique to the cell line (gray). (FIG. 10E) Motif changes of 9mers between baseline cell line and IFN-γ-treated cell line samples. (FIG. 10F) Frequencies of peptides presented on each class I HLA allele.

FIGS. 11A-11F - ORF screen implicates MYCL as a negative regulator of HLA-I in MCC. (FIG. 11A) Flow cytometric assessment of HLA-I surface expression (W6/32 antibody) in MCC-301 cells transduced with the human ORFeome v8.1 library lentivirus. Controls include MCC-301 cells transduced with a GFP ORF virus, a no-virus control, and un-transduced cells. (FIG. 11B) Violin plot of the log₂ normalized construct abundance scores for each sorted population of the ORF screen. Middle line indicates median; upper and lower lines indicate upper and lower quartiles, respectively. (FIG. 11C) Scatterplot of gene-level LFCs (average LFC of all constructs for a given gene) between two replicates of the ORF screen. Notable screen hits are highlighted. (FIG. 11D) Enrichment of the KEGG term ‘Antigen processing and presentation’ in GSEA analysis of gene upregulated in MKL-1 shMYCL cells relative to a scrambled shRNA control. (FIG. 11E) Differential expression analysis of MKL-1 cells transduced with one of two shRNAs against EP400 (shEP400-2 or shEP400-3), compared to a scrambled shRNA control. Red indicates HLA-I genes with LFC > 1 and p_{adj} < 0.01. Triangles indicate genes whose p_{adj} values were reported as zero by DeSeq2, and subsequently plotted at the lowest non-zero p_{adj} value in the dataset. (FIG. 11F) Brunello library pre- and post-amplification.

FIGS. 12A-12H - CRISPR screen identifies PRC1.1 as a negative regulator of HLA-I in MCC. (FIG. 12A) Violin plot of the log₂ normalized construct abundance scores for each sorted population of the CRISPR screen. Middle line indicates median; upper and lower lines indicate upper and lower quartiles, respectively. (FIG. 12B) Scatterplot showing concordance of gene-level LFCs (average LFC of all constructs for a given gene) between two replicates of the CRISPR screen. Notable screen hits are highlighted. (FIG. 12C) Average LFC enrichment of the 3 highest-scoring sgRNAs for USP7, BCORL1, and PCGF1, with the distribution of a set of control non-targeting or intergenic sgRNAs shown as a reference. (FIG. 12D) Flow cytometry for surface HLA-I in a double guide PCGF1 KO line after IFN-γ treatment. (FIG. 12E) TIDE analysis of PRC1.1 single-guide KO lines. Left: the percentage of cells with indels in each knockout line was determined using TIDE software⁴⁹. Right: Example TIDE analysis tracing of the PCGF1 sgRNA #2 KO line in MCC-301. (FIG. 12F) Western blot quantification of TAP1 and TAP2 in MKL-1 cells in response to varying concentrations of IFN-γ. (FIG. 12G) Genome browser view of BCOR and BCORL1 with ChIP-seq tracks for MAX, EP-400, MCPyV ST antigen, and activating histone marks H3K4me3 and H3K27Ac. (FIG. 12H) Comparison of genes identified in two screens.

FIGS. 13A-13C - Pharmacologic inhibition of USP7 upregulates HLA-I. (FIG. 13A) The GO terms “Histone ubiquitination” and “Histone H2A ubiquitination” are highly enriched within genes that exhibit co-dependency with USP7 in TP53-mut cancer cell lines by GSEA analysis. (FIG. 13B) Western blot for p53 in 3 MKL-1 p53 KO lines compared to control lines (WT, SCR, AAVS1). (FIG. 13C) Distribution of cell cycle phases, determined by flow cytometry, of MKL-1 p53 KO lines treated with XL177A, XL177B, or DMSO.

FIGS. 14A-14E - Characterization of MCC lines FIG. 14A) Pearson correlation plots between class 1 genes and NLRC5 generated from RNA-seq data from the 11 MCC cell lines. P-values not adjusted for multiple comparisons. FIG. 14B) ATAC-seq normalized read coverage in 8 of the MCC lines, focusing on the TSS +/- 5kb of class I genes and the housekeeping gene TBP. All datasets including those from GEO and ENCODE were normalized by RPKM (see Methods). FIG. 14C) Comparison of the percentage of peaks falling within the union DNase-1 hypersensitivity sites (DHS) between the MCC lines and datasets on Cistrome DB. Comparison to the median level (left) as well as the full distribution (right) are shown. FIG. 14D) Comparison of total, 5-fold and 10-fold enriched peak numbers across MCC lines with the median of Cistrome DB datasets. Dashed line represents peak number of 500. FIG. 14E) Graph showing peak conservation across samples.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR2: A Practical Approach (1995) (M.J. MacPherson, B.D. Hames, and G.R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/-10% or less, +/-5% or less, +/-1% or less, and +/-0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide novel epitopes for targeting MCPyV+ MCC. The epitopes were identified as binding to HLA class I molecules after stimulation to increase HLA expression. The epitopes identified herein can be used in an immunological composition, such as a vaccine, or can be targeted by a therapeutic antibody or immune cell (e.g., CAR T cell).

Cancers and viruses avoid immune surveillance through an array of mechanisms, including perturbation of human leukocyte antigen class I (HLA-I) antigen presentation. Merkel cell carcinoma (MCC) is an aggressive, HLA-I-low neuroendocrine skin cancer often caused by the Merkel cell polyomavirus (MCPyV). Through the characterization of 11 newly generated MCC cell lines, Applicants identified transcriptional suppression of several class I antigen presentation genes. To systematically identify regulators of HLA-I loss in MCC, Applicants performed parallel, genome-scale, gain- and loss-of-function screens in an MCPyV-positive line and identified MYCL and the noncanonical Polycomb repressive complex PRC1.1 as HLA-I repressors. Applicants observed physical interaction of MYCL with the MCPyV Small T viral antigen, suggestive of a mechanism of virally mediated HLA-I suppression. Applicants further identify the PRC1.1 component USP7 as a pharmacologic target to restore HLA-I expression in MCC

Key findings include: Loss of surface HLA I is a prominent feature in a panel of 11 novel Merkel cell carcinoma lines. These MCC lines exhibit alterations to NLRC5 and coordinated transcriptional downregulation of multiple class I pathway genes. Restoration of HLA class I by IFN-γ increases the diversity of the immunopeptidome and allows for detection of viral antigens. Genome-wide screens identify the Polycomb complex PRC1.1 and MYCL as novel regulators of HLA I expression in MCC, and pharmacologic inhibition of PRC1.1 component deubiquitylating enzyme, USP7 upregulates class I. PRC1.1 and MYCL may interact with MCPyV viral antigens to coordinate class I suppression. Applicants detected an HLA*A1:01-restricted class I epitope prediction (TSDKAIELY (SEQ ID NO:1); rank per HLAthena) derived from LT, which was detected only after IFN-γ treatment and not at baseline.

### Therapeutic Compositions and Methods of Use

### Antigenic Tumor Epitopes

Applicants identified a novel HLA class I epitope in MCPyV LT. The epitope of the invention includes epitopes in viral proteins of other viruses encoding an LT protein or other MCPyV viral strains or variants that are homologous to the epitope identified herein. In certain embodiments, the epitope is the epitope that corresponds to the amino acids in MCPyV LT and may have one or more variations (e.g., substitutions of amino acids with similar charge). For example MCPyV may be the Merkel cell polyomavirus isolate R17b (complete genome, NC_010277.2) and the epitope may be TSDKAIELY (SEQ ID NO:1). An exemplary sequence for MCPyV LT is shown below.

MDLVLNRKEREALCKLLEIAPNCYGNIPLMKAAFKRSCLKHHPDKGGNPV
IMMELNTLWSKFQQNIHKLRSDFSMFDEVDEAPIYGTTKFKEWWRSGGFS
FGKAYEYGPNPHGTNSRSRKPSSNASRGAPSGSSPPHSQSSSSGYGSFSA
SQASDSQSRGPDIPPEHHEEPTSSSGSSSREETTNSGRESSTPNGTSVPR
NSSRTDGTWEDLFCDESLSSPEPPSSSEEPEEPPSSRSSPRQPPSSSAEE
ASSSQFTDEEYRSSSFTTPKTPPPFSRKRKFGGSRSSASSASSASFTSTP
PKPKKNRETPVPTDFPIDLSDYLSHAVYSNKTVSCFAIYTTSDKAIELYD
KIEKFKVDFKSRHACELGCILLFITLSKHRVSAIKNFCSTFCTISFLICK
GVNKMPEMYNNLCKPPYKLLQENKPLLNYEFQEKEKEASCNWNLVAEFAC
EYELDDHFIILAHYLDFAKPFPCQKCENRSRLKPHKAHEAHHSNAKLFYE
SKSQKTICQQAADTVLAKRRLEMLEMTRTEMLCKKFKKHLERLRDLDTID
LLYYMGGVAWYCCLFEEFEKKLQKIIQLLTENIPKYRNIWFKGPINSGKT
SFAAALIDLLEGKALNINCPSDKLPFELGCALDKFMVVFEDVKGQNSLNK
DLQPGQGINNLDNLRDHLDGAVAVSLEKKHVNKKHQIFPPCIVTANDYFI
PKTLIARFSYTLHFSPKANLRDSLDQNMEIRKRRILQSGTTLLLCLIWCL
PDTTFKPCLQEEIKNWKQILQSEISYGKFCQMIENVEAGQDPLLNILIEE
EGPEETEETQDSGTFSQ (SEQ IDNO: 2).

### Immune System and Antigen Presentation

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B- lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

By “proteins or molecules of the major histocompatibility complex (MHC)”, “MHC molecules”, “MHC proteins” or “HLA proteins” is thus meant proteins capable of binding peptides resulting from the proteolytic cleavage of protein antigens and representing potential T- cell epitopes, transporting them to the cell surface and presenting them there to specific cells, in particular cytotoxic T-lymphocytes or T-helper cells. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin (B2M).

MHC molecules of class II consist of an a-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The a-chain and the β-chain are in particular HLA-DR, HLA-DQ and HLA-DP monomers.

Subject specific HLA alleles or HLA genotype of a subject may be determined by any method known in the art. In preferred embodiments, HLA genotypes are determined by any method described in International Patent Application number PCT/US2014/068746, published Jun. 11, 2015 as WO2015085147. Briefly, the methods include determining polymorphic gene types that may comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

### Immunological Compositions and Vaccines

In certain embodiments, the peptides of the present invention are used in a vaccine or immunological composition to treat any disease or condition described herein (e.g., tumor or infection). The term “vaccine” or “immunological composition” are used interchangeably and are meant to refer in the present context to a pooled sample of one or more antigenic peptides, for example at least one, at least two, at least three, at least four, at least five, or more antigenic peptides. A “vaccine” is to be understood as including a protective vaccine, which is a composition for generating immunity for the prophylaxis and/or treatment of diseases (e.g., neoplasia/tumor). A “vaccine” is also to be understood as including a tolerizing vaccine, which is a composition for reducing immunity for the prophylaxis and/or treatment of diseases (e.g., autoimmune disease). A protective vaccine may be formulated with antigenic epitopes specific for a pathogen or for a cancer cell. Accordingly, vaccines are medicaments which comprise antigens and are intended to be used in humans or animals for generating specific defense and protective substance by vaccination. A “vaccine composition” can include a pharmaceutically acceptable excipient, carrier or diluent.

The vaccine may include one or more peptides identified according to the present invention. For example, 1 to 10 peptides. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

The vaccine of the present invention may ameliorate a disease as described herein. By “ameliorate” is meant decrease, suppress, attenuate, diminish, arrest, or stabilize the development or progression of a disease (e.g., a neoplasia, tumor, infection, etc.).

The terms “treat,” “treated,” “treating,” “treatment,” and the like are meant to refer to reducing or ameliorating a disorder and/or symptoms associated therewith (e.g., a neoplasia or tumor). “Treating” may refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

In certain embodiments, a protective vaccine is used to treat cancer, in particular, a cancer caused by a virus expressing a large T antigen (LT). Additional examples of cancers and cancer conditions that can be treated with the therapy of this document include, but are not limited to a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin’s disease, Non-Hodgkin’s disease lymphoma, Burkitt’s lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi’s sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. Thus the population of subjects described herein may be suffering from one of the above cancer types. In other embodiments, the population of subjects may be all subjects suffering from solid tumors, or all subjects suffering from liquid tumors.

Cancers that can be treated using the therapy described herein may include among others cases which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

The therapy described herein is also applicable where the subject has no detectable neoplasia but is at high risk for disease recurrence.

Also of special interest is the treatment of patients in need thereof who have undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the claimed neoplastic vaccine or immunogenic composition compositions to delay disease relapse.

The present invention is based, at least in part, on the ability to present the immune system of the patient with one or more HLA allele specific peptides. In certain embodiments, the immune system of the patient is presented with a pool of tumor specific antigens in addition to the LT epitope identified. The application further provides novel antigenic peptides. Accordingly, provided herein are immunogenic compositions comprising a peptide having a sequence selected from XLXX₄XX₆X₇XX₉ (SEQ ID NO:3); wherein one or more of X₄ is E or D, X₆ is L, V, or I, X₇ is I, V, or A, and X₉ is L or V, and wherein X is any amino acid; XLXDXXX₇XX₉ (SEQ ID NO:4), wherein one or more of X₇ is L and X₉ is Y or F, and wherein X is any amino acid; XX₂X₃X₄XXXXY (SEQ ID NO:5), wherein one or more of X₂ is T, S, or L, X₃ is D or E and X₄ is I, V, or A, and wherein X is any amino acid; XLXXXX₆XXX₉ (SEQ ID NO:6); wherein one or more of X₆ is L or V and X₉ is V or L, and wherein X is any amino acid; XLXX₄XX₆XXX₉ (SEQ ID NO:7), wherein one or more of X₄ is E or D, X₆ is L or V and X₉ is V or L, and wherein X is any amino acid; XLDXXXXXX₉ (SEQ ID NO:8), wherein X₉ is L or V, and wherein X is any amino acid; XX₂XXXXLXX₉ (SEQ ID NO:9), wherein one or more of X₂ is L or V and X₉ is K, Y or R, and wherein X is any amino acid; X₁X₂XXXXXXR (SEQ ID NO:10), wherein one or more of X₁ is R or A and X₂ is V or L, and wherein X is any amino acid; EX₂XXXXXXX₉ (SEQ ID NO:11), wherein one or more of X₂ is V, T, or A and X₉ is V or L, and wherein X is any amino acid; XX₂XRXXXXX₉ (SEQ ID NO:12), wherein one or more of X₂ is P or A and X₉ is Y, F, or L, and wherein X is any amino acid; X₁EXXLXXXX₉ (SEQ ID NO: 13), wherein one or more of X₁ is A or E and X₉ is F, W, or L, and wherein X is any amino acid; X₁EXXLXLXX₉ (SEQ ID NO: 14), wherein one or more of X₁ is A or E and X9 is F, W, or L, and wherein X is any amino acid; DX₂XXXXXXX₉ (SEQ ID NO:15), wherein one or more of X₂ is P or A and X₉ is I, V, or L, and wherein X is any amino acid; and X₁YXXXXXXX₉ (SEQ ID NO:16), wherein one or more of X₁ is M, W, or V and X₉ is F or L, and wherein X is any amino acid.

### Producing Antigenic Peptides

One of skill in the art from this disclosure and the knowledge in the art will appreciate that there are a variety of ways in which to produce such tumor specific antigens or any other antigens. In general, such tumor specific antigens or antigens may be produced either in vitro or in vivo. Tumor specific antigens or antigens may be produced in vitro as peptides or polypeptides, which may then be formulated into a neoplasia vaccine or immunogenic composition and administered to a subject. As described in further detail herein, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific antigens or antigens may be produced in vivo by introducing molecules (e.g., DNA, RNA, viral expression systems, and the like) that encode tumor specific antigens or antigens into a subject, whereupon the encoded tumor specific antigens or antigens are expressed. The methods of in vitro and in vivo production of antigens or antigens is also further described herein as it relates to pharmaceutical compositions and methods of delivery of the therapy. By an isolated “polypeptide” or “peptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

In certain embodiments, the present invention includes modified antigenic or antigenic peptides. As used herein in reference to peptides, the terms “modified”, “modification” and the like refer to one or more changes that enhance a desired property of the antigenic peptide, where the change does not alter the primary amino acid sequence of the antigenic peptide. “Modification” includes a covalent chemical modification that does not alter the primary amino acid sequence of the antigenic peptide itself. Such desired properties include, for example, prolonging the in vivo half-life, increasing the stability, reducing the clearance, altering the immunogenicity or allergenicity, enabling the raising of particular antibodies, cellular targeting, antigen uptake, antigen processing, MHC affinity, MHC stability, or antigen presentation. Changes to an antigenic peptide that may be carried out include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. Modified peptides also include analogs. By “analog” is meant a molecule that is not identical, but has analogous functional or structural features. For example, a tumor specific neo-antigen polypeptide analog retains the biological activity of a corresponding naturally-occurring tumor specific neo-antigen polypeptide, while having certain biochemical modifications that enhance the analog’s function relative to a naturally-occurring polypeptide. Such biochemical modifications could increase the analog’s protease resistance, membrane permeability, or half-life, without altering, for example, ligand binding. An analog may include an unnatural amino acid.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Modified peptides may include a spacer or a linker. The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins or RNA sequences. However, in certain embodiments, the constituent amino acids of a spacer may be selected to influence some property of the molecule such as the folding, net charge, or hydrophobicity of the molecule.

Suitable linkers for use in an embodiment of the present invention are well known to those of skill in the art and include, but are not limited to, straight or branched-chain carbon linkers, heterocyclic carbon linkers, or peptide linkers. The linker is used to separate two antigenic peptides by a distance sufficient to ensure that, in a preferred embodiment, each antigenic peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40: 39-46; Murphy et al. (1986) Proc. Nat′l. Acad. Sci. USA 83 : 8258-62; U.S. Pat. No. 4,935,233; and U.S. Pat. No. 4,751,180.

The clinical effectiveness of protein therapeutics is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non- proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG- conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide sequence are generally soluble in water at room temperature, and have the general formula R(0-CH₂-CH₂)ₙO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, but certain embodiments have a molecular weight between 500 and 20,000 while other embodiments have a molecular weight between 4,000 and 10,000. The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to a polypeptide of the present disclosure via a terminal reactive group (a “spacer”). The spacer is, for example, a terminal reactive group which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N- hydroxy succinylimide. Another activated polyethylene glycol which may be bound to a free amino group is 2,4-bis(0-methoxypolyethyleneglycol)-6-chloro-s-triazine which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., -20° C.

The present disclosure also contemplates the use of PEG Mimetics. Recombinant PEG mimetics have been developed that retain the attributes of PEG (e.g., enhanced serum half- life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix’ XTEN technology; Mountain View, CA). This obviates the need for an additional conjugation step during the manufacturing process. Moreover, established molecular biology techniques enable control of the side chain composition of the polypeptide chains, allowing optimization of immunogenicity and manufacturing properties.

For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties of proteins and can also be important in protein stability, secretion, and subcellular localization. Proper glycosylation can be essential for biological activity. In fact, some genes from eucaryotic organisms, when expressed in bacteria (e.g., E. coli) which lack cellular processes for glycosylating proteins, yield proteins that are recovered with little or no activity by virtue of their lack of glycosylation.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O- linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the DNA level, particularly by mutating the DNA encoding the polypeptide at preselected bases such that codons are generated that will translate into the desired amino acids. Another means of increasing the number of carbohydrate moieties on the polypeptide is by chemical or enzymatic coupling of glycosides to the polypeptide.

Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Dihydrofolate reductase (DHFR) - deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more polypeptides of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake, incorporation and expression of exogenous genetic material (exogenous DNA) from its surroundings and taken up through the cell membrane(s). Transformation occurs naturally in some species of bacteria, but it can also be effected by artificial means in other cells.

Furthermore, albumin itself may be modified to extend its circulating half-life. Fusion of the modified albumin to one or more Polypeptides can be attained by the genetic manipulation techniques described above or by chemical conjugation; the resulting fusion molecule has a half-life that exceeds that of fusions with non-modified albumin. (See WO2011/051489).

Several albumin - binding strategies have been developed as alternatives for direct fusion, including albumin binding through a conjugated fatty acid chain (acylation). Because serum albumin is a transport protein for fatty acids, these natural ligands with albumin - binding activity have been used for half-life extension of small protein therapeutics. For example, insulin determir (LEVEMIR), an approved product for diabetes, comprises a myristyl chain conjugated to a genetically-modified insulin, resulting in a long- acting insulin analog.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule. A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein (e.g., diabetes).

A Polypeptide may also be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Purification methods such as cation exchange chromatography may be used to separate conjugates by charge difference, which effectively separates conjugates into their various molecular weights. For example, the cation exchange column can be loaded and then washed with -20 mM sodium acetate, pH -4, and then eluted with a linear (0 M to 0.5 M) NaCl gradient buffered at a pH from about 3 to 5.5, e.g., at pH -4.5. The content of the fractions obtained by cation exchange chromatography may be identified by molecular weight using conventional methods, for example, mass spectroscopy, SDS-PAGE, or other known methods for separating molecular entities by molecular weight.

In certain embodiments, the amino- or carboxyl- terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure contemplates the use of other modifications, currently known or developed in the future, of the Polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule’s characteristics. Various aspects of hesylation are described in, for example, U.S. Pat. Appln. Nos. 2007/0134197 and 2006/0258607.

### In Vitro Peptide/Polypeptide Synthesis

Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information’s Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). In certain embodiments, antigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding a antigenic peptide of the invention may be used to produce the antigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, MA).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The antigenic peptides may be provided in the form of RNA or cDNA molecules encoding the desired antigenic peptides. One or more antigenic peptides of the invention may be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific antigenic peptide fused in the same reading frame to a marker sequence that allows, for example, for purification of the encoded polypeptide, which may then be incorporated into the personalized neoplasia vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific antigenic peptides fused in the same reading frame to create a single concatamerized antigenic peptide construct capable of producing multiple antigenic peptides.

In certain embodiments, isolated nucleic acid molecules having a nucleotide sequence at least 60% identical, at least 65% identical, at least 70% identical, at least 75% identical, at least 80%) identical, at least 85% identical, at least 90% identical, at least 95% identical, or at least 96%), 97%, 98% or 99% identical to a polynucleotide encoding a tumor specific antigenic peptide of the present invention, can be provided. By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%), 97%), 98%), or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5%> of the total number of nucleotides in the reference sequence are allowed.

The isolated tumor specific antigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat′l. Acad. Sci. USA 81 :5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the tumor specific antigenic peptides. Recombinant expression vectors are replicable DNA constructs which have synthetic or cDNA-derived DNA fragments encoding a tumor specific antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as Ml 3 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23 : 175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione- S-transf erase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-FPLC) steps employing hydrophobic RP-FTPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### In Vivo Peptide/Polypeptide Synthesis

The present invention also contemplates the use of nucleic acid molecules as vehicles for delivering antigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment antigenic peptides may be administered to a patient in need thereof by use of an mRNA vaccine (see, e.g., Sahin, U, Kariko, K and Tureci, O (2014). mRNA-based therapeutics - developing a new class of drugs. Nat Rev Drug Discov 13: 759-780; Weissman D, Karikó K. mRNA: Fulfilling the Promise of Gene Therapy. Mol Ther. 2015;23(9):1416-1417. doi: 10.1038/mt.2015.138; Kowalski PS, Rudra A, Miao L, Anderson DG. Delivering the Messenger: Advances in Technologies for Therapeutic mRNA Delivery. Mol Ther. 2019;27(4):710-728. doi:10.1016/j.ymthe.2019.02.012; Magadum A, Kaur K, Zangi L. mRNA-Based Protein Replacement Therapy for the Heart. Mol Ther. 2019;27(4):785-793. doi:10.1016/j.ymthe.2018.11.018; Reichmuth AM, Oberli MA, Jaklenec A, Langer R, Blankschtein D. mRNA vaccine delivery using lipid nanoparticles Ther Deliv. 2016;7(5):319-334. doi:10.4155/tde-2016-0006; and Khalil AS, Yu X, Umhoefer JM, et al. Single-dose mRNA therapy via biomaterial-mediated sequestration of overexpressed proteins. Sci Adv. 2020;6(27):eaba2422). In an exemplary embodiment, mRNA encoding for an antigenic peptide is delivered using lipid nanoparticles (see, e.g., Reichmuth, et al., 2016) and administered directly to tumor tissue. In an exemplary embodiment, mRNA encoding for an antigenic peptide is delivered using biomaterial-mediated sequestration (see, e.g., Khalil, et al., 2020) and administered directly to tumor tissue.

In one embodiment antigens may be administered to a patient in need thereof by use of a plasmid. These are plasmids which usually consist of a strong viral promoter to drive the in vivo transcription and translation of the gene (or complementary DNA) of interest (Mor, et al., (1995), The Journal of Immunology 155 (4): 2039-2046). Intron A may sometimes be included to improve mRNA stability and hence increase protein expression (Leitner et al. (1997), The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999), Adv. Parasitol. Advances in Parasitology 42: 343-410; Robinson et al., (2000). Adv. Virus Res. Advances in Virus Research 55: 1-74; Bohmet al., (1996). Journal of Immunological Methods 193 (1): 29-40.). Multi cistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimizing vector design for maximal protein expression is essential (Lewis et al., (1999). Advances in Virus Research (Academic Press) 54: 129-88). One way of enhancing protein expression is by optimizing the codon usage of pathogenic mRNAs for eukaryotic cells. Another consideration is the choice of promoter. Such promoters may be the SV40 promoter or Rous Sarcoma Virus (RSV). Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (EVI) in skeletal muscle, or intradermally (ID), with DNA being delivered to the extracellular spaces. This can be assisted by el ectrop oration by temporarily damaging muscle fibres with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex Vivo Cytosolic Delivery of Functional Macromolecules to Immune Cells, Sharei et al, PLOS ONE | DOI: 10.1371/journal.pone.O1 18803 Apr. 13, 2015).

The method of delivery determines the dose of DNA required to raise an effective immune response. Saline injections require variable amounts of DNA, from 10 µg-1 mg, whereas gene gun deliveries require 100 to 1000 times less DNA than intramuscular saline injection to raise an effective immune response. Generally, 0.2 µg - 20 µg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshiaet al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition may include separate DNA plasmids encoding, for example, one or more antigenic peptides/polypeptides as identified in according to the invention. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more antigenic peptides of the invention may be encoded and expressed in vivo using a viral based system (e.g., an adenovirus system, an adeno associated virus (AAV) vector, a poxvirus, or a lentivirus). In one embodiment, the neoplasia vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan 15;207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Pat. Application No. 20080254008, hereby incorporated by reference). The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66: 1635-1640; Sommnerfelt et al., (1990) Virol. 176:58-59; Wilson et al., (1998) J. Virol. 63 :2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8: 275 - 285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson’s Disease, see, e.g., U.S. Pat. Publication No. 20120295960 and U.S. Pat. Nos. 7303910 and 7351585. Lentiviral vectors have also been disclosed for delivery to the Brain, see, e.g., U.S. Pat. Publication Nos. US20110293571; US20040013648, US20070025970, US20090111106 and U.S. Pat. No. US7259015. In another embodiment lentiviral vectors are used to deliver vectors to the brain of those being treated for a disease.

As to lentivirus vector systems useful in the practice of the invention, mention is made of U.S. Pats. Nos. 6428953, 6165782, 6013516, 5994136, 6312682, and 7,198,784, and documents cited therein.

In an embodiment herein the delivery is via an lentivirus. Zou et al. administered about 10 [symbol] of a recombinant lentivirus having a titer of 1 × 10⁹ transducing units (TU)/ml by an intrathecal catheter. These sort of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹ TU/ml, more preferably at least 10⁹ TU/ml. Other methods of concentration such as ultrafiltration or binding to and elution from a matrix may be used.

In other embodiments the amount of lentivirus administered may be 1.×.10⁵ or about 1.×.10⁵ plaque forming units (PFU), 5.×.10⁵ or about 5.×.10⁵ PFU, 1.×.10⁶ or about 1.×10⁶ PFU, 5.×.10⁶ or about 5.×.10⁶ PFU, 1.×.10⁷ or about 1.×.10⁷ PFU, 5.×.10⁷ or about 5.×.10⁷ PFU, 1.×.10⁸ or about 1.×.10⁸ PFU, 5.×.10⁸ or about 5.×.10⁸ PFU, 1.×.10⁹ or about 1.×.10⁹ PFU, 5.×.10⁹ or about 5.×.10⁹ PFU, 1.×.10¹⁰ or about 1 .×.10¹⁰ PFU or 5.×.10¹⁰ or about 5.×.10¹⁰ PFU as total single dosage for an average human of 75 kg or adjusted for the weight and size and species of the subject. One of skill in the art can determine suitable dosage. Suitable dosages for a virus can be determined empirically. Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Adl 1, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Adl 1 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3- defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both E1 a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

In an embodiment herein the delivery is via an adenovirus, which may be at a single booster dose containing at least 1 × 10⁵ particles (also referred to as particle units, pu) of adenoviral vector. In an embodiment herein, the dose preferably is at least about 1 × 10⁶ particles (for example, about 1 × 10⁶ -1 × 10¹² particles), more preferably at least about 1 × 10⁷ particles, more preferably at least about 1 × 10⁸ particles (e.g., about 1 × 10⁸ -1 × 10¹¹ particles or about 1 × 10⁸ -1 × 10¹² particles), and most preferably at least about 1 × 10⁹ particles (e.g., about 1 × 10⁹ -1 × 10¹⁰ particles or about 1 × 10⁹ -1 × 10¹² particles), or even at least about 1 × 10¹⁰ particles (e.g., about 1 × 10¹⁰ -1 × 10¹² particles) of the adenoviral vector. Alternatively, the dose comprises no more than about 1 × 10¹⁴ particles, preferably no more than about 1 × 10¹³ particles, even more preferably no more than about 1 × 10¹² particles, even more preferably no more than about 1 × 10¹¹ particles, and most preferably no more than about 1 × 10¹⁰ particles (e.g., no more than about 1 × 10⁹ articles). Thus, the dose may contain a single dose of adenoviral vector with, for example, about 1 × 10⁶ particle units (pu), about 2 × 10⁶ pu, about 4 × 10⁶ pu, about 1 × 10⁷ pu, about 2 × 10 pu, about 4 × 10 pu, about 1 × 10 pu, about 2 × 10 pu, about 4 × 10 pu, about 1 × 10⁹ pu, about 2 × 10⁹ pu, about 4 × 10⁹ pu, about 1 × 10¹⁰ pu, about 2 × 10¹⁰ pu, about 4 × 10¹⁰ pu, about 1 × 10¹¹ pu, about 2 × 10¹¹ pu, about 4 × 10¹¹ pu, about 1 × 10¹² pu, about 2 × 10¹² pu, or about 4 × 10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn’t integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA).

With regard to AAV vectors useful in the practice of the invention, mention is made of U.S. Pat. Nos. 5658785, 7115391, 7172893, 6953690, 6936466, 6924128, 6893865, 6793926, 6537540, 6475769 and 6258595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1 × 10¹⁰ to about 1 × 10⁵⁰ functional AAV/ml solution. The dosage may be adjusted to balance the therapeutic benefit against any side effects. In an embodiment herein, the AAV dose is generally in the range of concentrations from about 1 × 10 to 1 × 10 genomes AAV, from about 1 × 10 to 1 × 10 genomes AAV, from about 1 × 10¹⁰ to about 1 × 10¹⁶ genomes, or about 1 × 10¹¹ to about 1 × 10¹⁶ genomes AAV. A human dosage may be about 1 × 10¹¹ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2 × 10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment effectively activating a cellular immune response for a neoplasia vaccine or immunogenic composition can be achieved by expressing the relevant antigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety). In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardiet al., Hum Vaccin Immunother. 2012 Jul;8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature, such as: U.S. Pats. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith GL. Proc. Natl. Acad. Sci. 1983; 80(23): 7155-9, Smith GL. Nature 1983; 302: 490-5, Sullivan VJ. Gen. Vir. 1987; 68: 2587-98, Perkus M Journal of Leukocyte Biology 1995; 58: 1-13, Yilma TD. Vaccine 1989; 7: 484-485, Brochier B. Nature 1991 ; 354: 520-22, Wiktor, TJ. Proc. Natl Acd. Sci. 1984; 81 : 7194-8, Rupprecht, CE. Proc. Natl Acd. Sci. 1986; 83 : 7947-50, Poulet, H Vaccine 2007; 25(Jul): 5606-12, Weyer J. Vaccine 2009; 27(Nov): 7198-201, Buller, RM Nature 1985; 317(6040): 813-5, Buller RM. J. Virol. 1988; 62(3):866-74, Flexner, C. Nature 1987; 330(6145): 259-62, Shida, H. J. Virol. 1988; 62(12): 4474-80, Kotwal, GJ. J. Virol. 1989; 63(2): 600-6, Child, SJ. Virology 1990; 174(2): 625-9, Mayr A. Zentralbl Bakteriol 1978; 167(5,6): 375-9, Antoine G. Virology. 1998; 244(2): 365-96, Wyatt, LS. Virology 1998; 251(2): 334-42, Sancho, MC. J. Virol. 2002; 76(16); 8313-34, Gallego-Gomez, JC. J. Virol. 2003; 77(19); 10606-22), Goebel SJ. Virology 1990; (a,b) 179: 247-66, Tartaglia, J. Virol. 1992; 188(1): 217-32, Najera JL. J. Virol. 2006; 80(12): 6033-47, Najera, JL. J. Virol. 2006; 80: 6033-6047, Gomez, CE. J. Gen. Virol. 2007; 88: 2473-78, Mooij, P. Jour. Of Virol. 2008; 82: 2975- 2988, Gomez, CE. Curr. Gene Ther. 2011; 11 : 189-217, Cox,W. Virology 1993; 195: 845-50, Perkus, M. Jour. Of Leukocyte Biology 1995; 58: 1-13, Blanchard TJ. J Gen Virology 1998; 79(5): 1159-67, Amara R. Science 2001; 292: 69-74, Hel, Z., J. Immunol. 2001; 167: 7180-9, Gherardi MM. J. Virol. 2003; 77: 7048-57, Didierlaurent, A. Vaccine 2004; 22: 3395-3403, Bissht H. Proc. Nat. Aca. Sci. 2004; 101 : 6641-46, McCurdy LH. Clin. Inf. Dis 2004; 38: 1749-53, Earl PL. Nature 2004; 428: 182-85, Chen Z. J. Virol. 2005; 79: 2678-2688, Najera JL. J. Virol. 2006; 80(12): 6033-47, Nam JH. Acta. Virol. 2007; 51 : 125-30, Antonis AF. Vaccine 2007; 25: 4818-4827,B Weyer J. Vaccine 2007; 25: 4213-22, Ferrier-Rembert A. Vaccine 2008; 26(14): 1794-804, Corbett M. Proc. Natl. Acad. Sci. 2008; 105(6): 2046-51, Kaufman HL., J. Clin. Oncol. 2004; 22: 2122-32, Amato, RJ. Clin. Cancer Res. 2008; 14(22): 7504-10, Dreicer R. Invest New Drugs 2009; 27(4): 379-86, Kantoff PW.J. Clin. Oncol. 2010, 28, 1099-1 105, Amato RJ. J. Clin. Can. Res. 2010; 16(22): 5539-47, Kim, DW. Hum. Vaccine. 2010; 6: 784-791, Oudard, S. Cancer Immunol. Immunother. 2011; 60: 261-71, Wyatt, LS. Aids Res. Hum. Retroviruses. 2004; 20: 645-53, Gomez, CE. Virus Research 2004; 105: 11-22, Webster, DP. Proc. Natl. Acad. Sci. 2005; 102: 4836-4, Huang, X. Vaccine 2007; 25: 8874-84, Gomez, CE. Vaccine 2007a; 25: 2863-85, Esteban M. Hum. Vaccine 2009; 5: 867-871, Gomez, CE. Curr. Gene therapy 2008; 8(2): 97-120, Whelan, KT. Plos one 2009; 4(6): 5934, Scriba, TJ. Eur. Jour. Immuno. 2010; 40(1): 279-90, Corbett, M. Proc. Natl. Acad. Sci. 2008; 105: 2046-2051, Midgley, CM. J. Gen. Virol. 2008; 89: 2992-97, Von Krempelhuber, A. Vaccine 2010; 28: 1209-16, Perreau, M. J. Of Virol. 2011; Oct: 9854- 62, Pantaleo, G. Curr Opin HIV-AIDS. 2010; 5: 391-396, each of which is incorporated herein by reference.

In another embodiment the vaccinia virus is used in the neoplasia vaccine or immunogenic composition to express a antigen. (Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment ALVAC is used as a vector in a neoplasia vaccine or immunogenic composition. ALVAC is a canarypox virus that can be modified to express foreign transgenes and has been used as a method for vaccination against both prokaryotic and eukaryotic antigens (Horig H, Lee DS, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000;49:504-14; von Mehren M, Arlen P, Tsang KY, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000;6:2219-28; Musey L, Ding Y, Elizaga M, et al. HIV-1 vaccination administered intramuscularly can induce both systemic and mucosal T cell immunity in HIV-1 -uninfected individuals. J Immunol 2003; 171 : 1094-101; Paoletti E. Applications of pox virus vectors to vaccination: an update. Proc Natl Acad Sci U S A 1996;93 : 11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall JL, Hawkins MJ, Tsang KY, et al. Phase I study in cancer patients of a replication-defective avipox recombinant vaccine that expresses human carcinoembryonic antigen. J Clin Oncol 1999; 17:332-7).

In another embodiment a Modified Vaccinia Ankara (MVA) virus may be used as a viral vector for a antigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61(1): 19-29). Methods to make and use recombinant MVA has been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles of the vaccine or immunogenic composition are administered to patients in need thereof. Dosages of expressed antigen can range from a few to a few hundred micrograms, e.g., 5 to 500 µg. The vaccine or immunogenic composition can be administered in any suitable amount to achieve expression at these dosage levels. The viral particles can be administered to a patient in need thereof or transfected into cells in an amount of about at least 10³⁵ pfu; thus, the viral particles are preferably administered to a patient in need thereof or infected or transfected into cells in at least about 10⁴ pfu to about 10⁶ pfu; however, a patient in need thereof can be administered at least about 10⁸ pfu such that a more preferred amount for administration can be at least about 10⁷ pfu to about 10⁹ pfu. Doses as to NYVAC are applicable as to ALVAC, MVA, MVA-BN, and avipoxes, such as canarypox and fowlpox.

### Vaccine or Immunogenic Composition Adjuvant

Effective vaccine or immunogenic compositions advantageously include a strong adjuvant to initiate an immune response. As described herein, poly-ICLC, an agonist of TLR3 and the RNA helicase -domains of MDA5 and RIG3, has shown several desirable properties for a vaccine or immunogenic composition adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly- ICLC in combination with a NY-ESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile. In addition to a powerful and specific immunogen the antigen peptides may be combined with an adjuvant (e.g., poly- ICLC) or another anti -neoplastic agent. Without being bound by theory, these antigens are expected to bypass central thymic tolerance (thus allowing stronger anti -tumor T cell response), while reducing the potential for autoimmunity (e.g., by avoiding targeting of normal self- antigens). An effective immune response advantageously includes a strong adjuvant to activate the immune system (Speiser and Romero, Molecularly defined vaccines for cancer immunotherapy, and protective T cell immunity Seminars in Immunol 22: 144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants? Cancer J. 16:382-391 (2010)). Among the TLR agonists, poly-ICLC (a synthetic double- stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double- stranded RNA induces innate immune responses similar to a live viral vaccine in humans J Exp Med 208:2357 (2011)). In a preferred embodimentHiltonol®, a GMP preparation of poly-ICLC prepared by Oncovir, Inc, is utilized as the adjuvant. In other embodiments, other adjuvants described herein are envisioned. For instance oil-in-water, water-in-oil or multiphasic W/O/W; see, e.g., US 7,608,279 and Aucouturier et al, Vaccine 19 (2001), 2666-2672, and documents cited therein.

### Pharmaceutical Compositions/Methods of Delivery

The present invention is also directed to pharmaceutical compositions comprising an effective amount of one or more antigenic peptides as described herein (including a pharmaceutically acceptable salt, thereof), optionally in combination with a pharmaceutically acceptable carrier, excipient or additive.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific antigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n—COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific antigens provided herein, including those listed by Remington’s Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

When administered as a combination, the therapeutic agents (i.e. the antigenic peptides) can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a neoplasia. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient’s life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject’s life.

Surgical resection uses surgery to remove abnormal tissue in cancer, such as mediastinal, neurogenic, or germ cell tumors, or thymoma. In certain embodiments, administration of the composition is initiated following tumor resection. In other embodiments, administration of the neoplasia vaccine or immunogenic composition is initiated 1, 2, 3, 4, 5, 6, 7, 8, 9, 10, 11, 12, 13, 14, 15 or more weeks after tumor resection. Preferably, administration of the neoplasia vaccine or immunogenic composition is initiated 4, 5, 6, 7, 8, 9, 10, 11 or 12 weeks after tumor resection.

In certain embodiments, the vaccine of the present invention is administered to a subject one or more times. In certain embodiments, a subject is primed with a vaccine and then boosted after the initial vaccination. The term “prime/boost” or “prime/ boost dosing regimen” is meant to refer to the successive administrations of a vaccine or immunogenic or immunological compositions. The priming administration (priming) is the administration of a first vaccine or immunogenic or immunological composition type and may comprise one, two or more administrations. The boost administration is the second administration of a vaccine or immunogenic or immunological composition type and may comprise one, two or more administrations, and, for instance, may comprise or consist essentially of annual administrations. In certain embodiments, administration of the neoplasia vaccine or immunogenic composition is in a prime/ boost dosing regimen.

In certain embodiments, administration of the neoplasia vaccine or immunogenic composition is in a prime/ boost dosing regimen, for example administration of the neoplasia vaccine or immunogenic composition at weeks 1, 2, 3 or 4 as a prime and administration of the neoplasia vaccine or immunogenic composition is at months 2, 3 or 4 as a boost. In another embodiment heterologous prime-boost strategies are used to elicit a greater cytotoxic T-cell response (see Schneider et al., Induction of CD8+ T cells using heterologous prime-boost immunization strategies, Immunological Reviews Volume 170, Issue 1, pages 29-38, August 1999). In another embodiment DNA encoding antigens is used to prime followed by a protein boost. In another embodiment protein is used to prime followed by boosting with a virus encoding the antigen. In another embodiment a virus encoding the antigen is used to prime and another virus is used to boost. In another embodiment protein is used to prime and DNA is used to boost. In a preferred embodiment a DNA vaccine or immunogenic composition is used to prime a T-cell response and a recombinant viral vaccine or immunogenic composition is used to boost the response. In another preferred embodiment a viral vaccine or immunogenic composition is coadministered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun. 2007 Dec;75(12):5819-26. Epub 2007 Oct 1). The pharmaceutical compositions can be processed in accordance with conventional methods of pharmacy to produce medicinal agents for administration to patients in need thereof, including humans and other mammals.

Modifications of the antigenic peptides can affect the solubility, bioavailability and rate of metabolism of the peptides, thus providing control over the delivery of the active species. Solubility can be assessed by preparing the antigenic peptide and testing according to known methods well within the routine practitioner’s skill in the art.

In certain embodiments of the pharmaceutical composition the pharmaceutically acceptable carrier comprises water. In certain embodiments, the pharmaceutically acceptable carrier further comprises dextrose. In certain embodiments, the pharmaceutically acceptable carrier further comprises dimethylsulfoxide. In certain embodiments, the pharmaceutical composition further comprises an immunomodulator or adjuvant. In certain embodiments, the immunodulator or adjuvant is selected from the group consisting of poly-ICLC, STING agonist, 1018 ISS, aluminum salts, Amplivax, AS15, BCG, CP-870,893, CpG7909, CyaA, dSLEVI, GM-CSF, IC30, IC31, Imiquimod, ImuFact FMP321, IS Patch, ISS, ISCOMATRLX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila’s QS21 stimulon. In certain embodiments, the immunomodulator or adjuvant comprises poly-ICLC.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine or immunogenic composition of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Dimethylxanthenone-4-Acetic Acid, Journal of Immunology, 190:5216-25 and Kim et al. (2013) Anticancer Flavonoids are Mouse-Selective STING Agonists, 8: 1396-1401). The vaccine or immunological composition may also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It may also be a copolymer of maleic anhydride and ethylene cross-linked, for example, with divinyl ether (see U.S. Pat. No. 6,713,068 hereby incorporated by reference in its entirety)..

In certain embodiments, the pH modifier can stabilize the adjuvant or immunomodulator as described herein.

In certain embodiments, a pharmaceutical composition comprises: one to five peptides, dimethylsulfoxide (DMSO), dextrose, water, succinate, poly I: poly C, poly-L-lysine, carboxymethylcellulose, and chloride. In certain embodiments, each of the one to five peptides is present at a concentration of 300 µg/ml. In certain embodiments, the pharmaceutical composition comprises < 3% DMSO by volume. In certain embodiments, the pharmaceutical composition comprises 3.6 - 3.7% dextrose in water. In certain embodiments, the pharmaceutical composition comprises 3.6 - 3.7 mM succinate (e.g., as sodium succinate) or a salt thereof. In certain embodiments, the pharmaceutical composition comprises 0.5 mg/ml poly I: poly C. In certain embodiments, the pharmaceutical composition comprises 0.375 mg/ml poly- L-Lysine. In certain embodiments, the pharmaceutical composition comprises 1.25 mg/ml sodium carboxymethylcellulose. In certain embodiments, the pharmaceutical composition comprises 0.225% sodium chloride.

Pharmaceutical compositions comprise the herein-described tumor specific antigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present invention may vary with the condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric- coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as a fatty oil. In addition, dosage unit forms can contain various other materials which modify the physical form of the dosage unit, for example, coatings of sugar, shellac, or enteric agents. Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US Patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate- buffered saline (PBS), and 5% dextrose in water (D5W). In certain embodiments, the aqueous solvent further comprises dimethyl sulfoxide (DMSO), e.g., in an amount of about 1-4%, or 1-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier usually comprises sterile water or aqueous sodium chloride solution, though other ingredients including those which aid dispersion may be included. Of course, where sterile water is to be used and maintained as sterile, the compositions and carriers are also sterilized. Injectable suspensions may also be prepared, in which case appropriate liquid carriers, suspending agents and the like may be employed. Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub -cutaneous, transdermal (which may include a penetration enhancement agent), buccal and suppository administration, among other routes of administration, including through an eye or ocular route.

The neoplasia vaccine or immunogenic composition, and any additional agents, may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

In certain embodiments, the vaccine or immunogenic composition is administered intravenously or subcutaneously. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor specific antigenic peptides may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific antigenic peptides may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal antiinflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as HERCEPTIN and RITUXAN.

It should be understood that in addition to the ingredients particularly mentioned herein, the formulations of the present invention may include other agents conventional in the art having regard to the type of formulation in question, for example, those suitable for oral administration may include flavoring agents.

Pharmaceutically acceptable salt forms may be the preferred chemical form of compounds according to the present invention for inclusion in pharmaceutical compositions according to the present invention.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein is evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T.W. Greene and P.G.M. Wuts, Protective Groups in Organic Synthesis, 3rd. Ed., John Wiley and Sons (1999); L. Fieser and M. Fieser, Fieser and Fieser’s Reagents for Organic Synthesis, John Wiley and Sons (1999); and L. Paquette, ed., Encyclopedia of Reagents for Organic Synthesis, John Wiley and Sons (1995), and subsequent editions thereof.

The additional agents that may be included with the tumor specific neo-antigenic peptides of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Dosage. When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with neoplasia, e.g. cancer or tumors.

A preferred dose of an agent is the maximum that a patient can tolerate and not develop serious or unacceptable side effects. Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 mg/ml. The pharmaceutical compositions 5 typically should provide a dosage of from about 0.001 mg to about 2000 mg of compound per kilogram of body weight per day. For example, dosages for systemic administration to a human patient can range from 1-10 mg/kg, 20-80 mg/kg, 5-50 mg/kg, 75-150 mg/kg, 100-500 mg/kg, 250-750 mg/kg, 500-1000 mg/kg, 1-10 mg/kg, 5-50 mg/kg, 25-75 mg/kg, 50-100 mg/kg, 100- 250 mg/kg, 50-100 mg/kg, 250-500 mg/kg, 500-750 mg/kg, 750-1000 mg/kg, 1000-1500 mg/kg, 101500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about IµM of an agent is administered to a subject. In related embodiments, about 50-100 nM, 50-250 nM, 100-500 nM, 250-500 nM, 250-750 nM, 500-750 nM, 500 nM to IµM, or 750 nM to IµM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman’s The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Preferred unit dosage formulations are those containing a daily dose or unit, daily sub-dose, as herein discussed, or an appropriate fraction thereof, of the administered ingredient.

The dosage regimen for treating a disorder or a disease with the tumor specific antigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art.

The amount of compound included within therapeutically active formulations according to the present invention is an effective amount for treating the disease or condition. In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present invention has application for both human and veterinary use.

For oral administration to humans, a dosage of between approximately 0.1 to 100 mg/kg/day, preferably between approximately 1 and 100 mg/kg/day, is generally sufficient.

Where drug delivery is systemic rather than topical, this dosage range generally produces effective blood level concentrations of active compound ranging from less than about 0.04 to about 400 micrograms/cc or more of blood in the patient. The compound is conveniently administered in any suitable unit dosage form, including but not limited to one containing 0.001 to 3000 mg, preferably 0.05 to 500 mg of active ingredient per unit dosage form. An oral dosage of 10-250 mg is usually convenient.

According to certain exemplary embodiments, the vaccine or immunogenic composition is administered at a dose of about 10 µg to 1 mg per antigenic peptide. According to certain exemplary embodiments, the vaccine or immunogenic composition is administered at an average weekly dose level of about 10 µg to 2000 µg per antigenic peptide.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The invention provides for pharmaceutical compositions containing at least one tumor specific antigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington’s Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabi sulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container. Methods for formulating the pharmaceutical compositions of the present invention are conventional and well known in the art (see Remington and Remington’s). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel’s Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/ or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2- hy droxy ethyl -L-glutamine) and poly(lactic acid). Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects. Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZO E.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone. In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non- degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents. Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

Vaccine or immunogenic compositions. The present invention is directed in some aspects to pharmaceutical compositions suitable for the prevention or treatment of cancer. In one embodiment, the composition comprises at least an immunogenic composition, e.g., a neoplasia vaccine or immunogenic composition capable of raising a specific T-cell response. The neoplasia vaccine or immunogenic composition comprises antigenic peptides and/or antigenic polypeptides corresponding to tumor specific antigens as described herein.

A suitable neoplasia vaccine or immunogenic composition can preferably contain a plurality of tumor specific antigenic peptides. In an embodiment, the vaccine or immunogenic composition can include between 1 and 100 sets of peptides, more preferably between 1 and 50 such peptides, even more preferably between 10 and 30 sets peptides, even more preferably between 15 and 25 peptides. According to another preferred embodiment, the vaccine or immunogenic composition can include at least one peptides, more preferably 2, 3, 4, or 5 peptides, In certain embodiments, the vaccine or immunogenic composition can comprise 5, 6, 7, 8, 9, 10, 11, 12, 13, 14, 15, 16, 17, 18, 19, 20, 21, 22, 23, 24, 25, 26, 27, 28, 29, or 30 different peptides.

The optimum amount of each peptide to be included in the vaccine or immunogenic composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA injection include i.d., i.m., s.c, i.p. and i.v. For example, doses of between 1 and 500 mg 50 µg and 1.5 mg, preferably 10 µg to 500 µg, of peptide or DNA may be given and can depend from the respective peptide or DNA. Doses of this range were successfully used in previous trials (Brunsvig P F, et al., Cancer Immunol Immunother. 2006; 55(12): 1553- 1564; M. Staehler, et al., ASCO meeting 2007; Abstract No 3017). Other methods of administration of the vaccine or immunogenic composition are known to those skilled in the art.

In one embodiment of the present invention the different tumor specific antigenic peptides and/or polypeptides are selected for use in the neoplasia vaccine or immunogenic composition so as to maximize the likelihood of generating an immune attack against the neoplasias/tumors in a high proportion of subjects in the population. Without being bound by theory, it is believed that the inclusion of a diversity of tumor specific antigenic peptides can generate a broad scale immune attack against a neoplasia/tumor. In one embodiment, the selected tumor specific antigenic peptides/polypeptides are encoded by missense mutations. In a second embodiment, the selected tumor specific antigenic peptides/polypeptides are encoded by a combination of missense mutations and neoORF mutations. In a third embodiment, the selected tumor specific antigenic peptides/polypeptides are encoded by neoORF mutations.

In one embodiment in which the selected tumor specific antigenic peptides/polypeptides are encoded by missense mutations, the peptides and/or polypeptides are chosen based on their capability to associate with the MHC molecules of a high proportion of subjects in the population. Peptides/polypeptides derived from neoOR mutations can also be selected on the basis of their capability to associate with the MHC molecules of the patient population.

The vaccine or immunogenic composition is capable of raising a specific cytotoxic T-cells response and/or a specific helper T-cell response.

The vaccine or immunogenic composition can further comprise an adjuvant and/or a carrier. Examples of useful adjuvants and carriers are given herein. The peptides and/or polypeptides in the composition can be associated with a carrier such as, e.g., a protein or an antigen-presenting cell such as e.g. a dendritic cell (DC) capable of presenting the peptide to a T-cell. Adjuvants are any substance whose admixture into the vaccine or immunogenic composition increases or otherwise modifies the immune response to the mutant peptide. Carriers are scaffold structures, for example a polypeptide or a polysaccharide, to which the antigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Thl response.

Suitable adjuvants include, but are not limited to 1018 ISS, aluminum salts, Amplivax, AS15, BCG, CP-870,893, CpG7909, CyaA, dSLEVI, GM-CSF, IC30, IC31, Imiquimod, ImuFact FMP321, IS Patch, ISS, ISCOMATRIX, Juvlmmune, LipoVac, MF59, monophosphoryl lipid A, Montanide FMS 1312, Montanide ISA 206, Montanide ISA 50V, Montanide ISA-51, OK-432, OM-174, OM-197-MP-EC, ONTAK, PEPTEL. vector system, PLG microparticles, resiquimod, SRL172, Virosomes and other Virus-like particles, YF-17D, VEGF trap, R848, beta-glucan, Pam3Cys, Aquila’s QS21 stimulon (Aquila Biotech, Worcester, Mass., USA) which is derived from saponin, mycobacterial extracts and synthetic bacterial cell wall mimics, and other proprietary adjuvants such as Ribi’s Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasi s Tumor Immunol . 1996 (6):414-418).

Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endolysosomes. Different TLRs recognize distinct PAMPS. For example, TLR4 is activated by LPS contained in bacterial cell walls, TLR9 is activated by unmethylated bacterial or viral CpG DNA, and TLR3 is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine or immunogenic composition adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-10, as well as maintenance of high levels of co- stimulatory molecules in DCsl . Furthermore, poly-ICLC was recently directly compared to CpG in non-human primates (rhesus macaques) as adjuvant for a protein vaccine or immunogenic composition consisting of human papillomavirus (HPV)16 capsomers (Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009;5(4)).

CpG immuno stimulatory oligonucleotides have also been reported to enhance the effects of adjuvants in a vaccine or immunogenic composition setting. Without being bound by theory, CpG oligonucleotides act by activating the innate (non- adaptive) immune system via Toll-like receptors (TLR), mainly TLR9. CpG triggered TLR9 activation enhances antigen- specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Thl cells and strong cytotoxic T- lymphocyte (CTL) generation, even in the absence of CD4 T-cell help. The Thl bias induced by TLR9 stimulation is maintained even in the presence of vaccine adjuvants such as alum or incomplete Freund’s adjuvant (IF A) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, June 2006, 471-484). U.S. Pat. No. 6,406,705 B1 describes the combined use of CpG oligonucleotides, non-nucleic acid adjuvants and an antigen to induce an antigen- specific immune response. A commercially available CpG TLR9 antagonist is dSLEVI (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP- 547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony- stimulating factors, such as Granulocyte Macrophage Colony Stimulating Factor (GM-CSF, sargramostim).

Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the P1/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen- specific CD8+ T-cells. In a recent study in non-human primates, poly-ICLC was found to be essential for the generation of antibody responses and T-cell immunity to DC targeted or non- targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single s.c. administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-5 1, or with 1.4 mg poly-ICLC and Montanide. The generation of NY-ESO-1 -specific CD4+ and CD8+ T-cell and antibody responses were markedly enhanced with the addition of poly-ICLC and Montanide compared to OLP alone or OLP and Montanide.

A vaccine or immunogenic composition according to the present invention may comprise more than one different adjuvant. Furthermore, the invention encompasses a therapeutic composition comprising any adjuvant substance including any of those herein discussed. It is also contemplated that the peptide or polypeptide, and the adjuvant can be administered separately in any appropriate sequence. A carrier may be present independently of an adjuvant. The carrier may be covalently linked to the antigen. A carrier can also be added to the antigen by inserting DNA encoding the carrier in frame with DNA encoding the antigen. The function of a carrier can for example be to confer stability, to increase the biological activity, or to increase serum half-life. Extension of the half-life can help to reduce the number of applications and to lower doses, thus are beneficial for therapeutic but also economic reasons. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier may be a physiologically acceptable carrier acceptable to humans and safe. However, tetanus toxoid and/or diptheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments the vaccine or immunogenic composition according to the present invention additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail herein, the MHC class I or II molecule may readily be loaded with the selected antigen in vitro.

CD8+ cell activity may be augmented through the use of CD4+ cells. The identification of CD4 T+ cell epitopes for tumor antigens has attracted interest because many immune based therapies against cancer may be more effective if both CD8+ and CD4+ T lymphocytes are used to target a patient’s tumor. CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type 1 (TH1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells non- specifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the invention that such CD4+ cells may be applicable at three levels that vary in their tumor specificity: 1) a broad level in which universal CD4+ epitopes (e.g., tetanus toxoid) may be used to augment CD8+ cells; 2) an intermediate level in which native, tumor-associated CD4+ epitopes may be used to augment CD8+ cells; and 3) a patient specific level in which antigen CD4+ epitopes may be used to augment CD8+ cells in a patient specific manner. Although current algorithms for predicting CD4 epitopes are limited in accuracy, it is a reasonable expectation that many long peptides containing predicted CD8 neoepitopes will also include CD4 epitopes. CD4 epitopes are longer than CD8 epitopes and typically are 10 -12 amino acids in length although some can be longer (Kreiter et al, Mutant MHC Class II epitopes drive therapeutic immune responses to cancer, Nature (2015). Thus the neoanti genie epitopes described herein, either in the form of long peptides (>25 amino acids) or nucleic acids encoding such long peptides, may also boost CD4 responses in a tumor and patient-specific manner (level (3) above).

CD8+ cell immunity may also be generated with antigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201 -restricted gplOO melanoma antigen-derived peptides with autologous peptide pulsed CD40L/IFN-g-activated mature DCs via an IL-12p70-producing patient DC vaccine (see e.g., Carreno et al (2013) L-12p70-producing patient DC vaccine elicits Tel -polarized immunity, Journal of Clinical Investigation, 123(8):3383-94 and Ali et al. (2009) In situ regulation of DC subsets and T cells mediates tumor regression in mice, Cancer Immunotherapy, 1(8): 1-10). It is contemplated within the scope of the invention that antigen loaded DCs may be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-12), tumor necrosis factor (TNF), interferon gamma-induced protein 10 (IP- 10), interleukin 1 (IL-1), and type I interferons (IFN), and minimal interleukin 10 (IL-10) production. DCs may be differentiated from frozen peripheral blood mononuclear cells (PBMCs) obtained by leukapheresis, while PBMCs may be isolated by Ficoll gradient centrifugation and frozen in aliquots.

Illustratively, the following 7 day activation protocol may be used. Day 1— PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM- CSF) to differentiate to immature DCs. On Day 6, immature DCs are pulsed with the keyhole limpet hemocyanin (KLH) protein which serves as a control for the quality of the vaccine and may boost the immunogenicity of the vaccine. The DCs are stimulated to mature, loaded with peptide antigens, and incubated overnight. On Day 7, the cells are washed, and frozen in 1 ml aliquots containing 4-20 × 10(6) cells using a controlled-rate freezer. Lot release testing for the batches of DCs may be performed to meet minimum specifications before the DCs are injected into patients (see e.g., Sabado et al. (2013) Preparation of tumor antigen-loaded mature dendritic cells for immunotherapy, J. Vis Exp. Aug 1;(78).doi: 10.3791/50085).

A DC vaccine may be incorporated into a scaffold system to facilitate delivery to a patient. Therapeutic treatment of a patients neoplasia with a DC vaccine may utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs may interact with T cells to generate a potent cytotoxic T lymphocyte response to the cancer antigens. Implantable biomaterials may be used to generate a potent cytotoxic T lymphocyte response against a neoplasia in a patient specific manner. The biomaterial-resident dendritic cells may then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1(8): 1-10; Ali et al. (2009) Infection-mimicking materials to program dendritic cells in situ. Nat Mater 8: 151-8), and such a vaccine is currently being tested in a Phase I clinical trial recently initiated at the Dana-Farber Cancer Institute. This approach has also been shown to lead to regression of glioblastoma, as well as the induction of a potent memory response to prevent relapse, using the C6 rat glioma model.24 in the current proposal. The ability of such an implantable, biomatrix vaccine delivery scaffold to amplify and sustain tumor specific dendritic cell activation may lead to more robust anti -tumor immunosensitization than can be achieved by traditional subcutaneous or intra-nodal vaccine administrations.

The present invention may include any method for loading a antigenic peptide onto a dendritic cell. One such method applicable to the present invention is a microfluidic intracellular delivery system. Such systems cause temporary membrane disruption by rapid mechanical deformation of human and mouse immune cells, thus allowing the intracellular delivery of biomolecules (Sharei et al., 2015, PLOS ONE).

Preferably, the antigen presenting cells are dendritic cells. Suitably, the dendritic cells are autologous dendritic cells that are pulsed with the antigenic peptide. The peptide may be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. In certain embodiments the dendritic cells are targeted using CD141, DEC205, or XCR1 markers. CD141+XCR1+ DCs were identified as a subset that may be better suited to the induction of anti-tumor responses (Bachem et al., J. Exp. Med. 207, 1273-1281 (2010); Crozat et al., J. Exp. Med. 207, 1283-1292 (2010); and Gallois & Bhardwaj, Nature Med. 16, 854-856 (2010)).

Thus, in one embodiment of the present invention the vaccine or immunogenic composition containing at least one antigen presenting cell is pulsed or loaded with one or more peptides of the present invention. Alternatively, peripheral blood mononuclear cells (PBMCs) isolated from a patient may be loaded with peptides ex vivo and injected back into the patient. As an alternative the antigen presenting cell comprises an expression construct encoding a peptide of the present invention. The polynucleotide may be any suitable polynucleotide and it is preferred that it is capable of transducing the dendritic cell, thus resulting in the presentation of a peptide and induction of immunity.

The inventive pharmaceutical composition may be compiled so that the selection, number and/or amount of peptides present in the composition covers a high proportion of subjects in the population. The selection may be dependent on the specific type of cancer, the status of the disease, earlier treatment regimens, and, of course, the HLA-haplotypes present in the patient population.

Pharmaceutical compositions comprising the peptide of the invention may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use can depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 µg to about 50,000 µg of peptide for a 70 kg patient, followed by boosting dosages or from about 1.0 µg to about 10,000 µg of peptide pursuant to a boosting regimen over weeks to months depending upon the patient’s response and condition and possibly by measuring specific CTL activity in the patient’s blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The invention provides compositions for parenteral administration which comprise a solution of the peptides and vaccine or immunogenic compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A liposome suspension containing a peptide may be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant can, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

The peptides and polypeptides of the invention can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963).

The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Generally, a plasmid for a vaccine or immunological composition can comprise DNA encoding an antigen (e.g., one or more antigens) operatively linked to regulatory sequences which control expression or expression and secretion of the antigen from a host cell, e.g., a mammalian cell; for instance, from upstream to downstream, DNA for a promoter, such as a mammalian virus promoter (e.g., a CMV promoter such as an hCMV or mCMV promoter, e.g., an early-intermediate promoter, or an SV40 promoter—see documents cited or incorporated herein for useful promoters), DNA for a eukaryotic leader peptide for secretion (e.g., tissue plasminogen activator), DNA for the antigen(s), and DNA encoding a terminator (e.g., the 3′ UTR transcriptional terminator from the gene encoding Bovine Growth Hormone or bGH polyA). A composition can contain more than one plasmid or vector, whereby each vector contains and expresses a different antigen. Mention is also made of Wasmoen U.S. Pat. No. 5,849,303, and Dale U.S. Pat. No. 5,811,104, whose text may be useful. DNA or DNA plasmid formulations can be formulated with or inside cationic lipids; and, as to cationic lipids, as well as adjuvants, mention is also made of Loosmore U.S. Patent Application 2003/0104008. Also, teachings in Audonnet U.S. Pat. Nos. 6,228,846 and 6,159,477 may be relied upon for DNA plasmid teachings that can be employed in constructing and using DNA plasmids that contain and express in vivo.

The nucleic acids can also be delivered complexed to cationic compounds, such as cationic lipids. Lipid-mediated gene delivery methods are described, for instance, in W01996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest (e.g., mRNA) can also be used for delivery (see, e.g., Kiken et al, 2011; Su et al, 2011; see also US 8278036; Halabi et al. J Clin Oncol (2003) 21 : 1232-1237; Petsch et al, Nature Biotechnology 2012 Dec 7;30(12): 1210-6).

Viral vectors as described herein can also be used to deliver the antigenic peptides of the invention. Vectors can be administered so as to have in vivo expression and response akin to doses and/or responses elicited by antigen administration.

A preferred means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally- occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g. LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF- β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted herein, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used is dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope- specific CTL lines. Cytolysis, detected by 51 Cr release, indicates production of MHC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g. FM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides may be used to elicit CTL ex vivo, as well. The resulting CTL, can be used to treat chronic tumors in patients in need thereof that do not respond to other conventional forms of therapy, or does not respond to a peptide vaccine approach of therapy. Ex vivo CTL responses to a particular tumor antigen are induced by incubating in tissue culture the patient’s CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they destroy their specific target cell (i.e., a tumor cell). In order to optimize the in vitro conditions for the generation of specific cytotoxic T cells, the culture of stimulator cells are maintained in an appropriate serum-free medium.

Prior to incubation of the stimulator cells with the cells to be activated, e.g., precursor CD8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that allows about 200, and preferably 200 or more, human Class I MHC molecules loaded with peptide to be expressed on the surface of each stimulator cell. Preferably, the stimulator cells are incubated with >2 µg/ml peptide. For example, the stimulator cells are incubates with > 3, 4, 5, 10, 15, or more µg/ml peptide.

Resting or precursor CD8+ cells are then incubated in culture with the appropriate stimulator cells for a time period sufficient to activate the CD8+ cells. Preferably, the CD8+ cells are activated in an antigen- specific manner. The ratio of resting or precursor CD8+ (effector) cells to stimulator cells may vary from individual to individual and may further depend upon variables such as the amenability of an individual’s lymphocytes to culturing conditions and the nature and severity of the disease condition or other condition for which the within- described treatment modality is used. Preferably, however, the lymphocyte: stimulator cell ratio is in the range of about 30: 1 to 300: 1. The effector/stimulator culture may be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/MHC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatability complex molecules on cells allows the induction of primary cytotoxic T lymphocyte responses.

Since mutant cell lines do not exist for every human MHC allele, it is advantageous to use a technique to remove endogenous MHC- associated peptides from the surface of APC, followed by loading the resulting empty MHC molecules with the immunogenic peptides of interest. The use of non-transformed (non-tumorigenic), noninfected cells, and preferably, autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8 - 10 residues, 2) a transmembrane heavy polymorphic protein chain which bears the peptide-binding site in its a1 and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destablize p2microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell’s metabolic rate. It is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic state which is critical for antigen presentation. Mild acid solutions of pH 3 such as glycine or citrate -phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions do not affect the cell’s viability or metabolic state. The mild acid treatment is rapid since the stripping of the endogenous peptides occurs in two minutes at 4° C. and the APC is ready to perform its function after the appropriate peptides are loaded. The technique is utilized herein to make peptide- specific APCs for the generation of primary antigen- specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof) may be utilized to bind their appropriate complementary ligand. Antibody- tagged molecules may then be extracted from the stimulator-effector cell admixture via appropriate means, e.g., via well-known immunoprecipitation or immunoassay methods.

Effective, cytotoxic amounts of the activated CD8+ cells can vary between in vitro and in vivo uses, as well as with the amount and type of cells that are the ultimate target of these killer cells. The amount can also vary depending on the condition of the patient and should be determined via consideration of all appropriate factors by the practitioner. Preferably, however, about 1 X 10⁶ to about 1 X 10¹², more preferably about 1 X 10⁸ to about 1 X 10¹¹, and even more preferably, about 1 X 10⁹ to about 1 X 10¹⁰ activated CD8+ cells are utilized for adult humans, compared to about 5 X 10⁶ - 5 X 10⁷ cells used in mice.

Preferably, as discussed herein, the activated CD 8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells are not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

The present invention provides methods of inducing a neoplasia/tumor specific immune response in a subject, vaccinating against a neoplasia/tumor, treating, alleviating a symptom of cancer, preventing or treating an infection, treating an autoimmune disease, or preventing transplant rejection in a subject by administering the subject a plurality of antigenic peptides or composition of the invention. According to the invention, the herein-described vaccine or immunogenic composition may be used for a patient that has been diagnosed as having cancer, or at risk of developing cancer.

The claimed combination of the invention is administered in an amount sufficient to induce a CTL response.

Any compositions or methods provided herein can be combined with one or more of any of the other compositions and methods provided herein.

The tumor specific antigen peptides and pharmaceutical compositions described herein can also be administered in a combination therapy with another agent, for example a therapeutic agent. By “agent” is meant any small molecule chemical compound, antibody, nucleic acid molecule, or polypeptide, or fragments thereof. In certain embodiments, the additional agents can be, but are not limited to, chemotherapeutic agents, anti-angiogenesis agents and agents that reduce immune-suppression.

“Combination therapy” is intended to embrace administration of therapeutic agents (e.g. antigenic peptides described herein) in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of antigenic peptides administered at the same or different times, or they can be formulated as a single, co-formulated pharmaceutical composition comprising the peptides. As another example, a combination of the present invention (e.g., a pooled sample of tumor specific antigens) may be formulated as separate pharmaceutical compositions that can be administered at the same or different time. As used herein, the term “simultaneously” is meant to refer to administration of one or more agents at the same time. For example, in certain embodiments, the antigenic peptides are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

The neoplasia vaccine or immunogenic composition can be administered before, during, or after administration of the additional agent. In embodiments, the neoplasia vaccine or immunogenic composition is administered before the first administration of the additional agent. In other embodiments, the neoplasia vaccine or immunogenic composition is administered after the first administration of the additional therapeutic agent (e.g., 1, 2, 3, 4, 5, 6, 7, 8, 9, 10, 11, 12, 13, 14 days or more). In embodiments, the neoplasia vaccine or immunogenic composition is administered simultaneously with the first administration of the additional therapeutic agent.

The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such ashydroxy angiostatin Kl-3, DL-a-Difluorom ethyl - ornithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-di oxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (-)-Deguelin, 5,6-Dichlorobenzimidazole I-P-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-17-demethoxygeldanamycin, 4-Amino-1,8- naphthalimide, Apigenin, Brefeldin A, Cimetidine, Dichloromethylene-diphosphonic acid, Leuprolide (Leuprorelin), Luteinizing Hormone-Releasing Hormone, Pifithrin-a, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cytarabine, dacarbazine (DTIC), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent may be a monoclonal antibody or small molecule such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 1311-tositumomab, ado-trastuzumab emtansine, lapatinib (Tykerb®), pertuzumab (Perjeta™), ado-trastuzumab emtansine (Kadcyla™), regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatinib dimaleate (Gilotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), pembrolizumab (Keytruda®), carfilzomib (Kyprolis®), Ramucirumab (Cyramza™), Cabozantinib (Cometriq™), vandetanib (Caprelsa®), Optionally, the therapeutic agent is a antigen. The therapeutic agent may be a cytokine such as interferons (INFs), interleukins (ILs), or hematopoietic growth factors. The therapeutic agent may be INF-a, IL-2, Aldesleukin, IL-2, Erythropoietin, Granulocyte-macrophage colony-stimulating factor (GM-CSF) or granulocyte colony-stimulating factor. The therapeutic agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vonnostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). Aditionally, the therapeutic agent may be an epigenetic targeted drug such as FIDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zolinza), Romidepsin (Istodax), or Ruxolitinib (Jakafi). For prostate cancer treatment, a preferred chemotherapeutic agent with which anti- CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone- treated murine T cell hybridomas (Nocentini et al. Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622.2004). The observation that GITR stimulation has immunostimulatory effects in vitro and induced autoimmunity in vivo prompted the investigation of the antitumor potency of triggering this pathway. A review of Modulation Of Ctla 4 And Gitr For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAG3, ICOS, PDL1 or KIR (Page et a, Annual Review of Medicine 65:27 (2014)). In further additional embodiments, the checkpoint inhibitor is targeted at a member of the TNFR superfamily such as CD40, OX40, CD 137, GITR, CD27 or TEVI-3. In some cases targeting a checkpoint inhibitor is accomplished with an inhibitory antibody or similar molecule. In other cases, it is accomplished with an agonist for the target; examples of this class include the stimulatory targets OX40 and GITR.

In certain embodiments, the one or more additional agents are synergistic in that they increase immunogenicity after treatment. In one embodiment the additional agent allows for lower toxicity and/or lower discomfort due to lower doses of the additional therapeutic agents or any components of the combination therapy described herein. In another embodiment the additional agent results in longer lifespan due to increased effectiveness of the combination therapy described herein. Chemotherapeutic treatments that enhance the immunological response in a patient have been reviewed (Zitvogel et al., Immunological aspects of cancer chemotherapy. Nat Rev Immunol. 2008 Jan;8(l):59-73). Aditionally, chemotherapeutic agents can be administered safely with immunotherapy without inhibiting vaccine specific T-cell responses (Perez et al., A new era in anticancer peptide vaccines. Cancer May 2010). In one embodiment the additional agent is administered to increase the efficacy of the therapy described herein. In one embodiment the additional agent is a chemotherapy treatment. In one embodiment low doses of chemotherapy potentiate delayed-type hypersensitivity (DTH) responses. In one embodiment the chemotheray agent targets regulatory T-cells. In one embodiment cyclophosphamide is the therapeutic agent. In one embodiment cyclophosphamide is administered prior to vaccination. In one embodiment cyclophosphamide is administered as a single dose before vaccination (Walter et al., Multipeptide immune response to cancer vaccine IMA901 after single-dose cyclophosphamide associates with longer patient survival. Nature Medicine; 18:8 2012). In another embodiment, cyclophosphamide is administered according to a metronomic program, where a daily dose is administered for one month (Ghiringhelli et al., Metronomic cyclophosphamide regimen selectively depletes CD4+CD25+ regulatory T cells and restores T and NK effector functions in end stage cancer patients. Cancer Immunol Immunother 2007 56:641-648). In another embodiment taxanes are administered before vaccination to enhance T- cell and NK-cell functions (Zitvogel et al., 2008, Nat. Rev. Immunol., 8(l):59-73). In another embodiment a low dose of a chemotherapeutic agent is administered with the therapy described herein. In one embodiment the chemotherapeutic agent is estramustine. In one embodiment the cancer is hormone resistant prostate cancer. A >50% decrease in serum prostate specific antigen (PSA) was seen in 8.7% of advanced hormone refractory prostate cancer patients by personalized vaccination alone, whereas such a decrease was seen in 54% of patients when the personalized vaccination was combined with a low dose of estramustine (Itoh et al., Personalized peptide vaccines: A new therapeutic modality for cancer. Cancer Sci 2006; 97: 970-976). In another embodiment glucocorticoids are administered with or before the therapy described herein (Zitvogel et al., 2008, Nat. Rev. Immunol., 8(l):59-73). In another embodiment glucocorticoids are administered after the therapy described herein. In another embodiment Gemcitabine is administered before, simultaneously, or after the therapy described herein to enhance the frequency of tumor specific CTL precursors (Zitvogel et al., 2008, Nat. Rev. Immunol., 8(1):59- 73). In another embodiment 5-fluorouracil is administered with the therapy described herein as synergistic effects were seen with a peptide based vaccine (Zitvogel et al., 2008, Nat. Rev. Immunol., 8(l):59-73). In another embodiment an inhibitor of Braf, such as Vemurafenib, is used as an additional agent. Braf inhibition has been shown to be associated with an increase in melanoma antigen expression and T-cell infiltrate and a decrease in immunosuppressive cytokines in tumors of treated patients (Frederick et al., BRAF inhibition is associated with enhanced melanoma antigen expression and a more favorable tumor microenvironment in patients with metastatic melanoma. Clin Cancer Res. 2013; 19: 1225-1231). In another embodiment an inhibitor of tyrosine kinases is used as an additional agent. In one embodiment the tyrosine kinase inhibitor is used before vaccination with the therapy described herein. In one embodiment the tyrosine kinase inhibitor is used simultaneously with the therapy described herein. In another embodiment the tyrosine kinase inhibitor is used to create a more immune permissive environment. In another embodiment the tyrosine kinase inhibitor is sunitinib or imatinib mesylate. It has previously been shown that favorable outcomes could be achieved with sequential administration of continuous daily dosing of sunitinib and recombinant vaccine (Farsaci et al., Consequence of dose scheduling of sunitinib on host immune response elements and vaccine combination therapy. Int J Cancer; 130: 1948-1959). Sunitinib has also been shown to reverse type-1 immune suppression using a daily dose of 50 mg/day (Finke et al., Sunitinib Reverses Type-1 Immune Suppression and Decreases T-Regulatory Cells in Renal Cell Carcinoma Patients. Clin Cancer Res 2008; 14(20)). In another embodiment targeted therapies are administered in combination with the therapy described herein. Doses of targeted therapies has been described previously (Alvarez, Present and future evolution of advanced breast cancer therapy. Breast Cancer Research 2010, 12(Suppl 2):S1). In another embodiment temozolomide is administered with the therapy described herein. In one embodiment temozolomide is administered at 200 mg/day for 5 days every fourth week of a combination therapy with the therapy described herein. Results of a similar strategy have been shown to have low toxicity (Kyte et al., Tel om erase Peptide Vaccination Combined with Temozolomide: A Clinical Trial in Stage IV Melanoma Patients. Clin Cancer Res; 17(13) 2011). In another embodiment the therapy is administered with an additional therapeutic agent that results in lymphopenia. In one embodiment the additional agent is temozolomide. An immune response can still be induced under these conditions (Sampson et al., Greater chemotherapy-induced lymphopenia enhances tumor-specific immune responses that eliminate EGFRvIII-expressing tumor cells in patients with glioblastoma. Neuro-Oncology 13(3):324-333, 2011).

Patients in need thereof may receive a series of priming vaccinations with a mixture of tumor-specific peptides. Additionally, over a 4 week period the priming may be followed by two boosts during a maintenance phase. All vaccinations are subcutaneously delivered. The vaccine or immunogenic composition is evaluated for safety, tolerability, immune response and clinical effect in patients and for feasibility of producing vaccine or immunogenic composition and successfully initiating vaccination within an appropriate time frame. The first cohort can consist of 5 patients, and after safety is adequately demonstrated, an additional cohort of 10 patients may be enrolled. Peripheral blood is extensively monitored for peptide-specific T-cell responses and patients are followed for up to two years to assess disease recurrence.

Administering a combination therapy consistent with standard of care. In another aspect, the therapy described herein provides selecting the appropriate point to administer a combination therapy in relation to and within the standard of care for the cancer being treated for a patient in need thereof. The studies described herein show that the combination therapy can be effectively administered even within the standard of care that includes surgery, radiation, or chemotherapy. The standards of care for the most common cancers can be found on the website of National Cancer Institute (www.cancer.gov/cancertopics). The standard of care is the current treatment that is accepted by medical experts as a proper treatment for a certain type of disease and that is widely used by healthcare professionals. Standard or care is also called best practice, standard medical care, and standard therapy. Standards of Care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The combination therapy described herein can be incorporated within the standard of care. The combination therapy described herein may also be administered where the standard of care has changed due to advances in medicine.

Incorporation of the combination therapy described herein may depend on a treatment step in the standard of care that can lead to activation of the immune system. Treatment steps that can activate and function synergistically with the combination therapy have been described herein. The therapy can be advantageously administered simultaneously or after a treatment that activates the immune system.

Incorporation of the combination therapy described herein may depend on a treatment step in the standard of care that causes the immune system to be suppressed. Such treatment steps may include irradiation, high doses of alkylating agents and/or methotrexate, steroids such as glucosteroids, surgery, such as to remove the lymph nodes, imatinib mesylate, high doses of T F, and taxanes (Zitvogel et al., 2008, Nat. Rev. Immunol., 8(l):59-73). The combination therapy may be administered before such steps or may be administered after.

In one embodiment the combination therapy may be administered after bone marrow transplants and peripheral blood stem cell transplantation. Bone marrow transplantation and peripheral blood stem cell transplantation are procedures that restore stem cells that were destroyed by high doses of chemotherapy and/or radiation therapy. After being treated with high- dose anticancer drugs and/or radiation, the patient receives harvested stem cells, which travel to the bone marrow and begin to produce new blood cells. A “mini-transplant” uses lower, less toxic doses of chemotherapy and/or radiation to prepare the patient for transplant. A “tandem transplant” involves two sequential courses of high-dose chemotherapy and stem cell transplant. In autologous transplants, patients receive their own stem cells. In syngeneic transplants, patients receive stem cells from their identical twin. In allogeneic transplants, patients receive stem cells from their brother, sister, or parent. A person who is not related to the patient (an unrelated donor) also may be used. In some types of leukemia, the graft-versus-tumor (GVT) effect that occurs after allogeneic BMT and PBSCT is crucial to the effectiveness of the treatment. GVT occurs when white blood cells from the donor (the graft) identify the cancer cells that remain in the patient’s body after the chemotherapy and/or radiation therapy (the tumor) as foreign and attack them. Immunotherapy with the combination therapy described herein can take advantage of this by vaccinating after a transplant. Additionally, the transferred cells may be presented with antigens of the combination therapy described herein before transplantation.

In one embodiment the combination therapy is administered to a patient in need thereof with a cancer that requires surgery. In one embodiment the combination therapy described herein is administered to a patient in need thereof in a cancer where the standard of care is primarily surgery followed by treatment to remove possible micro-metastases, such as breast cancer. Breast cancer is commonly treated by various combinations of surgery, radiation therapy, chemotherapy, and hormone therapy based on the stage and grade of the cancer. Adjuvant therapy for breast cancer is any treatment given after primary therapy to increase the chance of long-term survival. Neoadjuvant therapy is treatment given before primary therapy. Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery, a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy. Even in early-stage breast cancer, cells may break away from the primary tumor and spread to other parts of the body (metastasize). Therefore, doctors give adjuvant therapy to kill any cancer cells that may have spread, even if they cannot be detected by imaging or laboratory tests.

In one embodiment the combination therapy is administered consistent with the standard of care for Ductal carcinoma in situ (DCIS). The standard of care for this breast cancer type is: 1. Breast-conserving surgery and radiation therapy with or without tamoxifen; 2. Total mastectomy with or without tamoxifen; 3. Breast-conserving surgery without radiation therapy. The combination therapy may be administered before breast conserving surgery or total mastectomy to shrink the tumor before surgery. In another embodiment the combination therapy can be administered as an adjuvant therapy to remove any remaining cancer cells.

In another embodiment patients diagnosed with stage I, II, IIIA, and Operable IIIC breast cancer are treated with the combination therapy as described herein. The standard of care for this breast cancer type is: 1. Local -regional treatment: Breast-conserving therapy (lumpectomy, breast radiation, and surgical staging of the axilla), Modified radical mastectomy (removal of the entire breast with level I— II axillary dissection) with or without breast reconstruction, Sentinel node biopsy. 2. Adjuvant radiation therapy postmastectomy in axillary node-positive tumors: For one to three nodes: unclear role for regional radiation (infra/supraclavicular nodes, internal mammary nodes, axillary nodes, and chest wall). For more than four nodes or extranodal involvement: regional radiation is advised. 3. Adjuvant systemic therapy. In one embodiment the combination therapy is administered as a neoadjuvant therapy to shrink the tumor. In another embodiment the combination is administered as an adjuvant systemic therapy.

In another embodiment patients diagnosed with inoperable stage IIIB or IIIC or inflammatory breast cancer are treated with the combination therapy as described herein. The standard of care for this breast cancer type is: 1. Multimodality therapy delivered with curative intent is the standard of care for patients with clinical stage IIIB disease. 2. Initial surgery is generally limited to biopsy to permit the determination of histology, estrogen-receptor (ER) and progesterone-receptor (PR) levels, and human epidermal growth factor receptor 2 (HER2/neu) overexpression. Initial treatment with anthracycline-based chemotherapy and/or taxane-based therapy is standard. For patients who respond to neoadjuvant chemotherapy, local therapy may consist of total mastectomy with axillary lymph node dissection followed by postoperative radiation therapy to the chest wall and regional lymphatics. Breast-conserving therapy can be considered in patients with a good partial or complete response to neoadjuvant chemotherapy. Subsequent systemic therapy may consist of further chemotherapy. Hormone therapy should be administered to patients whose tumors are ER- positive or unknown. All patients should be considered candidates for clinical trials to evaluate the most appropriate fashion in which to administer the various components of multimodality regimens.

In one embodiment the combination therapy is administered as part of the various components of multimodality regimens. In another embodiment the combination therapy is administered before, simultaneously with, or after the multimodality regimens. In another embodiment the combination therapy is administered based on synergism between the modalities. In another embodiment the combination therapy is administered after treatment with anthracycline-based chemotherapy and/or taxane-based therapy (Zitvogel et al., 2008, Nat. Rev. Immunol., 8(l):59-73). Treatment after administering the combination therapy may negatively affect dividing effector T-cells. The combination therapy may also be administered after radiation.

In another embodiment the combination therapy described herein is used in the treatment in a cancer where the standard of care is primarily not surgery and is primarily based on systemic treatments, such as Chronic Lymphocytic Leukemia (CLL).

In another embodiment patients diagnosed with stage I, II, III, and IV Chronic Lymphocytic Leukemia are treated with the combination therapy as described herein. The standard of care for this cancer type is: 1. Observation in asymptomatic or minimally affected patients, 2. Rituximab, 3. Ofatumomab, 4. Oral alkylating agents with or without corticosteroids, 5. Fludarabine, 2-chlorodeoxyadenosine, or pentostatin, 6. Bendamustine, 7. Lenalidomide and 8. Combination chemotherapy. Combination chemotherapy regimens include the following: Fludarabine plus cyclophosphamide plus rituximab. o Fludarabine plus rituximab as seen in the CLB-9712 and CLB-9011 trials, o Fludarabine plus cyclophosphamide versus fludarabine plus cyclophosphamide plus rituximab, Pentostatin plus cyclophosphamide plus rituximab as seen in the MAYO-MC0183 trial, for example, Ofatumumab plus fludarabine plus cyclophosphamide, CVP: cyclophosphamide plus vincristine plus prednisone, CHOP: cyclophosphamide plus doxorubicin plus vincristine plus prednisone, Fludarabine plus cyclophosphamide versus fludarabine as seen in the E2997 trial [NCT00003764] and the LRF-CLL4 trial, for example, Fludarabine plus chlorambucil as seen in the CLB-9011 trial, for example. 9. Involved-field radiation therapy. 10. Alemtuzumab 11. Bone marrow and peripheral stem cell transplantations are under clinical evaluation. 12. Ibrutinib

In one embodiment the combination therapy is administered before, simultaneously with or after treatment with Rituximab or Ofatumomab. As these are monoclonal antibodies that target B-cells, treatment with the combination therapy may be synergistic. In another embodiment the combination therapy is administered after treatment with oral alkylating agents with or without corticosteroids, and Fludarabine, 2-chlorodeoxyadenosine, or pentostatin, as these treatments may negatively affect the immune system if administered before. In one embodiment bendamustine is administered with the combination therapy in low doses based on the results for prostate cancer described herein. In one embodiment the combination therapy is administered after treatment with bendamustine.

In another embodiment, therapies targeted to specific recurrent mutations in genes that include extracellular domains are used in the treatment of a patient in need thereof suffering from cancer. The genes may advantageously be well -expressed genes. Well expressed may be expressed in “transcripts per million” (TPM). A TPM greater than 100 is considered well expressed. Well expressed genes may be FGFR3, ERBB3, EGFR, MUC4, PDGFRA, MMP12, TMEM52, and PODXL. The therapies may be a ligand capable of binding to an extracellular antigen epitope. Such ligands are well known in the art and may include therapeutic antibodies or fragments thereof, antibody-drug conjugates, engineered T cells, or aptamers. Engineered T cells may be chimeric antigen receptors (CARs). Antibodies may be fully humanized, humanized, or chimeric. The antibody fragments may be a nanobody, Fab, Fab′, (Fab′)2, Fv, ScFv, diabody, triabody, tetrabody, Bis-scFv, minibody, Fab2, or Fab3 fragment. Antibodies may be developed against tumor-specific neoepitopes using known methods in the art.

### Adoptive Cell Transfer (ACT)

In certain embodiments, immune cells specific to an identified antigenic peptide that binds to a subject specific HLA allele is used in treatment. For example, CD8+ T cells or NK cell that express a TCR or CAR specific for the peptide, or dendritic cells that are loaded with one or more peptides are transferred to a subject in need thereof. In certain embodiments, T cells are isolated that interact with the peptide and expanded ex vivo. The expanded cells can then be administered back to a subject (i.e., autologous T cells).

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep 4;8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 Jun;24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically redirected peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific antigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific antigens in chronic lymphocytic leukemia. Blood. 2014 Jul 17;124(3):453-62).

In certain embodiments, an additional antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar 8; Berdej a JG, et al. Durable clinical responses in heavily pretreated patients with relapsed/refractory multiple myeloma: updated results from a multicenter study of bb2121 anti-Bcma CAR T cell therapy. Blood. 2017;130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v⅞ (cluster of differentiation 44, exons ⅞); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD 117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein);, fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a antigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms’ tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD 171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target (see, e.g., Gong Y, Klein Wolterink RGJ, Wang J, Bos GMJ, Germeraad WTV. Chimeric antigen receptor natural killer (CAR-NK) cell design and engineering for cancer therapy. J Hematol Oncol. 2021;14(1):73; Guedan S, Calderon H, Posey AD Jr, Maus MV. Engineering and Design of Chimeric Antigen Receptors. Mol Ther Methods Clin Dev. 2018;12:145-156; and Petersen CT, Krenciute G. Next Generation CAR T Cells for the Immunotherapy of High-Grade Glioma. Front Oncol. 2019;9:69). While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRy (scFv-CD3ζ or scFv-FcRγ; see U.S. Pat. No. 7,741,465; U.S. Pat. No. 5,912,172; U.S. Pat. No. 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. No. 8,906,682; U.S. Pat. No. 8,399,645; U.S. Pat. No. 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of US 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of US 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ. I.D. No. 17). Alternatively, when the zeta sequence lies between the CD28 sequence and the antigen-binding element, intracellular domain of CD28 can be used alone (such as amino sequence set forth in SEQ ID NO: 9 of US 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of US 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 18) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM _006139 (sequence version 1, 2 or 3) starting with the amino acid sequence IEVMYPPPY (SEQ ID NO: 18) and continuing all the way to the carboxy-terminus of the protein. The sequence is reproduced herein:

IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS (SEQ ID NO: 19).

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3C, 4-1BB-CD27-CD3ζ; CD27-4-1BB-CD3ζ; CD28-CD27-FcεRI gamma chain; or CD28-FcsRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 Mar;78:145-150; and Jin et al., CD70, a novel target of CAR T-cell therapy for gliomas, Neuro Oncol. 2018 Jan 10;20(1):55-65). CD70 is expressed by diffuse large B-cell and follicular lymphoma and also by the malignant cells of Hodgkins lymphoma, Waldenstrom’s macroglobulinemia and multiple myeloma, and by HTLV-1- and EBV-associated malignancies. (Agathanggelou et al. Am.J.Pathol. 1995;147: 1152-1160; Hunter et al., Blood 2004; 104:4881. 26; Lens et al., J Immunol. 2005;174:6212-6219; Baba et al., J Virol. 2008;82:3843-3852.) In addition, CD70 is expressed by non-hematological malignancies such as renal cell carcinoma and glioblastoma. (Junker et al., J Urol. 2005;173:2150-2153; Chahlavi et al., Cancer Res 2005;65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (US 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, US 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 Oct; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul 15; 112(2):362-73; and Martin-Orozco N, et al., T helper 17 cells promote cytotoxic T cell activation in tumor immunity. Immunity. 2009 Nov 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don’t Forget the Fuel, Front. Immunol., 03 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs CS, Rosenberg SA. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10): 1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don’t Forget the Fuel, Front. Immunol., 03 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection

The administration of the cells or population of cells can consist of the administration of 10⁴- 10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Pat. Publication No. 20130071414; PCT Patent Publication WO2011146862; PCT Patent Publication WO2014011987; PCT Patent Publication WO2013040371; Zhou et al. BLOOD, 2014, 123/25:3895 - 3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1;23(9):2255-2266. doi: 10.1158/1078-0432.CCR-16-1300. Epub 2016 Nov 4; Qasim et al., 2017, Molecular remission of infant B-ALL after infusion of universal TALEN gene-edited CAR T cells, Sci Transl Med. 2017 Jan 25;9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1;112(12):4746-54). Thus, to prevent rejection of allogeneic cells, the host’s immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD 137, GITR, CD27 or TIM-3.

Additional immune checkpoints include Src homology 2 domain-containing protein tyrosine phosphatase 1 (SHP-1) (Watson HA, et al., SHP-1: the next checkpoint target for cancer immunotherapy? Biochem Soc Trans. 2016 Apr 15;44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD- L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms’ tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient’s immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pats. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer’s instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37° C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶ /ml. In other embodiments, the concentration used can be from about 1×10⁵ /ml to 1×10⁶ /ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to -80° C. at a rate of 1° per minute and stored in the vapor phase of a liquid nitrogen storage tank. Other methods of controlled freezing may be used as well as uncontrolled freezing immediately at -20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Pat. Publication No. US 20040224402 entitled, Generation and Isolation of Antigen-Specific T Cells, or in U.S. Pat. Nos. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled β-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Pat. Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Antibodies

In certain embodiments, the one or more agents is an antibody specific for the epitope identified herein. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG -IgG1, IgG2, IgG3, and IgG4 that have been identified in humans and higher mammals by the heavy chains of the immunoglobulins, V1 - γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins— harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 µM. Antibodies with affinities greater than 1 x 10⁷ M⁻¹ (or a dissociation coefficient of 1 µM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Bi-Specific Antibodies

In certain embodiments, the one or more therapeutic agents can be bi-specific antigen-binding constructs, e.g., bi-specific antibodies (bsAb) or BiTEs, that bind two antigens (see, e.g., Suurs et al., A review of bispecific antibodies and antibody constructs in oncology and clinical challenges. Pharmacol Ther. 2019 Sep;201:103-119; and Huehls, et al., Bispecific T cell engagers for cancer immunotherapy. Immunol Cell Biol. 2015 Mar; 93(3): 290-296). The bi-specific antigen-binding construct includes two antigen-binding polypeptide constructs, e.g., antigen binding domains, wherein at least one polypeptide construct specifically binds to a tumor surface protein. In some embodiments, the antigen-binding construct is derived from known antibodies or antigen-binding constructs. In some embodiments, the antigen- binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In certain embodiments, immune cells can be engaged to tumor cells. In certain embodiments, tumor cells are targeted with a bsAb having affinity for both the tumor (e.g., LT antigen) and a payload. By means of an example, an agent, such as a bi-specific antibody, capable of specifically binding to a gene product expressed on the cell surface of the immune cells (e.g., CD3, CD8, CD28, CD16) and a tumor cell (e.g., TSDKAIELY (SEQ ID NO:1)) may be used for targeting polyfunctional immune cells to tumor cells. Immune cells targeted to a tumor may include T cells or Natural Killer cells.

### Antibody Drug Conjugates

In certain embodiments, the one or more therapeutic agents can be an antibody drug conjugate specific for the identified epitope. The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8.

In certain embodiments, the ADC specifically binds to a gene product expressed on the cell surface of a tumor cell. By means of an example, an agent, such as an antibody, capable of specifically binding to a gene product expressed on the cell surface of the tumor cells may be conjugated with a therapeutic or effector agent for targeted delivery of the therapeutic or effector agent to the immune cells.

Examples of such therapeutic or effector agents include immunomodulatory classes as discussed herein, such as without limitation a toxin, drug, radionuclide, cytokine, lymphokine, chemokine, growth factor, tumor necrosis factor, hormone, hormone antagonist, enzyme, oligonucleotide, siRNA, RNAi, photoactive therapeutic agent, anti-angiogenic agent and pro-apoptotic agent.

Non-limiting examples of drugs that may be included in the ADCs are mitotic inhibitors (e.g., maytansinoid DM4), antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

Example toxins include ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, or Pseudomonas endotoxin.

Example radionuclides include ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³ Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo or ^{99m}Tc. Preferably, the radionuclide may be an alpha-particle-emitting radionuclide.

Example enzymes include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase or acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target’s activity, e.g., through binding, aptamers may block their target’s ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′ -modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

### Vaccine or Immunogenic Composition Kits and Co-Packaging

In an aspect, the invention provides kits containing any one or more of the elements discussed herein to allow administration of the therapy. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit may advantageously allow the provision of all elements of the systems of the invention. Kits can involve vector(s) and/or particle(s) and/or nanoparticle(s) containing or encoding RNA(s) for 1-50 or peptides to be administered to an animal, mammal, primate, rodent, etc., with such a kit including instructions for administering to such a eukaryote; and such a kit can optionally include any of the anti -cancer agents described herein. The kit may include any of the components above (e.g. vector(s) and/or particle(s) and/or nanoparticle(s) containing or encoding RNA(s) for 1-50 or more peptides, as well as instructions for use with any of the methods of the present invention. In one embodiment the kit contains at least one vial with an immunogenic composition or vaccine. In one embodiment the kit contains at least one vial with an immunogenic composition or vaccine and at least one vial with an anticancer agent. In one embodiment kits may comprise ready to use components that are mixed and ready to administer. In one aspect a kit contains a ready to use immunogenic or vaccine composition and a ready to use anti-cancer agent. The ready to use immunogenic or vaccine composition may comprise separate vials containing different pools of immunogenic compositions. The immunogenic compositions may comprise one vial containing a viral vector or DNA plasmid and the other vial may comprise immunogenic protein. The ready to use anticancer agent may comprise a cocktail of anticancer agents or a single anticancer agent. Separate vials may contain different anticancer agents. In another embodiment a kit may contain a ready to use anti -cancer agent and an immunogenic composition or vaccine in a ready to be reconstituted form. The immunogenic or vaccine composition may be freeze dried or lyophilized. The kit may comprise a separate vial with a reconstitution buffer that can be added to the lyophilized composition so that it is ready to administer. The buffer may advantageously comprise an adjuvant or emulsion according to the present invention. In another embodiment the kit may comprise a ready to reconstitute anticancer agent and a ready to reconstitute immunogenic composition or vaccine. In this aspect both may be lyophilized. In this aspect separate reconstitution buffers for each may be included in the kit. The buffer may advantageously comprise an adjuvant or emulsion according to the present invention. In another embodiment the kit may comprise single vials containing a dose of immunogenic composition and anti-cancer agent that are administered together. In another aspect multiple vials are included so that one vial is administered according to a treatment timeline. One vial may only contain the anti-cancer agent for one dose of treatment, another may contain both the anti-cancer agent and immunogenic composition for another dose of treatment, and one vial may only contain the immunogenic composition for yet another dose. In a further aspect the vials are labeled for their proper administration to a patient in need thereof. The immunogen or anti-cancer agents of any embodiment may be in a lyophilized form, a dried form or in aqueous solution as described herein. The immunogen may be a live attenuated virus, protein, or nucleic acid as described herein.

In one embodiment the anticancer agent is one that enhances the immune system to enhance the effectiveness of the immunogenic composition or vaccine. In a preferred embodiment the anti-cancer agent is a checkpoint inhibitor. In another embodiment the kit contains multiple vials of immunogenic compositions and anti-cancer agents to be administered at different time intervals along a treatment plan. In another embodiment the kit may comprise separate vials for an immunogenic composition for use in priming an immune response and another immunogenic composition to be used for boosting. In one aspect the priming immunogenic composition could be DNA or a viral vector and the boosting immunogenic composition may be protein. Either composition may be lyophilized or ready for administering. In another embodiment different cocktails of anti-cancer agents containing at least one anticancer agent are included in different vials for administration in a treatment plan.

### Combination Therapy

In certain embodiments, the therapeutic methods described herein are enhanced by administration of one or more treatments that enhance expression of HLA Class I molecules on a tumor. In one example embodiment, the treatment is interferon gamma therapy (see, e.g., US9296804B2; and Miller CH, Maher SG, Young HA. Clinical Use of Interferon-gamma. Ann N Y Acad Sci. 2009;1182:69-79). In one example embodiment, the treatment is a USP7 inhibitor, such as, XL177A (see, e.g., Bhattacharya S, Chakraborty D, Basu M, Ghosh MK. Emerging insights into HAUSP (USP7) in physiology, cancer and other diseases. Signal Transduct Target Ther. 2018;3:17. Published 2018 Jun 29. doi:10.1038/s41392-018-0012-y; and Schauer NJ, Liu X, Magin RS, et al. Selective USP7 inhibition elicits cancer cell killing through a p53-dependent mechanism. Sci Rep. 2020;10(1):5324).

In certain embodiments, the therapeutic methods described herein are administered in combination with a current treatment for MCC. Different types of treatments are available for patients with Merkel cell carcinoma. Some treatments are standard (the currently used treatment), and some are being tested in clinical trials. A treatment clinical trial is a research study meant to help improve current treatments or obtain information on new treatments for patients with cancer. When clinical trials show that a new treatment is better than the standard treatment, the new treatment may become the standard treatment. The standard treatment for MCC includes: surgery (wide local excision and/or lymph node dissection), radiation therapy, chemotherapy and immunotherapy (e.g., checkpoint blockade therapy, such as Nivolumab or Ipilimumab).

### Diagnostic Methods

The invention provides biomarkers (e.g., malignant cell specific HLA class I epitopes) for the identification, diagnosis, prognosis and manipulation of cell properties, for use in a variety of diagnostic and/or therapeutic indications. In certain embodiments, detecting a tumor marker may indicate that a subject has cancer. In certain embodiments, detecting a tumor marker or may indicate prognosis for a subject suffering from cancer.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from cancer or for identifying patients that will respond to specific treatments based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject’s risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value > second value; or decrease: first value < second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥ 50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR-), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss GK, et al., Direct multiplexed measurement of gene expression with color-coded probe pairs. Nat Biotechnol. 2008 Mar;26(3):317-25).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multiwell assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes″, Elsevier Science Publishers B.V. (1993) and Kricka, “Nonisotopic DNA Probe Techniques”, Academic Press, San Diego, Calif. (1992).

### Sequencing and Nucleic Acid Profiling

In certain embodiments, the invention involves targeted nucleic acid profiling (e.g., sequencing, quantitative reverse transcription polymerase chain reaction, and the like) (see e.g., Geiss GK, et al., Direct multiplexed measurement of gene expression with color-coded probe pairs. Nat Biotechnol. 2008 Mar;26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan;12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 Oct;14(10):955-958; International patent application number PCT/US2016/059239, published as WO2017164936 on Sep. 28, 2017; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22;348(6237):910-4. doi: 10.1126/science.aab1601. Epub 2015 May 7; US20160208323A1; US20160060691A1; and WO2017156336A1).

Although the present invention and its advantages have been described in detail, it should be understood that various changes, substitutions and alterations can be made herein without departing from the spirit and scope of the invention as defined in the appended claims.

Further embodiments are illustrated in the following Examples which are given for illustrative purposes only and are not intended to limit the scope of the invention.

### EXAMPLES

### Example 1- Systematic Evaluation and Modulation of HLA Class I Downregulation in Merkel Cell Carcinoma

### Reliable Generation of MCC Cell Lines From Primary Patient Samples

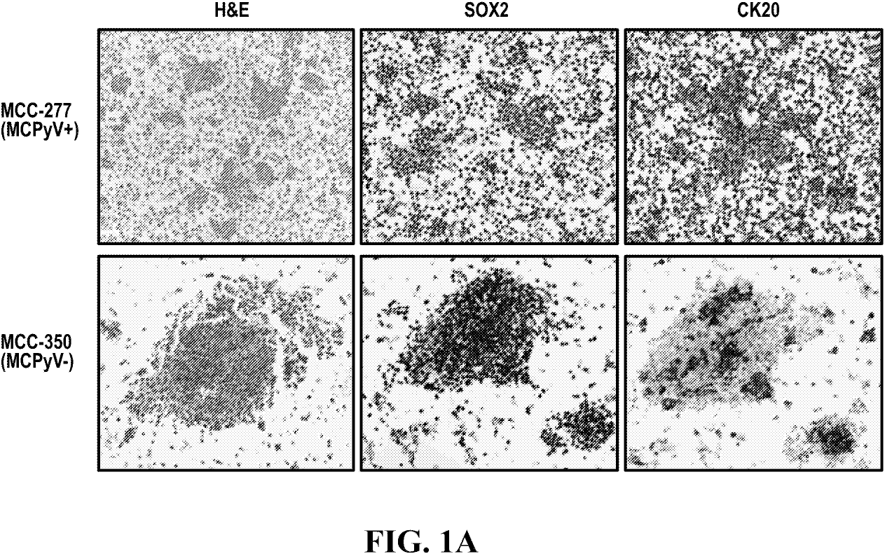

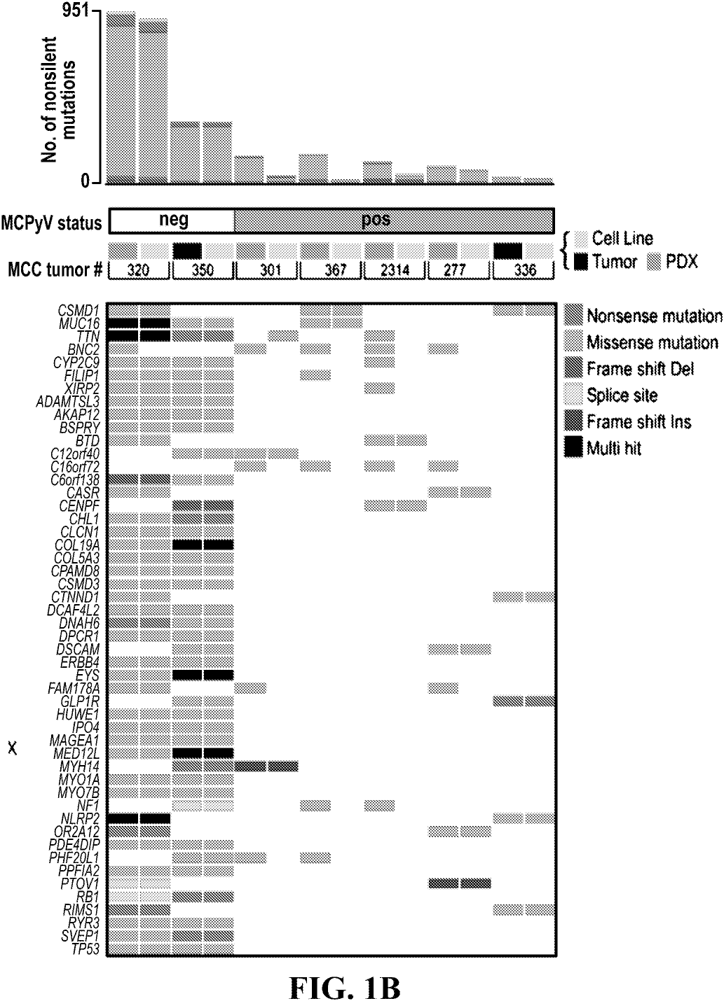

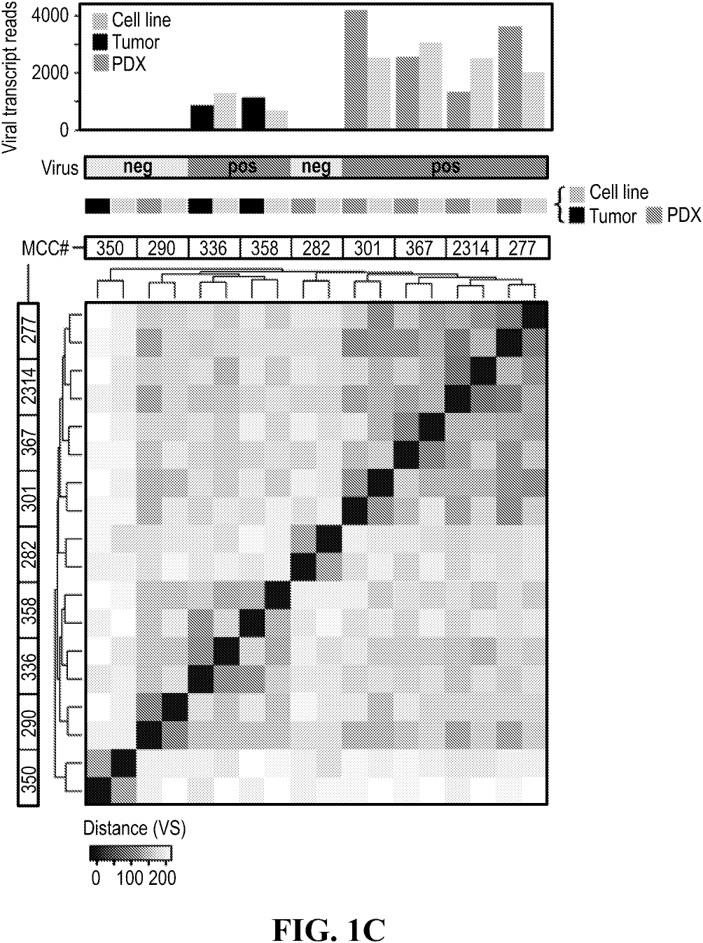

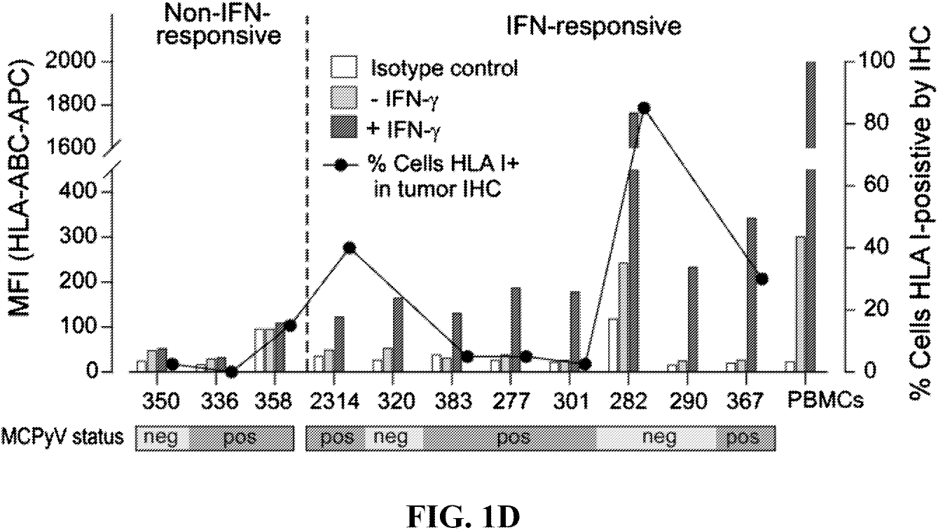

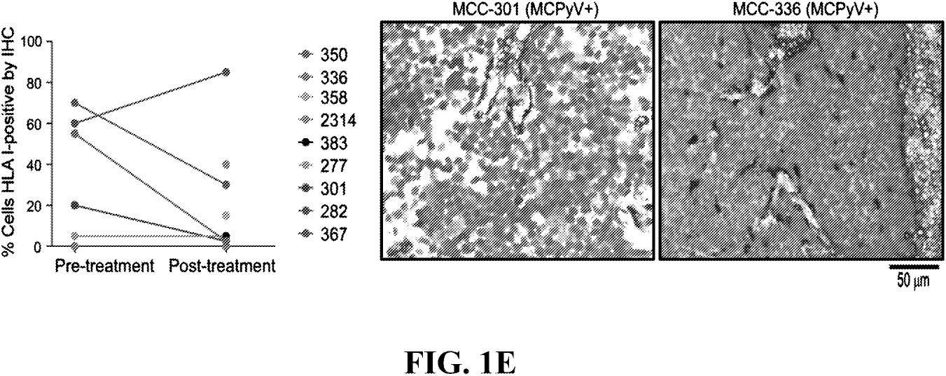

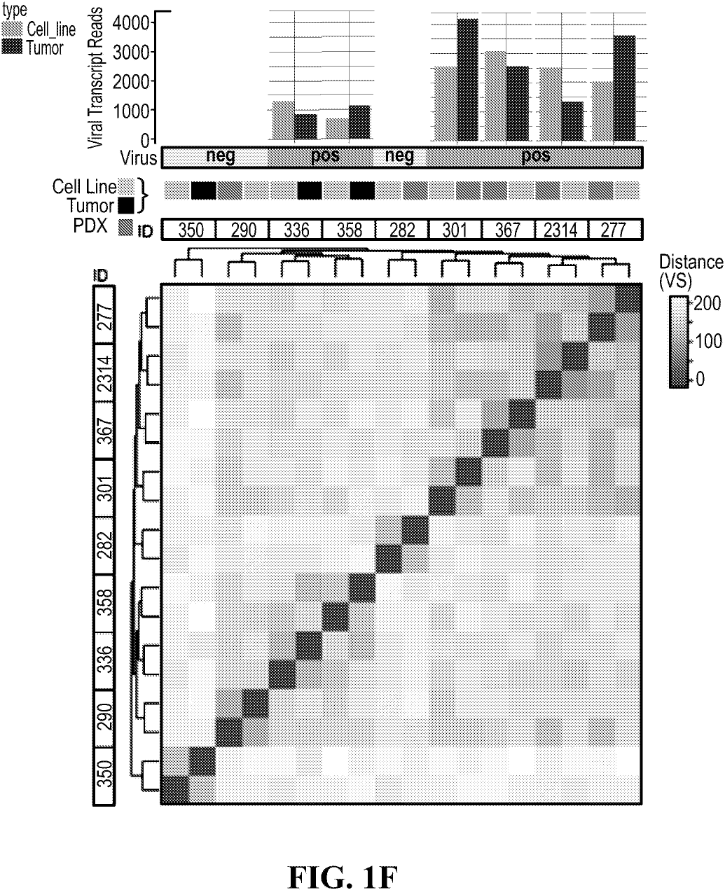

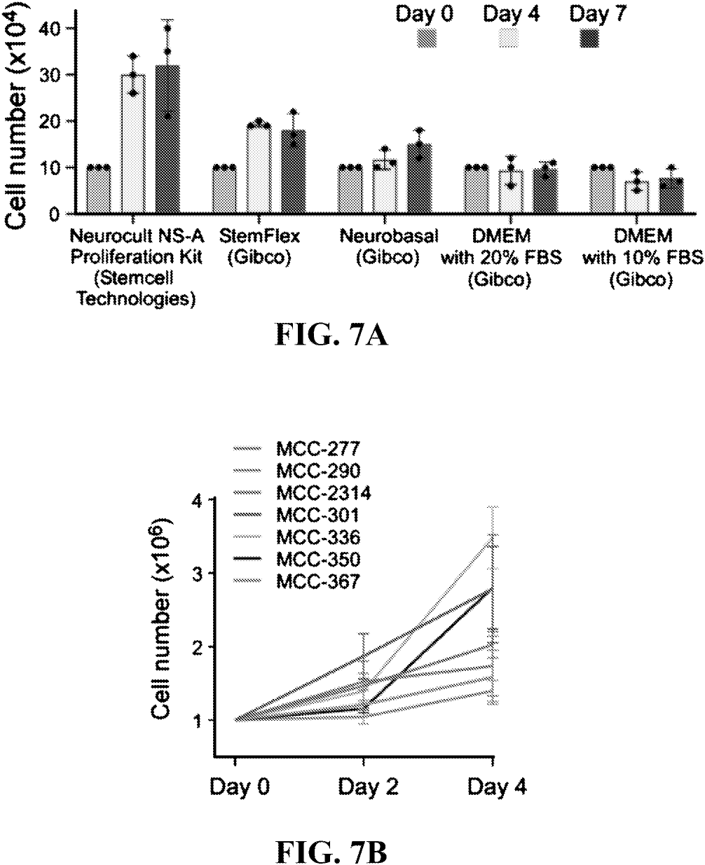

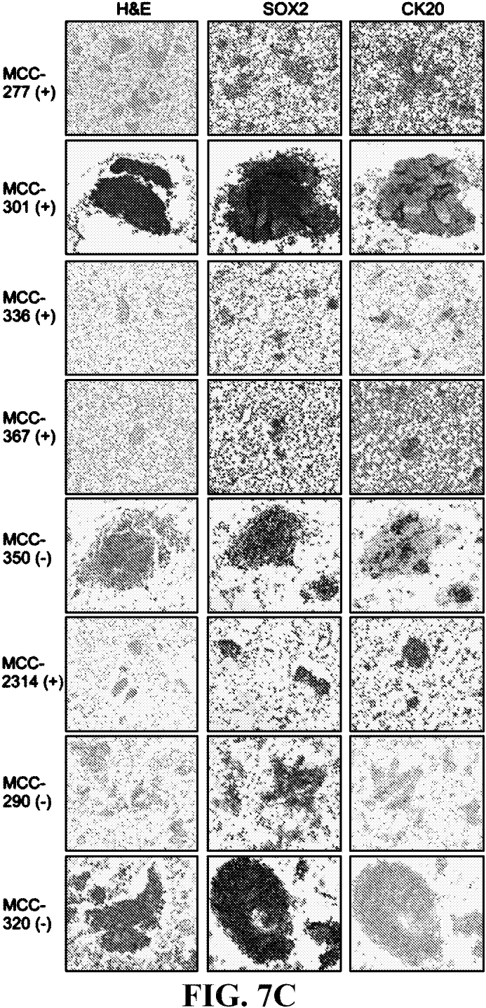

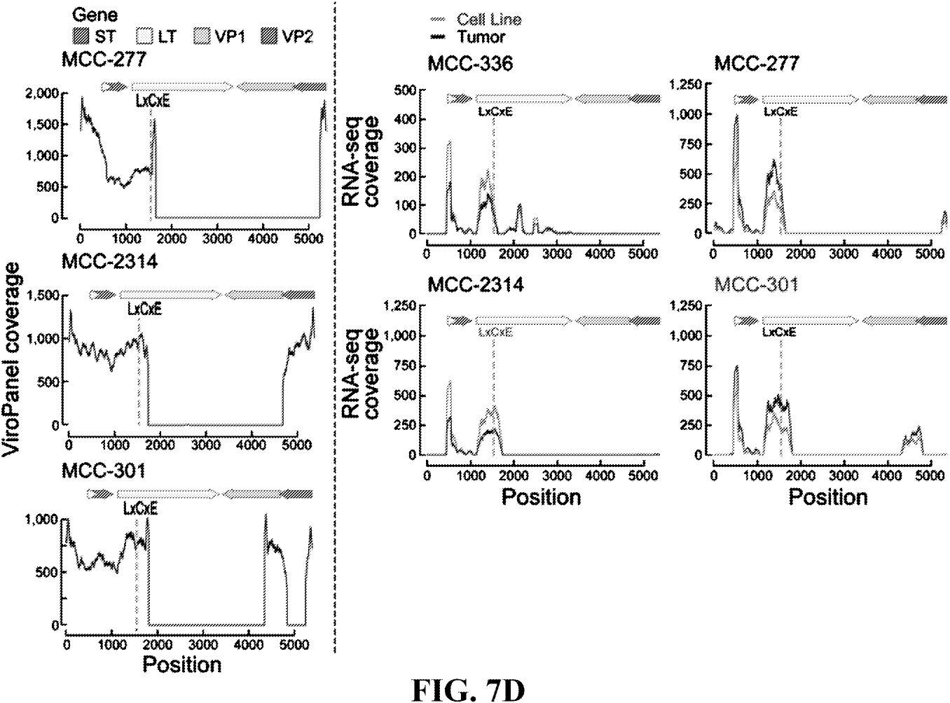

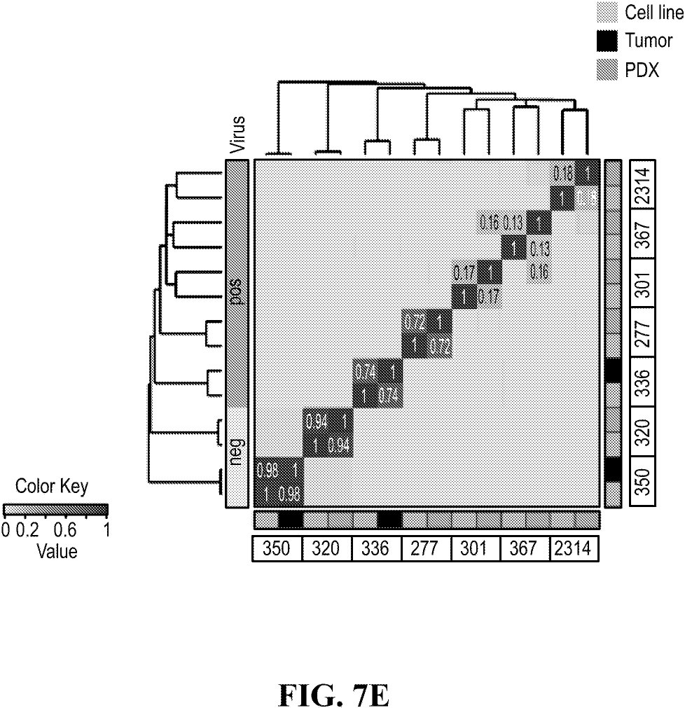

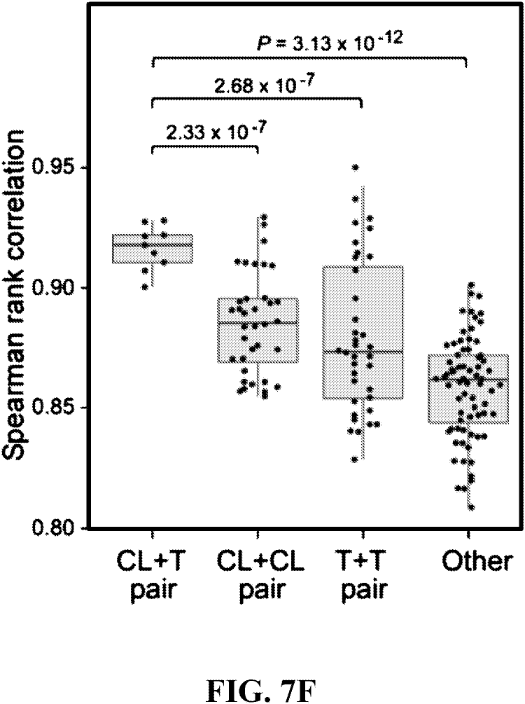

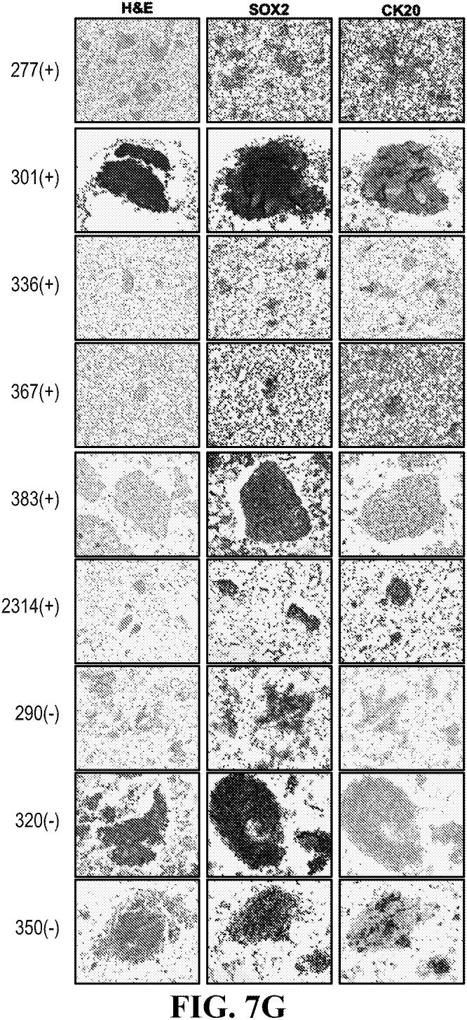

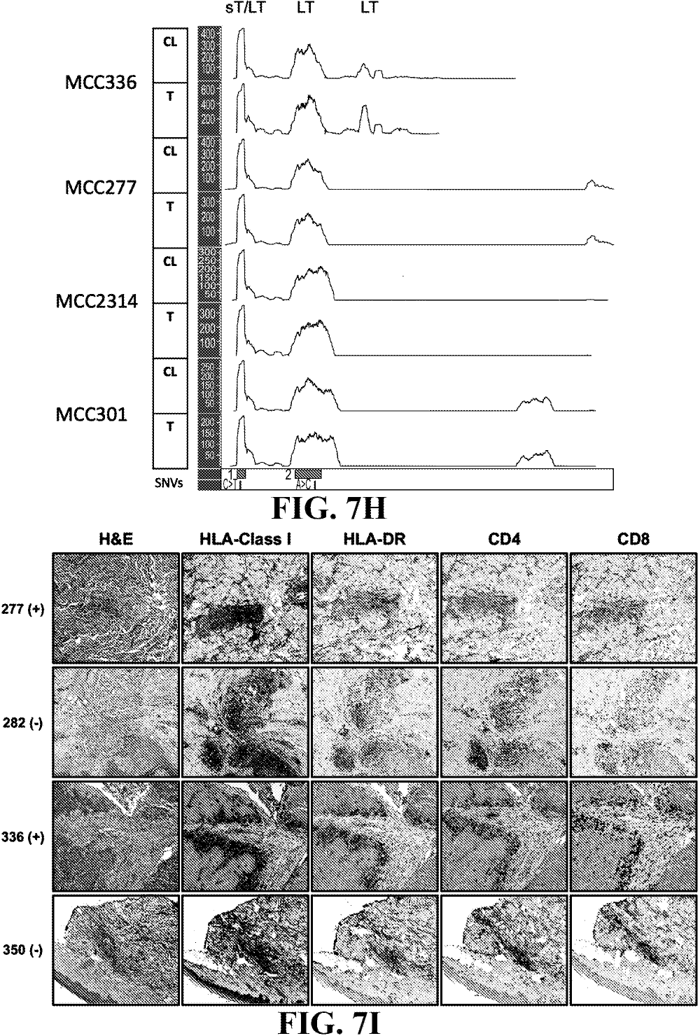

Applicants noted that many established MCC lines, typically cultured in an RPMI-1640 based media formulation (Leonard, Bell, and Kearsley 1993; Houben et al. 2010; Dresang et al. 2013; Schrama et al. 2019), have been multiply passaged in vitro and commonly lack associated archival primary tumor material and clinical data. To establish a series of lines directly from patient specimens, Applicants sought to optimize conditions to generate a reliable approach to propagate MCC in vitro. Since MCC tumors exhibit neuroendocrine histology and another panel of MCC lines had been successfully established in a modified neural crest stem cell medium (Verhaegen et al. 2014), Applicants hypothesized that culturing them in a neuronal stem cell media that Applicants previously used to establish glioblastoma multiforme tumor cell lines (Keskin et al. 2019) would facilitate cell line establishment. Indeed, of 5 media formulations tested on the MCC-336 tumor specimen, Neurocult NS-A Proliferation medium with growth factor supplementation consistently provided the highest in vitro growth rate, tripling cell numbers after seven days in culture (FIG. 7A). Using this method, Applicants established a total of 11 stable cell lines from biopsies (n=4) or patient-derived xenograft (PDX) materials (n=7) (Table 1). Consistent with previously established MCC lines, these cell lines were observed to grow mostly in tight clusters in suspension and stained positive for CK20 and SOX2, classical immunohistochemical markers of MCC (FIG. 1A; FIG. 7G). Using a hybrid-capture -based sequencing approach, Applicants determined that 7 of 11 lines were positive for MCPyV, while 4 were MCPyV-; (Methods, Supplemental tables).

For 7 of 11 patients, matched PBMCs were available from which germline DNA was extracted. Applicants hence performed whole-exome sequencing (WES) of DNA from matched primary tumor, cell line, and germline source for these lines as well as RNA-sequencing (RNAseq). These studies revealed the cell lines to display genetic alterations characteristic of MCC, as well as genomic and transcriptional similarity between corresponding tumor and cell lines. Specifically, MCPyV- and MCPyV+ samples exhibited the expected contrasting high (median 647 non-silent coding mutations per cell line, range 354-940) and low (median 40, range 18-73) mutational burdens (FIG. 1B), respectively, and the two analyzed MCPyV- lines both contained mutations in RBI and TP53, consistent with previous studies (Goh et al. 2016; Knepper et al. 2019a). Within the MCPyV+ samples, transcripts mapping to the MCPyV ST and LT antigens were detectable in both tumor and cell line RNA-seq data (FIG. 1C; FIG. 7H). By unsupervised hierarchical clustering analysis of MCC tumors and cell lines based on mutational profiles (FIG. 7D) and RNA-seq (FIG. 1F), Applicants observed each cell line to associate most closely with its corresponding parent tumor, rather than to cluster by sample type, confirming that the cell lines faithfully recapitulate the tumors from which they were derived. Of note, PDX-derived tumor samples did exhibit higher mutational burdens than their corresponding cell lines (FIG. 1B), likely due to murine cell contamination.

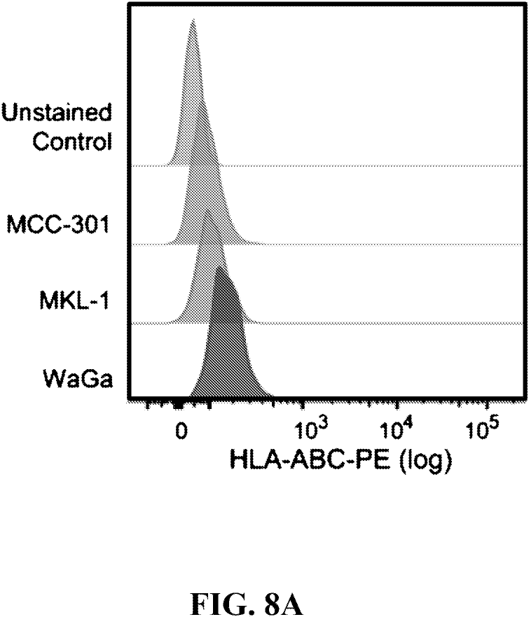

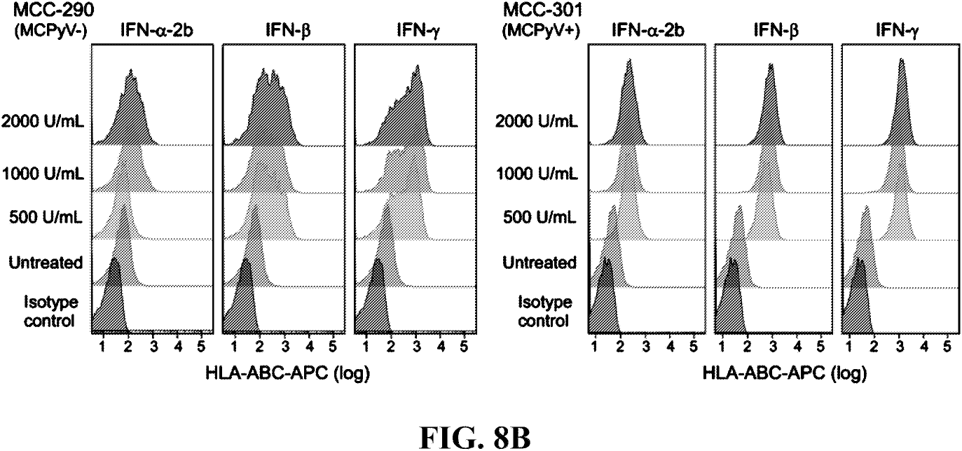

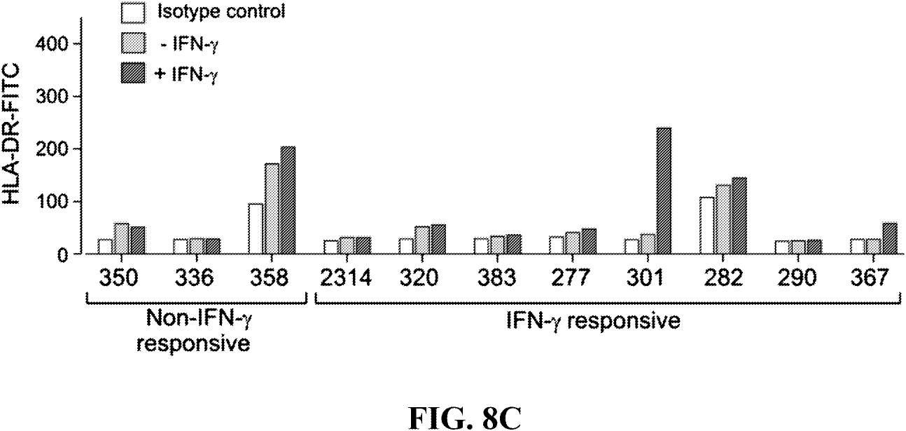

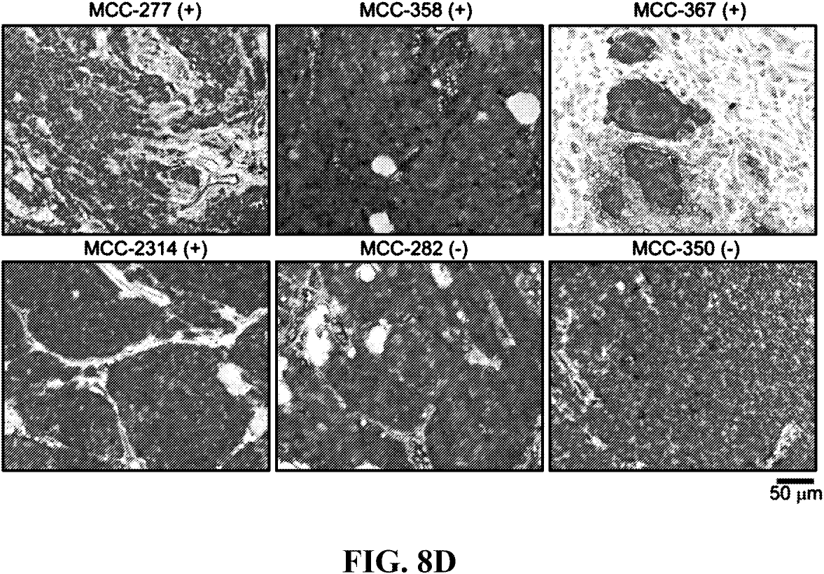

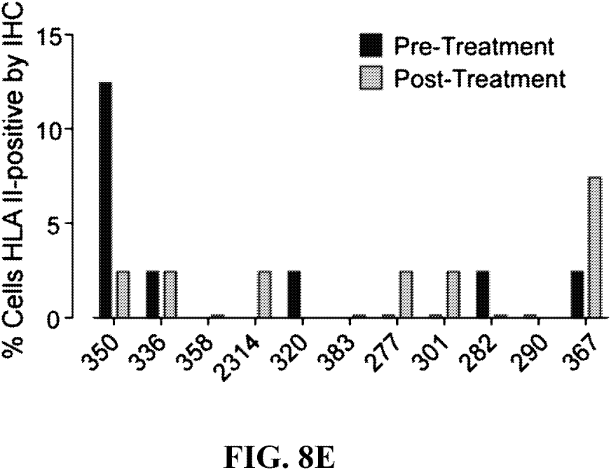

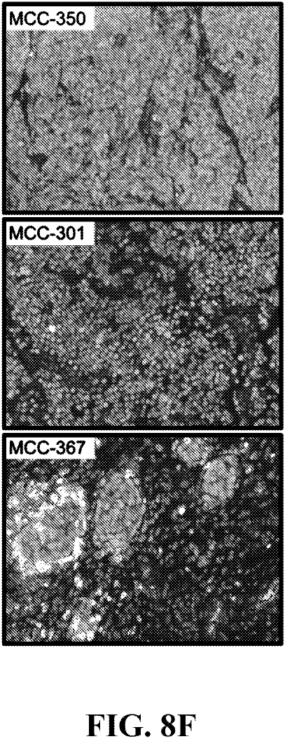

In addition to the consistency in the genetic and transcriptional profiles of the generated cell lines in relation to parental tumors, Applicants also observed that the lines displayed consistent defects in surface HLA I surface expression like their parental tumors. By flow cytometry using a pan-class I anti-HLA-ABC antibody, all 11 lines strikingly exhibited low, nearly absent HLA I (FIG. 1D). Such absence of in situ HLA class I expression on MCC cells was confirmed by immunohistochemical staining of the parental tumors for 4 lines (FIG. 7I). Moreover, the low class I surface expression by flow cytometry was on par with commonly used MCPyV+ lines MKL-1 and WaGa (FIG. 8A). Three lines were not responsive to IFN-γ exposure (MCC-336, -350, -358), whereas 8 lines exhibited HLA I surface expression that could be induced by IFN-γ (median 5.7-fold increase by MFI, range 2.5 - 12.4). For two lines, Applicants further confirmed that HLA I could be upregulated by IFN-α-2b and IFN-β(FIG. 8B) and another line (MCC-301) had inducible HLA-DR expression with IFN-γ as well (FIG. 8C). These data suggest that the majority of MCC samples have reversible HLA class I pathway defects at the transcriptional rather than at the genomic level.

### MCC Lines Exhibit Transcriptional Downregulation of Multiple Class I Genes With underlying NLRC5 Alterations

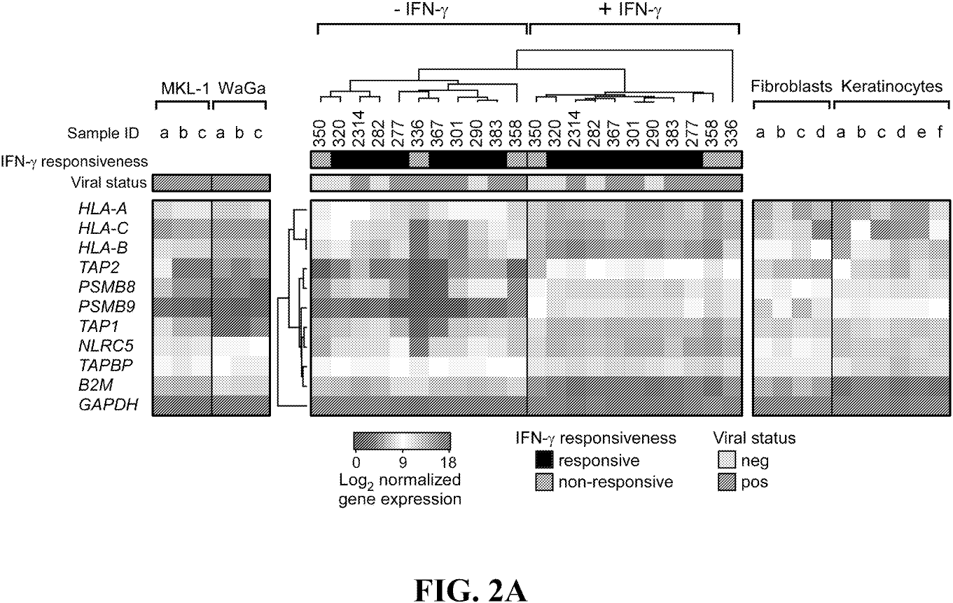

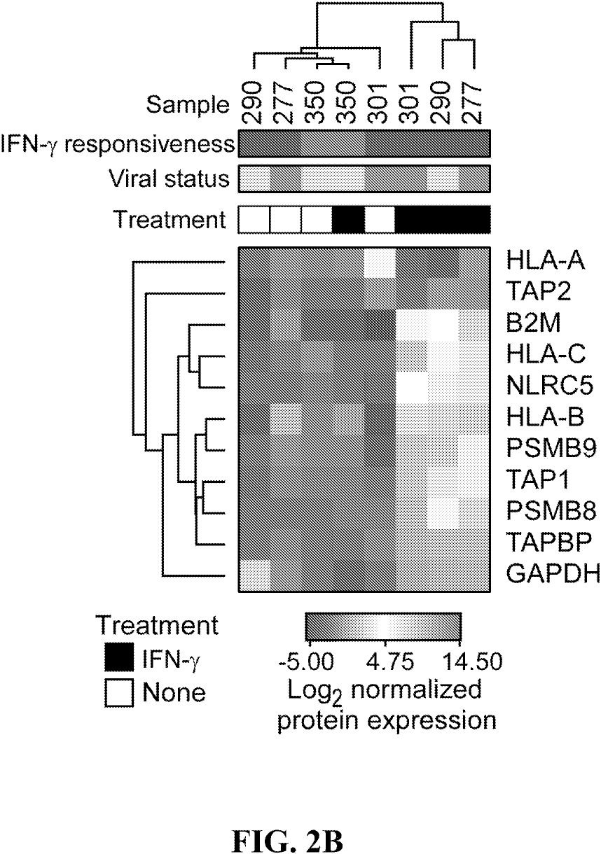

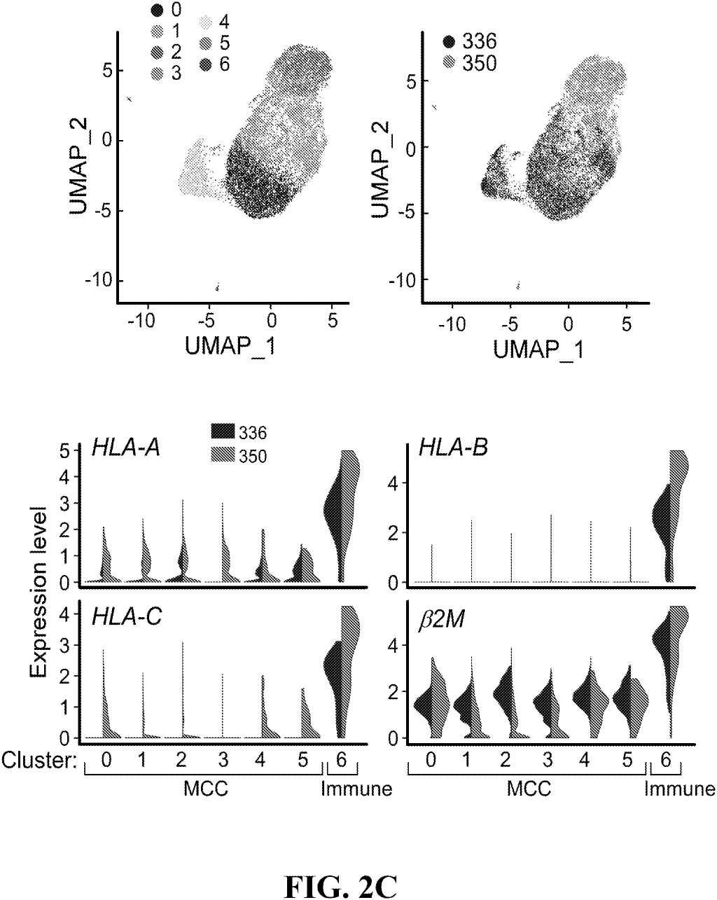

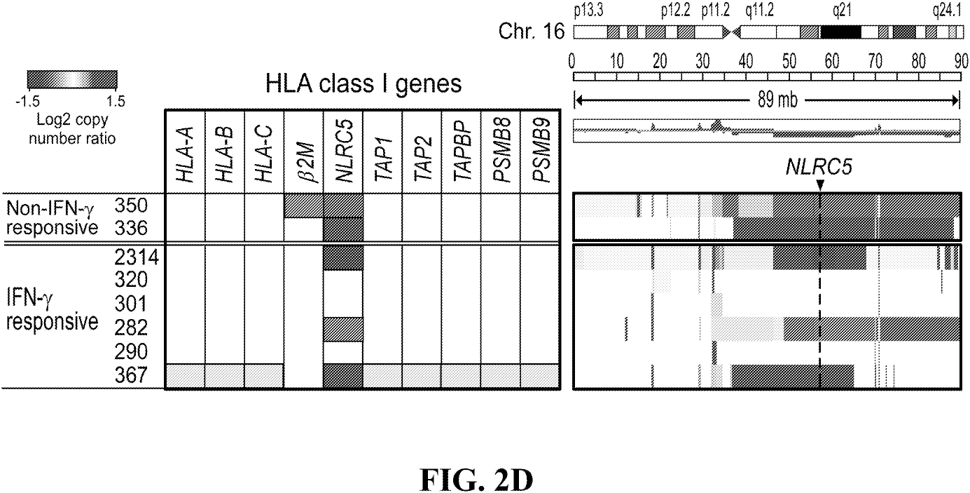

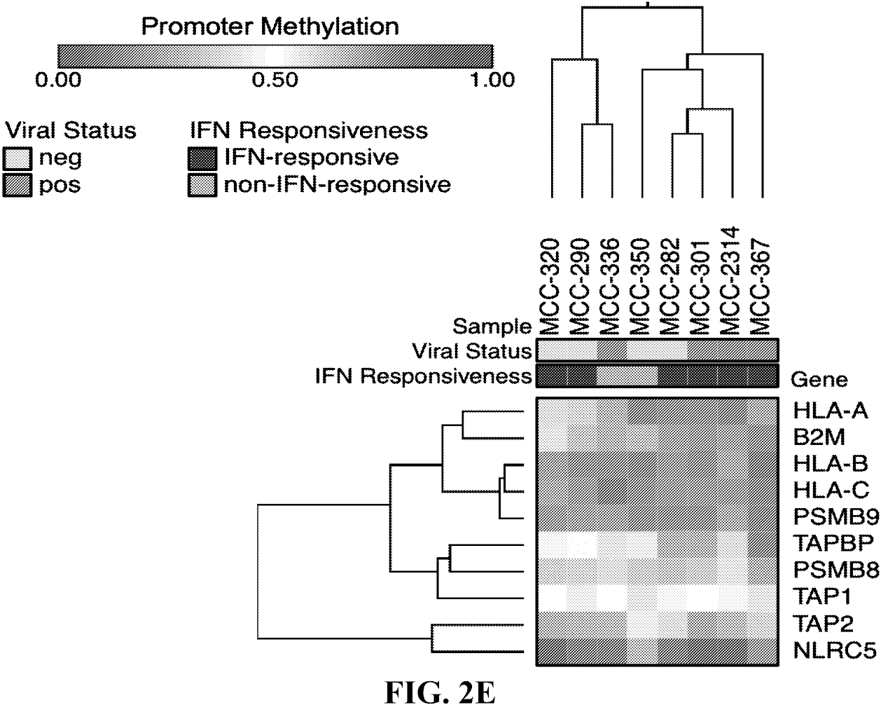

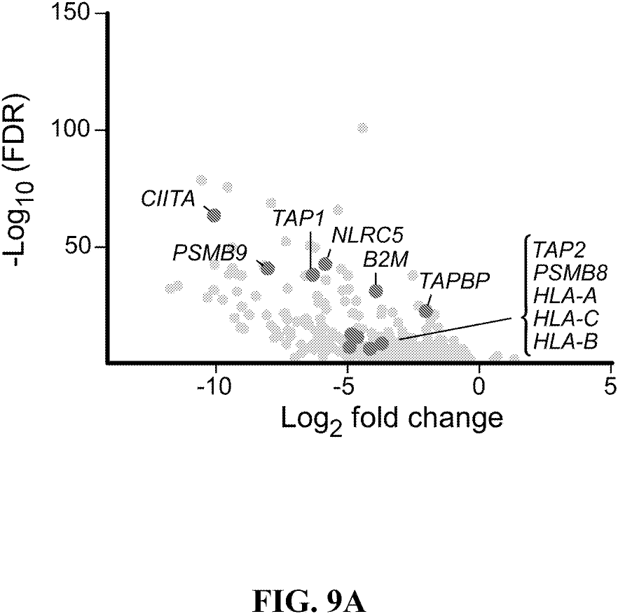

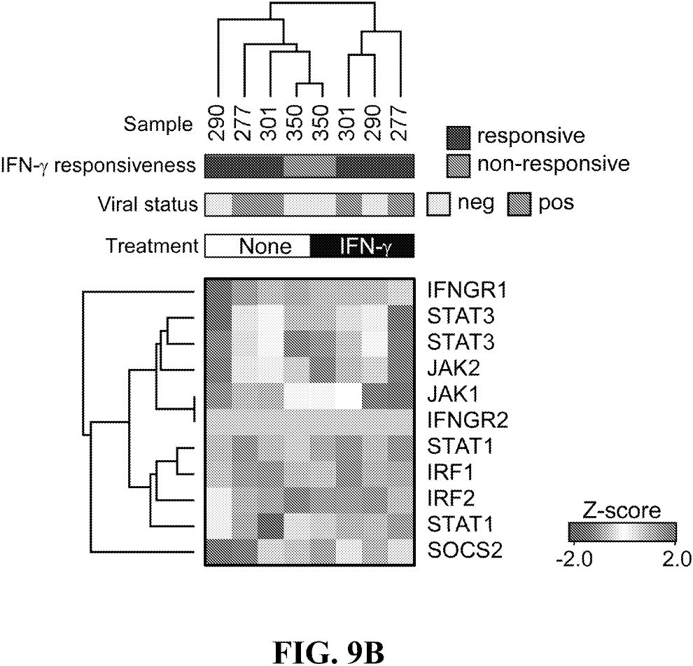

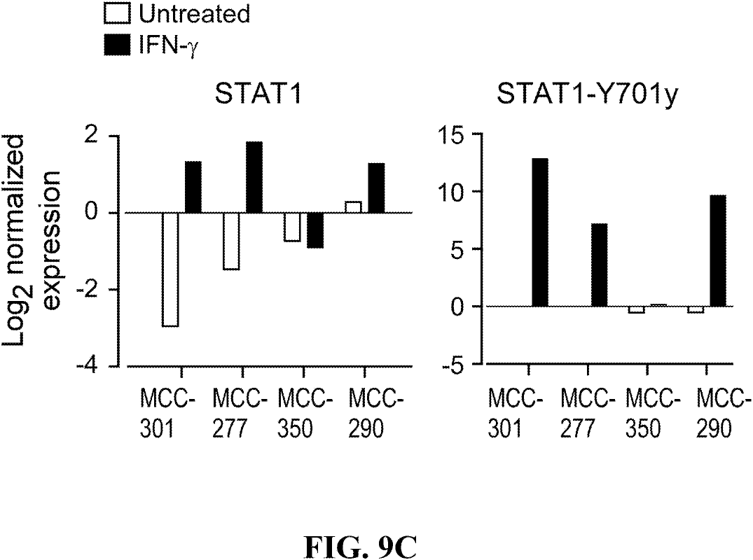

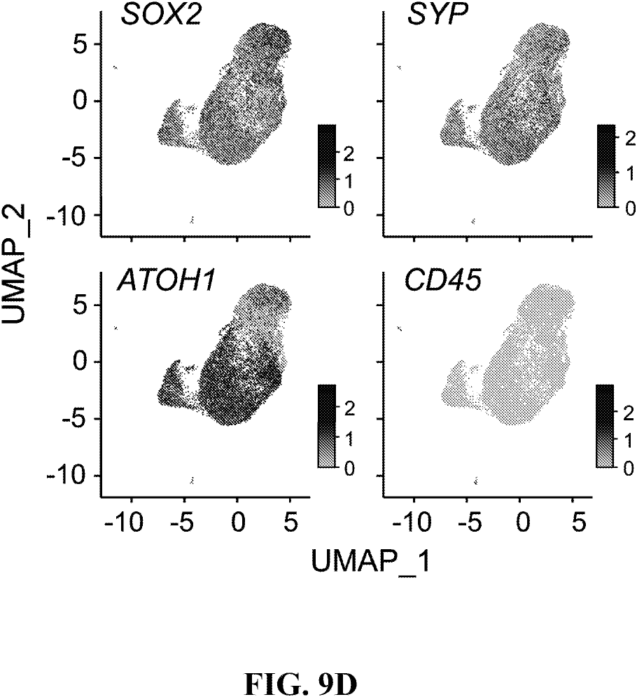

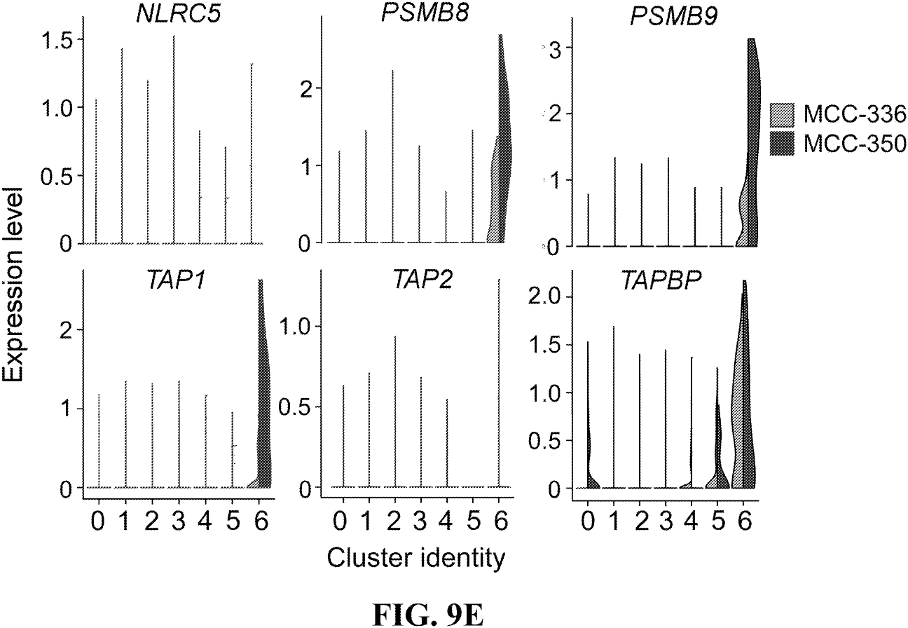

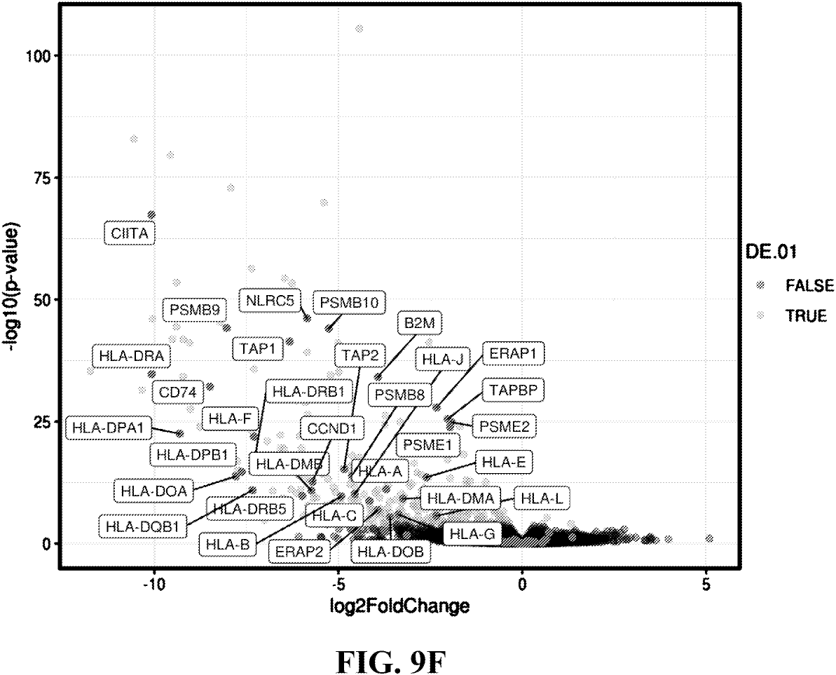

To elucidate the mechanisms of impaired HLA I surface expression in the MCC lines, Applicants performed an in-depth genomic and epigenomic characterization for a subset of both virus-positive and -negative lines for which Applicants had material available (Table 3). To define the alterations in gene expression in MCC after IFN-γ exposure, Applicants evaluated transcript expression in all 11 MCC lines at baseline and after IFN-γ stimulation. Applicants further compared the expression of the MCC lines to epidermal keratinocytes and dermal fibroblasts (Butterfield et al. 2017; Swindell et al. 2017), since they are leading candidates for the cell-of-origin of MCPyV- and MCPyV+ MCC, respectively (Sunshine et al. 2018), and both reside within the skin. At baseline, the MCC lines exhibited low mRNA expression of several class I pathway genes, most notably HLA-B, TAP1, TAP2, PSMB8, and PSMB9, with a generally similar expression profile in MKL-1 and WaGa, two well-studied MCC lines (FIG. 2A). IFN-γ treatment markedly upregulated class I genes in 11 of 12 MCC lines, a trend which was confirmed in matched proteomes in 4 MCC lines (FIG. 2B). MCC lines that were non-IFN-responsive by flow cytometry (FIG. 1D) exhibited variable defects, such as lack of IFN-induced HLA-A, -B, and -C mRNA upregulation (MCC-336) and lack of IFN-induced STAT/p-STAT protein expression (FIG. 2A; FIG. 9B; FIG. 9C),

To investigate the degree of heterogeneity in the HLA I downregulation observed in bulk transcriptome sequencing of MCC cells, Applicants evaluated HLA expression on 2 fresh MCC biopsies (MCC350 [MCPyV-] and MCC336 [MCPyV+]) by high-throughput droplet-based single-cell transcriptome analysis. Reads from both samples were aligned to hg19 using Cellranger, and transcript quantities were analyzed using the Seurat pipeline (see Methods). Following sample QC, the cells were grouped using Louvain clustering. From the genes identified across the two samples, 7 distinct transcriptionally defined clusters were detected. Immune cells, identified by CD45 expression, comprised cluster 6, while clusters 0-5 were MCC cells, identified by the expression of SOX2, SYP, and ATOH1 (FIG. 2C; FIG. 9D). All MCC clusters displayed nearly absent HLA-B, TAP½, PSMB8/9, and NLRC5 expression and low HLA-A and -C expression (FIG. 2C; FIG. 9E), consistent with the aforementioned bulk characterization of surface HLA I expression in MCC cell lines. By contrast, cluster 6 (immune cells) displayed higher expression of HLA class I transcripts.

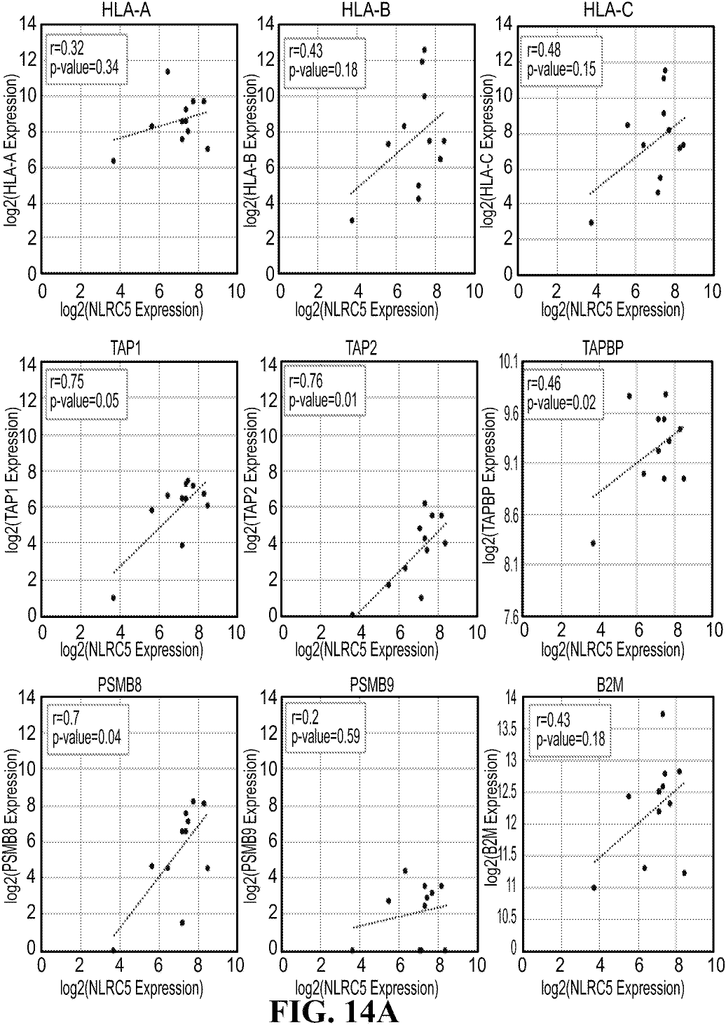

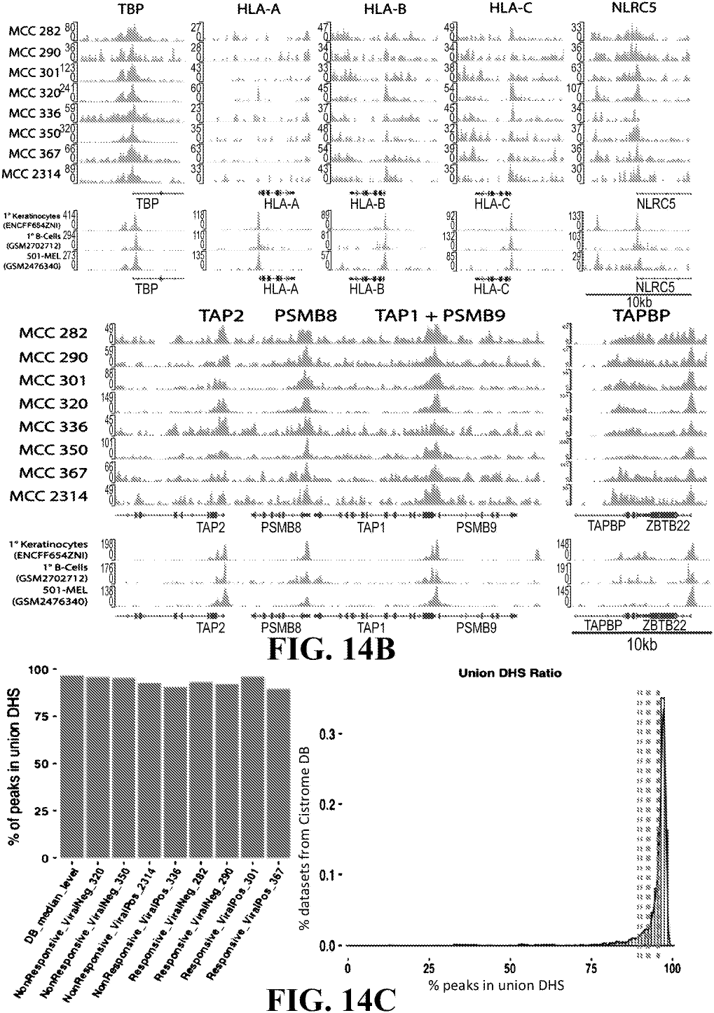

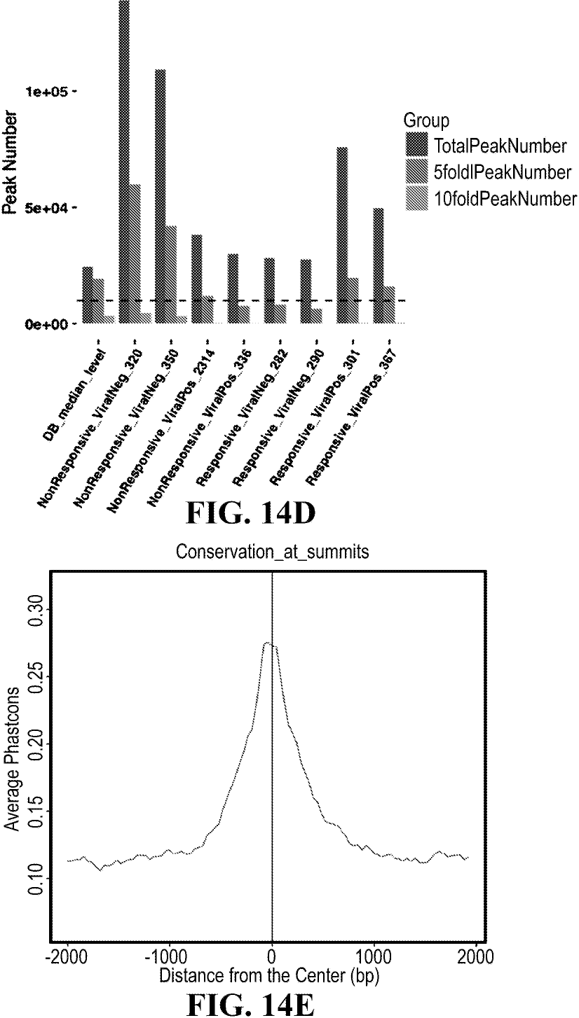

Given the marked RNA- and protein-level downregulation of multiple class I genes, Applicants first sought to identify a possible genetic basis for these observations. By WES, none of the MCC lines harbored any notable somatic mutations in 17 canonical HLA I pathway genes with the exception of an HLA-F mutation in MCC320 (Table 2). For the 3 non-IFN-γ-responsive lines (MCC-336, -350, -358), no mutations in the key interferon signaling pathway genes (IFNGRs, JAKs, STATs) were found (FIG. 9F). However, loss of NLRC5 was detected in 5 of 8 lines for which copy number variation analysis was performed (FIG. 2D). NLRC5 is a transcriptional activator of several HLA I pathway components (i.e., HLA-A, -B, -C, -E, -F, B2M, TAP1, and PSMB9 (LMP2) (Vijayan et al. 2019) that localizes to conserved S/X/Y regions in the promoters of these genes, and Applicants observed positive correlation between NLRC5 and these other class I genes in the MCC lines (FIG. 14A). By analysis of matched whole-genome bisulfite sequencing, Applicants also detected NLRC5 promoter hypermethylation compared to other class I antigen presentation genes (FIG. 2E), suggesting an additional mechanism by which NLRC5 might be suppressed in MCC. Consistent with these observations, NLRC5 copy number loss and promoter methylation have been recently recognized as a common alterations across diverse cancers (Yoshihama et al. 2016). ATAC-seq data generated from 8 of the MCC cell lines were benchmarked against datasets on Cistrome DB (ref PMID: 30462313) for quality control (FIGS. 14C-D) and revealed inaccessible chromatin and lack of clear promoter peaks at the transcription start site of several class 1 genes, including HLA-A, HLA-B and to a lesser extent HLA-C and NLRC5 (FIG. 14B), providing further evidence of epigenetic downregulation of class 1 expression.

### IFN-γ-induced HLA I Upregulation Is Associated With Shifts in the HLA Peptidome

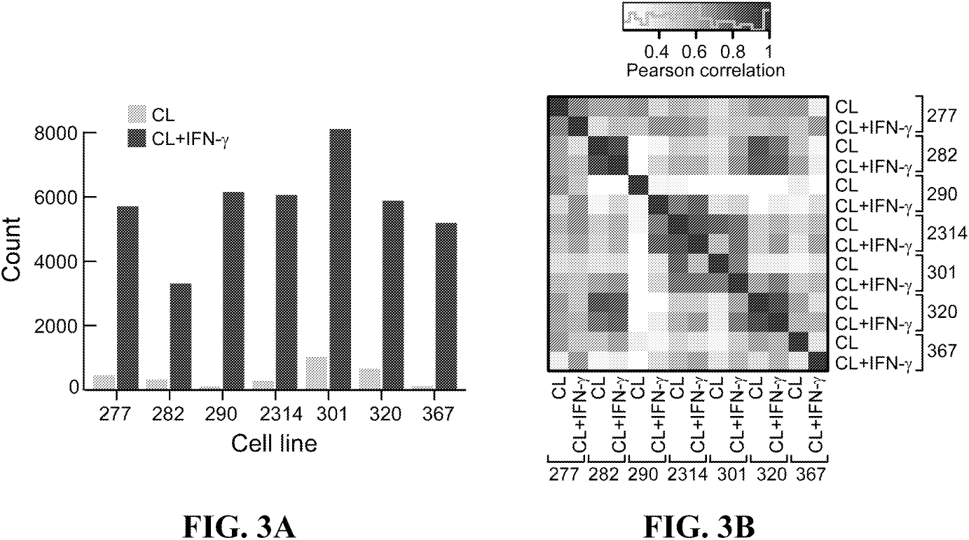

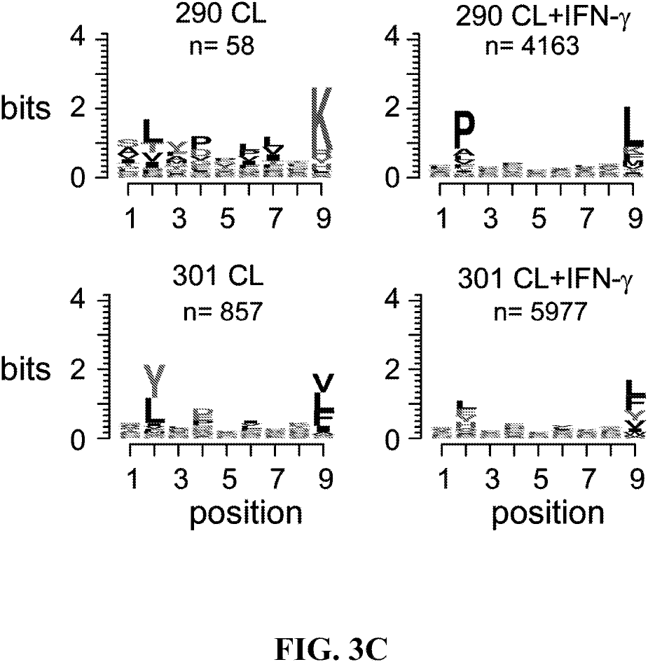

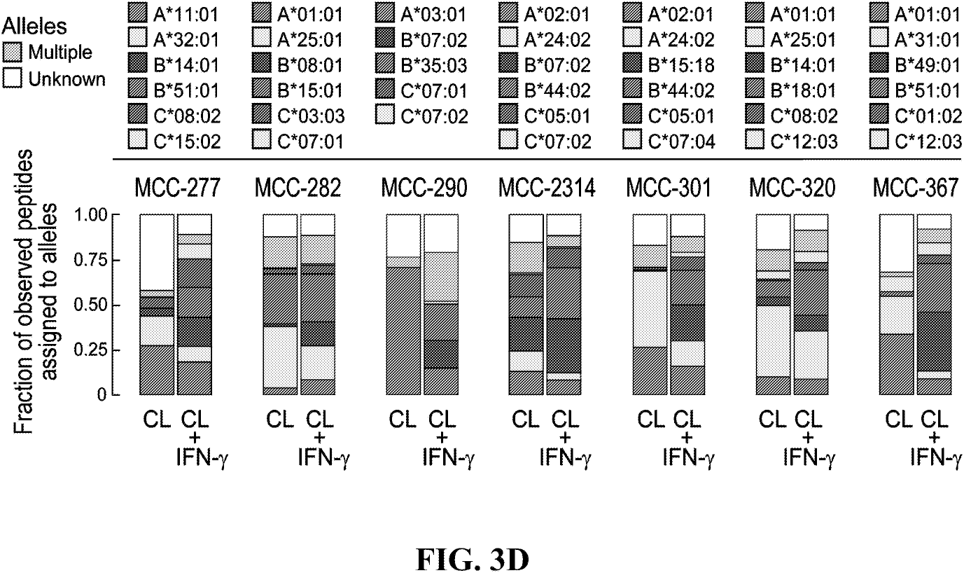

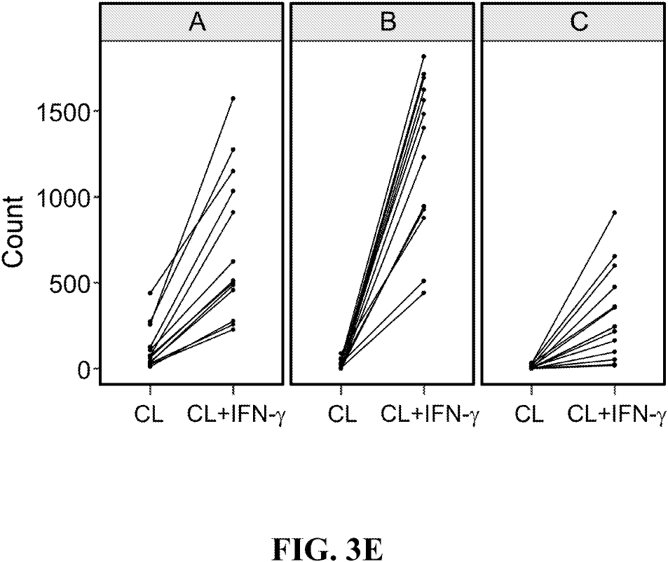

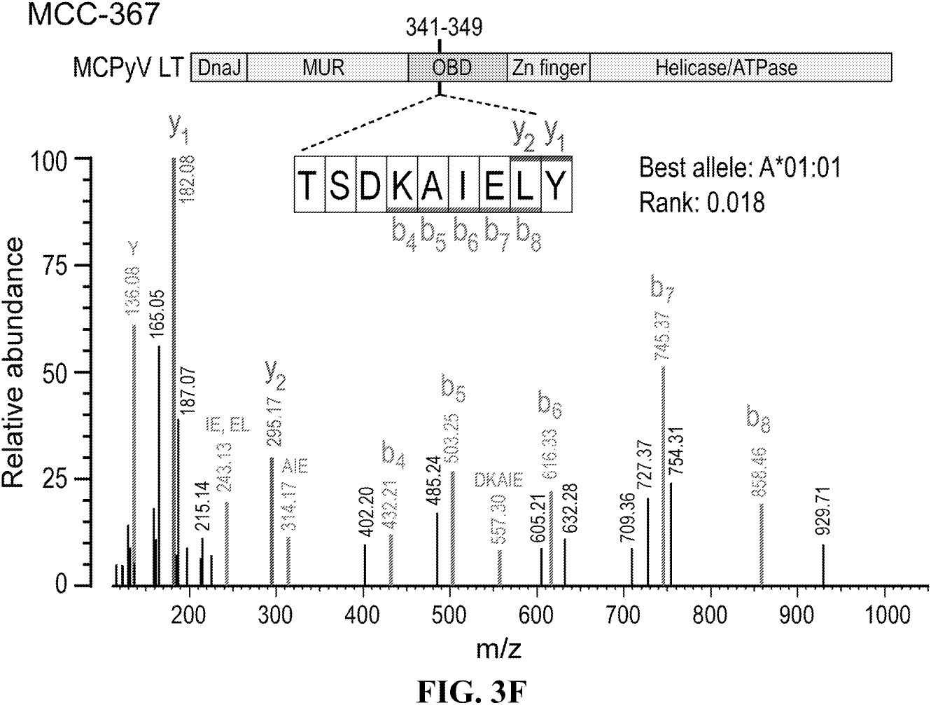

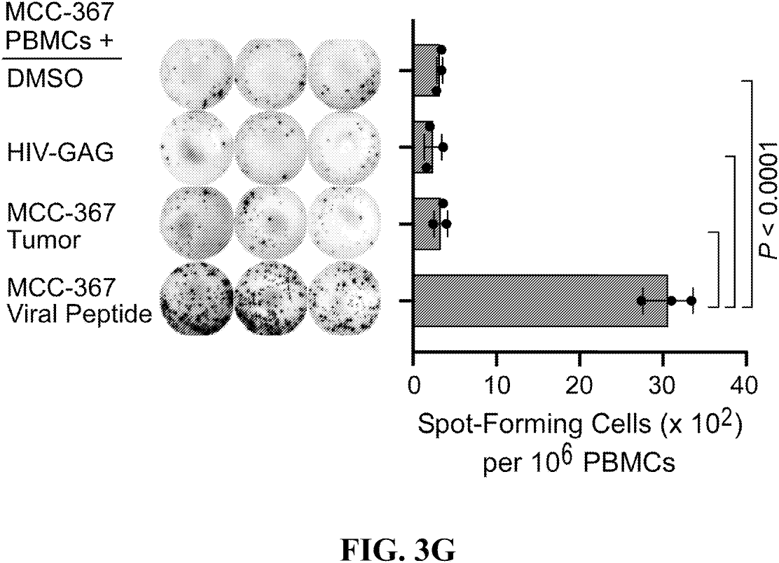

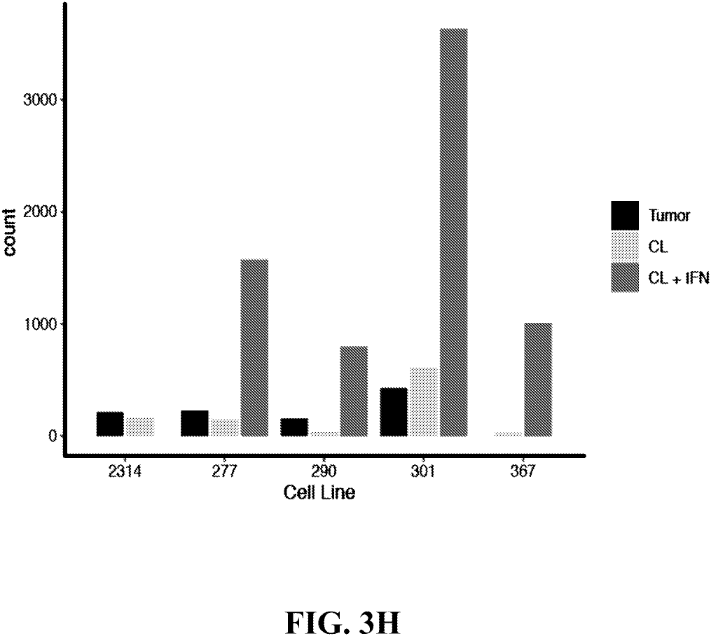

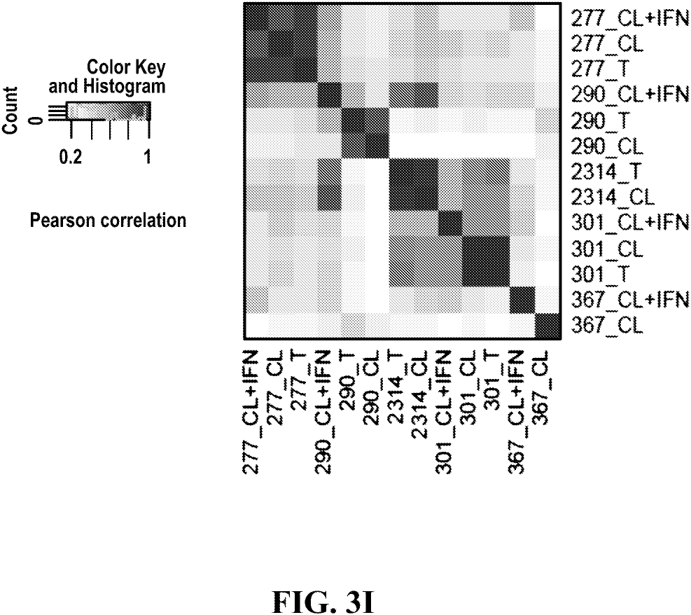

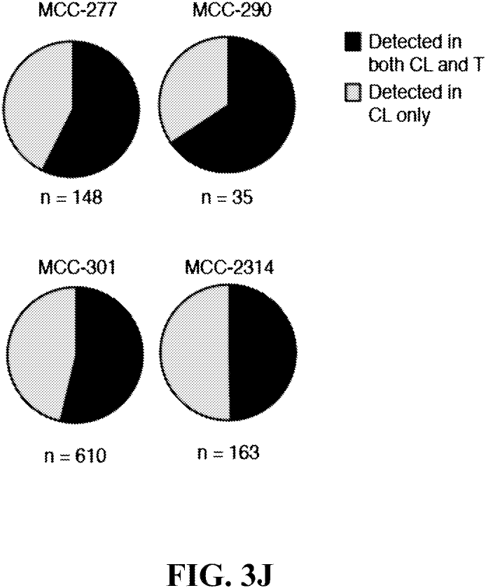

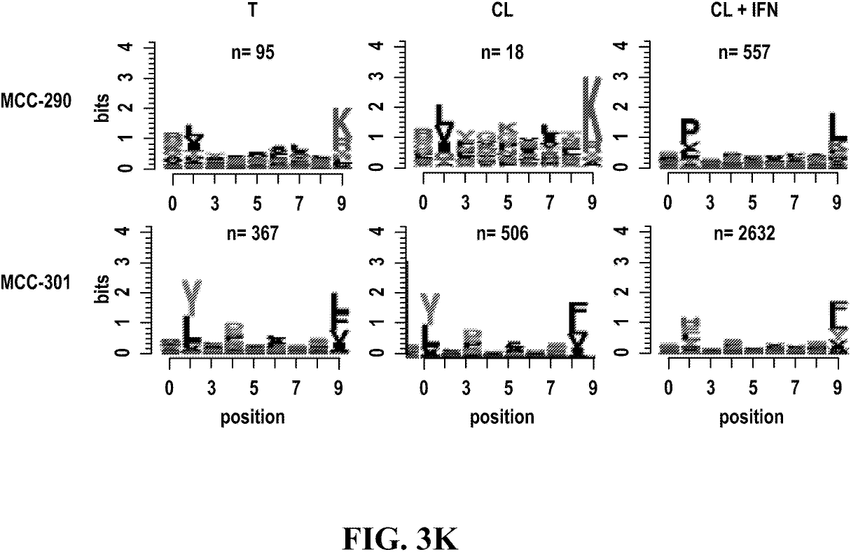

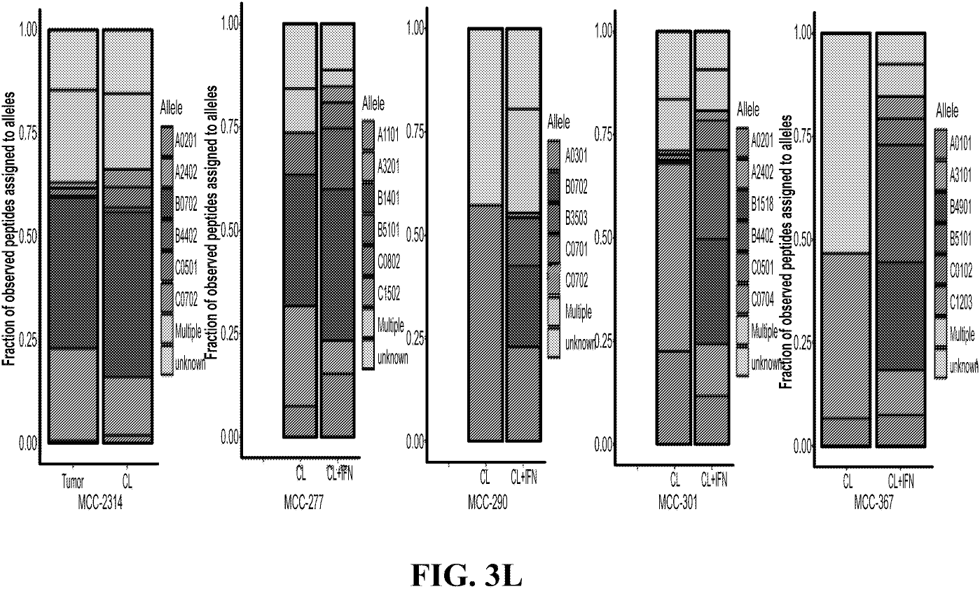

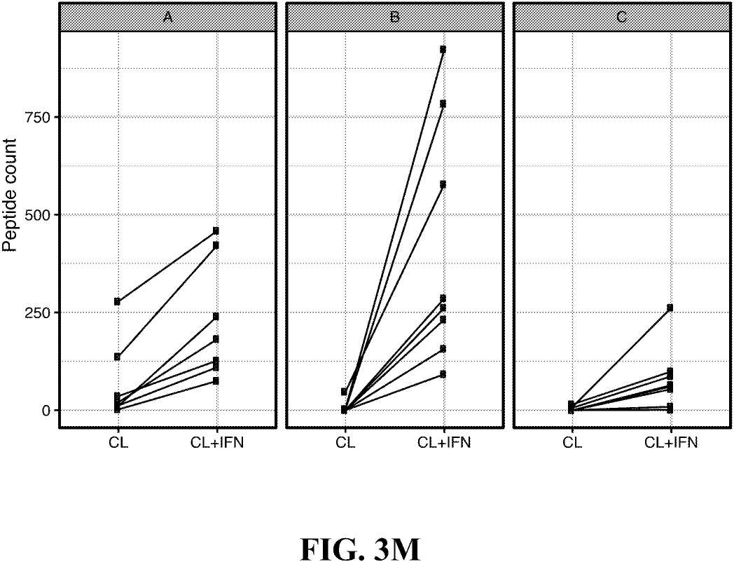

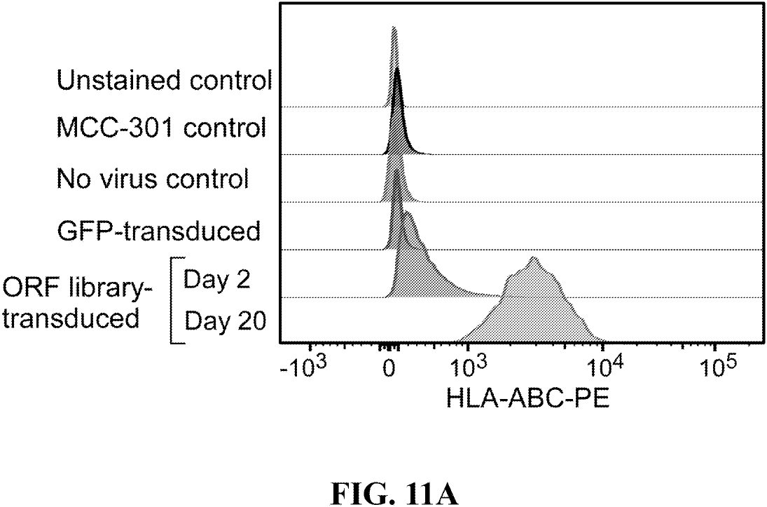

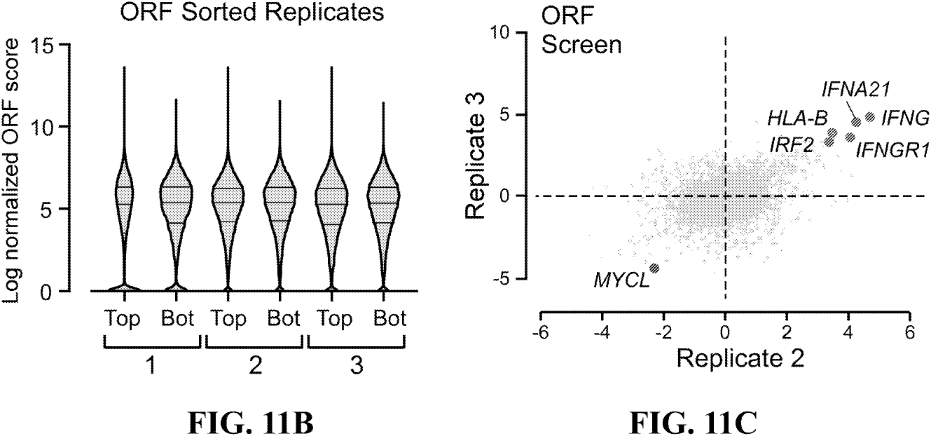

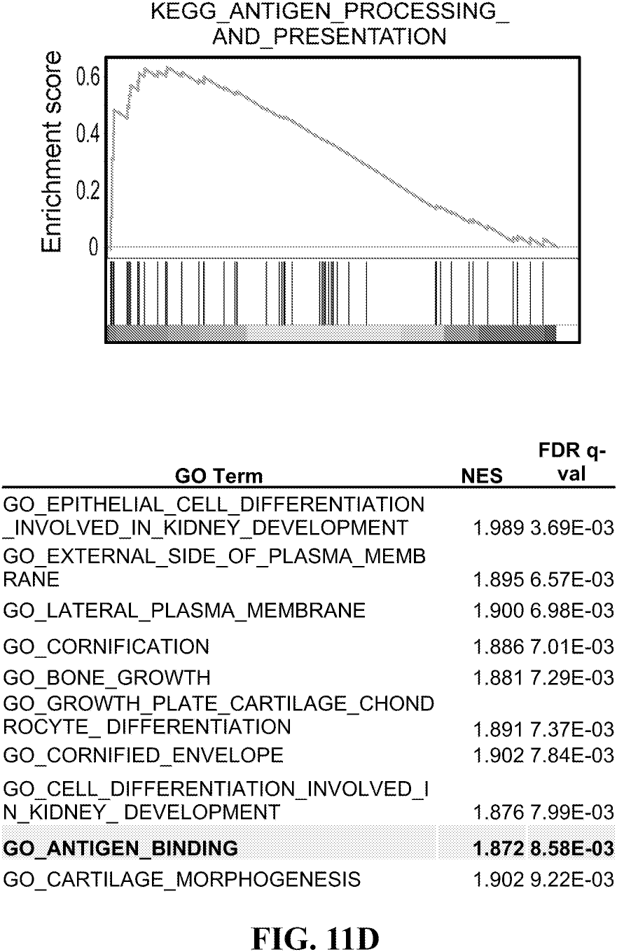

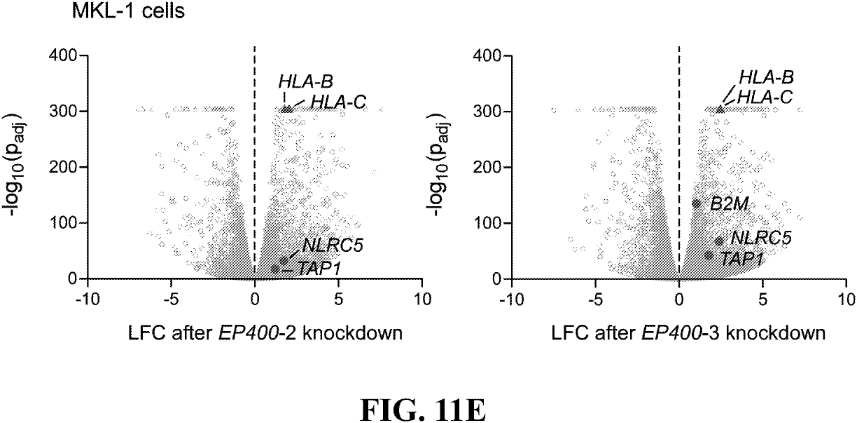

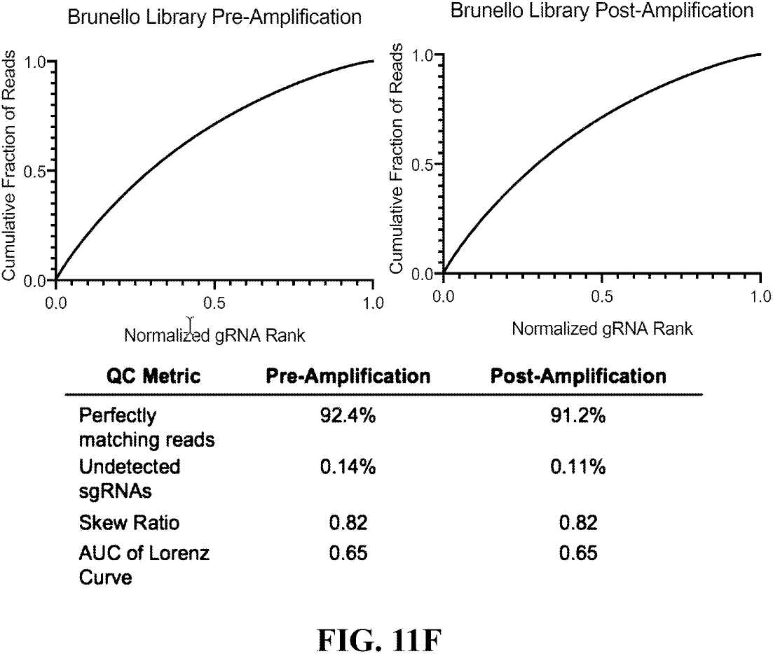

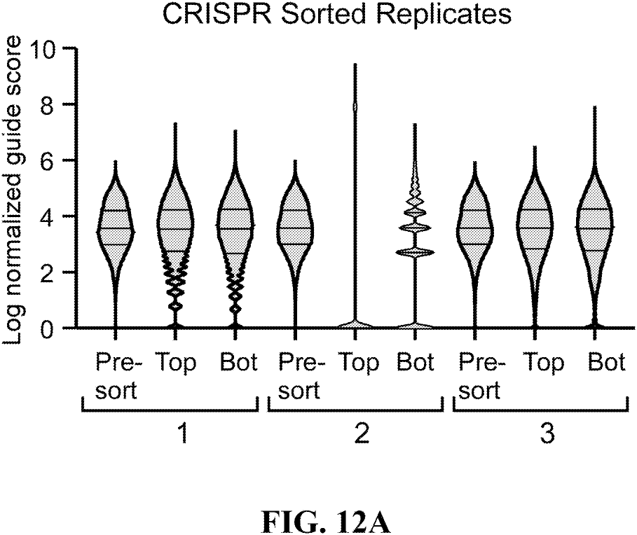

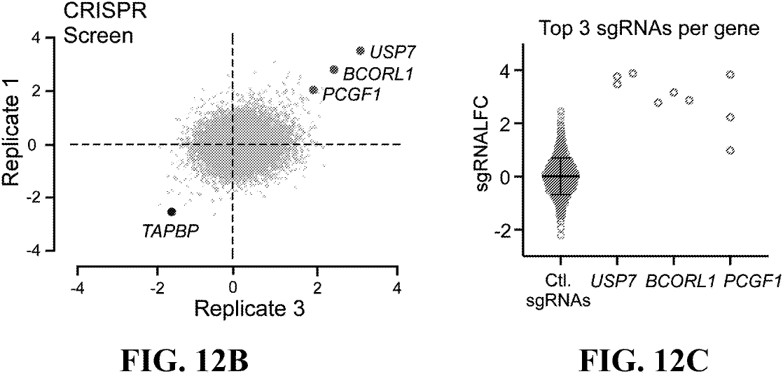

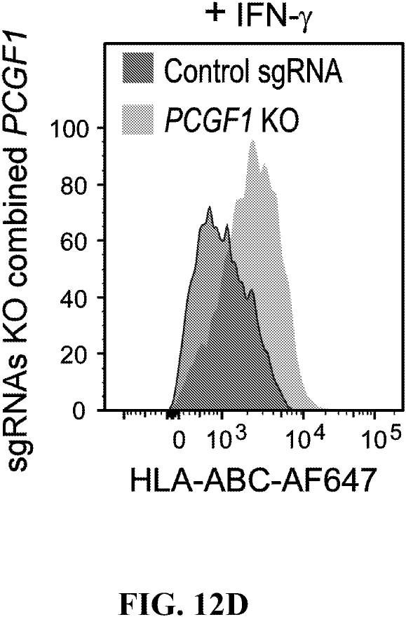

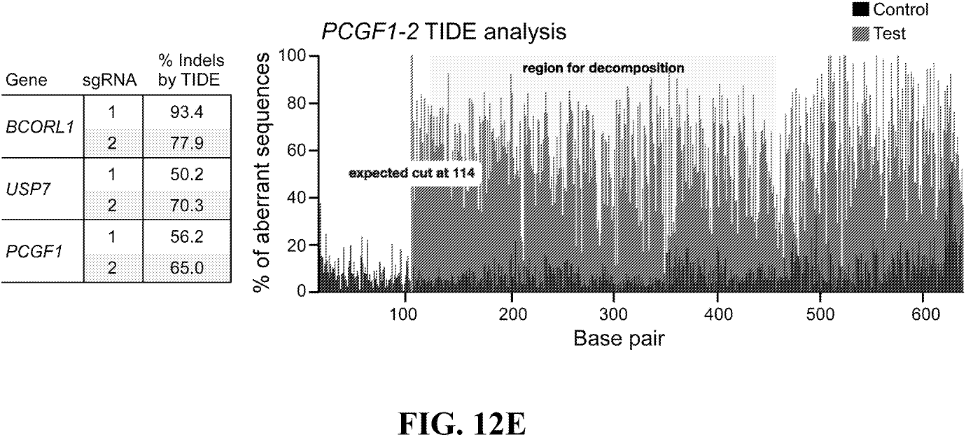

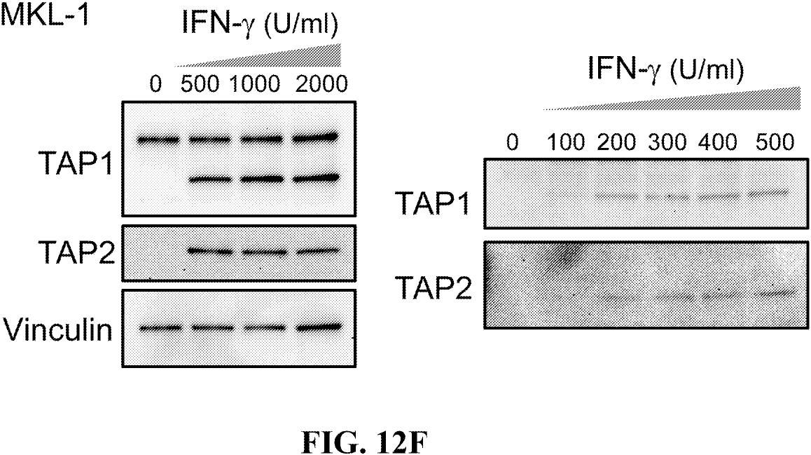

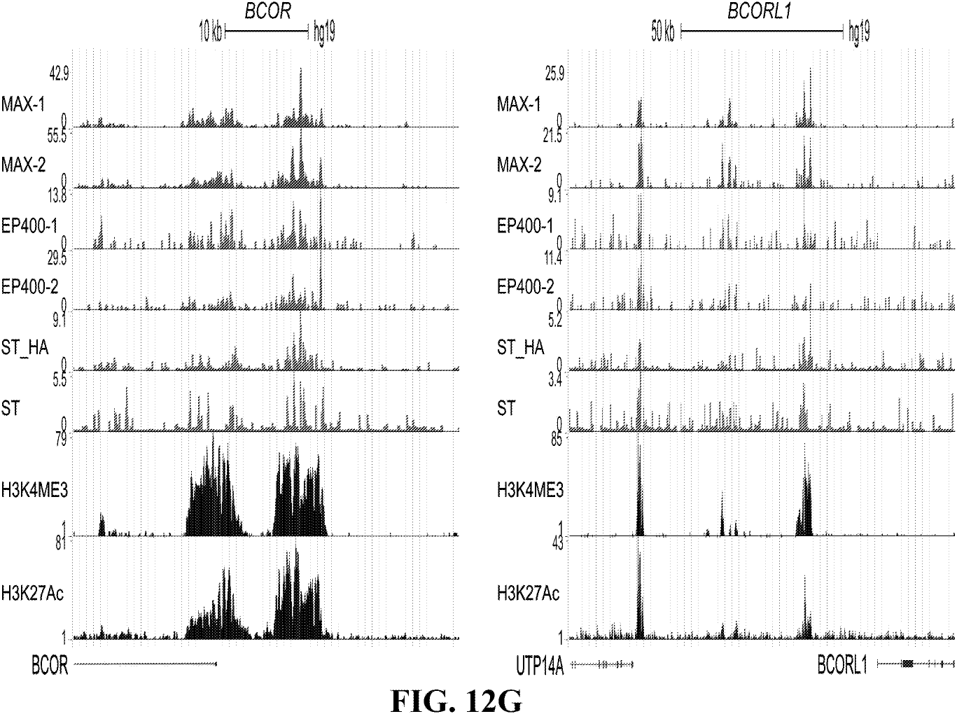

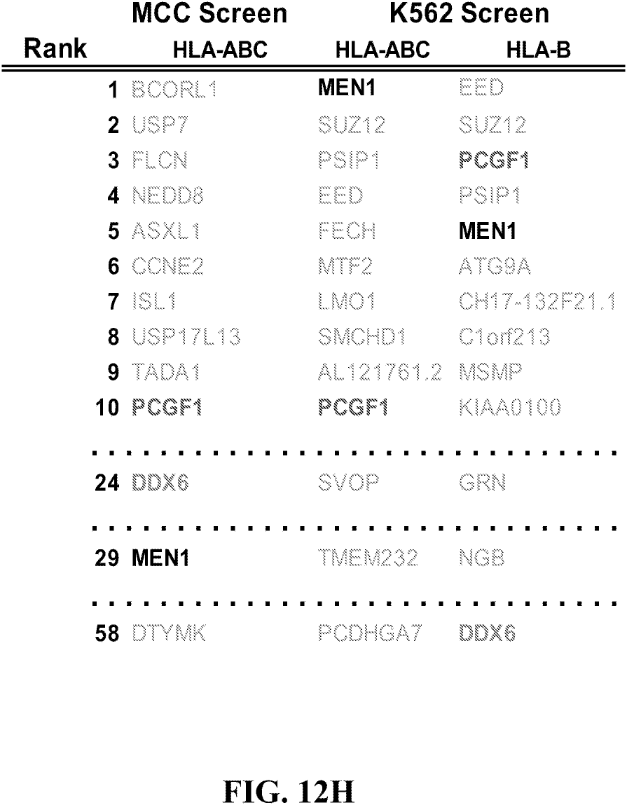

Diminished expression of HLA I would be expected to result in a lower number and diversity of HLA-presented peptides in MCC, impacting the immunogenicity of the tumor. Indeed, using the standard workflows for direct detection of class I bound peptides by mass spectrometry, following immunoprecipitation of tumor cell lysates with a pan-HLA class I antibody (FIG. 11F; see Methods), Applicants detected similarly low total peptide counts at baseline in parental tumors and cell lines. Following IFN-γ stimulation, a median 25-fold increase in the abundance of class I bound peptides was detected across 4 cell lines that were thus treated (FIG. 3H). Whereas Applicants observed a high level of correlation in the immunopeptidomes between the tumors and cell lines at baseline, Applicants observed lower correlations between cell lines before and after IFN-γ treatment (FIG. 3I). To further explore these observations, Applicants inferred the most likely HLA-allele to which the identified peptides were bound. The inferred frequencies of peptides presented on each class I HLA allele were similar between corresponding tumors and cell lines, and cell line peptidomes shared more than 50% of their peptides with corresponding tumor peptidomes (FIG. 3J, FIGS. 11, 12). When comparing cell lines +/- IFN-γ, Applicants found that IFN-γ treatment not only resulted in increased surface expression of HLA and thus more peptides but also altered the peptide motif landscape (FIG. 3K). These shifts corresponded with dramatic changes in the frequencies of peptides mapping to each HLA allele, most notably an increase in HLA-B-presented peptides (FIGS. 3L, M). This is consistent with reports that interferons upregulate HLA-B more strongly than HLA-A (PMID: 8265591, 8530148).

For the MCPyV+ lines, Applicants hypothesized that this upregulation of HLA I following IFN-γ stimulation would lead to increased ability to present MCPyV-specific epitopes. For one such line, MCC-367, Applicants detected an HLA*A1:01-restricted class I epitope prediction (TSDKAIELY (SEQ ID NO:1); rank per HLAthena) derived from LT, which was detected only after IFN-γ treatment and not at baseline (FIG. 3F).

### Complementary Genome-scale Loss- and Gain-of-Function Screens Identify Known and Novel Potential Regulators of HLA I Expression in MCC

Although Applicants identified NLRC5 copy number loss and promoter methylation as a contributory factor in enforcing the silencing of the HLA I pathway, Applicants observed that at least three lines (MCC-290, -301, -320) exhibited normal NLRC5 copy number and had low levels of HLA I expression. Hence, Applicants sought to identify alternative pathways and mechanisms underlying the high degree of HLA I surface loss and downregulation of multiple class I components.

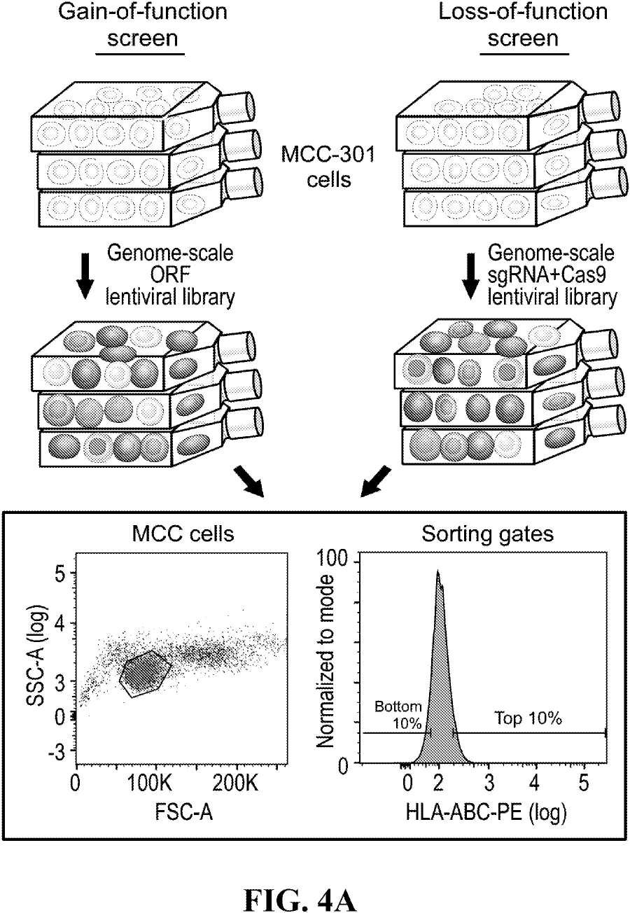

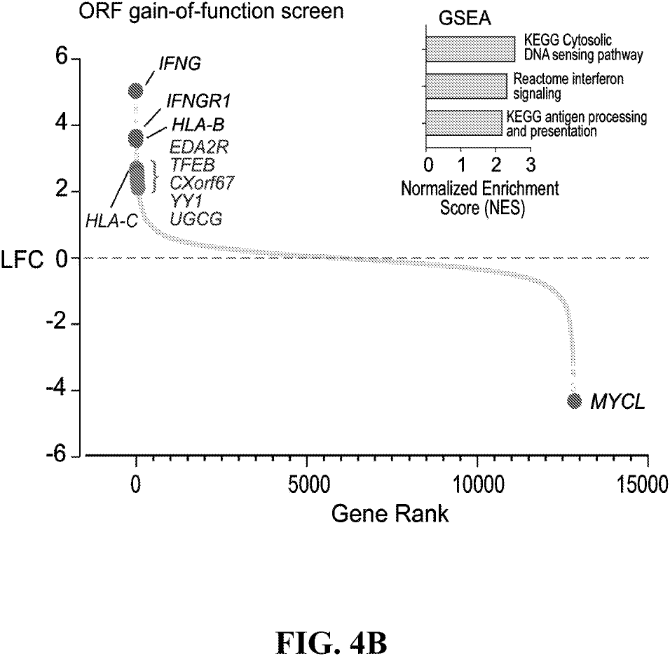

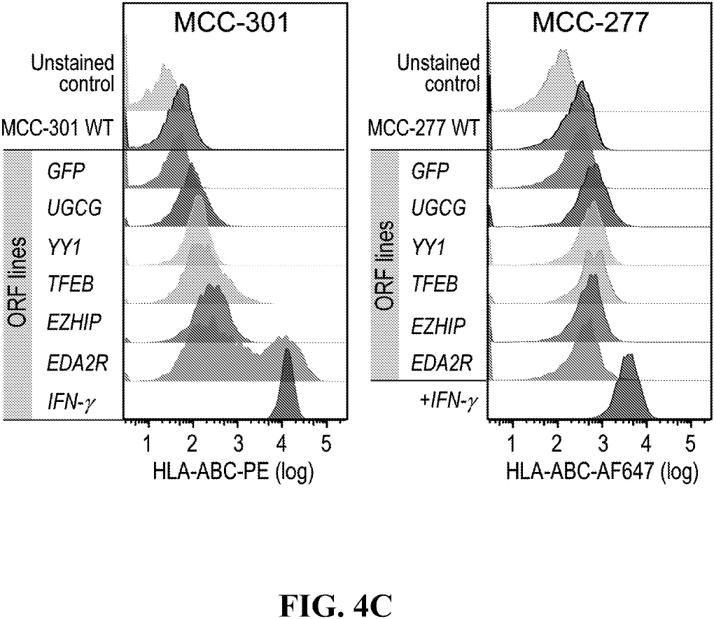

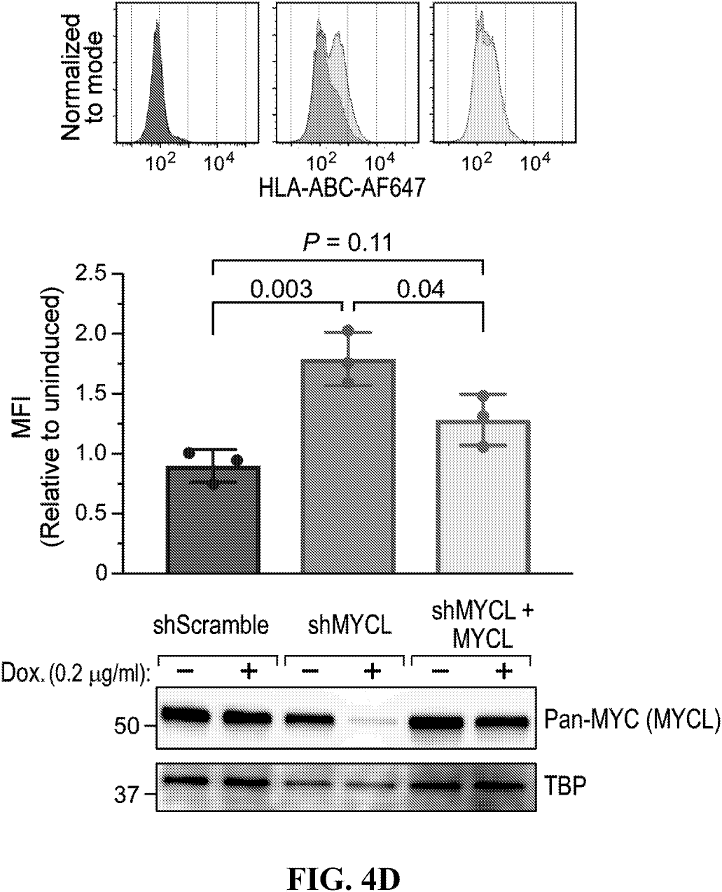

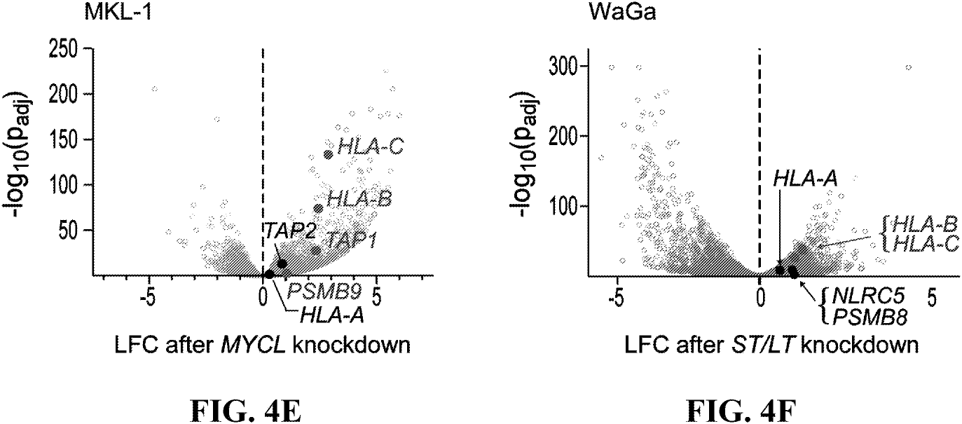

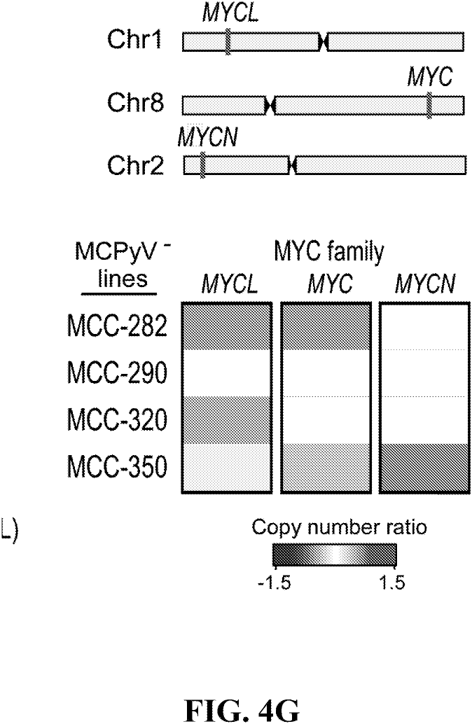

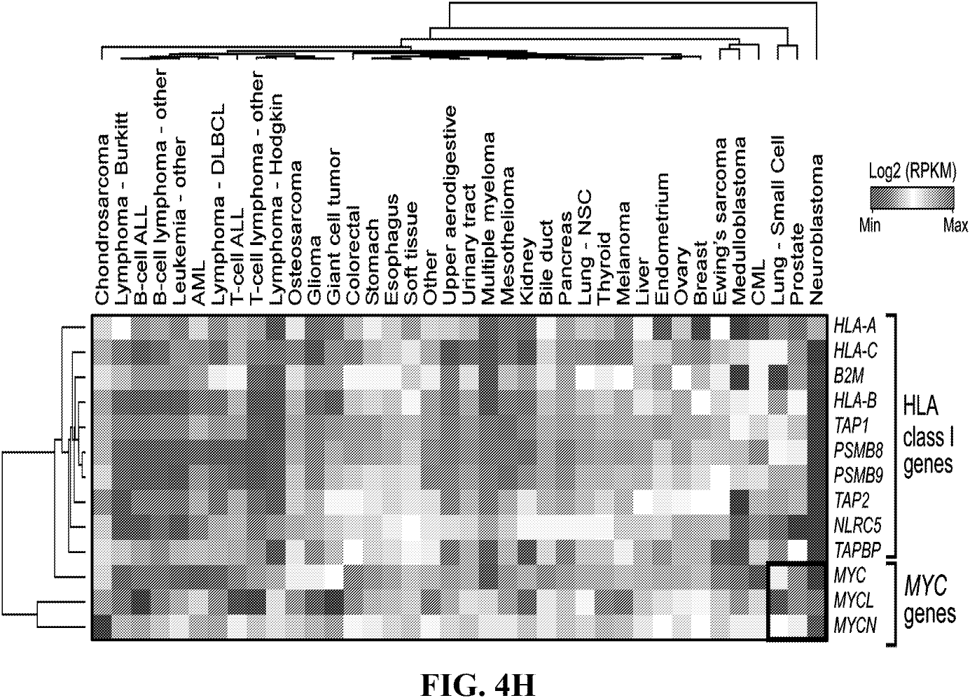

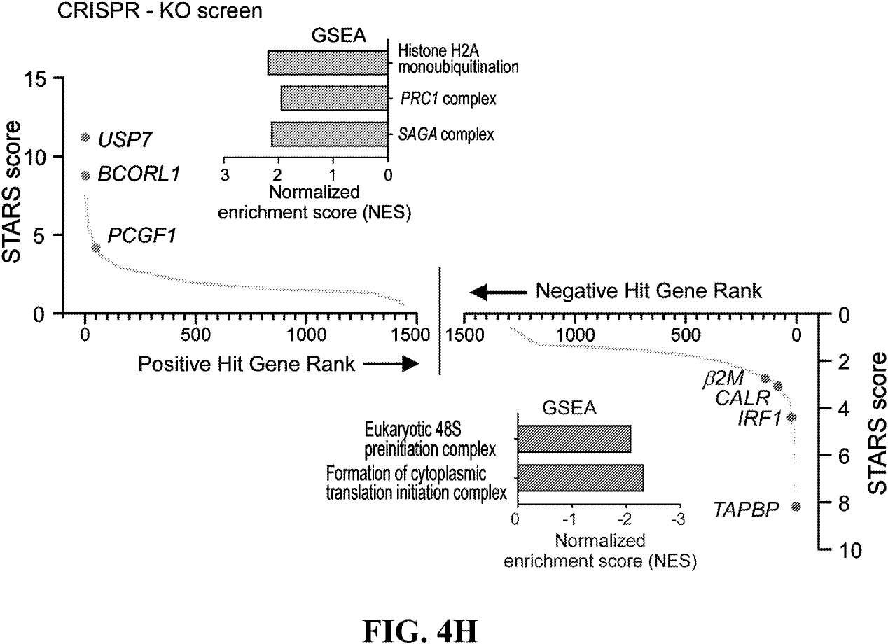

To this end, Applicants designed paired genome-scale CRISPR-KO loss-of-function and open reading frame (ORF) gain-of-function screens to systematically identify novel regulators of HLA I surface expression in MCC. These screens were conducted in the virus-positive MCC-301 line due to its robust growth rate, but also because of its low mutational background, enabling focusing on the role of deregulated genes. Applicants also hypothesized that the novel impacted pathways identified in this MCPyV+ context would be mirrored in MCPyV- MCC, wherein HLA I suppression might be achieved through somatic mutations affecting these same pathways. In brief, MCC-301 cells were transduced at a low multiplicity of infection (MOI) with genome-wide lentiviral libraries containing either ORF or Cas9+sgRNA constructs. After staining cells with an anti-HLA-ABC antibody, the HLA I-high and HLA I-low populations were isolated by flow cytometry-based cell sorting, with each screen performed in biologic triplicate (FIG. 4A). Of note, transduction with the ORF library but not the CRISPR library led to a population-wide increase in HLA I surface expression, presumably due to interferon secretion from interferon-related gene ORF-expressing cells. Applicants confirmed this was an ORF library-specific effect and not due to the process of lentiviral transduction, as GFP-transduced cells did not exhibit an increase in surface HLA I (FIG. 11F). To perform gene-level ranking, for the ORF screen Applicants used the median construct log2-fold change from 3 replicates, while for the CRISPR screen, Applicants discarded one replicate which had poor sample quality and averaged the remaining two high-quality replicates (see Methods).

The ORF screen produced 75 hits with a greater than twofold increase in median log2-fold change (enrichment in HLA I-high vs HLA I-low). As expected, these hits were highly enriched for interferon and HLA I pathway genes by Gene Set Enrichment Analysis (GSEA) (Subramanian et al. 2005) (FIG. 4B). The top hit was IFNG, with an additional five of the top 15 in interferon signaling pathway genes. In addition, HLA-B and -C were hits #9 and #37, respectively. Strikingly, MYCL was found to be the top negative hit (FIG. 4B). MYCL is a central transcription factor in MCPyV+ MCC, as ST binds and recruits MYCL to the EP400 chromatin modifier complex to enact widespread epigenetic changes necessary for oncogenesis (Cheng et al. 2017b; Park et al. 2019, 2020).

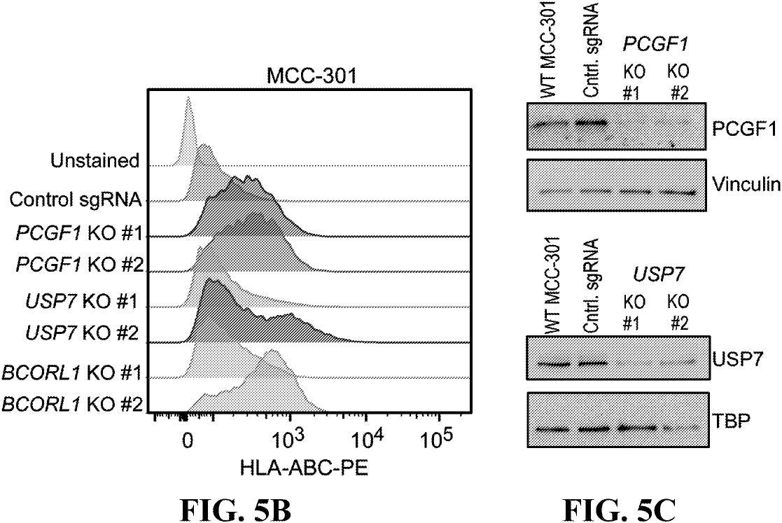

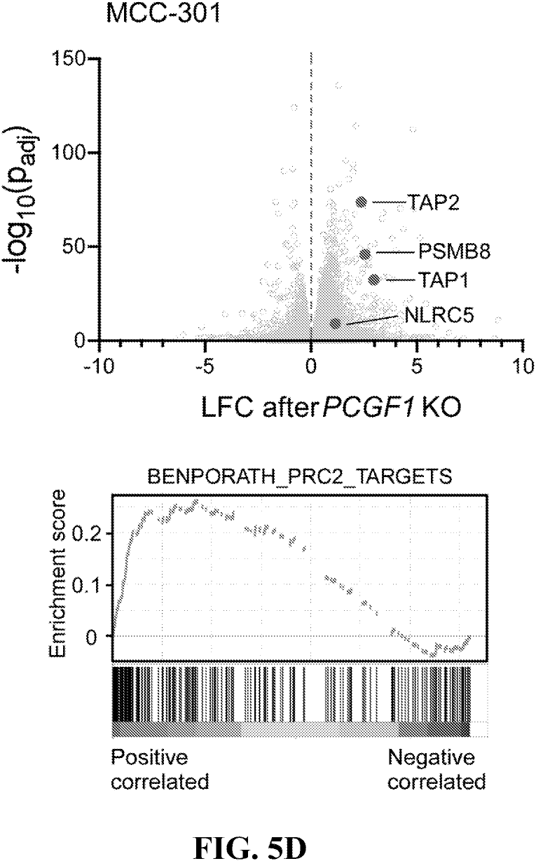

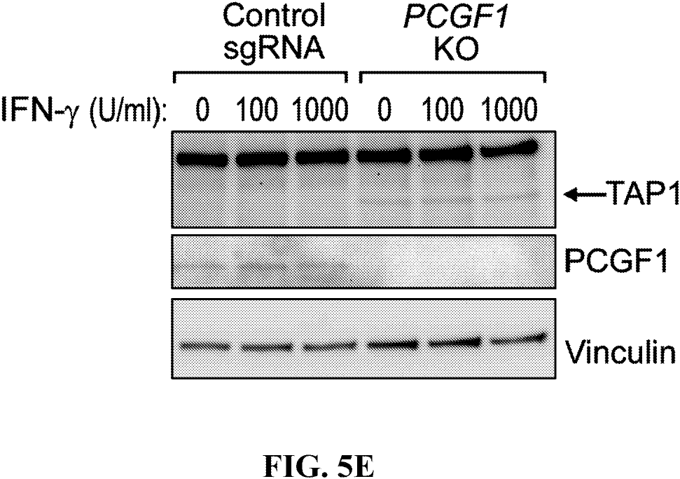

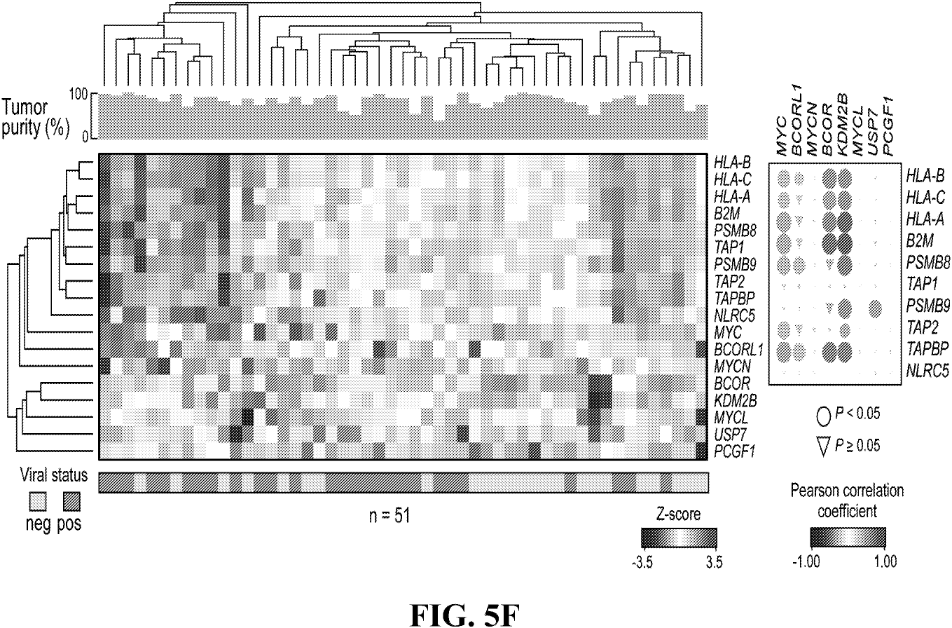

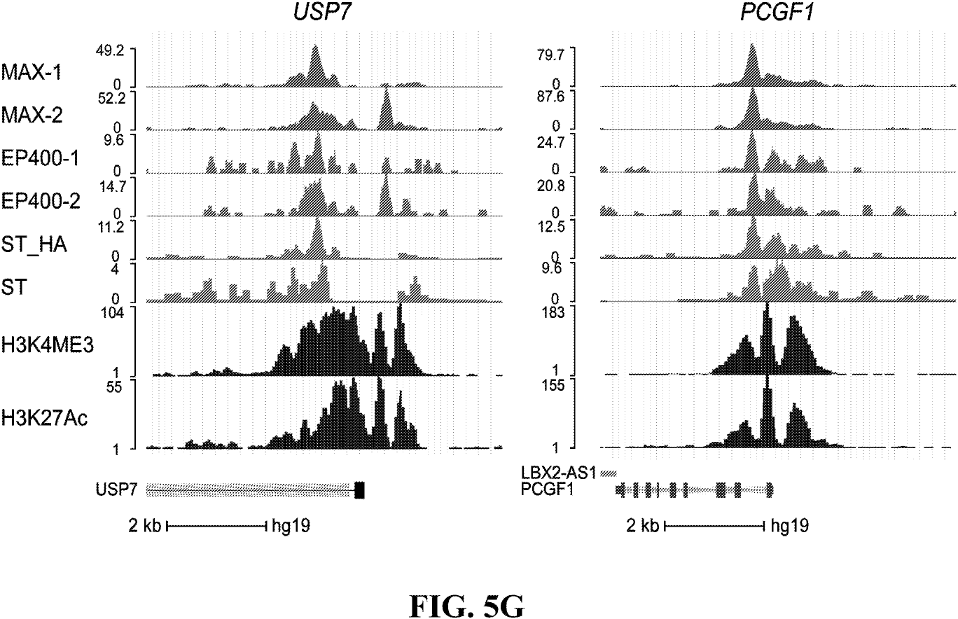

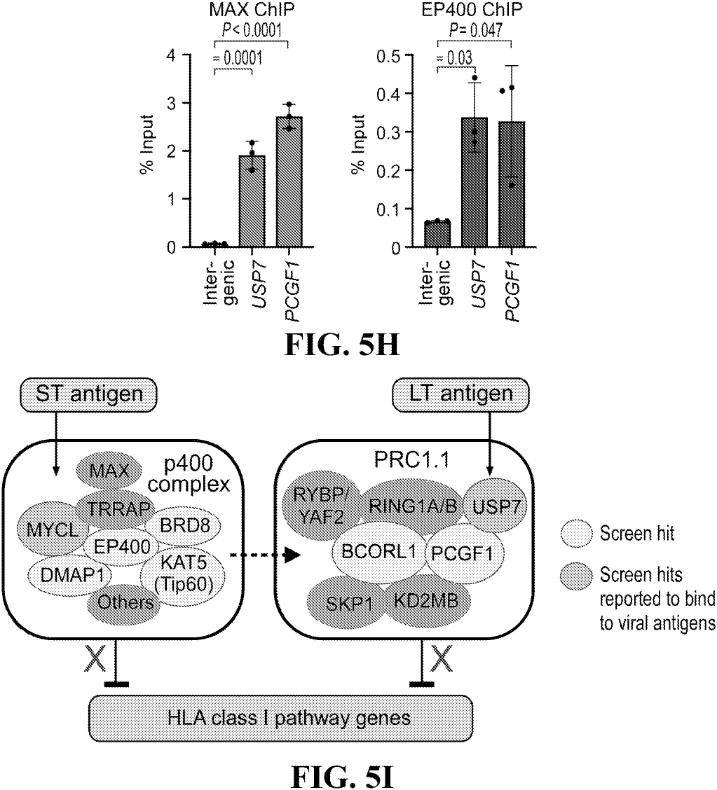

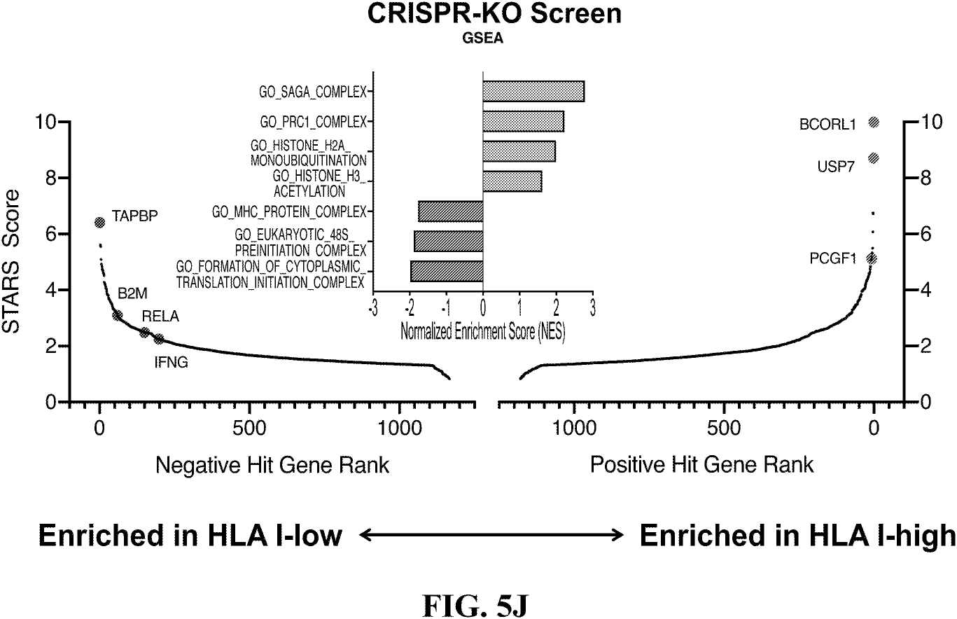

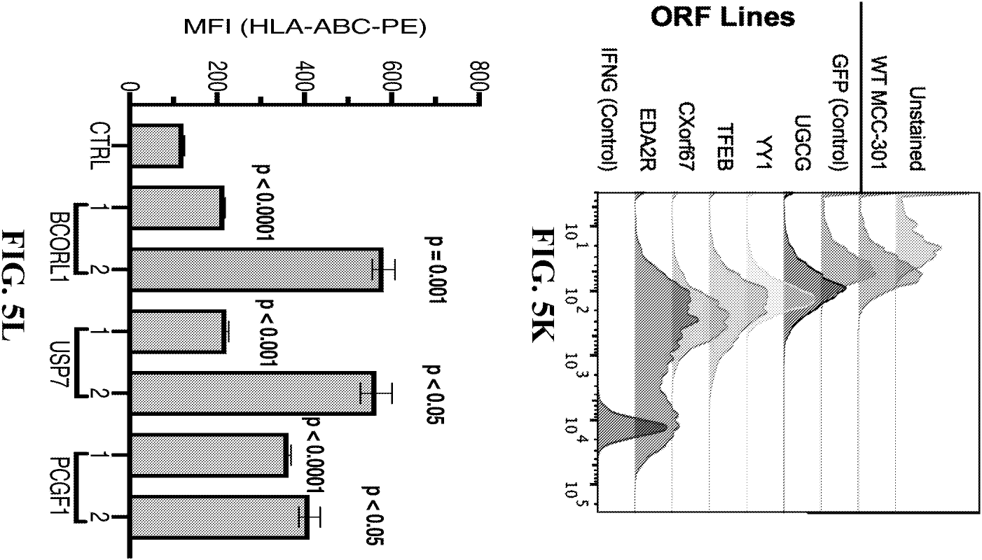

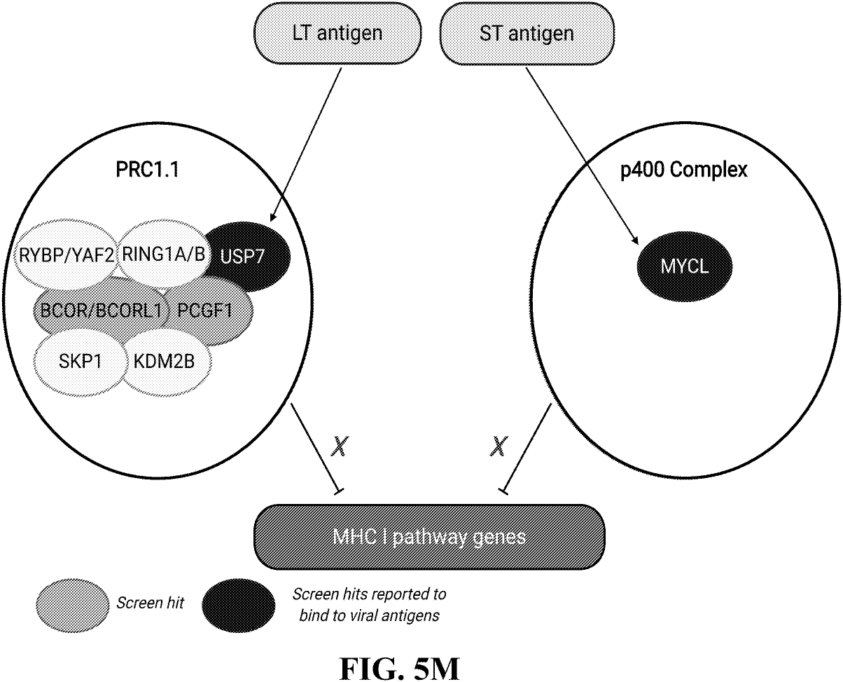

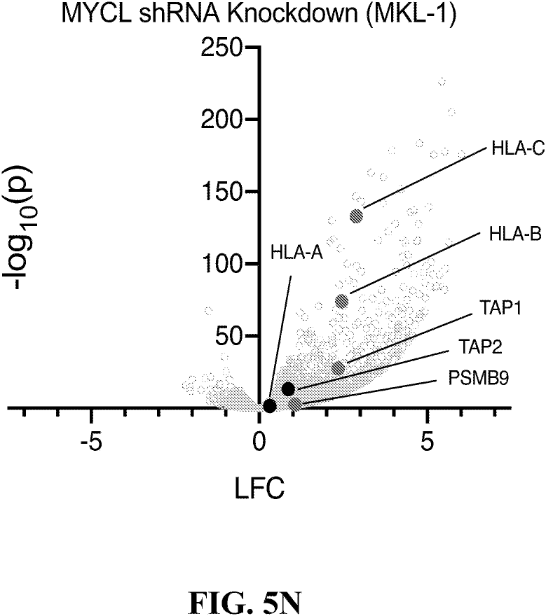

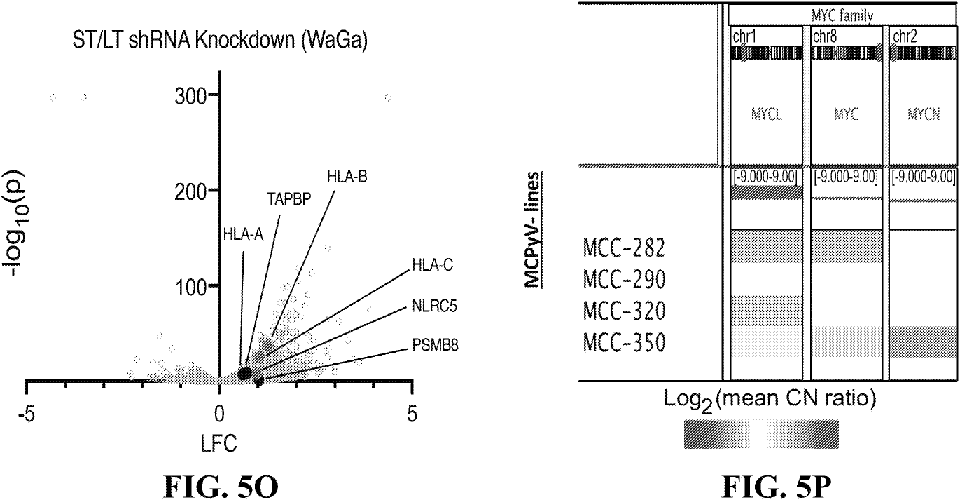

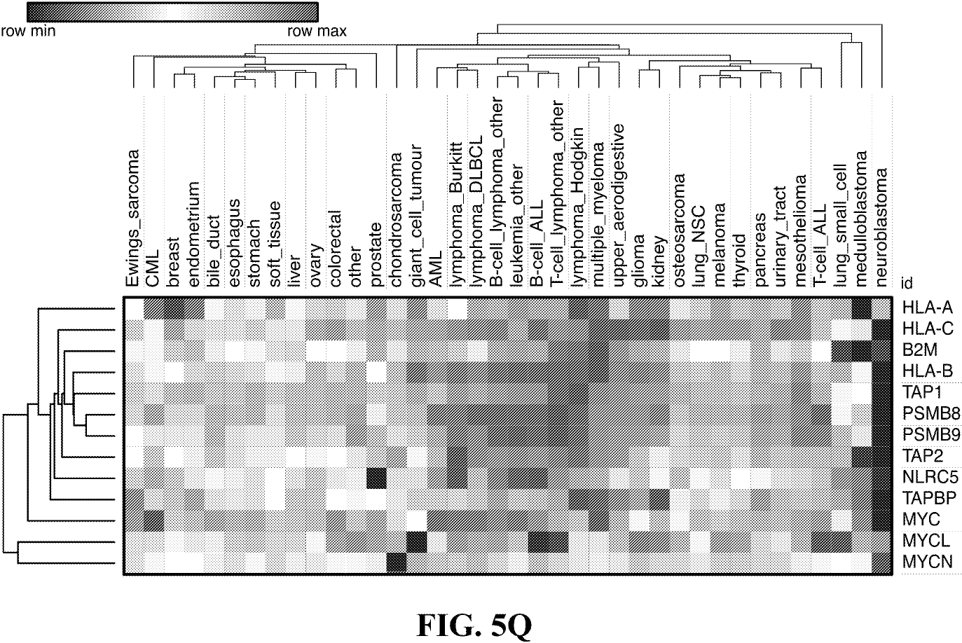

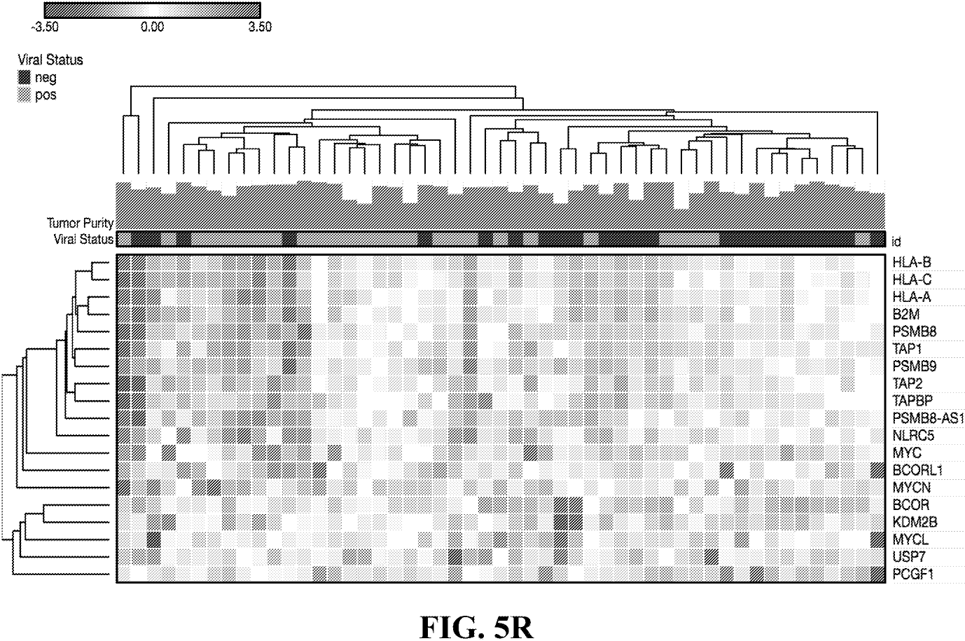

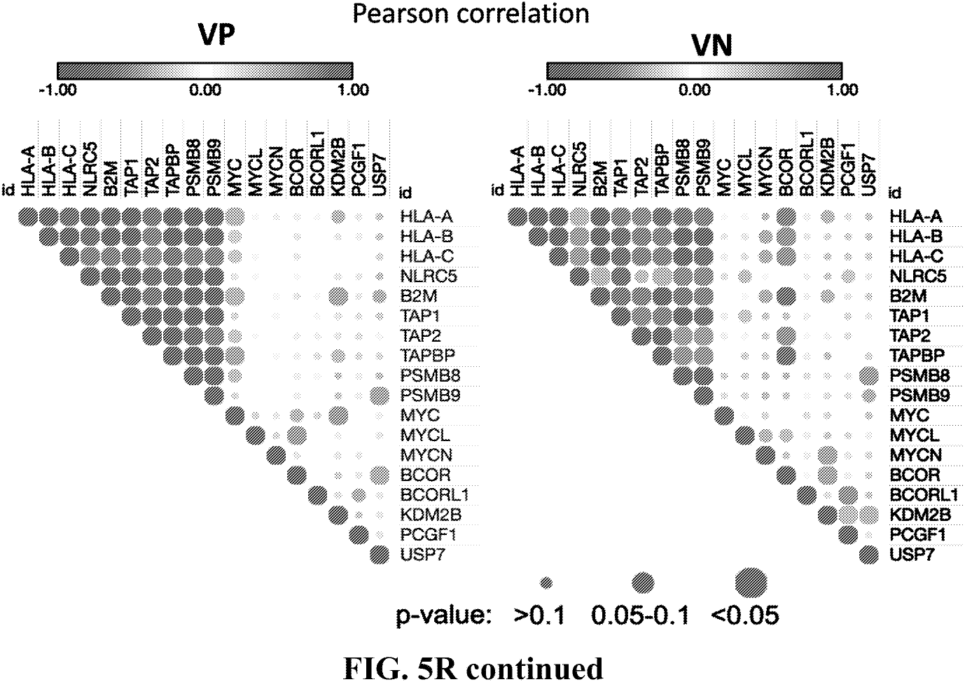

The CRISPR-KO screen also identified several known components of the HLA class I pathway. Sequencing of the CRISPR library-transduced cells prior to FACS confirmed that adequate sgRNA representation was present (FIGS. 11B, 12A). Positive and negative hits were then ranked according to the STARS algorithm (Doench et al. 2016). The top negative hit (gene whose knockout resulted in the highest enrichment in the HLA I-low population) was TAPBP (FIG. 5J), a key class I pathway component that acts as a chaperone for partially folded HLA I heavy chains and facilitates binding between unbound HLA I and TAP. Applicants also identified other notable negative hits including class I genes B2M and CALR and IFN pathway gene IRF1. GSEA showed enrichment for expected gene sets (‘GO_HLA_PROTEIN_COMPLEX’), as well as gene sets related to protein translation. Of the CRISPR positive hits, Applicants recurrently identified several components of the Polycomb repressive complex PRC1.1, including the top two hits of the screen: BCORL1 (#1), USP7 (#2), and PCGF1 (#46). For each, Applicants observed >4.5-fold enrichment for at least 2 sgRNAs of these genes. PRC1.1 is a noncanonical Polycomb repressive complex that silences gene expression through ubiquitination of H2AK119 in CpG islands. In addition to the screen hits, other components of the PRC1.1 complex include KDM2B, SKP1, RING1A/B, RYBP/YAF2, and BCOR (interchangeable with BCORL1) (van den Boom et al. 2016).

Applicants first confirmed that the notable positive and negative hits in both screens exhibited high concordance between at least 2 replicates. Then, to validate ORF screen positive hits, Applicants generated single ORF overexpression lines in MCC-301, focusing on the top 71 hits not related to interferon or HLA I pathways. By flow cytometry, Applicants validated that 8 of 71 candidate hits (11.3%) upregulated MFI (HLA-ABC) by greater than 2-fold compared to GFP-transduced control while also maintaining viability after transduction, including TFEB, CXorf67, and YY1 (FIG. 5K). Within the ORF negative hits, Applicants chose to validate MYCL in the HLA I-positive IMR90 fibroblast line instead of MCC-301, since the putative suppressive effects of MYCL on HLA I might not be fully exemplified in the already HLA I-low MCC lines. Flow cytometry for surface HLA I in IMR90 fibroblasts expressing doxycycline-inducible MYCL showed MYCL-overexpressing IMR90 fibroblasts.

For the CRISPR screen, Applicants performed a targeted validation of top hits by generating a series of MCC-301 KO lines using the two highest-scoring sgRNAs against PRC1.1 components (BCORL1, PCGF1, USP7), ASXL1 (a Polycomb interacting protein), and FLCN (a negative regulator of validated ORF hit TFEB). Genome editing by Cas9 was confirmed by Sanger sequencing using TIDE (PMID: 25300484), and functional knockdown was confirmed by Western blot or qRT-PCR. Knockout of each gene increased surface HLA I expression by flow cytometry, relative to MCC-301 transduced with a control non-targeting sgRNA (FIG. 5L; FIG. 11D).

In aggregate, review of the top and bottom 100 hits across the parallel screens revealed 2 hits encoding proteins that have been reported to directly interact with MCPyV, MYCL (DeCaprio) and PRC1.1 component USP7 (31801860) (FIG. 5M). These were selected for more in-depth characterization.

### MYCL Mediates HLA I Suppression in MCC

After confirming that MYCL overexpression can reduce HLA I in the HLA I-high IMR90 fibroblast line, Applicants investigated if MYCL inactivation is sufficient to restore class I in an HLA I-low MCC line. Applicants introduced a MYCL shRNA into the MKL-1 cell line (MCPyV+) and performed flow cytometry and RNA-seq. Comparison of MYCL knockdown to a scrambled shRNA control and a >2-fold increase in expression of several class I genes including HLA-B, -C, and TAP1 (FIG. 5N). Based on this result and previous reports that ST binds and potentiates MYCL function through the ST-EP400-MYCL complex (29028833), Applicants hypothesized that viral antigen inactivation might also upregulate class I. After transducing the WaGa cell line (MCPyV+) with an shRNA that targets shared exons of ST and LT leading to inactivation of both MCPyV viral antigens, Applicants observed a similar but more modest upregulation of class I genes, including >1.5 fold increase in HLA-B, -C, and NLRC5 (FIG. 5O).

### MYCL Is Relevant to MCPyV- MCC and Other Cancers

To determine if the HLA I-suppressive effects of MYCL generalized to viral-negative MCC as well, Applicants evaluated the copy number status of MYCL in MCPyV- MCC. Copy number gain of chromosome, encompassing MYCL, was previously reported as one of the more common copy number alterations in MCC (Kelly G. Paulson et al. 2009). Indeed, 3 of the 4 virus-negative MCC lines exhibited some degree gain in MYCL copy number (FIG. 5P), suggesting a mechanism by which MCPyV- MCC may enhance MYCL signaling in the absence of viral antigens. To determine if this mechanism might be employed by other cancers, Applicants queried publicly available RNA-seq data from the Cancer Cell Line Encyclopedia (Ghandi et al. 2019). Cancer cell lines with lower expression of HLA I pathway components such as SCLC and neuroblastoma also frequently featured overexpression of MYCL and MYCN, respectively (FIG. 5Q).

Lastly, Applicants examined the association between expression of HLA class I genes and the screen hits in an RNA-seq cohort of 52 MCC tumors, including both MCPyV+ and MCPyV-. To account for the potential of immune cell infiltration confounding the bulk class I expression data, Applicants used ESTIMATE (Yoshihara et al. 2013) to calculate tumor purity. While MYCL was not associated with class I expression, Applicants did observe a negative correlation between several class I genes and PRC 1.1 components KDM2B and USP7 in MCPyV+ MCC, and BCOR and USP7 in MCPyV- MCC (p < 0.05; FIG. 5R). These findings motivated Applicants to further investigate the relationship of PRC1.1 to MYCL and to HLA class I genes.

### USP7 May Be Regulated by ST-MYCL-EP400 and Exhibits Co-dependency With PRC1.1

Upon reanalysis of previously generated ChIP-seq data (29028833), Applicants observed that components of the ST-MYCL-EP400 complex also directly bind to the promoter of PRC1.1 component USP7 (FIG. 5G); Applicants confirmed this result by ChIP qPCR. These results suggested the possibility that USP7 could act downstream of MYCL in regulating HLA I. Since USP7 has myriad functions (in particular, regulation of p53 through MDM2 deubiquitylation) and since its role in PRC 1.1 was only recently discovered (www.biorxiv.org/content/10.1101/221093v3), Applicants also wondered whether the effect of USP7 on HLA I was in fact mediated by PRC1.1. Applicants first leveraged data within the Cancer Dependency Map (www.biorxiv.org/content/10.1101/720243vl.full,

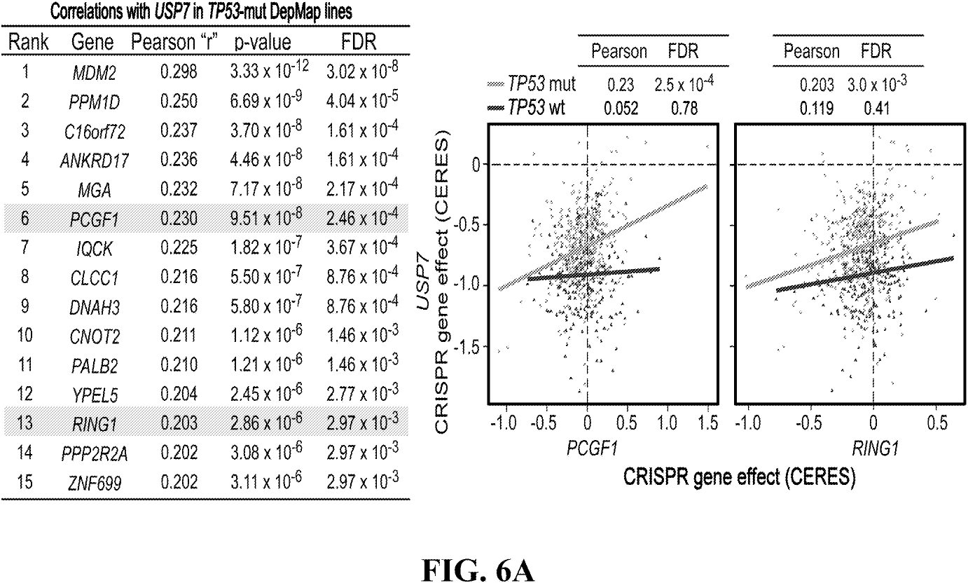

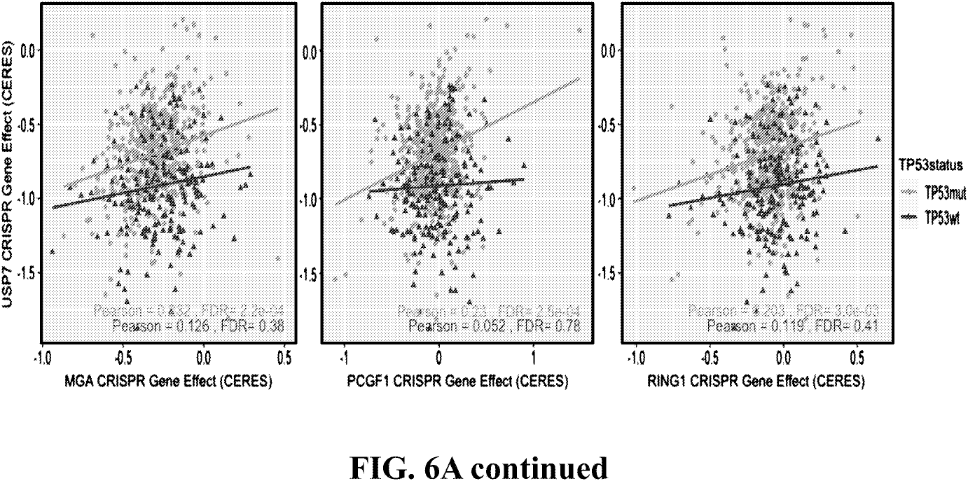

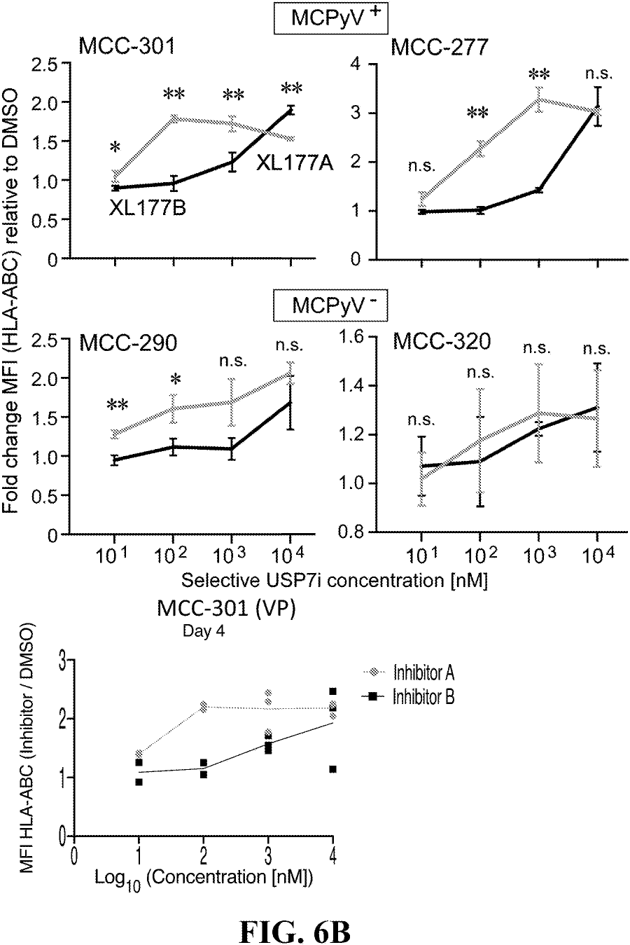

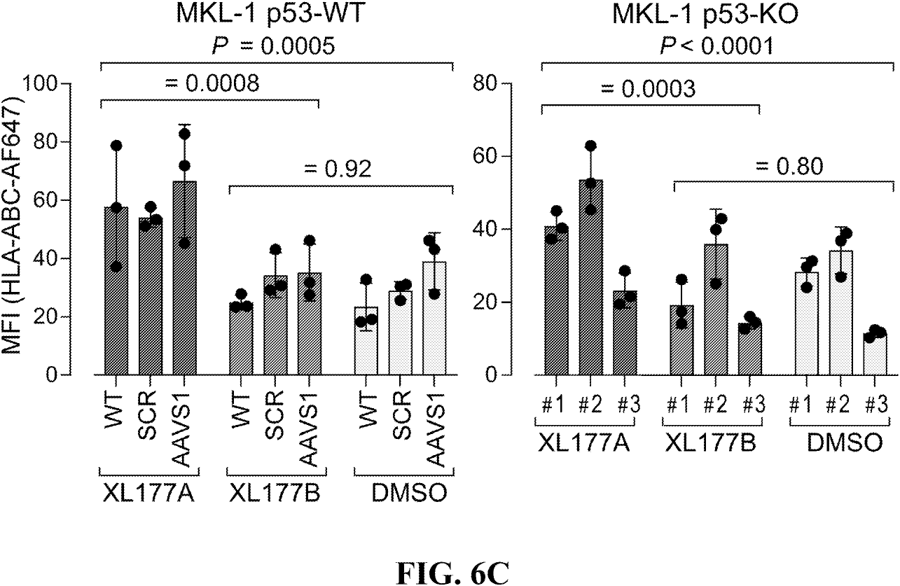

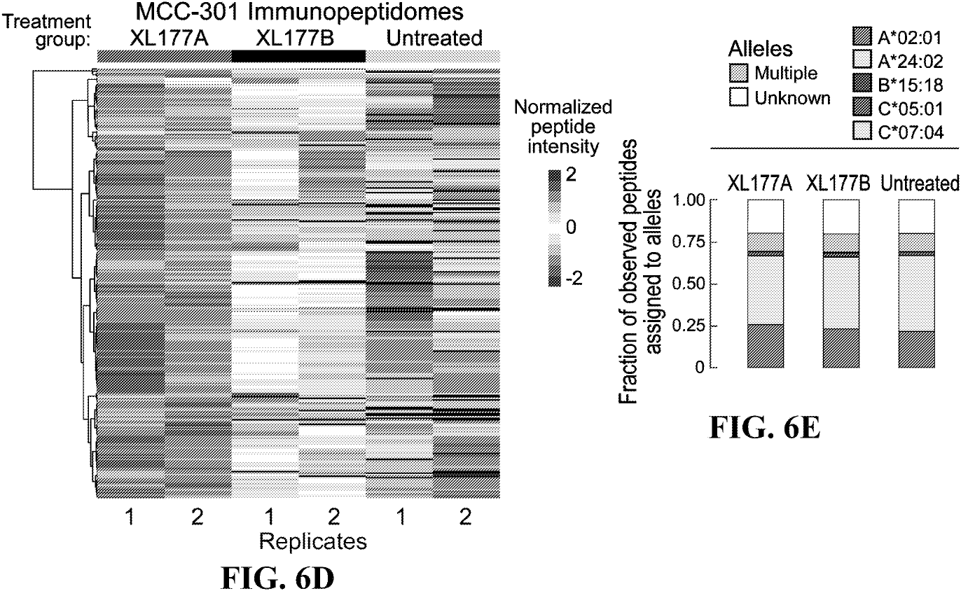

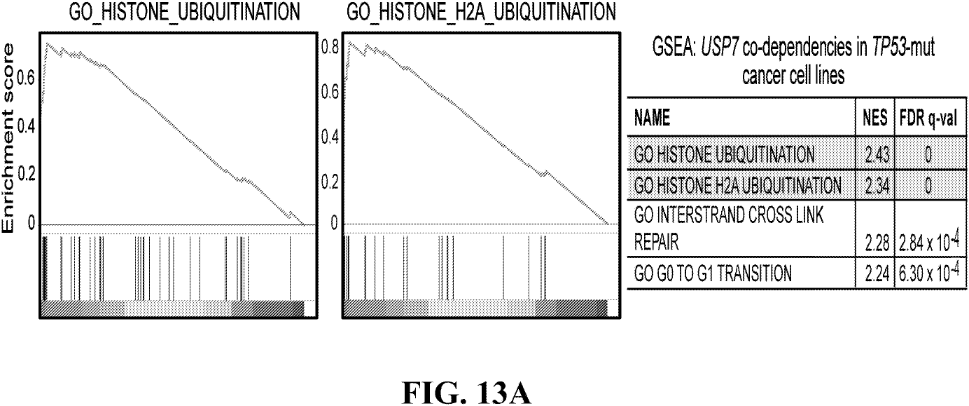

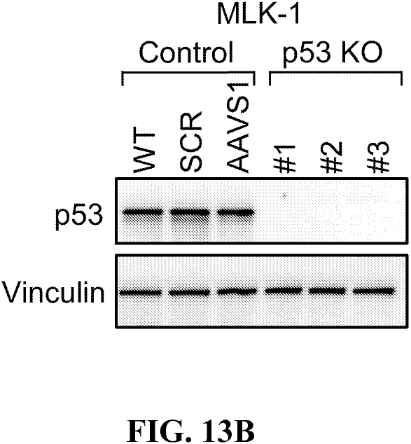

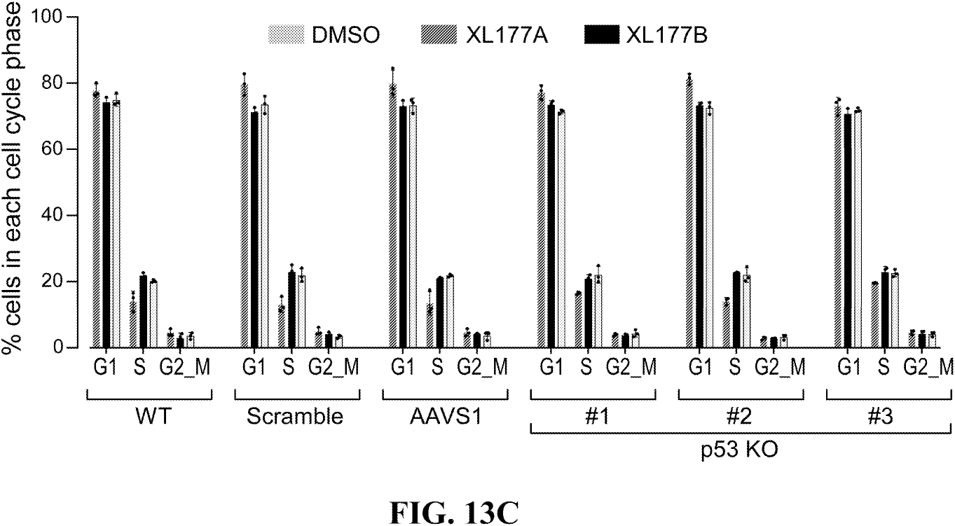

10.1038/ng.3984; and DepMap, Broad (2020): DepMap 20Q2 Public. figshare. Dataset doi:10.6084/m9.figshare.12280541.v3) to identify genes whose survival dependency correlated with that of USP7 across cancer cell lines, with the rationale that survival co-dependency implies that such genes may function in the same complex or pathway. While TP53-WT lines did not exhibit significant co-dependency of USP7 with Polycomb genes, within TP53-mut lines Applicants observed that PRC1.1 genes PCGF1 and RING1, as well as PRC1.6 component MGA, were highly correlated with USP7 (correlation ranking of 6, 13, and 5, respectively) (FIG. 6A). Furthermore, GSEA analysis revealed GO_HISTONE_UBIQUITINATION as the most enriched gene set within USP7 co-dependent genes in TP53-mut cell lines (FIG. 13A). These results suggest that although its primary role is p53 regulation, USP7 also plays an important role in PRC1.1.

### Pharmacologic Inhibition of USP7 Restores HLA I in a PRC1.1 Dependent-Manner

Applicants thus sought to pharmacologically inhibit USP7 with the goal of identifying a potential therapeutic for class I upregulation. To this end, Applicants tested XL177A, a potent, covalent USP7 inhibitor (PMID 32210275), in the MCC-301 line (Burhlage ref). After 4 days of treatment at 100 nM, Applicants observed a greater than twofold increase in MFI (HLA-ABC) by flow cytometry (FIG. 5Q). Applicants next inhibited USP7 in an MCC-301 PCGF1-KO line, reasoning that USP7 inhibition would not affect HLA I if USP7 was acting through PRC1.1 to suppress class I genes. Flow cytometry for surface class I demonstrated the effects of USP7 inhibitor on PCGF1KO. Based on the ChIP-seq evidence that the EP400-ST-MYCL complex binds to the USP7 promoter, Applicants also suspected that MYCL might also be at least partially dependent on USP7 for HLA suppression. Applicants assessed by flow cytometry if USP7 inhibition could reverse MYCL-mediated suppression of HLA I in the IMR90-MYCL overexpression line.

### Example 1 Discussion

Understanding regulators of HLA I in MCC has the potential to provide broad insights mechanisms of class I antigen presentation suppression in the setting of both viral infection and cancer. Through generation and genomic characterization of 11 robust MCC cell lines, Applicants showed that loss of surface HLA I is underpinned by transcriptional downregulation of multiple class I pathway genes and alterations to NLRC5. Through genome-wide screens in an MCPyV+ MCC line, Applicants then identified novel upstream regulators of HLA I including PRC1.1 and MYCL, which may mediate viral antigen-driven HLA I suppression.

Previous studies in MCC have demonstrated low surface HLA I and transcriptional loss of TAP½ and PSMB8/9 (LMP7/2) (Ritter et al. 2017). Applicants confirmed downregulation of these class I genes and showed that the HLA class I transcriptional activator NLRC5 is also a target for alteration, exhibiting both copy number (CN) loss and promoter methylation in many of the new MCC cell lines. NLRC5 expression is known to correlate with expression of several class I genes across many cancers, and NLRC5 CN loss was observed in 28.6% of a TCGA cohort of 7,730 cancer patients (Yoshihama et al. 2016). However, given that NLRC5 is still expressed in these MCC lines, albeit at lower levels relative to normal tissue controls, Applicants hypothesized that there could be other epigenetic regulators orchestrating class I downregulation in MCC, perhaps due to viral antigen signaling. Pharmacologic inhibition of such an HLA regulator could increase the immunogenicity of MCC tumors, as evidenced by the ability to detect HLA-presented viral epitopes following IFN-γ treatment.

Thus, Applicants performed genome-scale gain- and loss-of-function screens and found that PRC1.1 and MYCL are negative regulators of HLA I surface expression in MCC. MYCL is an intriguing candidate regulator of HLA I that is activated in virus-positive MCC by ST antigen and frequently amplified in virus-negative MCC (Cheng et al. 2017c; Knepper et al. 2019b; Kelly G. Paulson et al. 2009; Starrett et al. 2020) . Additionally, prior studies have documented the ability of MYC and MYCN to suppress HLA I surface expression in melanoma and neuroblastoma, respectively, though the precise mechanism was not elucidated (Peltenburg, Dee, and Schrier 1993; Bernards, Dessain, and Weinberg 1986). Based upon the known interaction between MYCL and ST and the experiments demonstrating that knockdown of either one upregulates class I genes, Applicants posit that MCPyV could suppress class I through ST interactions with MYCL. Given the ability of ST to recruit MYCL and the EP400 complex to transactivate a large number of downstream target genes, it is likely that one or more of these target genes contributes to repression of MHC I (Cheng et al. 2017b). One example of a ST-MYCL-EP400 downstream target gene that may play a role in repression of MHC I is USP7, a component of the PRC1.1 complex.

PRC1.1 belongs to a family of Polycomb complexes, which are repressive chromatin modifiers that act in tandem. In the traditional model, PRC2 deposits repressive H3K27me3 marks on unmethylated CpG islands, and these marks subsequently recruit canonical PRC1, which ubiquitinates H2AK119 (Blackledge, Rose, and Klose 2015). Several non-canonical PRC1 variant complexes have also been identified, one of which is PRC1.1, which can target unmethylated CpG islands independently of PRC2 (Isshiki and Iwama 2018). Polycomb complexes are important in cancer, having been implicated as both oncogenes and tumor suppressors (Koppens and van Lohuizen 2016), and PRC2 inhibitors have shown promise in early clinical trials in lymphomas and sarcomas (Genta, Pirosa, and Stathis 2019). The connection between Polycomb complexes and HLA class I regulation is a new and promising development: PRC2 was recently identified as a repressor of HLA I through an independent CRISPR screen in the leukemia cell line K562 (Burr et al. 2019), and this work establishes a novel connection to the PRC1.1 complex as well. Within the context of MCC, it has been shown that epigenetic modifiers such as histone deacetylase inhibitors can upregulate class I, but this work identifies some of the specific players involved in crafting the epigenetic landscape around class I genes. Burr et al validated PRC2 KO-mediated HLA I upregulation in one MCC line as well, lending further credence to the significance of Polycomb complexes in MCC. The screen and Burr et al’s screens identified several overlapping hits, including PCGF1, perhaps suggesting a coordination between PRC1.1 and PRC2 to suppress class I (FIG. 12H). The studies showing class I upregulation with a small-molecule USP7 inhibitor provides a potential avenue for pharmacologic targeting of PRC1.1.

However, it is important to consider that the role of PRC1.1 and MYCL in HLA I regulation may be highly context- and cell-type-dependent. Although PRC1.1 targets unmethylated CpG islands, it is unknown if there are additional factors that refine its specificity. In keeping with this context dependency, another limitation of this study is that the genome-wide screens were performed in a single, MCPyV⁺ MCC line (MCC-301). However, Applicants do observe an inverse correlation between HLA class I and several PRC1.1 components within a large cohort of 52 MCC tumors. Moreover, the identification of another Polycomb complex in Burr et al 2019′s K562 CRISPR screen further suggests a convergent biology.

Future experiments will be directed towards crystallizing the possible connection between MCPyV viral antigens, MYCL, and PRC1.1. While it is known that MYCL interacts with ST to increase levels of PRC1.1 component USP7 and USP7 binds to LT, MYC interactome profiling has also demonstrated that PRC1.1 physically interacts with MYC through the MBIV domain, which is conserved in MYCL (Kalkat et al. 2018). While MYC family proteins are typically associated with broad transcriptional activation, they also can pair with binding partners such as G9a or MIZ-1 to enact repression (Tu et al. 2018; Zhang et al. 2006), though the full repertoire of MYC-interacting repressors has yet to be characterized. Applicants speculate that MYCL could potentiate PRC1.1 function either through physical interaction or transcriptional activation to enact transcriptional repression of class I genes and will seek to understand this connection further.

In conclusion, HLA I loss is an important mechanism of immune evasion in viral infections and cancer, and a better understanding of these mechanisms can help identify targets for restoration of HLA I. Through genome-scale screens in MCC, Applicants identified PRC1.1 and MYCL as novel suppressors of HLA I surface expression. These results identify potential therapeutic targets and highlight two ways by which MCPyV viral antigens may modulate HLA class I genes.

### Example 1 References

Bernards, Rene, Scott K. Dessain, and Robert A. Weinberg. 1986. “N-Myc Amplification Causes down-Modulation of MHC Class I Antigen Expression in Neuroblastoma.” Cell 47 (5): 667-74.

Blackledge, Neil P., Nathan R. Rose, and Robert J. Klose. 2015. “Targeting Polycomb Systems to Regulate Gene Expression: Modifications to a Complex Story.” Nature Reviews. Molecular Cell Biology 16 (11): 643-49.

Boom, Vincent van den, Henny Maat, Marjan Geugien, Aida Rodriguez López, Ana M. Sotoca, Jennifer Jaques, Annet Z. Brouwers-Vos, et al. 2016. “Non-Canonical PRC1.1 Targets Active Genes Independent of H3K27me3 and Is Essential for Leukemogenesis.” Cell Reports 14 (2): 332-46.

Burr, Marian L., Christina E. Sparbier, Kah Lok Chan, Yih-Chih Chan, Ariena Kersbergen, Enid Y. N. Lam, Elizabeth Azidis-Yates, et al. 2019. “An Evolutionarily Conserved Function of Polycomb Silences the MHC Class I Antigen Presentation Pathway and Enables Immune Evasion in Cancer.” Cancer Cell 36 (4): 385-401.e8.

Butterfield, Russell J., Diane M. Dunn, Ying Hu, Kory Johnson, Carsten G. Bönnemann, and Robert B. Weiss. 2017. “Transcriptome Profiling Identifies Regulators of Pathogenesis in Collagen VI Related Muscular Dystrophy.” PloS One 12 (12): e0189664.

Cheng, Jingwei, Donglim Esther Park, Christian Berrios, Elizabeth A. White, Reety Arora, Rosa Yoon, Timothy Branigan, et al. 2017a. “Merkel Cell Polyomavirus Recruits MYCL to the EP400 Complex to Promote Oncogenesis.” PLoSPathogens 13 (10): e1006668.

. 2017b. “Merkel Cell Polyomavirus Recruits MYCL to the EP400 Complex to Promote Oncogenesis.” PLoSPathogens 13 (10): e1006668.

. 2017c. “Merkel Cell Polyomavirus Recruits MYCL to the EP400 Complex to Promote Oncogenesis.” PLoSPathogens 13 (10): e1006668.

Daily, Kenneth, Amy Coxon, Jonathan S. Williams, Chyi-Chia R. Lee, Daniel G. Coit, Klaus J. Busam, and Isaac Brownell. 2015. “Assessment of Cancer Cell Line Representativeness Using Microarrays for Merkel Cell Carcinoma.” The Journal of Investigative Dermatology 135 (4): 1138-46.

Doench, John G., Nicolo Fusi, Meagan Sullender, Mudra Hegde, Emma W. Vaimberg, Katherine F. Donovan, Ian Smith, et al. 2016. “Optimized sgRNA Design to Maximize Activity and Minimize off-Target Effects of CRISPR-Cas9.” Nature Biotechnology 34 (2): 184-91.

Dresang, Lindsay R., Anna Guastafierro, Reety Arora, Daniel Normolle, Yuan Chang, and Patrick S. Moore. 2013. “Response of Merkel Cell Polyomavirus-Positive Merkel Cell Carcinoma Xenografts to a Survivin Inhibitor.” PloS One 8 (11): e80543.

Genta, Sofia, Maria Cristina Pirosa, and Anastasios Stathis. 2019. “BET and EZH2 Inhibitors: Novel Approaches for Targeting Cancer.” Current Oncology Reports 21 (2): 13.

Ghandi, Mahmoud, Franklin W. Huang, Judit Jane-Valbuena, Gregory V. Kryukov, Christopher C. Lo, E. Robert McDonald 3rd, Jordi Barretina, et al. 2019. “Next-Generation Characterization of the Cancer Cell Line Encyclopedia.” Nature 569 (7757): 503-8.

Goh, Gerald, Trent Walradt, Vladimir Markarov, Astrid Blom, Nadeem Riaz, Ryan Doumani, Krista Stafstrom, et al. 2016. “Mutational Landscape of MCPyV-Positive and MCPyV-Negative Merkel Cell Carcinomas with Implications for Immunotherapy.” Oncotarget 7 (3): 3403-15.

Hesbacher, Sonja, Lisa Pfitzer, Katharina Wiedorfer, Sabrina Angermeyer, Andreas Borst, Sebastian Haferkamp, Claus-Jürgen Scholz, Marion Wobser, David Schrama, and Roland Houben. 2016. “RB1 Is the Crucial Target of the Merkel Cell Polyomavirus Large T Antigen in Merkel Cell Carcinoma Cells.” Oncotarget 7 (22): 32956-68.

Houben, Roland, Masahiro Shuda, Rita Weinkam, David Schrama, Huichen Feng, Yuan Chang, Patrick S. Moore, and Jürgen C. Becker. 2010. “Merkel Cell Polyomavirus-Infected Merkel Cell Carcinoma Cells Require Expression of Viral T Antigens.” Journal of Virology 84 (14): 7064-72.

Isshiki, Yusuke, and Atsushi Iwama. 2018. “Emerging Role of Noncanonical Polycomb Repressive Complexes in Normal and Malignant Hematopoiesis.” Experimental Hematology 68 (December): 10-14.

Kalkat, Manpreet, Diana Resetca, Corey Lourenco, Pak-Kei Chan, Yong Wei, Yu-Jia Shiah, Natasha Vitkin, et al. 2018. “MYC Protein Interactome Profiling Reveals Functionally Distinct Regions That Cooperate to Drive Tumorigenesis.” Molecular Cell 72 (5): 836-48.e7.

Keskin, Derin B., Annabelle J. Anandappa, Jing Sun, Itay Tirosh, Nathan D. Mathewson, Shuqiang Li, Giacomo Oliveira, et al. 2019. “Neoantigen Vaccine Generates Intratumoral T Cell Responses in Phase Ib Glioblastoma Trial.” Nature 565 (7738): 234-39.

Knepper, Todd C., Meagan Montesion, Jeffery S. Russell, Ethan S. Sokol, Garrett M. Frampton, Vincent A. Miller, Lee A. Albacker, et al. 2019a. “The Genomic Landscape of Merkel Cell Carcinoma and Clinicogenomic Biomarkers of Response to Immune Checkpoint Inhibitor Therapy.” Clinical Cancer Research: An Official Journal of the American Association for Cancer Research 25 (19): 5961-71.

. 2019b. “The Genomic Landscape of Merkel Cell Carcinoma and Clinicogenomic Biomarkers of Response to Immune Checkpoint Inhibitor Therapy.” Clinical Cancer Research: An Official Journal of the American Association for Cancer Research 25 (19): 5961-71.

Koppens, M., and M. van Lohuizen. 2016. “Context-Dependent Actions of Polycomb Repressors in Cancer.” Oncogene 35 (11): 1341-52.

Leonard, J. H., J. R. Bell, and J. H. Kearsley. 1993. “Characterization of Cell Lines Established from Merkel-Cell (‘small-Cell’) Carcinoma of the Skin.” International Journal of Cancer. Journal International Du Cancer 55 (5): 803-10.

Liu, Xi, Jennifer Hein, Simon C. W. Richardson, Per H. Basse, Tuna Toptan, Patrick S. Moore, Ole V. Gjoerup, and Yuan Chang. 2011. “Merkel Cell Polyomavirus Large T Antigen Disrupts Lysosome Clustering by Translocating Human Vam6p from the Cytoplasm to the Nucleus.” The Journal of Biological Chemistry 286 (19): 17079-90.

Park, Donglim Esther, Jingwei Cheng, Christian Berrios, Joan Montero, Marta Cortes-Cros, Stephane Ferretti, Reety Arora, Michelle L. Tillgren, Prafulla C. Gokhale, and James A. DeCaprio. 2019. “Dual Inhibition of MDM2 and MDM4 in Virus-Positive Merkel Cell Carcinoma Enhances the p53 Response.” Proceedings of the National Academy of Sciences of the United States of America 116 (3): 1027-32.

Park, Donglim Esther, Jingwei Cheng, John P. McGrath, Matthew Y. Lim, Camille Cushman, Selene K. Swanson, Michelle L. Tillgren, et al. 2020. “Merkel Cell Polyomavirus Activates LSD1-Mediated Blockade of Non-Canonical BAF to Regulate Transformation and Tumorigenesis.” Nature Cell Biology, April. doi.org/10.1038/s41556-020-0503-2.

Paulson, Kelly G., Bianca D. Lemos, Bin Feng, Natalia Jaimes, Pablo F. Peñas, Xiaohui Bi, Elizabeth Maher, et al. 2009. “Array-CGH Reveals Recurrent Genomic Changes in Merkel Cell Carcinoma Including Amplification of L-Myc.” The Journal of Investigative Dermatology 129 (6): 1547-55.

Paulson, K. G., A. Tegeder, C. Willmes, J. G. Iyer, O. K. Afanasiev, D. Schrama, S. Koba, et al. 2014. “Downregulation of MHC-I Expression Is Prevalent but Reversible in Merkel Cell Carcinoma.” Cancer Immunology Research 2 (11): 1071-79.

Peltenburg, L. T., R. Dee, and P. I. Schrier. 1993. “Downregulation of HLA Class I Expression by c-Myc in Human Melanoma Is Independent of Enhancer A.” Nucleic Acids Research 21 (5): 1179-85.

Ritter, Cathrin, Kaiji Fan, Annette Paschen, Sine Reker Hardrup, Soldano Ferrone, Paul Nghiem, Selma Ugurel, David Schrama, and Jürgen C. Becker. 2017. “Epigenetic Priming Restores the HLA Class-I Antigen Processing Machinery Expression in Merkel Cell Carcinoma.” Scientific Reports 7 (1): 1.

Schrama, David, Eva-Maria Sarosi, Christian Adam, Cathrin Ritter, Ulrike Kaemmerer, Eva Klopocki, Eva-Maria König, Jochen Utikal, Jürgen C. Becker, and Roland Houben. 2019. “Characterization of Six Merkel Cell Polyomavirus-Positive Merkel Cell Carcinoma Cell Lines: Integration Pattern Suggest That Large T Antigen Truncating Events Occur before or during Integration.” International Journal of Cancer. Journal International Du Cancer 145 (4): 1020-32.

Starrett, Gabriel J., Manisha Thakuria, Tianqi Chen, Christina Marcelus, Jingwei Cheng, Jason Nomburg, Aaron R. Thorner, et al. 2020. “Clinical and Molecular Characterization of Virus-Positive and Virus-Negative Merkel Cell Carcinoma.” Genome Medicine 12 (1): 30.

Subramanian, Aravind, Pablo Tamayo, Vamsi K. Mootha, Sayan Mukherjee, Benjamin L. Ebert, Michael A. Gillette, Amanda Paulovich, et al. 2005. “Gene Set Enrichment Analysis: A Knowledge-Based Approach for Interpreting Genome-Wide Expression Profiles.” Proceedings of the National Academy of Sciences of the United States of America 102 (43): 15545-50.

Sunshine, J. C., N. S. Jahchan, J. Sage, and J. Choi. 2018. “Are There Multiple Cells of Origin of Merkel Cell Carcinoma?” Oncogene 37 (11): 1409-16.

Swindell, William R., Mrinal K. Sarkar, Yun Liang, Xianying Xing, Jaymie Baliwag, James T. Elder, Andrew Johnston, Nicole L. Ward, and Johann E. Gudjonsson. 2017. “RNA-Seq Identifies a Diminished Differentiation Gene Signature in Primary Monolayer Keratinocytes Grown from Lesional and Uninvolved Psoriatic Skin.” Scientific Reports 7 (1): 18045.

Szklarczyk, Damian, Annika L. Gable, David Lyon, Alexander Junge, Stefan Wyder, Jaime Huerta-Cepas, Milan Simonovic, et al. 2019. “STRING v11: Protein-Protein Association Networks with Increased Coverage, Supporting Functional Discovery in Genome-Wide Experimental Datasets.” Nucleic Acids Research 47 (D1): D607-13.

Tu, William B., Yu-Jia Shiah, Corey Lourenco, Peter J. Mullen, Dharmendra Dingar, Cornelia Redel, Aaliya Tamachi, et al. 2018. “MYC Interacts with the G9a Histone Methyltransferase to Drive Transcriptional Repression and Tumorigenesis.” Cancer Cell 34 (4): 579-95.e8.

Verhaegen, Monique E., Doris Mangelberger, Jack W. Weick, Tracy D. Vozheiko, Paul W. Harms, Kevin T. Nash, Elsa Quintana, et al. 2014. “Merkel Cell Carcinoma Dependence on Bcl-2 Family Members for Survival.” The Journal of Investigative Dermatology 134 (8): 2241-50.

Vijayan, Saptha, Tabasum Sidiq, Suhail Yousuf, Peter J. van den Elsen, and Koichi S. Kobayashi. 2019. “Class I Transactivator, NLRC5: A Central Player in the MHC Class IPathway and Cancer Immune Surveillance.” Immunogenetics 71 (3): 273-82.

Yoshihama, Sayuri, Jason Roszik, Isaac Downs, Torsten B. Meissner, Saptha Vijayan, Bjoern Chapuy, Tabasum Sidiq, Margaret A. Shipp, Gregory A. Lizee, and Koichi S. Kobayashi. 2016. “NLRC5/MHC Class I Transactivator Is a Target for Immune Evasion in Cancer.” Proceedings of the National Academy of Sciences of the United States of America 113 (21): 5999-6004.

Yoshihara, Kosuke, Maria Shahmoradgoli, Emmanuel Martinez, Rahulsimham Vegesna, Hoon Kim, Wandaliz Torres-Garcia, Victor Treviño, et al. 2013. “Inferring Tumour Purity and Stromal and Immune Cell Admixture from Expression Data.” Nature Communications 4: 2612.

Zhang, Jian, Fuyang Li, Xinping Liu, Lan Shen, Junye Liu, Jin Su, Wei Zhang, et al. 2006. “The Repression of Human Differentiation-Related Gene NDRG2 Expression by Myc via Miz-1-Dependent Interaction with the NDRG2 Core Promoter.” The Journal of Biological Chemistry 281 (51): 39159-68.

### Example 2 - Reversal of Viral and Epigenetic HLA Class I Repression in Merkel Cell Carcinoma

### Reliable Generation of MCC Cell Lines From Primary Patient Samples

Since many established MCC lines have been multiply passaged in vitro and lack associated archival primary tumor material (19-22), Applicants sought to establish a reliable approach to generate MCC lines. Although MCC is typically cultured in RPMI-1640 media, Applicants hypothesized that a neuronal stem cell media that Applicants previously used to establish glioblastoma cell lines (23) would facilitate cell line establishment, based on the neuroendocrine histology of MCC and a prior report of successful MCC line generation with a neural crest stem cell medium (24). Of 5 media formulations tested, NeuroCult NS-A Proliferation medium with growth factor supplementation consistently provided the highest in vitro growth rate, tripling cell numbers after seven days in culture (FIG. 7A) and facilitating reliable growth of multiple MCC tumor cell lines (FIG. 7B). Using this method, Applicants established 11 stable cell lines from biopsies (n=4) or patient-derived xenograft (PDX) materials (n=7) (Table 1). Consistent with established classical MCC lines (25), these lines grew mostly in tight clusters in suspension and stained positive for MCC markers SOX2 and CK20, except for CK20 negativity in MCC-320 (FIG. 1A; FIG. 7CB). Applicants determined that 7 of the 11 lines (63.6%) were MCPyV+ using ViroPanel (26) (Methods, FIG. 7D).

Applicants performed whole-exome sequencing (WES) on tumor DNA from 7 of 11 patients for whom matched cell line and germline DNA were available. MCPyV- (n=2) and MCPyV+ (n=5) samples exhibited contrasting high (median 647 non-silent coding mutations per cell line, range 354-940) and low (median 40, range 18-73) TMBs (FIG. 1B), respectively, as expected. The two analyzed MCPyV- lines contained mutations in RBI and TP53, consistent with previous studies (27, 28). A median of 94.4% of cell line mutations were detected in the corresponding tumor or PDX samples (range 51-100%), and tumor-cell line pairs were associated most closely with each other based on mutational profiles (FIG. 7E). Of note, several PDX-derived tumor samples (Table 1) exhibited higher mutational burdens than their corresponding cell lines (FIG. 1B), likely due to variants associated with murine cell contamination. Corresponding RNA-sequencing (RNA-seq) of available matched tumors and cell line pairs detected MCPyV ST and LT antigen transcripts in all MCPyV+ samples (FIG. 1C; FIG. 7D). By unsupervised hierarchical clustering of these transcriptomes, each cell line associated most closely with its corresponding parent tumor (mean pairwise Spearman correlation 0.92) (FIG. 1C; FIG. 7F), rather than clustering by sample type, confirming that these cell lines faithfully recapitulate their parent tumors.

Applicants observed that 10 of 11 MCC lines strikingly exhibited low, nearly absent, surface HLA-I by flow cytometry (FIG. 1D). This low surface HLA-I was similar to well-studied MCPyV+ lines MKL-1 and WaGa (FIG. 8A). Three lines (MCC-336, -350, -358) did not appreciably upregulate HLA-I after IFN-γ exposure (≤1.15-fold increase in MFI), whereas 8 lines exhibited a ≥2.5 fold increase (median 5.7, range 2.5-12.4). Applicants further confirmed in two lines that IFN-α-2b and IFN-β upregulate HLA-I (FIG. 8B), while IFN-γ also upregulated HLA-DR expression in the MCC-301 cell line (FIG. 8C).

These cell line results were consistent with the immunohistochemistry (IHC) characterization of HLA-I expression on 9 corresponding parental tumors, in which the majority (6 of 9) displayed HLA-I-positive staining in less than 15% of tumor cells (FIG. 1D; FIG. 8D), as well as minimal HLA class II (FIG. 8E). The tumor-infiltrating CD8⁺ T cell density (median 56.6 cells/mm², range 0-1031.8) was on par with previous reports for MCC (29) (FIG. 8F). Moreover, the availability of serial formalin-fixed paraffin-embedded (FFPE) tumor samples allowed for assessing changes in HLA-I expression over time. All cell lines except MCC-290 were derived from post-treatment tumors, most commonly radiation ± cisplatin/etoposide (Table 1), and pre-treatment samples were available for 6 patients. In 5 of 6 cases, the post-treatment specimen demonstrated fewer HLA-I-positive cells than the paired pre-treatment specimens (FIG. 1E), further implicating HLA-I loss as a mechanism of therapeutic resistance.

### MCC Lines Exhibit Transcriptional Downregulation of Multiple Class I Genes and NLRC5 Alterations

To elucidate the mechanisms of impaired HLA-I surface expression in the MCC lines, Applicants performed an in-depth genomic and transcriptional characterization for a subset of MCPyV+ and MCPyV- lines for which material was available. To define class I APM transcriptional alterations, Applicants evaluated the transcriptomes of all 11 MCC lines before and after IFN-γ stimulation. At baseline, the MCC lines exhibited low expression of HLA-B, TAP1, TAP2, PSMB8, and PSMB9, compared to control epidermal keratinocytes and dermal fibroblasts (30, 31), which are candidates for the cell-of-origin of MCPyV- and MCPyV+ MCC, respectively (32) (FIG. 2A). IFN-γ treatment markedly upregulated class I gene transcripts (FIG. 9A), a trend which was confirmed in matched proteomes in 4 MCC lines (FIG. 2B). Non-IFN-γ-responsive lines (FIG. 1D) exhibited variable defects, such as lack of IFN-induced HLA-A, -B, and -C mRNA upregulation in MCC-336 (FIG. 2A) and global lack of IFN-induced HLA-I and IFN pathway upregulation at the protein level in MCC-350, including lack of STAT1 phosphorylation (FIG. 2B; FIGS. 9B-C).

To investigate the heterogeneity in the HLA-I downregulation observed in the bulk RNA-seq data, Applicants performed high-throughput, droplet-based single-cell transcriptome sequencing of 2 fresh MCC biopsies (MCC-350 [MCPyV-] and MCC-336 [MCPyV+]). From a total of 15,808 cells (mean 4,231.9.00 genes/cells) identified across the two samples, 7 distinct transcriptionally defined clusters were detected. CD45+ immune cells comprised cluster 6, while clusters 0-5 were MCC cells, identified by the expression of SOX2, SYP, and ATOH1 (FIG. 2C; FIG. 9D). All MCC clusters displayed nearly absent HLA-B, TAP½, PSMB8/9, and NLRC5 expression and low HLA-A and -C expression (FIG. 2C; FIG. 9E), consistent with the bulk RNA-seq data. By contrast, cluster 6 (immune cells) displayed an average 21-fold higher levels of HLA-A, -B, and -C transcripts.

Given the marked RNA- and protein-level downregulation of class I genes at baseline, Applicants sought to identify a possible genetic basis for these observations. By WES, no MCC lines harbored any notable mutations in class I APM genes, except for HLA-F and -H mutations in MCC-320 (Table 2). While a total of 32 mutations were detected in IFN pathway genes across all analyzed lines, only 2 were predicted as probably damaging by Polyphen and no mutations were detected in IFNGR½, JAK½, STAT1, or IRF½ (Table 2). However, copy number loss of NLRC5 was detected in 5 of 8 lines (62.5%) analyzed (FIG. 2D). NLRC5 is a transcriptional activator of several class I pathway genes that localize to conserved S/X/Y regions in their promoters (33). NLRC5 copy number loss has been recently recognized as a common alteration across many cancers (34).

### IFN-γ-induced HLA-I Upregulation Is Associated With Shifts in the HLA Peptidome

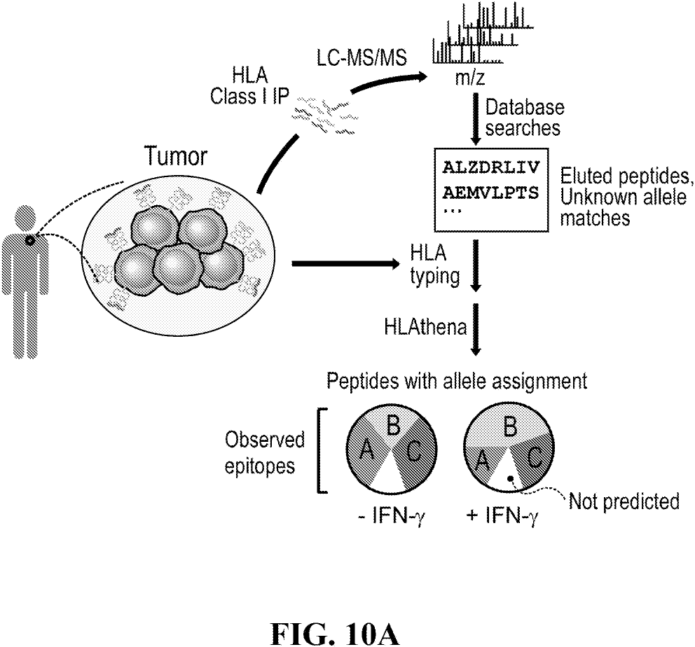

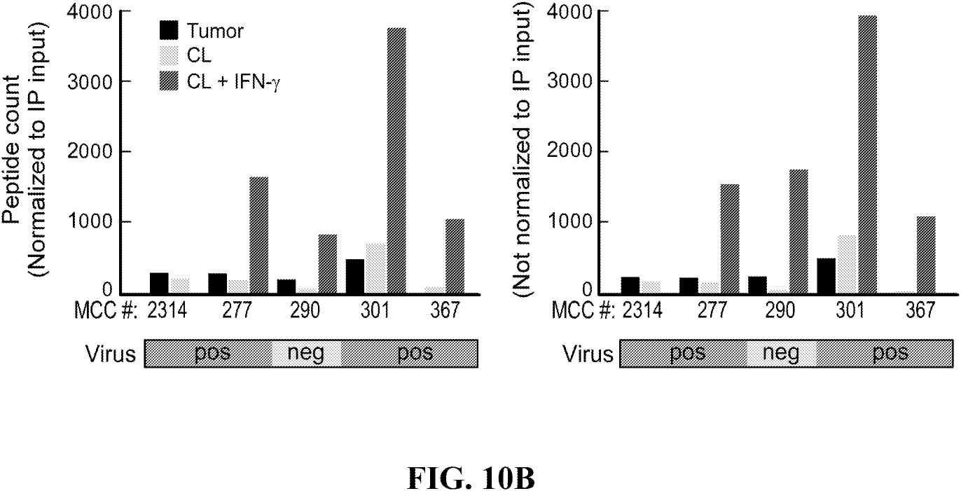

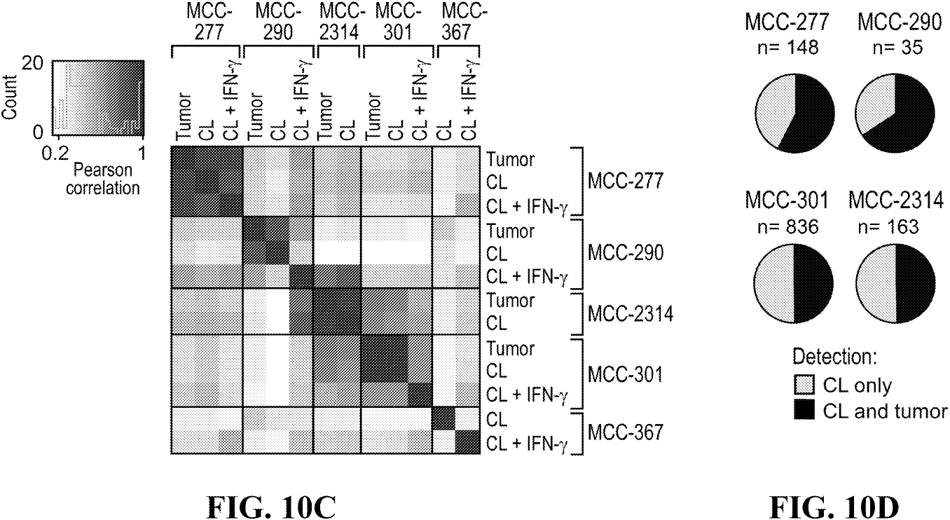

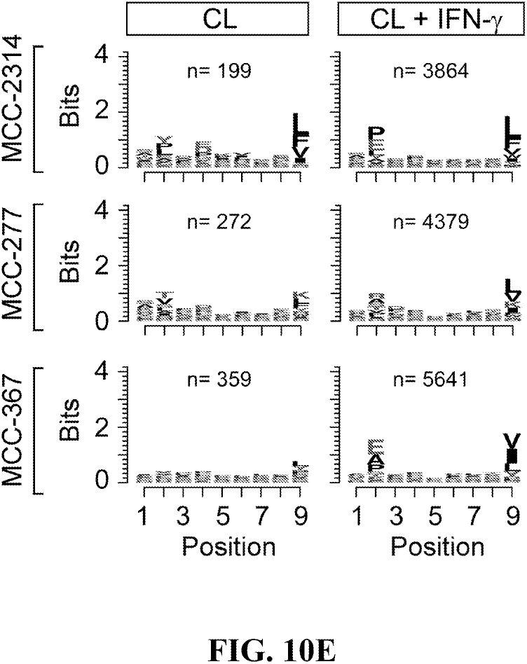

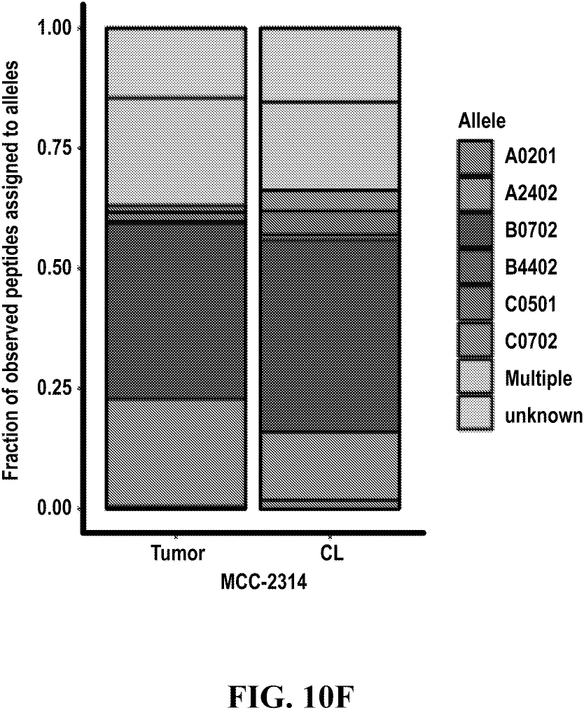

Diminished expression of HLA-I would be expected to result in a lower number and diversity of HLA-presented peptides in MCC, impacting the immunogenicity of the tumor. Indeed, using the workflows for direct detection of class I-bound peptides by liquid chromatography tandem mass spectrometry (LC-MS/MS) (Methods) (35), after immunoprecipitation of tumor cell lysates with a pan-HLA-I antibody (FIG. 10A), Applicants detected similarly low total peptide counts at baseline in parental tumors and cell lines (FIG. 10B). Following IFN-γ stimulation, a median 12-fold increase in the abundance of class I bound peptides was detected across 7 cell lines using comparable input material for immunoprecipitation (FIG. 3A, FIG. 10B, Methods). The baseline immunopeptidome amino acid signature between the cell lines and parental tumors were highly correlated (FIG. 10C), and the cell line peptidomes shared more than 50% of their peptides with the corresponding tumor peptidomes (FIG. 10D). In contrast, Applicants observed lower correlations before and after IFN-γ treatment and altered overall binding motifs with IFN-γ exposure (FIGS. 3B-C, FIG. 10E). To further explore these observations, Applicants inferred the most likely HLA allele bound by the identified peptides. When comparing cell lines with and without IFN-γ treatment, Applicants observed dramatic changes in the frequencies of peptides mapping to each HLA allele, most notably an increase in HLA-B-presented peptides (FIGS. 3D-E). This is consistent with previous observations (35), in which Applicants reported that IFNs upregulate HLA-B more strongly than HLA-A, attributable to HLA-B having two IFN-responsive elements in its promoter (36, 37).

For the MCPyV+ lines, Applicants hypothesized that this upregulation of HLA-I following IFN-γ stimulation would lead to increased ability to present MCPyV-specific epitopes. Indeed, for the MCPyV+ line MCC-367, Applicants detected a peptide sequence derived from the origin-binding domain (OBD) of LT antigen (TSDKAIELY (SEQ ID NO: 1)), which was predicted as a strong binder for the HLA*A01:01 allele of that cell line (rank = 0.018, HLAthena) (35) (FIG. 3F, Methods). Applicants subsequently confirmed reactivity against this MCC-367 derived epitope by autologous T cells by ELISpot assay, demonstrating the immunogenicity of this epitope (FIG. 3G).

### Complementary Genome-Scale Gain- and Loss-of-Function Screens to Identify Novel Regulators of HLA-I in MCC

The simultaneous transcriptional downregulation of multiple class I APM genes suggested that this suppression was coordinated by upstream regulators. While NLRC5 copy number loss was a notable event, it was only observed in 5 of 8 lines (62.5%) studied, and thus Applicants suspected the presence of other regulators. To this end, Applicants conducted paired genome-scale open reading frame (ORF) gain-of-function and CRISPR-Cas9 knock out (KO) loss-of-function screens in the MCPyV+ MCC-301 line to systematically identify novel regulators of HLA-I surface expression in MCC. Applicants chose MCC-301 for three reasons. First, the low TMB of MCPyV+ MCC increases the likelihood of a homogeneous mechanism of HLA-I suppression, which might relate to viral antigen signaling or cell-type specific factors. Second, IFN-γ-mediated inducibility of HLA-I largely excludes the possibility of hard-wired genomic alterations that would prohibit HLA-I upregulation. Last, such screens necessitate cell lines with robust growth such as MCC-301 (FIG. 7B).

MCC-301 cells were transduced at a low multiplicity of infection with genome-scale ORF (38) or Cas9+sgRNA (39) lentiviral libraries (Methods). After staining cells with an anti-HLA-ABC antibody, HLA-I-high and HLA-I-low populations underwent fluorescence activated cell sorting (FACS)-based cell isolation, with each screen performed in triplicate (FIG. 4A). Constructs were ranked according to their median log₂-fold change (LFC) enrichment in the HLA-I-high versus HLA-I-low populations and for the CRISPR screen, sgRNA rankings were aggregated into gene-level rankings using the STARS algorithm (39) (Methods).

### MYCL Identified as a Mediator of HLA-I Suppression in MCC via ORF Screen

The ORF screen produced 75 hits with a >4-fold enrichment in HLA-I-high versus HLA-I-low populations. As expected, these hits were highly enriched for IFN and HLA-I pathway genes by Gene Set Enrichment Analysis (GSEA) (40) (FIG. 4B). The top hit was IFNG, with IFN pathway genes comprising 4 of the top 12 hits (33%). HLA-B and -C were ranked #10 and #38. Of note, transduction with the ORF library led to a population-wide increase in HLA-I, presumably due to IFN secretion from cells transduced with IFN gene ORFs. Applicants confirmed this was an ORF library-specific effect and not due to lentiviral transduction, as GFP-transduced cells did not exhibit an increase in surface HLA-I (FIG. 11F). Furthermore, Applicants confirmed that these notable hits exhibited high concordance between at least 2 replicates (FIGS. 11B-C).

Applicants validated the many highly enriched positive hits by generating 71 single ORF overexpression lines in MCC-301, focusing on the top positive hits not directly related to IFN or HLA-I pathways. By flow cytometry, 8 of 71 candidate hits (11.3%) upregulated surface HLA-I by > 2-fold compared to a GFP control while also maintaining viability after transduction, including Polycomb-related genes EZHIP (CXorf67) and YY1 (FIG. 4C). As further validation, Applicants transduced these ORFs into the MCPyV+ MCC-277 line and confirmed increased levels of HLA-I (FIG. 4C). In contrast to the genes that increased levels of HLA-I, MYCL was the top negative hit (FIG. 4B). MYCL is an important transcription factor in MCPyV+ MCC, as ST binds and recruits MYCL to the EP400 chromatin modifier complex to enact widespread epigenetic changes necessary for oncogenesis (15, 41, 42). As validation, Applicants observed that MYCL knockdown in MKL-1 cells resulted in an increase in surface HLA-I by flow cytometry compared to a scrambled shRNA control (P = 0.003), an effect which was negated by rescue expression of exogenous MCYL (FIG. 4D).

To further investigate how MYCL affects HLA-I surface expression, Applicants performed RNA-seq of the MKL-1 MYCL shRNA line. Compared to the scrambled shRNA control line, Applicants observed a >2-fold increase in expression of class I genes including HLA-B, HLA-C, TAP1, and PSMB9, with enrichment for the signature of antigen processing/presentation by GSEA (q=0.04; FIG. 4E, FIG. 11D). Since ST binds and potentiates MYCL function through the ST-EP400-MYCL complex (15), Applicants suspected that viral antigen inactivation might also upregulate class I. To further expand the scope of these findings, Applicants selected another established MCPyV+ MCC line, WaGa, to transduce with an shRNA that targets shared exons of ST and LT, leading to inactivation of both MCPyV viral antigens. Applicants observed a similar but more modest upregulation of class I genes, including > 1.5-fold increases in HLA-B, HLA-C, and NLRC5 (FIG. 4F). Moreover, knockdown of EP400 in MKL-1 with two different shRNAs resulted in >3-fold increases in HLA-B and HLA-C (FIG. 11E). These findings thus implicate the continued expression of ST-EP400-MYCL complex components in the downregulation of HLA-I in MCC.

To determine if the HLA-I-suppressive effects of MYCL generalized to MCPyV- MCC and other cancers, Applicants evaluated the copy number status ofMYCL in MCPyV- MCC. Copy number gain of chromosome 1p, encompassingMYCL, was previously reported as one of the more common copy number alterations in MCC (28, 43). Three of the 4 (75%) MCPyV- MCC lines exhibited MYCL copy number gain (copy number ratio 1.16-1.56; FIG. 4G), suggesting a mechanism by which MCPyV- MCC may enhance MYCL signaling in the absence of viral antigens. To determine ifMYCL is related to HLA-Iexpression in other cancers, Applicants queried publicly available RNA-seq data from the Cancer Cell Line Encyclopedia (44). Notably, other neuroendocrine cancers such as small cell lung carcinoma and neuroblastoma with lower expression of HLA-I pathway components also frequently featured overexpression of MYC family members MYCL and MYCN, respectively (FIG. 4H). Overall, MYCL exhibited negative correlation with average HLA-I gene expression (Pearson correlation r = -0.33, P = 0.04).

### PRC1.1 Complex Identified as a Novel Negative Regulator of HLA-I in MCC by CRISPR Loss-of-Function Screen

The CRISPR-KO screen also identified several class I APM genes. The top negative hit was TAPBP (FIG. 5A), a chaperone for partially folded HLA-I heavy chains that facilitates binding between unbound HLA-I and TAP (45). Other notable negative hits included IFN pathway gene IRF1 (#21) and class I genes CALR (#84) and B2M (#141). Having previously identified MYCL in the ORF screen, Applicants observed other ST-MYCL-EP400 complex members within the CRISPR positive hits included BRD8 (#51), DMAP1 (#93), KAT5 (#619), and EP400 (#886). In addition, Applicants identified several components of the Polycomb repressive complex 1.1 (PRC1.1) within the CRISPR positive hits, including the top two hits of the screen: USP7 (#1), BCORL1 (#2), and PCGF1 (#50). For these genes, Applicants observed high concordance between two CRISPR replicates (FIGS. 12A-B; Methods) and a >4.5-fold enrichment for at least 2 of the 4 sgRNAs (FIG. 12C). PRC1.1 is a noncanonical Polycomb repressive complex that silences gene expression through mono-ubiquitination of H2AK119 in CpG islands. Other components of PRC1.1 include KDM2B, SKP1, RING1A/B, RYBP/YAF2, and BCOR (which can substitute for BCORL1) (46). In aggregate, review of the top hits across the parallel screens revealed several hits related to Polycomb repressive complexes: PRC1.1 components USP7,BCORL1, and PCGF1; ORF hits EZHIP, which is an inhibitor of Polycomb repressive complex 2 (PRC2)(47), and YY1 (48); and PRC2 components EED and SUZ12 (CRISPR positive hits #162 and #409).

Applicants subsequently generated a series of MCC-301 KO lines against PRC1.1 genes USP7, BCORL1, and PCGF1. Compared to a non-targeting sgRNA control line, knockout of each gene increased baseline surface HLA-I expression levels as assessed by flow cytometry (FIG. 5B). PCGF1 knockout increased IFN-γ-induced HLA-I upregulation as well (FIG. 12D). Gene editing and protein knockout were confirmed by Sanger sequencing using TIDE (49) (FIG. 12E) and by western blot (FIG. 5C), in genes for which antibodies were available.

To define the specific class I APM gene expression changes associated with PRC1.1 loss of function, Applicants generated RNA-seq data from a PCGF1-KO line and a non-targeting sgRNA control line in MCC-301, since previous studies demonstrated that PCGF1 is essential for PRC1.1 function (50). Genes upregulated in the PCGF1-KO line were significantly enriched for the “PRC2 target genes” signature (FIG. 5D), consistent with the known role of PRC1.1 in coordinating with PRC2 to repress target genes. Strikingly, Applicants noticed a >5-fold increase in expression of the class I APM genes TAP1, TAP2, and PSMB8, with a more modest increase in the class I transactivator NLRC5 (FIG. 5D). For further confirmation, Applicants observed increased protein expression of TAP1 by Western blot both at baseline and after IFN-γ treatment in the PCGF1-KO line (FIG. 5E). Applicants then evaluated an RNA-seq cohort of 51 MCC tumor biopsies to examine the association between expression of HLA-I genes and PRC1.1. To account for the potential of immune cell infiltration, which might confound measurement of bulk class I expression, Applicants applied ESTIMATE (51) to calculate tumor purity (median 87% purity, range 41-99%). Applicants observed a negative correlation between several class I genes and PRC1.1 components BCOR and KDM2B (P < 0.05; FIG. 5F).

To explore if there is a relationship between MYCL and PRC1.1, Applicants reanalyzed previously generated ChIP-seq data in MKL-1 cells (15). Applicants observed that components of the ST-MYCL-EP400 complex were bound to the promoters of PRC1.1 genes USP7 and PCGF1, but not BCOR or BCORL1 (FIG. 5G, FIG. 12G). The binding of MAX and EP400 to USP7 and PCGF1 was further confirmed by ChIP qPCR (FIG. 5H). These results indicate that PRC1.1 may act downstream of MYCL. Moreover, bothMYCL and PRC1.1 component USP7 encode proteins that have been reported to directly interact with MCPyV ST and LT viral antigens, respectively (15, 52), suggesting a model by which viral antigens may coordinate via MYCL and PRC1.1 to suppress HLA-I surface expression (FIG. 5I).

### Pharmacologic Inhibition of USP7 Restores HLA-I in MCC

Selective small-molecule inhibitors of the PRC1.1 component USP7 have been previously developed (53, 54). However, since USP7 has many functions, such as regulation of p53 through MDM2 deubiquitination, and since its association with PRC1.1 was recently discovered (55-57), Applicants queried the extent of USP7′s role in PRC1.1. By examining the Cancer Dependency Map (58-60), Applicants identified genes whose survival dependency correlated with that of USP7 across cancer cell lines, with the rationale that survival co-dependency implies that such genes may function within the same complex or pathway. While TP53-wildtype (WT) lines did not exhibit co-dependency between USP7 and Polycomb genes, TP53-mutant lines showed a high correlation between USP7 and PRC1.1 genes PCGF1 and RING1 (6^{th} and 13^{th} highest correlation coefficients, FDR = 2.46 × 10⁻⁴ and 2.97 × 10⁻³, respectively) (FIG. 6A). Furthermore, GSEA analysis revealed histone ubiquitination as the most enriched gene set within USP7 co-dependent genes in TP53-mutant cell lines (FIG. 13A). These results further support the notion that USP7 plays an important role in PRC1.1 function.

Applicants therefore assessed the activity of XL177A, a potent and irreversible USP7 inhibitor, compared to XL177B, the enantiomer of XL177A which is 500-fold less potent but exhibits on-target activity at higher doses (54). Two MCPyV+ lines (MCC-301 and -277) and two MCPyV- lines (MCC-290 and -320) were treated for 3 days at varying inhibitor concentrations. At 100 nM, Applicants observed a mean 2.0-fold (range 1.78 - 2.27) increase in expression of surface HLA-I by flow cytometry relative to DMSO in the two MCPyV+ lines. Within the MCPyV- lines, Applicants noted a more modest increase in HLA-I levels in MCC-290 but not MCC-320 (FIG. 6B). Given USP7′s prominent role in p53 regulation, Applicants assessed if USP7′s effect on HLA was p53-dependent. Notably, XL177A treatment of both TP53-KO and TP53-WT lines in MKL-1 increased surface HLA-I relative to XL177B and DMSO (FIG. 6C; FIG. 13B). Moreover, while USP7 inhibition did induce slight cell cycle shifts from S to G1 phase, this effect was similar in both TP53-WT and TP53-KO contexts (FIG. 13C). To evaluate the functional consequences of USP7 inhibition on HLA-I presentation, Applicants analyzed the HLA-I-bound peptidomes of MCC-301 cells treated with XL177A and XL177B. XL177A-treated cells exhibited higher abundances of displayed peptides compared to XL177B and untreated cells (FIG. 6D). Out of 282 peptides whose abundance significantly differed (P < 0.05) between two of the three conditions, 270 peptides (95.7%) were more abundant in XL177A compared to untreated cells. Notably, XL177A treatment did not affect the frequency of peptides displayed on each respective HLA-I gene (HLA-A, -B, -C) (FIG. 6E). This was consistent with the prior observation that PCGF1 KO mostly upregulated other class I genes related to peptide processing such as TAP½ and PSMB8, rather than the HLA-A, -B, and -C genes themselves.

### Example 2 Discussion

Surface HLA-I loss is a widespread mechanism of immune evasion in cancer and facilitates resistance to immunotherapy (1-8). As a virally driven cancer, MCPyV+ MCC provides a highly informative substrate to study mechanisms by which viral antigens corrupt normal physiology. Just as the MCPyV LT antigen inactivates RB1 to phenocopy RB1 mutations commonly seen in other cancers (14), Applicants suspected that MCPyV viral antigens also suppress class I antigen presentation through derangement of regulatory mechanisms that might be phenocopied in other cancers including MCPyV- MCC tumors. Through unbiased genome-scale screens for regulators of HLA-I, Applicants identified MYCL, which acts as part of the ST-MYCL-EP400 complex in MCPyV+ MCC and is frequently amplified in MCPyV- MCC (15, 28, 43, 61). The ST antigen recruits MYCL to the EP400 complex to enact widespread epigenetic changes necessary for MCC oncogenesis, and the results identify a novel function of ST in suppressing HLA-I by MYCL activity. The effect of MYC family proteins on HLA generalizes to other cancers as well, as MYC and MYCN can suppress HLA-I in melanoma and neuroblastoma, respectively (62, 63).

The identification of PRC1.1 in the CRISPR screen clearly confirms the importance of epigenetic regulatory mechanisms in suppressing HLA-I. PRC1.1 is a noncanonical Polycomb complex that mono-ubiquitinates H2AK119 within CpG islands, facilitating recruitment of PRC2 which deposits suppressive H3K27 trimethylation marks. PRC2 was recently identified as an HLA-I repressor through independent CRISPR screens in leukemia (64) and lymphoma cell lines (65), and this work establishes a novel connection to PRC1.1. Those screens also identified PCGF1, while Applicants identified PRC2 subunits in the CRISPR screen and PRC2 inhibitor EZHIP (47) in the ORF screen. Thus, the studies advance an emerging model by which cancers co-opt the Polycomb epigenetic machinery to suppress class I antigen presentation.

Reversal of HLA-I loss is crucial for an effective anti-tumor cytotoxic T cell response, and, of high clinical interest, an HLA-I-upregulating drug could augment response to immunotherapy such as checkpoint blockade. The small-molecule USP7 inhibitor studies provide a promising avenue for pharmacologic upregulation of HLA-I in MCC via PRC1.1 inhibition. In contrast to the nonspecific, inflammatory mechanism by which IFN-y upregulates HLA-I, USP7 inhibition reverses the underlying tumor-intrinsic, epigenetic defects in class I antigen presentation via disruption of PRC1.1. Thus, USP7 inhibition raises baseline tumor HLA-I expression without the requirement of an inflammatory microenvironment which may only temporarily increase HLA-I expression.

The studies raise rich questions for future research. The USP7 and PCCGF1 promoter occupation by the ST-MYCL-EP400 complex suggests a possible unifying mechanism by which MCPyV ST antigen co-opts MYCL to increase expression of PRC1.1, which subsequently suppresses class I APM gene expression. Future studies will be directed towards elucidating this connection. Furthermore, the effects of USP7 inhibition across other cancers may be tested to determine its scope. Applicants anticipate that continued in vitro and in vivo validation can pave the way for clinical use of USP7 inhibitors as an HLA-I-restoring adjunct across many cancers.

### Example 3 - Methods

Study Design. The overall objective of this study was to determine the molecular mechanisms of HLA-I downregulation in MCC and to identify novel targets for restoring HLA-I in MCC. This was examined via controlled laboratory experiments in a panel of 11 novel MCC cell lines, derived either directly from frozen tumor biopsies or from mouse patient-derived xenografts. Informed consent was obtained from patients under IRB protocol #09-156 at the Dana-Farber Cancer Institute, and the patients’ clinical annotations are listed in Table 1. No sample size calculations were performed, as the sample size of 11 MCC lines was based on availability of MCC tumor specimens for cell line generation. One additional MCC cell line MCC-275 was generated and sequenced but was excluded from this manuscript due to concerns that it had been contaminated by another cell line during cell line generation. Determination of which tumors and cell lines underwent any type of sequencing in this study was based solely on which specimens had adequate material available at time of the experiment. No randomization was performed, and blinding was not relevant to this study as there were no human or animal randomized trials conducted. Laboratory experiments were performed in duplicate or triplicate when possible. Means, standard deviations, and number of replicates are reported in the manuscript. The definition and handling of outliers, when applicable for a given experiment, are described in the corresponding methods subsection.

Generation of tumor cell lines. For the panel of novel MCC lines, MCC tumor samples were obtained from either patient biopsy or patient-derived xenografts from mice, which were generated as previously described (66) and in accordance with the Dana-Farber Cancer Institute Institutional Animal Care and Use Committee (IUCAC). The tissue was minced manually, suspended in a solution of 2 mg/ml collagenase I (Sigma Aldrich), 2 mg/ml hyaluronidase (Sigma Aldrich) and 25 ug/ml DNase I (Roche Life Sciences), transferred to a 15 mL conical tube, and incubated on an orbital shaker at low speed for 30 min. After digestion, the single-cell suspension was passed through a 100 micron strainer, washed, and cultured in tissue culture flasks containing media from NeuroCult NS-A Human Proliferation Kit (StemCell Technologies) supplemented with 0.02% Heparin (StemCell Technologies), 20 ng/ml hEGF (Miltenyi Biotec) and 20 ng/ml hFGF-2 (Miltenyi Biotec). When available, excess tumor single cell suspensions were frozen in 90% FBS and 10% DMSO and banked in liquid nitrogen. Established cell lines were tested as mycoplasma free (Venor™ GeM Mycoplasma Detection Kit, Sigma Aldrich). Cell lines were authenticated as MCC through immunohistochemical staining using antibodies against CK20 and SOX2 (FIG. 1A; FIG. 7B). Cell lines were authenticated as derivatives of original tumor samples by HLA typing, which was available for 7 of the 11 lines (Table 4). Cell line sexes are described in Table 1. All MCC cell lines were maintained in media from NeuroCult NS-A Proliferation Kit supplemented with 0.02% heparin, 20 ng/mL hEGF, and 20 ng/mL hFGF2. Other media used for cell culture optimization included StemFlex (Gibco); Neurobasal (Gibco) supplemented with 0.02% heparin (StemCell Technologies), 20 ng/mL hEGF (Miltenyi Biotec), and 20 ng/mL hFGF2 (Miltenyi Biotec); DMEM GlutaMAX (Gibco) supplemented with 10% FBS (Gibco), 1% penicillin/streptomycin (Gibco), 1 mM sodium pyruvate (Life Technologies), 10 mM HEPES (Life Technologies), and 55 nM β-mercaptoethanol (Gibco); and RPMI-1640 (Gibco) supplemented with 20% FBS (Gibco) and 1% penicillin/streptomycin (Gibco).

MKL-1 and WaGa lines were grown in RPMI-1640 medium supplemented with 10% fetal bovine serum (FBS; Gibco) and 1% penicillin/streptomycin (Gibco).

Histology and immunohistochemistry. All IHC was performed on the Leica Bond III automated staining platform. From the cell lines, up to 10 million MCC cells were pelleted, fixed in formaldehyde, washed with PBS, and mounted on a paraffin block. For single stains, 5-micron sections were cut and stained for SOX2 or CK20. The Leica Biosystems Refine Detection Kit was used with citrate antigen retrieval for SOX2 (Abcam #97959, polyclonal, 1: 100 dilution) and with EDTA antigen retrieval for Cytokeratin 20 (CK20; Dako #M7019, clone Ks20.8, 1:50 dilution). For dual immunohistochemical staining of the archival tumor specimens, Applicants used MCC marker SOX2 (CST, D6D9, 1:50 dilution; red chromogen) and either HLA class I (Abcam, EMR8-5, 1:6,000 dilution; brown chromogen) or HLA class II (Dako M0775, CR3/43, 1:750 dilution; brown chromogen) using an automated staining system (Bond III, Leica Biosystems) according to the manufacturer’s protocol, as previously described (67). The proportion of SOX2+ MCC cells that exhibited HLA I or HLA II membranous staining was evaluated by consensus of two board-certified pathologists.

Immunofluorescence. Staining was performed overnight on BOND RX fully automated stainers (Leica Biosystems). 5-µm thick formalin-fixed paraffin-embedded tumor tissue sections were baked for 3 hours at 60° C. before loading into the BOND RX. Slides were deparaffinized (BOND DeWax Solution, Leica Biosystems, Cat. AR9590) and rehydrated through a series of graded ethanol to deionized water. Antigen retrieval was performed in BOND Epitope Retrieval Solution 1 (ER1; pH 6) or 2 (ER2; pH 9) (Leica Biosystems, Cat. AR9961, AR9640) at 95° C. Deparaffinization, rehydration and antigen retrieval were all pre-programmed and executed by the BOND RX. Next, slides were serially stained with primary antibodies for: SOX2 (clone B6D9, Cell Signaling, dilution 1:200; Opal 690 1:100), CD8 (clone 4B11, Leica, dilution 1:200; Opal 480 1:150), PD-L1 (clone E1L3N, Cell Signaling, dilution 1:300; Opal 520 1:150), and PD-1 (clone EPR4877[2], Abcam, dilution 1:300; Opal 620 1:300) with ER1 for 20 min; and FOXP3 (clone D608R, Cell Signaling, dilution 1:100; Opal 570 1:300) with ER2 solution for 40 min. Each primary antibody was incubated for 30 minutes. Subsequently, anti-mouse plus anti-rabbit Opal Polymer Horseradish Peroxidase (Akoya Biosciences, Cat. ARH1001EA) was applied as a secondary label with an incubation time of 10 minutes. Signal for antibody complexes was labeled and visualized by their corresponding Opal Fluorophore Reagents (Akoya) by incubating the slides for 10 minutes. Slides were incubated in Spectral DAPI solution (Akoya) for 10 minutes, air dried, and mounted with Prolong Diamond Anti-fade mounting medium (Life Technologies, Cat. P36965) and imaged using the Vectra Polaris multispectral imaging platform (Vectra Polaris, Akoya Biosciences). Representative tumor regions of interest were identified by the pathologist and 2-6 fields of view were acquired per sample. Images were spectrally unmixed and cell identification was performed using the supervised machine learning algorithms within Inform 2.4 (Akoya) with pathologist supervision as previously described (67).

Flow cytometry. Cells were dissociated with Versene and incubated with 5 µL Human TruStain FcX (Fc Receptor Blocking Solution; Biolegend # 422302) per million cells in 100 µL at room temperature for 10 min. Fluorophore-conjugated antibodies or respective isotype controls were added and incubated for another 30 min at 4° C. Cells were then washed once with PBS and resuspended in PBS or 4% paraformaldehyde and analyzed on an LSR Fortessa cytometer. For HLA-I and HLA-II detection, the following antibodies were used: HLA-ABC (W6/32 clone) conjugated to PE (BioLegend # 311406), APC (BioLegend # 311410), or AF647 (Santa Cruz Biotechnology # sc32235 AF647), and HLA-DR-FITC (BioLegend # 307604).

Whole exome sequencing and mutation calling. Genomic DNA samples were sheared using a Broad Institute-developed protocol optimized for ~180bp size distribution. Kapa Hyperprep kits were used to construct libraries in a process optimized for somatic samples, including end repair, adapter ligation with forked adaptors containing unique molecular indexes, and addition of P5 and P7 sample barcodes via PCR. SPRI purification was performed and resulting libraries were quantified with Pico Green. Libraries were normalized and equimolar pooling was performed to prepare multiplexed sets for hybridization. Automated capture was performed, followed by PCR of the enriched DNA. SPPI purification was used for cleanup. Multiplex pools were then quantified with Pico Green and DNA fragment size was estimated using Bioanalyzer. Final libraries were quantitated by qPCR and loaded onto an Illumina flowcell across an adequate number of lanes to achieve ≥85% of target bases covered at ≥50× depth, with a range from 130-160× mean coverage of the targeted region.

Exome-sequencing BAM files were downloaded from the Broad Genomics Firecloud/Terra platform using the Google Cloud Storage command line tool gsutil version 4.5 (https://github.com/GoogleCloudPlatform/gsutil/). GATK version 4.1.2.0(68) was used to: (1) call mutations from reference on normal BAMs with Mutect2 command (69) using a max MNP distance of 0, (2) build a panel of normals from VCF files of called normal mutations using the CreateSomaticPanelOfNormals command, and (3) call mutations between pairs of both tumor and cell line with compared to their respective normal counterpart using the Mutect2 command. For these steps, the following annotations were used: b37 reference sequence downloaded from ftp.broadinstitute.org/bundle/b37/human_glk_v37.fasta, germline resource VCF downloaded from ftp.broadinstitute.org/bundle/beta/Mutect2/af-only-gnomad.raw.sites.b37.vcf.gz, and intervals list downloaded from github.com/broadinstitute/gatk/blob/master/src/test/resources/large/whole_exome_illumina_codi ng_v1.Homo_sapiens_assembly19.targets.interval_list. Called variants were filtered with the GATK FilterMutectCalls command, and variants labeled as PASS were extracted and included in downstream analyses.

Next, VCF files of passing variants were annotated as MAF files using vcf2maf version 1.16.17 (downloaded from github.com/mskcc/vcf2maf/tree/5453f802d2f1f261708fe21C9d47b66d13e19737) and Variant Effect Predictor version 95 installed from github.com/Ensembl/ensembl-vep/archive/release/95.3.tar.gz (70). R Bioconductor package maftools (71) was used to generate oncoplots of mutations by gene and sample.

Patient HLA allotype was assessed using standard class I and class IIPCR-based typing (Brigham and Women’s Hospital Tissue Typing Laboratory).

Whole genome sequencing and copy number analysis. Whole genome sequencing was performed by Admera Health. Reads were quality and adapter trimmed using TrimGalore with default settings. Trimmed reads were aligned against a fusion reference containing hg38 and MCPyV (NCBI accession number: NC_010277) using bowtie2 -very-sensitive. Copy number variant analysis was performed with GATK4 CNV recommended practices. A panel of normals was generated from 17 normal blood whole genomes to call CNVs from tumors. All CNV calls that mapped to hg38 were visualized using the Integrative Genomics Viewer from Broad Institute (software.broadinstitute.org/software/igv/).

RNA sequencing and analysis. For samples from the MCC tumors and newly generated cell lines, RNA was first assessed for quality using the Agilent Bioanalyzer (DV200 metric). 100 ng of RNA were used as the input for first strand cDNA synthesis using Superscript III reverse transcriptase and Illumina’s TruSeq RNA Access Sample Prep Kit. Synthesis of the second strand of cDNA was followed by indexed adapter ligation with UMI (unique molecular index) adaptors. Subsequent PCR amplification enriched for adapted fragments. Amplified libraries were quantified, normalized, pooled, and hybridized with exome targeting oligos. Following hybridization, bead clean-up, elution, and PCR was performed to prepare library pools for sequencing on Illumina flowcell lanes. Transcriptomes were sequenced to a coverage of at least 50 million reads in pairs.

For fibroblast and keratinocyte control lines, raw FASTQ files were downloaded from the Sequence Read Archive using R Bioconductor package SRAdb (71, 72) with accession codes SRP 126422 (4 replicates from control samples ‘NN’) and SRP131347 (6 replicates with condition: control and genotype: control). Raw FASTQ files for MKL-1 and WaGa were obtained from the control shScr MKL-1 and WaGa cell lines that are described below (Methods: MKL-1 shMYCL and WaGa shSTILTline generation and sequencing). FASTQ files from fibroblasts, keratinocytes, MKL-1, and WaGa were then aligned using STAR version 2.7.3a (73), using the index genome reference file downloaded from

ftp.ebi.ac.uk/pub/databases/gencode/Gencode_human/release_19/GRCh37.pl3.geno
me.fa.gz, the transcript annotation file downloaded from
https:/data.broadinstitute.org/snowman/hg19/star/gencode.v19.annotation.gtf, and with the
following options: --twopassMode Basic, --outSAMstrandField intronMotif, --alignIntronMax
1000000, --alignMatesGapMax 1000000, --sjdbScore 2, --outSAMtype BAM Unsorted, --
outSAMattributes NH HINM MD AS XS, --outFilterType BySJout, --outSAMunmapped Within,
--genomeLoad NoSharedMemory, --outFilterScoreMinOverLread 0, --
outFilterMatchNminOverLread 0, --outFilterMismatchNmax 999, and outFilterMultimapNmax
20. Duplicates were marked with picard MarkDuplicates version 2.22.0-SNAPSHOT.

RNA-sequencing BAM files for MCC tumor and cell line samples were downloaded from the Broad Genomics Firecloud/Terra platform using the Google Cloud Storage command line tool gsutil version 4.5 (github.com/GoogleCloudPlatform/gsutill).

Gene counts were obtained from BAM files using featureCounts version 2.0.0 (74). Very lowly expressed genes with average count across samples less than 1 were excluded from analysis. Between-sample distance metrics (FIG. 1C) were computed using the Euclidean distance on the vectors of variance-stabilized counts obtained from the vst function in the DESeq2 R Bioconductor package (73, 75).

Differential expression analysis was carried out between IFN-γ plus and minus samples (adjusting for viral status as a covariate) using the negative binomial GLM Wald test of DESeq2, where significance was assessed using the p-values adjusted for multiple comparisons under default settings. To account for potential global gene expression differences among sample groups, RUVg (76) was used to estimate latent factors of unwanted variation from the list of housekeeping genes downloaded from www.tau.ac.il/~elieis/HKG/HK_genes.txt. The largest factor of unwanted variation was then used as a covariate in the DESeq2 models to adjust for latent variation unrelated to library size. The normalized counts adjusted for the latent factors of variation returned by RUVg were visualized in FIG. 2A.

MCPyV viral DNA and RNA detection. DNA detection of MCPyV in MCC tumor samples was performed with ViroPanel as previously described (61). For viral transcript quantification of RNA-seq, the Merkel Cell Polyomavirus reference sequence was downloaded from https://www.ebi.ac.uk/ena/data/view/EU375804&display=fasta. Reads that did not map to the human reference sequence were extracted from RNA-seq and ViroPanel BAM files of tumor and cell line using SAMtools view version 1.10 (77) and realigned to a modified Merkel Cell Polyomavirus reference sequence (HM355825.1, recircularized such that the reference sequence ends when the VP2 coding sequence ends) using BWA version 0.7.17-r1188 (78). Coverage at each position was assessed with samtools using the command ‘samtools depth -aa -d0’, and coverage depth was plotting in R version 3.5.1 using the ggplot2 and gggenes packages.

Single-cell RNA sequencing. Tumor samples from MCC-336 (MCPyV+) and MCC-350 (MCPyV-) were processed for single cell RNA-seq (scRNAseq). Cells were thawed and washed twice in RPMI and 10% FBS before undergoing dead cell depletion (Miltenyi 130-090-101). Viable MCC tumor cells were resuspended in PBS with 0.04% BSA at the cell concentration of 1,000 cells/µL. 17,000 cells were loaded onto a 10× Genomics Chromium™ instrument (10× Genomics) according to the manufacturer’s instructions. The scRNAseq libraries were processed using Chromium™ single cell 5′ library & gel bead kit (10× Genomics). Quality control for amplified cDNA libraries and final sequencing libraries were performed using Bioanalyzer High Sensitivity DNA Kit (Agilent). ScRNAseq libraries were normalized to 4 nM concentration and pooled, and then the pooled libraries were sequenced on Illumina NovaSeq S4 platform. The sequencing parameters were: Read 1 of 150bp, Read 2 of 150bp, and Index 1 of 8bp. Reads from both samples were demultiplexed and aligned to hg19 using Cell Ranger (v. 3.0.2) (79) and the transcript quantities were co-analyzed using the Seurat (v. 3.1.5) R package (80). Only cells expressing >1,500 and <7,500 genes and <10 % mitochondrial genes were kept for further analysis, leaving a total of 15,808 cells sequenced to a mean depth of 4,231.9.00 genes/cell. The data were normalized and the top 2,000 variable features were identified. Subsequently, the data were scaled while regressing out variation from gene count, mitochondrial percentage, and cell cycle stage. This was followed by principal component analysis, batch correction using Harmony (v. 1.0) (81), UMAP analysis, and finally, Louvain clustering at resolution = 0.3. The immune cell cluster was identified by the expression of CD45 (PTPRC) and MCC clusters were identified by expression of ATOH1, SYP, and SOX2.

Immunoprecipitation and mass spectrometry analysis, and peptide identification. Up to 40 million or 0.2 g of MCC cells were immunoprecipitated. Briefly, MCC cells were harvested and lysed in ice-cold lysis buffer containing 40 M Tris (pH 8.0), 1 mM EDTA (pH 8.0), 0.1 M sodium chloride, Triton X-100, 0.06 M octyl β-d-glucopyranoside, 100 U/mL DNAse I, 1 mM phenylmethanesulfonyl fluoride (all from Sigma Aldrich), and protease inhibitor cocktail (Roche Diagnostics). Cell lysate was centrifuged at 12,700 rpm at 4° C. for 22 min. Lysate supernatant was coupled with Gammabind Plus sepharose beads (GE Healthcare) and incubated with 10 µg of HLA-I antibody (Clone W6/32, Santa Cruz Biotechnologies) at 4° C. under rotary agitation for 3 h. After incubation, the lysate-bead-antibody mixture was briefly centrifuged and the supernatant was discarded. Beads were washed with lysis buffer, consisting of wash buffer containing 40 mM Tris (pH 8.0), 1 mM EDTA (pH 8.0), 0.1 M sodium chloride, 0.06 M octyl β-d-glucopyranoside, and 20 mM Tris buffer, without protease inhibitors. Gel loading tips (Fisherbrand) were used to remove as much fluid from beads as possible. Peptides of up to three immunoprecipitations were combined, acid eluted, and analyzed using LC/MS-MS as described previously (35, 82). Briefly, peptides were resuspended in 3% acetonitrile with 5% formic acid and loaded onto an analytical column (20-30 cm with 1.9 µm C18 Reprosil beads, Dr. Maisch HPLC GmbH);, packed in-house). Peptides were eluted in a 6-30% gradient (EasyLC 1000 or 1200, Thermo Fisher Scientific) and analyzed on a QExactive Plus, Fusion Lumos, or Orbitrap Exploris 480 (Thermo Fisher Scientific). For Lumos measurements, peptides were also subjected to fragmentation if they were singly charged. For Orbitrap Exploris measurements (2 immunoprecipitations pooled, +/- IFN-y, FIGS. 3) and detection of the large T antigen peptide (3 immunoprecipitations of the MCC-367 cell line treated with IFN-γ) peptides were further fractionated using stage tip basic reverse phase separation with 2 punches of SDB-XC material (Empore 3 M) and increasing concentrations of acetonitrile (5%, 10% and 30% in 0.1% NH4OH, pH 10). Fractions were analyzed on a Fusion Lumos or Orbitrap Exploris 480 equipped with a FAIMSpro interface (83).

Immunopeptidomes of USP7 inhibitor treated cell lines were eluted as described above, followed by labeling with TMT6 reagent (Thermo Fisher; 126-USP7iA, 127-WT, 128 USP7iA, 129 WT, 130-USP7iB, 131 USP7iB) and then pooled for subsequent fractionation using basic reversed phase fractionation with increasing concentrations of acetonitrile (10%, 15% and 50%) in 5 mM ammonium formate (pH 10) and analysis on an Orbitrap Exploris 480 with FAIMSpro. Data acquisition parameters were as above with NCE set to 34 and 2 second dynamic exclusion.

Mass spectra were interpreted using Spectrum Mill software package v7.1 pre-Release (Broad Institute, Cambridge, MA). MS/MS spectra were excluded from searching if they did not have a precursor MH+ in the range of 600-4000, had a precursor charge >5, or had a minimum of <5 detected peaks. Merging of similar spectra with the same precursor m/z acquired in the same chromatographic peak was disabled. MS/MS spectra were searched against a protein sequence database that contained 90,904 entries, including all UCSC Genome Browser genes with hg19 annotation of the genome and its protein coding transcripts (52,788 entries), common human virus sequences (30,181 entries), recurrently mutated proteins observed in tumors from 26 tissues (4,595 entries), 264 common laboratory contaminants as well as protein sequences containing somatic mutations detected in MCC cell lines (3,076 entries). MS/MS search parameters included: no-enzyme specificity; ESI-QEXACTIVE-HCD-HLA-v3 instrument scoring; fixed modification: cysteinylation of cysteine; variable modifications: oxidation of methionine, carbamidomethylation of cysteine and pyroglutamic acid at peptide N-terminal glutamine; precursor mass tolerance of ±10 ppm; product mass tolerance of ±10 ppm, and a minimum matched peak intensity of 30%. Peptide spectrum matches (PSMs) for individual spectra were automatically designated as confidently assigned using the Spectrum Mill auto-validation module to apply target-decoy based FDR estimation at the PSM level of <1% FDR. Peptide auto-validation was done separately for each sample with an auto thresholds strategy to optimize score and delta Rank1 - Rank2 score thresholds separately for each precursor charge state (1 through 4) across all LC-MS/MS runs per sample. Score threshold determination also required that peptides had a minimum sequence length of 7, and PSMs had a minimum backbone cleavage score of 5. Peptide and PSM exports were filtered for contaminants including potential carry over tryptic peptides and peptides identified in a blank bead sample. For TMT-labeled samples, peptides derived from keratin proteins were removed and TMT intensity values were normalized to the global median. P-values were calculated using in house software based on the limma package in R.

Whole proteome analysis and interpretation. Protein expression of MCC cell lines was assessed as described previously (84). Briefly, cell pellets of MCC cell lines with and without IFN-y treatment were lysed in 8 M Urea and digested to peptides using LysC and Trypsin (Promega). 400 µg peptides were labeled with TMT10 reagents (Thermo Fisher, 126-MCC-290, 127N - MCC-350 _IFN, 127C MCC-275IFN, 128N MCC-275, 128C MCC-350, 129N_MCC-301_IFN, 129C - MCC-277_IFN, 130N-MCC-290_IFNy, 130C MCC-277, 131 MCC-301) and then pooled for subsequent fractionation and analysis. Pooled peptides were separated into 24 fractions using offline high pH reversed phase fractionation. 1 µg per fraction was loaded onto an analytical column (20-30 cm with 1.9 µm C18 Reprosil beads [Dr. Maisch HPLC GmbH], packed in-house, PicoFrit 75 µM inner diameter, 10 µM emitter [New Objective]). Peptides were eluted with a linear gradient (EasyNanoLC 1000 or 1200, Thermo Scientific) ranging from 6-30% Buffer B (either 0.1% formic acid or 0.5% AcOH and 80% or 90% acetonitrile) over 84 min 30-90% Buffer B over 9 min, and held at 90% Buffer B for 5 min at 200 nl/min. During data dependent acquisition, peptides were analyzed on a Fusion Lumos (Thermo Scientific). Full scan MS was acquired at a 60,000 from 300 - 1,800 m/z. AGC target was set to 4e5 and 50 ms. The top 20 precursors per cycle were subjected to HCD fragmentation at 60,000 resolution with an isolation width of 0.7 m/z, 34 NCE, 3e4 AGC target, and 50 ms max injection time. Dynamic exclusion was enabled with a duration of 45 sec.

Spectra were searched using Spectrum Mill against the database described above excluding MCC variants, specifying Trypsin/allow P (allows K-P and R-P cleavage) as digestion enzyme and allowing 4 missed cleavages, and ESI-QEXACTIVE-HCD-v3. Carbamidomethylation of cysteine was set as a fixed modification. TMT labeling was required at lysine, but peptide N-termini were allowed to be either labeled or unlabeled. Variable modifications searched include acetylation at the protein N-terminus, oxidized methionine, pyroglutamic acid, deamidated asparagine, and pyrocarbamidomethyl cysteine. Match tolerances were set to 20 ppm on MS1 and MS2 level. PSMs score thresholding used the Spectrum Mill auto-validation module to apply target-decoy based FDR in 2 steps: at the peptide spectrum match (PSM) level and the protein level. In step 1 PSM-level auto-validation was done first using an auto-thresholds strategy with a minimum sequence length of 8; automatic variable range precursor mass filtering; and score and delta Rank1 - Rank2 score thresholds optimized to yield a PSM-level FDR estimate for precursor charges 2 through 4 of <1.0% for each precursor charge state in each LC-MS/MS run. To achieve reasonable statistics for precursor charges 5-6, thresholds were optimized to yield a PSM-level FDR estimate of <0.5% across all LC runs per experiment (instead of per each run), since many fewer spectra are generated for the higher charge states. In step 2, protein-polishing auto-validation was applied to each experiment to further filter the PSMs using a target protein-level FDR threshold of zero, the protein grouping method expand subgroups, top uses shared (SGT) with an absolute minimum protein score of 9. TMT10 reporter ion intensities were corrected for isotopic impurities in the Spectrum Mill protein/peptide summary module using the afRICA correction method which implements determinant calculations according to Cramer’s Rule (85) and correction factors obtained from the reagent manufacturer’s certificate of analysis (www.thermofisher.com/order/catalog/product/90406) for lot number TB266293.

ELISpot. Matching patient peripheral blood mononuclear cells (PBMCs) from patient MCC-367 were thawed, and 10⁷ cells per well were seeded in 24 well plates overnight. Cells were stimulated with 10 ug/ml of the LT antigen peptide TSDKAIELY (SEQ ID NO:1) (identified in the MCC-367 HLA peptidome, FIG. 3F) in complete DMEM supplemented with 10% Human serum and 20 ng/ml IL-7 (PeproTech). After 3 days of stimulation, cells were supplemented with 20 units/mL IL-2 (PeproTech). After 10 days of stimulation, cells were cytokine deprived overnight. 50,000 cells per well were stimulated in an IFN- γ ELISpot assay with 10 ug/ml of the TSDKAIELY (SEQ ID NO:1) peptide. DMSO and an HIV-GAG peptide were used as negative controls. CEF (Mabtech) and PHA (Sigma Aldrich) were used as positive controls (not shown). ELISpot and T cell culture methods were described in detail previously (23, 86).

ORF Screen. The human ORFeome version 8.1 lentiviral library (38), which contains 16,172 unique ORFs mapping to 13,833 genes, was supplied as a gift from the Broad Genetic Perturbations Platform. 75 million MCC-301 cells were transduced with ORFeome lentivirus to achieve an infection rate of approximately 30-40%. Two days later, transduced cells were selected with three days of 0.5 ug/mL puromycin (Santa Cruz Biotechnology #SC-10871) treatment. Between 7-10 days after transduction, cells were stained with an anti-HLA-ABC-PE antibody (W6/32 clone, Biolegend #311405) and sorted on a BD FACSAria II, gating for the top and bottom 10% of HLA-ABC-PE staining. Sorted cells were washed with PBS, flash frozen, and stored at -80° C. Subsequently, genomic DNA containing stably integrated ORF sequences was isolated from the sorted cell pellets. The screen was performed in triplicate. Isolated genomic DNA was then used as a template for indexed PCR amplification of the construct barcode region. Pooled PCR products were purified and run on an Illumina HiSeq.

CRISPR-KO Screen. The Brunello human CRISPR knockout pooled plasmid library (Doench et al. 2016) (1-vector system) was a gift from David Root and John Doench (Addgene #73179). 50 ng of the Brunello plasmid library was electroporated into ElectroMAX Stbl4 competent cells (ThermoFisher #11635018) and incubated overnight at 30° C. on 24.5 × 24.5 cm agar bioassay plates. 20 hours later, colonies were harvested and pooled, and the amplified plasmid DNA (pDNA) was extracted and purified. To confirm that library diversity was maintained after amplification, sgRNA barcode construct regions were PCR amplified in pre- and post-amplification library aliquots. PCR products were purified and sequenced on an Illumina MiSeq. Sequencing data from pre- and post-amplification aliquots were compared to ensure similar diversity. To produce lentivirus, HEK-293T cells were transfected with pDNA, VSV-G, and psPAX2 plasmids using the TransIT-LT1 transfection reagent (Mirus #MIR2300). Lentivirus was harvested 48 hours post-transfection and flash frozen. To titrate lentivirus, 1.5 million cells MCC-301 cells were transduced with 100, 200, 300, 500, and 700 µL of virus. From each condition, half of the cells were selected with 0.5 µg/mL puromycin (Santa Cruz Biotechnology #SC-10871) while the other half were left untreated. Infection rates were calculated by comparing live cell counts in selected and unselected conditions.

Lentiviral transduction and FACS screening were performed in triplicate analogously to the ORF screen with the following exceptions: 150 million MCC-301 cells were transduced per replicate, and cells were sorted 10-14 days after transduction. Additionally, a representative pellet (40 million cells) after transduction but before flow cytometry selection was harvested and sequenced from all three replicates to assess sgRNA representation (FIG. 11B; FIG. 12A).

Screen Data Analysis. Unprocessed FASTQ reads were converted to log₂-normalized scores for each construct using PoolQ v2.2.0 (portals.broadinstitute.org/gpp/public/software/poolq). For each of the three replicates, log₂-fold changes (LFCs) between the normalized count scores of the HLA-I-high and HLA-I-low populations were calculated for each construct.

For the ORF screen, ORF constructs were then ranked based on their median LFC values, and corresponding p values were calculated using a hypergeometric distribution model (https://portals.broadinstitute.org/gpp/public/analysis-tools/crispr-gene-scoring). In cases where there were multiple ORFs mapping to one gene, LFC values were averaged across all constructs to generate a gene-level value. Sample quality for each sorted population was assessed by calculating log-normalized ORF construct scores (log₂ (ORF construct reads / total reads x 10⁶ + 1) and confirming than the mean construct frequency was no less than 10% of the expected frequency if all constructs were equally represented (corresponding to mean log-normalized score cutoff of 2.84) (FIG. 11B).

For the CRISPR screen, using equivalent cutoff criteria as above corresponding log-normalized score cutoff of 3.80), replicate 2 was discarded because the mean log-normalized score of the replicate 2 HLA-I-high sorted population was only 0.413 (FIG. 12A). Subsequently, LFC values for each sgRNA were averaged between replicate 1 and 3 only and then input into the STARS software (https://portals.broadinstitute.org/gpp/public/analysis-tools/crispr-gene-scoring) (39), which employs a binomial distribution model to rank genes based on the ranks of their corresponding individual sgRNAs.

For GSEA analysis, ranked ORF and CRISPR lists were generated by averaging the LFC values of all constructs mapping to or targeting a particular gene and ranking genes based on this average LFC. These ranked lists were then used as input for GSEAPreranked (enrichment statistic - weighted; max gene set size - 500; min gene set size - 15).

Generation of ORF lines. Single ORF constructs cloned into the pLX_TRC317 plasmid were a gift from the Broad Institute Genetic Perturbation Platform (portals.broadinstitute.org/gpp/public/). ORF plasmids, psPAX2, and VSV-G were transfected into HEK-293T cells to produce lentivirus. MCC-301 and MCC-277 cells were transduced with individual ORF lentivirus in 2 µg/mL polybrene, and spinfection was performed at 2,000 rpm for 2 hours at 30° C. Two days after transduction, transduced cells were selected with three days of 0.5 µg/mL puromycin treatment. Flow cytometry was performed as described above (see Methods: Flow cytometry) using either a PE-conjugated HLA-ABC (W6/32) antibody (BioLegend #311406) for MCC-301 lines or a AF647-conjugated HLA-ABC (W6/32) antibody (Santa Cruz Biotechnology #sc24637) for MCC-277 lines.

Generation of CRISPR KO lines. Forward and reverse oligos with the sequence 5′ CACCG----sgRNA sequence--- 3′ and 5′ AAAC—reverse complement of sgRNA ---C 3’ were synthesized by Eton Biosciences. Forward and reverse oligos were annealed and phosphorylated, producing BsmBI-compatible overhangs. LentiCRISPRv2 vector (Addgene #52961) was digested with BsmBI, dephosphorylated with shrimp alkaline phosphatase, and gel purified. Vector and insert were ligated at a 1:8 ratio with T7 DNA ligase at room temperature and transformed into Stbl3 chemically competent cells (ThermoFisher #C737303). Correct sgRNA cloning was confirmed via Sanger sequencing using the following primer: 5′-GATACAAGGCTGTTAGAGAGATAATT-3′ (SEQ ID NO: 20). Lentivirus was produced in HEK-293T cells (psPAX2, VSV-G, and cloned CRISPR plasmid), and MCC-301 cells were transduced with single construct lentivirus for single knockout lines, or with two lentivirus pools containing two different sgRNAs against the same gene for double knockout lines. Transduction was performed in the same manner as for the CRISPR-KO library. To validate gene editing for the single knockout lines, genomic DNA was extracted from both single knockout lines and WT MCC-301. Genomic DNA was then used as a template for PCR, with primers designed to flank the putative sgRNA binding sites. PCR products were purified and Sanger sequenced at Eton Biosciences. The percent of edited cells was then determined by TIDE (49) using WT MCC-301 as a reference. Flow cytometry was performed as described above (see Methods: Flow cytometry) using either a PE-conjugated HLA-ABC (W6/32) antibody (BioLegend #311406) for single knockout lines or a AF647-conjugated HLA-ABC (W6/32) antibody (Santa Cruz Biotechnology #sc24637) for double knockout lines.

Western blot analysis. Briefly, 1 million MCC-301 cells were transduced with single lentiviral constructs against a non-targeting control, PCGF1, BCORL1 or USP7. Two days after transduction, cells were subjected to selection with 0.5 ug/mL puromycin treatment for three days. For IFN-γ treatments, MCC-301 cell lines were treated with indicated doses of IFN- γ for 24 hours before harvesting for Western Blot analysis. Cells were collected by centrifugation, washed in PBS and lysed in EBC buffer (50 mM Tris-HCl, 200 mM NaCl, 0.5% NP-40, 0.5 mM EDTA) supplemented with protease and phosphatase inhibitors (Millipore) and 2-Mercaptoethanol (Bio-Rad) to obtain whole cell extracts. The cell extracts were clarified by centrifugation. The protein content of each sample was determined using BioRad BradFord assay following the addition of 6X Laemmli buffer (Boston bioproducts) and boiling of the samples at 95° C. for 5 minutes. A 4-20% gradient gel (Bio-Rad) was run for the analysis and the proteins were transferred to a 0.2 um Nitrocellulose membrane (Bio-Rad). The membrane was blocked using 5% milk in TBST at Room temperature for 1 hour followed by incubation with appropriate primary antibodies [USP7 (Life Technologies # PA534911), PCGF1 (E8, Santa Cruz Biotechnology # SC-515371), TAP1 (Cell Signaling Technology # 12341S), TAP2 (Cell Signaling Technology # 12259S), p53 (Santa Cruz Biotechnology # SC-126), pan-MYC (Abeam # ab195207), Vinculin (Sigma # V9131), TBP (Cell Signaling Technology # 8515S)] diluted according to manufacturer’s specifications in 5% milk in TBST at 4° C. overnight. The next day, membranes were washed thrice with TBST and incubated with the appropriate secondary antibody (Bethyl, Goat anti-mouse # A90-116P or Goat anti-Rabbit # A120-101P) diluted in 1% milk in TBST for one hour at room temperature. The membrane was washed thrice with TBST and incubated briefly with Immobilon Western Chemiluminescent (Millipore) HRP substrate followed by visualization of the signal on the G-box imaging system (Syngene). Raw Western Blot images were processed for visualization using the ImageJ software.

MKL-1 shMYCL and WaGa shST/LT RNA-seq and flow cytometry. A scramble shRNA constitutively expressed from the lentiviral PLKO vector (shScr) has been reported before (Addgene #1864). The MYCL and EP400 shRNA target sequences were designed using Block-iT RNAi Designer (Life Technologies). MYCL target -

GACCAAGAGGAAGAATCACAA (SEQ ID NO: 21)

; shEP400-2 target -

GCTGCGAAGAAGCTCGTTAGA (SEQ ID NO: 22)

, shEP400-3 target -

GGAGCAGCTTACACCAATTGA (SEQ ID NO: 23)

. Annealed forward and reverse oligos of shScr, shMYCL, shEP400-2, and shEP400-3 (Table S7) were cloned between AgeI/EcoRI sites of the doxycycline inducible shRNA vector Tet-pLKO-puro (a gift from Dmitri Wiederschain, Addgene #21915). 293T cells were transfected with the Tet-PLKO-puro plasmids plus psPAX2 packaging and VSV-G envelope plasmids (Addgene #12260 and #12259) to generate lentiviral particles for MKL-1 cell transduction. Transduced MKL-1 cells were selected with 1 µg puromycin for 4 days to generate Dox-inducible MKL-1 shScr, shMYCL, shEP400-2, and shEP400-3 lines. The Dox-inducible WaGa shST/LT line was a gift from Roland Houben (14).

For RNA-seq, cells were treated with dox as follows: MKL-1 shMYCL and shScr - 2 days Dox, MKL-1 shEP400-2, -3 and shScr - 6 days Dox, WaGa shST/LT cells with or without Dox - 6 days. Total RNA was extracted using RNeasy Plus Mini Kit (Qiagen). mRNA was isolated with NEB- Next Poly(A) mRNA Magnetic Isolation Module (New England BioLabs). Sequencing libraries were prepared with NEBNext mRNA library Prep Master Mix Set for Illumina (New England BioLabs) and passed Qubit, Bioanalyzer, and qPCR QC analyses. 50 cycles single-end sequencing was performed on the Illumina HiSeq 2000 system. Reads were mapped to the hg19 genome by TOPHAT. HTSeq was used to create a count file containing gene names (87). The R package DESeq2 was used to normalize counts and calculate total reads per million (TPM) and determine differential gene expression. Quality control was performed by inspecting a MA plot of differentially expressed genes. RNA-seq data are available from the Gene Expression Omnibus with accession number GSE69878. For GSEA analysis, genes were ranked based on their LFC value from DESeq2. These ranked lists were then used as input for GSEAPreranked (enrichment statistic - weighted; max gene set size - 500; min gene set size - 15).

For flow cytometry, shMYCL and shScr MKL-1 cells were treated with 0.2 µg/mL doxycycline for 7 days, refreshing with doxycycline-containing media every 3 days. In addition, shMYCL cells containing a constitutively expressed (Addgene, #17486) shRNA-resistant MYCL (shMYCL+MYCL) construct were identically treated. Single cell suspensions were prepared non-enzymatically via treatment with Versene (Gibco 15040066). Cells were incubated with Human True-Stain FcX (BioLegend # 422302), followed by staining with an anti-HLA-A/B/C antibody (SCBT, #32235) or isotype-matched IgG control (SCBT, #24637) conjugated to Alexa Fluor 647. Stained cells were strained through a 100 µm filter and fluorescence was measured via flow cytometry (BD, LSR Fortessa). Single cells were selected utilizing FSC-H/FSC-A discrimination and the geometric mean of Alexa Fluor 647 fluorescence was calculated from the single cell population.

ChIP-Seq and ChIP-qPCR. ChIP-seq data for MAX, EP400, ST, H3K4me3, and H3K27ac were generated as previously described (15). For ChIP-qPCR, the following primers were designed using PrimerQuest (IdtDNA) based on ChIP-seq data displayed in UCSC genome browser (Table S7). qPCR was performed using the Brilliant III ultra-fast SYBR green qPCR master mix (Agilent) on the AriaMx Real-time PCR System (Agilent) by following the instruction manual.

MCC Tumor RNA-seq Cohort. Tumor biopsies were collected from 52 patients at the DFCI and preserved for RNA isolation via addition of RNAlater (Sigma-Aldrich). Preserved tissue was homogenized via TissueRuptor (QIAGEN) and RNA was harvested via AllPrep DNA/RNA Mini Kit (QIAGEN). RNA was submitted for library construction utilizing the NEBNext Ultra II RNA Library Prep Kit for Illumina (NEB). Paired-end sequencing was performed on the NovaSeq 6000 system for 150 cycles in each direction (Novogene). Raw paired-end sequencing data were broadly assessed for quality via FastQC (www.bioinformatics.babraham.ac.uk/projects/fastqc/). Samples passing quality control were quantified to the transcript level via Salmon (88) utilizing Ensembl gene annotations for the GRCh38.p13 genome assembly. Normalized gene-level counts were prepared with TxImport and DESeq2 (75, 89). To identify virus-positive or virus-negative samples, paired-end reads were mapped to the MCPyV genome (R17b isolate) via BWA (78) and those sample containing MCPyV-specific reads (>100) were considered virus-positive. For the RNA-seq heatmap, z-scores of the log₂-normalized gene-level counts were calculated. One tumor sample was subsequently discarded as an outlier because the z-score was >3.5 or < -3.5 in 7 of the 18 genes analyzed in this sample (for comparison, the range of z-scores for all 18 genes in all other samples was -3.45 to 2.47). The remaining 51 tumor samples were subsequently clustered by Euclidian distance to generate the RNA-seq heatmap. Tumor purity was determined using the ESTIMATE R Package (51). Tumor purity percentage was calculated from the ESTIMATE score using the equation: cos(0.6049872018+0.0001467884 × ESTIMATE score) as published.

PCGF1-KO RNA-seq and Western Blots. RNA was extracted from three technical replicates of the MCC-301 PCGF1-KO #2 line (second-highest scoring guide RNA) and of an MCC-301 line transduced with a non-targeting sgRNA control and Cas9. Sample preparation and sequencing was performed as described above in “RNA sequencing and analysis”. Subsequently, raw FASTQ files were broadly assessed for sequencing quality via FastQC (Babraham Institute), with those of passing quality used for further analysis. Salmon (88) was used to map raw reads to the decoy-aware transcriptome of GRCh38p.13 v99 (Ensembl) with the following stipulations: --writeUnmappedNames, --seqBias, --gcBias, --validateMappings. Raw transcript-level counts were converted to gene-level counts via TxImport (90) and differential gene expression analysis was performed using DeSeq2 (91).

For TAP1 Western blots, IFN-_{γ} titration was first performed in MKL-1 cells (FIG. 12F) to determine the IFN-_{γ} range over which TAP1 expression became detectable. Concentrations of 0, 100, and 1,000 U/mL IFN-_{γ} were subsequently used for TAP1 Western blots in MCC-301 PCGFI-KO and control sgRNA lines.

Cell cycle analysis. 1 million MKL-1 control or p53 KO cells were plated and treated with DMSO, XL177A (100 nM) or XL177B (100 nM) for three days. During the last hour of the three- day treatment, the cells were pulsed with 10 µM EdU nucleotide. The cells were collected by centrifugation, treated with Accutase™ (Stem Cell Technologies) to break apart clumps, washed with PBS and fixed using 4% Formaldehyde solution in PBS at Room temperature for 15 mins. Cells were washed with 1% BSA in PBS and resuspended in 70% ice cold ethanol and incubated at -20° C. overnight for additional fixing and permeabilization. The cells were stored in 70% ethanol at -20° C. until the day the data was acquired. On the day of data acquisition, the cells were collected by centrifugation and washed twice with PBS. The incorporated EdU in the cells were labeled with a CLICK reaction cocktail (1 mM CuSO4, 100 µM THPTA, 100 mM sodium ascorbate, and 2.2 µM Alexa 647 azide in PBS) at room temperature with rocking for 30 minutes. The samples were then washed with 1% BSA in PBS once followed by two washes with PBS and incubated with a 1 µg/ml DAPI, 100 ng/ml RNase A solution for one hour at Room temperature to stain the DNA. The samples were then passed through strainer tubes and analyzed using a BD Fortessa analyzer. The flow cytometry data was analyzed using the FlowJo Software. The percentage of cells in each cell cycle phase was represented using GraphPad PRISM software.

USP7 inhibitor experiments. For MCC-301 USP7 inhibitor experiments, two and a half million MCC cells were plated in a T25 flask and incubated with the USP7 inhibitor XL177A and control enantiomer XL177B at 10 µM, 1 µM, 100 nM, and 10 nM. Cells were incubated for 3 to 4 days. Post incubation, one million cells were treated with Versene (Gibco) to dissociate cell clusters. Surface Fc receptors were blocked with 5 µL Human TruStain FcX (Biolegend # 422302). Surface HLA-I was stained with 5 µL of Pan HLA-Class I antibody (Clone W6/32, Santa Cruz Biotechnologies) for 30 minutes in dark at 4° C. Cells were washed with PBS and fixed with 4% paraformaldehyde fixation buffer (Biolegend). Cells were analyzed on a BD LSRFortessa. MCC-301 data are representative of 4 independent experiments. To perform statistical analysis, for each cell line, one-way ANOVA was first performed on the MFIs of the DMSO group and all experimental groups. Then, individual Welch t-tests were performed for each concentration, comparing the fold-changes ofMFI (inhibitor) / mean MFI (DMSO control) between XL177A and XL177B.

For MKL-1 USP7 inhibitor experiments, p53-WT control lines (WT, scrambled, AAVS1) and three p53-KO lines were treated with USP7 inhibitors and assessed by flow cytometry for surface HLA I as described above for MCC-301. Because the root mean squared error differed considerably between the control lines and the p53-KO lines (12.2894 and 6.69844), the two groups were analyzed separately by two-way ANOVAs, and drug treatment was found to be a statistically significant source of variation in MFI in both cases (P = 0.0003 in controls and P < 0.0001 in p53-KO lines). ANOVA was followed by post hoc Tukey’s multiple comparisons tests between XL177A, XL I 77B, and DMSO treatments to generate the p-values displayed in FIG. 6C.

Dependency Map Correlations. The DepMap 20Q2 CRISPR dependency data were downloaded from www.depmap.org/portal/download. TP53 mutation status was assigned using the Cell-Line Selector tool on the DepMap Portal based on criteria of at least one coding mutation. Pearson coefficients were calculated using test. cor in R, and two-sided p-values outputted by this function were converted into FDR using p.adjust. Plots were generated using ggplot2, tidyverse, gridExtra, cowplot, and scales. GSEA was performed using a gene list ranked by -log(p-val) multiplied by (-1) if the Pearson correlation was negative.

Quantification and Statistical Analysis. All flow cytometry bar graphs show mean fluorescence intensity of three technical or biological replicates, except for FIG. 1D and Extended Data FIG. 2C which show one sample. Error bars indicates standard deviation, unless otherwise stated. P-value of 0.05 was used as the significance threshold in all experiments. Specific statistical tests used in each figure are mentioned in the figure legends and/or the methods section.

Specific software with version number, along with details of all statistical analyses are listed in the respective methods sections above. No randomization procedures or sample size calculations were carried out as part of the study. All analysis code including specific parameter settings for whole exome sequencing analysis, RNA-seq analysis, MCPyV viral transcript detection, and WGBS promoter signal extraction are made available in a GitHub repository under an MIT license at www.github.com/kdkorthauer/MCC. All analyses in R were carried out using version 3.6.2.

ATAC-seq. Differential peak analysis: Differential ATAC-seq peaks between (1) viral positive and negative samples, and (2) IFNg responsive and non-responsive (split into top four and bottom four) were called using the DiffBind R Bioconductor package (Ross-Innes et al. 2012). Significance was assessed using the using adjusted p-values from the negative binomial GLM Wald test of DESeq2, which is called by DiffBind. Peaks were annotated by the gene with the nearest TSS using the ChIPpeakAnno (Zhu et al. 2010) and the TxDb.Hsapiens.UCSC.hg38.knownGene (TxDb.Hsapiens.UCSC.hg19.knownGene ) R Bioconductor packages.

Comparison ATAC-Seq datasets for visualization were retrieved from GEO (GSM2702712 - primary B-cells; GSM2476340 - 501MEL cell line) and ENCODE (ENCFF654ZNI - primary fetal foreskin keratinocyte). To visualize ATAC-Seq tracks, all BAM files were normalized identically using bamCoverage from deepTools (academic.oup.com/nar/article/44/Wl/W160/2499308) with a 10 nucleotide bin size and normalization method of reads per kilobase of transcript per million reads (RPKM). Resulting bigwig files were visualized in the Integrative Genome Viewer (www.ncbi.nlm.nih.gov/pmc/articles/PMC3346182/).

Averaging over promoter regions: Bismark methylation count output files (.cov) were strand-collapsed using the bsseq Bioconductor package (Hansen et al. 2012). CpG sites covered by at least 1 read in fewer than 4 samples were excluded from further analysis. Promoter regions (2000 basepair upstream, 200 basepair downstream) of all transcripts annotated by the TxDb.Hsapiens.UCSC.hg38.knownGene (TxDb.Hsapiens.UCSC.hg19.knownGene) R Bioconductor package. Then, raw methylation levels (methylated counts divided by coverage) for all sites within each promoter region of all transcripts matching each gene symbol were averaged.

### References and Notes

1. C. C. Chang, M. Campoli, S. Ferrone, Classical and nonclassical HLA class I antigen and NK Cell-activating ligand changes in malignant cells: current challenges and future directions. Advances in cancer research 93, 189-234 (2005).

2. Y. Nie, G. Yang, Y. Song, X. Zhao, C. So, J. Liao, L. D. Wang, C. S. Yang, DNA hypermethylation is a mechanism for loss of expression of the HLA class I genes in human esophageal squamous cell carcinomas. Carcinogenesis 22, 1615-1623 (2001).

3. S. A. Shukla, M. S. Rooney, M. Rajasagi, G. Tiao, P. M. Dixon, M. S. Lawrence, J. Stevens, W. J. Lane, J. L. Dellagatta, S. Steelman, C. Sougnez, K. Cibulskis, A. Kiezun, N. Hacohen, V. Brusic, C. J. Wu, G. Getz, Comprehensive analysis of cancer-associated somatic mutations in class I HLA genes. Nature biotechnology 33, 1152-1158 (2015).

4. J. M. Zaretsky, A. Garcia-Diaz, D. S. Shin, H. Escuin-Ordinas, W. Hugo, S. Hu-Lieskovan, D. Y. Torrejon, G. Abril-Rodriguez, S. Sandoval, L. Barthly, J. Saco, B. Homet Moreno, R. Mezzadra, B. Chmielowski, K. Ruchalski, I. P. Shintaku, P. J. Sanchez, C. Puig-Saus, G. Cherry, E. Seja, X. Kong, J. Pang, B. Berent-Maoz, B. Comin-Anduix, T. G. Graeber, P. C. Tumeh, T. N. M. Schumacher, R. S. Lo, A. Ribas, Mutations Associated with Acquired Resistance to PD-1 Blockade in Melanoma. New England Journal of Medicine 375, 819-829 (2016).

5. S. J. Rodig, D. Gusenleitner, D. G. Jackson, E. Gjini, A. Giobbie-Hurder, C. Jin, H. Chang, S. B. Lovitch, C. Horak, J. S. Weber, J. L. Weirather, J. D. Wolchok, M. A. Postow, A. C. Pavlick, J. Chesney, F. S. Hodi, MHC proteins confer differential sensitivity to CTLA-4 and PD-1 blockade in untreated metastatic melanoma. Science translational medicine 10, (2018).

6. M. Sade-Feldman, Y. J. Jiao, J. H. Chen, M. S. Rooney, M. Barzily-Rokni, J.-P. Eliane, S. L. Bjorgaard, M. R. Hammond, H. Vitzthum, S. M. Blackmon, D. T. Frederick, M. Hazar-Rethinam, B. A. Nadres, E. E. Van Seventer, S. A. Shukla, K. Yizhak, J. P. Ray, D. Rosebrock, D. Livitz, V. Adalsteinsson, G. Getz, L. M. Duncan, B. Li, R. B. Corcoran, D. P. Lawrence, A. Stemmer-Rachamimov, G. M. Boland, D. A. Landau, K. T. Flaherty, R. J. Sullivan, N. Hacohen, Resistance to checkpoint blockade therapy through inactivation of antigen presentation. Nature Communications 8, 1136 (2017).

7. S. Gettinger, J. Choi, K. Hastings, A. Truini, I. Datar, R. Sowell, A. Wurtz, W. Dong, G. Cai, M. A. Melnick, V. Y. Du, J. Schlessinger, S. B. Goldberg, A. Chiang, M. F. Sanmamed, I. Melero, J. Agorreta, L. M. Montuenga, R. Lifton, S. Ferrone, P. Kavathas, D. L. Rimm, S. M. Kaech, K. Schalper, R. S. Herbst, K. Politi, Impaired HLA Class I Antigen Processing and Presentation as a Mechanism of Acquired Resistance to Immune Checkpoint Inhibitors in Lung Cancer. Cancer Discov 7, 1420-1435 (2017).

8. K. G. Paulson, V. Voillet, M. S. McAfee, D. S. Hunter, F. D. Wagener, M. Perdicchio, W. J. Valente, S. J. Koelle, C. D. Church, N. Vandeven, H. Thomas, A. G. Colunga, J. G. Iyer, C. Yee, R. Kulikauskas, D. M. Koelle, R. H. Pierce, J. H. Bielas, P. D. Greenberg, S. Bhatia, R. Gottardo, P. Nghiem, A. G. Chapuis, Acquired cancer resistance to combination immunotherapy from transcriptional loss of class I HLA. Nature Communications 9, 3868 (2018).

9. L. Ni, J. Lu, Interferon gamma in cancer immunotherapy. Cancer Medicine 7, 4509-4516 (2018).

10. F. Castro, A. P. Cardoso, R. M. Goncalves, K. Serre, M. J. Oliveira, Interferon-Gamma at the Crossroads of Tumor Immune Surveillance or Evasion. Front Immunol 9, 847 (2018).

11. M. Mojic, K. Takeda, Y. Hayakawa, The Dark Side of IFN-y: Its Role in Promoting Cancer Immunoevasion. Int J Mol Sci 19, 89 (2017).

12. D. Schrama, W. K. Peitsch, M. Zapatka, H. Kneitz, R. Houben, S. Eib, S. Haferkamp, P. S. Moore, M. Shuda, J. F. Thompson, U. Trefzer, C. Pföhler, R. A. Scolyer, J. C. Becker, Merkel cell polyomavirus status is not associated with clinical course of Merkel cell carcinoma. The Journal of investigative dermatology 131, 1631-1638 (2011).

13. A. S. Moshiri, R. Doumani, L. Yelistratova, A. Blom, K. Lachance, M. M. Shinohara, M. Delaney, O. Chang, S. McArdle, H. Thomas, M. M. Asgari, M. L. Huang, S. M. Schwartz, P. Nghiem, Polyomavirus-Negative Merkel Cell Carcinoma: A More Aggressive Subtype Based on Analysis of 282 Cases Using Multimodal Tumor Virus Detection. The Journal of investigative dermatology 137, 819-827 (2017).

14. S. Hesbacher, L. Pfitzer, K. Wiedorfer, S. Angermeyer, A. Borst, S. Haferkamp, C. J. Scholz, M. Wobser, D. Schrama, R. Houben, RB1 is the crucial target of the Merkel cell polyomavirus Large T antigen in Merkel cell carcinoma cells. Oncotarget 7, 32956-32968 (2016).

15. J. Cheng, D. E. Park, C. Berries, E. A. White, R. Arora, R. Yoon, T. Branigan, T. Xiao, T. Westerling, A. Federation, R. Zeid, B. Strober, S. K. Swanson, L. Florens, J. E. Bradner, M. Brown, P. M. Howley, M. Padi, M. P. Washburn, J. A. DeCaprio, Merkel cell polyomavirus recruits MYCL to the EP400 complex to promote oncogenesis. PLoSpathogens 13, e1006668 (2017).

16. K. G. Paulson, A. Tegeder, C. Willmes, J. G. Iyer, 0. K. Afanasiev, D. Schrama, S. Koba, R. Thibodeau, K. Nagase, W. T. Simonson, A. Seo, D. M. Koelle, M. Madeleine, S. Bhatia, H. Nakajima, S. Sano, J. S. Hardwick, M. L. Disis, M. A. Cleary, J. C. Becker, P. Nghiem, Downregulation of MHC-I Expression Is Prevalent but Reversible in Merkel Cell Carcinoma. Cancer Immunology Research 2, 1071 (2014).

17. C. Ritter, K. Fan, A. Paschen, S. Reker Hardrup, S. Ferrone, P. Nghiem, S. Ugurel, D. Schrama, J. C. Becker, Epigenetic priming restores the HLA class-I antigen processing machinery expression in Merkel cell carcinoma. Scientific reports 7, 2290 (2017).

18. K. Daily, A. Coxon, J. S. Williams, C. R. Lee, D. G. Coit, K. J. Busam, I. Brownell, Assessment of cancer cell line representativeness using microarrays for Merkel cell carcinoma. The Journal of investigative dermatology 135, 1138-1146 (2015).

19. J. H. Leonard, J. R. Bell, J. H. Kearsley, Characterization of cell lines established from merkel-cell (“small-cell”) carcinoma of the skin. International journal of cancer 55, 803-810 (1993).

20. R. Houben, M. Shuda, R. Weinkam, D. Schrama, H. Feng, Y. Chang, P. S. Moore, J. C. Becker, Merkel cell polyomavirus-infected Merkel cell carcinoma cells require expression of viral T antigens. Journal of virology 84, 7064-7072 (2010).

21. L. R. Dresang, A. Guastafierro, R. Arora, D. Normolle, Y. Chang, P. S. Moore, Response of Merkel cell polyomavirus-positive merkel cell carcinoma xenografts to a survivin inhibitor. PloS one 8, e80543 (2013).

22. D. Schrama, E. M. Sarosi, C. Adam, C. Ritter, U. Kaemmerer, E. Klopocki, E. M. Konig, J. Utikal, J. C. Becker, R. Houben, Characterization of six Merkel cell polyomavirus-positive Merkel cell carcinoma cell lines: Integration pattern suggest that large T antigen truncating events occur before or during integration. International journal of cancer 145, 1020-1032 (2019).

23. D. B. Keskin, A. J. Anandappa, J. Sun, I. Tirosh, N. D. Mathewson, S. Li, G. Oliveira, A. Giobbie-Hurder, K. Felt, E. Gjini, S. A. Shukla, Z. Hu, L. Li, P. M. Le, R. L. Allesoe, A. R. Richman, M. S. Kowalczyk, S. Abdelrahman, J. E. Geduldig, S. Charbonneau, K. Pelton, J. B. Iorgulescu, L. Elagina, W. Zhang, O. Olive, C. McCluskey, L. R. Olsen, J. Stevens, W. J. Lane, A. M. Salazar, H. Daley, P. Y. Wen, E. A. Chiocca, M. Harden, N. J. Lennon, S. Gabriel, G. Getz, E. S. Lander, A. Regev, J. Ritz, D. Neuberg, S. J. Rodig, K. L. Ligon, M. L. Suva, K. W. Wucherpfennig, N. Hacohen, E. F. Fritsch, K. J. Livak, P. A. Ott, C. J. Wu, D. A. Reardon, Neoantigen vaccine generates intratumoral T cell responses in phase Ib glioblastoma trial. Nature 565, 234-239 (2019).

24. M. E. Verhaegen, D. Mangelberger, J. W. Weick, T. D. Vozheiko, P. W. Harms, K. T. Nash, E. Quintana, P. Baciu, T. M. Johnson, C. K. Bichakjian, A. A. Dlugosz, Merkel cell carcinoma dependence on bcl-2 family members for survival. The Journal of investigative dermatology 134, 2241-2250 (2014).

25. A. Harold, Y. Amako, J. Hachisuka, Y. Bai, M. Y. Li, L. Kubat, J. Gravemeyer, J. Franks, J. R. Gibbs, H. J. Park, E. Ezhkova, J. C. Becker, M. Shuda, Conversion of Sox2-dependent Merkel cell carcinoma to a differentiated neuron-like phenotype by T antigen inhibition. Proceedings of the National Academy of Sciences 116, 20104 (2019).

26. M. K. Slevin, B. M. Wollison, W. Powers, R. T. Burns, N. Patel, M. D. Ducar, G. J. Starrett, E. P. Garcia, D. K. Manning, J. Cheng, G. J. Hanna, K. M. Kaye, P. Van Hummelen, A. Nag, A. R. Thorner, J. A. DeCaprio, L. E. MacConaill, ViroPanel: Hybrid Capture and Massively Parallel Sequencing for Simultaneous Detection and Profiling of Oncogenic Virus Infection and Tumor Genome. The Journal of molecular diagnostics : JMD 22, 476-487 (2020).

27. G. Goh, T. Walradt, V. Markarov, A. Blom, N. Riaz, R. Doumani, K. Stafstrom, A. Moshiri, L. Yelistratova, J. Levinsohn, T. A. Chan, P. Nghiem, R. P. Lifton, J. Choi, Mutational landscape of MCPyV-positive and MCPyV-negative Merkel cell carcinomas with implications for immunotherapy.

28. T. C. Knepper, M. Montesion, J. S. Russell, E. S. Sokol, G. M. Frampton, V. A. Miller, L. A. Albacker, H. L. McLeod, Z. Eroglu, N. I. Khushalani, V. K. Sondak, J. L. Messina, M. J. Schell, J. A. DeCaprio, K. Y. Tsai, A. S. Brohl, The Genomic Landscape of Merkel Cell Carcinoma and Clinicogenomic Biomarkers of Response to Immune Checkpoint Inhibitor Therapy. Clin Cancer Res 25, 5961-5971 (2019).

29. N. C. Blessin, P. Spriestersbach, W. Li, T. Mandelkow, D. Dum, R. Simon, C. Hube-Magg, F. Lutz, F. Viehweger, M. Lennartz, C. Fraune, V. Nickelsen, W. Fehrle, C. Gobel, S. Weidemann, T. Clauditz, P. Lebok, K. Moller, S. Steurer, J. R. Izbicki, G. Sauter, S. Minner, F. Jacobsen, A. M. Luebke, F. Buscheck, D. Hoflmayer, W. Wilczak, E. Burandt, A. Hinsch, Prevalence of CD8(+) cytotoxic lymphocytes in human neoplasms. Cellular oncology (Dordrecht) 43, 421-430 (2020).

30. R. J. Butterfield, D. M. Dunn, Y. Hu, K. Johnson, C. G. Bonnemann, R. B. Weiss, Transcriptome profiling identifies regulators of pathogenesis in collagen VI related muscular dystrophy. PloS one 12, e0189664 (2017).

31. W. R. Swindell, M. K. Sarkar, Y. Liang, X. Xing, J. Baliwag, J. T. Elder, A. Johnston, N. L. Ward, J. E. Gudjonsson, RNA-seq identifies a diminished differentiation gene signature in primary monolayer keratinocytes grown from lesional and uninvolved psoriatic skin. Scientific reports 7, 18045 (2017).

32. J. C. Sunshine, N. S. Jahchan, J. Sage, J. Choi, Are there multiple cells of origin of Merkel cell carcinoma? Oncogene 37, 1409-1416 (2018).

33. S. Vijayan, T. Sidiq, S. Yousuf, P. J. van den Elsen, K. S. Kobayashi, Class I transactivator, NLRCS: a central player in the MHC class I pathway and cancer immune surveillance. Immunogenetics 71, 273-282 (2019).

34. S. Yoshihama, J. Roszik, I. Downs, T. B. Meissner, S. Vijayan, B. Chapuy, T. Sidiq, M. A. Shipp, G. A. Lizee, K. S. Kobayashi, NLRC5/MHC class I transactivator is a target for immune evasion in cancer. Proceedings of the National Academy of Sciences 113, 5999 (2016).

35. S. Sarkizova, S. Klaeger, P. M. Le, L. W. Li, G. Oliveira, H. Keshishian, C. R. Hartigan, W. Zhang, D. A. Braun, K. L. Ligon, P. Bachireddy, I. K. Zervantonakis, J. M. Rosenbluth, T. Ouspenskaia, T. Law, S. Justesen, J. Stevens, W. J. Lane, T. Eisenhaure, G. Lan Zhang, K. R. Clauser, N. Hacohen, S. A. Carr, C. J. Wu, D. B. Keskin, A large peptidome dataset improves HLA class I epitope prediction across most of the human population. Nature biotechnology 38, 199-209 (2020).

36. A. Javitt, E. Barnea, M. P. Kramer, H. Wolf-Levy, Y. Levin, A. Admon, Y. Merbl, Pro-inflammatory Cytokines Alter the Immunopeptidome Landscape by Modulation of HLA-B Expression. Front Immunol 10, 141-141 (2019).

37. J. Girdlestone, Regulation of HLA class I loci by interferons. Immunobiology 193, 229-237 (1995).

38. X. Yang, J. S. Boehm, X. Yang, K. Salehi-Ashtiani, T. Hao, Y. Shen, R. Lubonja, S. R. Thomas, O. Alkan, T. Bhimdi, T. M. Green, C. M. Johannessen, S. J. Silver, C. Nguyen, R. R. Murray, H. Hieronymus, D. Balcha, C. Fan, C. Lin, L. Ghamsari, M. Vidal, W. C. Hahn, D. E. Hill, D. E. Root, A public genome-scale lentiviral expression library of human ORFs. Nature methods 8, 659-661 (2011).

39. J. G. Doench, N. Fusi, M. Sullender, M. Hegde, E. W. Vaimberg, K. F. Donovan, I. Smith, Z. Tothova, C. Wilen, R. Orchard, H. W. Virgin, J. Listgarten, D. E. Root, Optimized sgRNA design to maximize activity and minimize off-target effects of CRISPR-Cas9. Nature biotechnology 34, 184-191 (2016).

40. A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: A knowledge-based approach for interpreting genome-wide expression profiles. Proceedings of the National Academy of Sciences 102, 15545 (2005).

41. D. E. Park, J. Cheng, C. Berrios, J. Montero, M. Cortes-Cros, S. Ferretti, R. Arora, M. L. Tillgren, P. C. Gokhale, J. A. DeCaprio, Dual inhibition of MDM2 and MDM4 in virus-positive Merkel cell carcinoma enhances the p53 response. Proceedings of the National Academy of Sciences 116, 1027 (2019).

42. D. E. Park, J. Cheng, J. P. McGrath, M. Y. Lim, C. Cushman, S. K. Swanson, M. L. Tillgren, J. A. Paulo, P. C. Gokhale, L. Florens, M. P. Washburn, P. Trojer, J. A. DeCaprio, Merkel cell polyomavirus activates LSD1-mediated blockade of non-canonical BAF to regulate transformation and tumorigenesis. Nature cell biology 22, 603-615 (2020).

43. K. G. Paulson, B. D. Lemos, B. Feng, N. Jaimes, P. F. Peñas, X. Bi, E. Maher, L. Cohen, J. H. Leonard, S. R. Granter, L. Chin, P. Nghiem, Array-CGH reveals recurrent genomic changes in Merkel cell carcinoma including amplification of L-Myc. The Journal of investigative dermatology 129, 1547-1555 (2009).

44. M. Ghandi, F. W. Huang, J. Jane-Valbuena, G. V. Kryukov, C. C. Lo, E. R. McDonald, 3rd, J. Barretina, E. T. Gelfand, C. M. Bielski, H. Li, K. Hu, A. Y. Andreev-Drakhlin, J. Kim, J. M. Hess, B. J. Haas, F. Aguet, B. A. Weir, M. V. Rothberg, B. R. Paolella, M. S. Lawrence, R. Akbani, Y. Lu, H. L. Tiv, P. C. Gokhale, A. de Weck, A. A. Mansour, C. Oh, J. Shih, K. Hadi, Y. Rosen, J. Bistline, K. Venkatesan, A. Reddy, D. Sonkin, M. Liu, J. Lehar, J. M. Korn, D. A. Porter, M. D. Jones, J. Golji, G. Caponigro, J. E. Taylor, C. M. Dunning, A. L. Creech, A. C. Warren, J. M. McFarland, M. Zamanighomi, A. Kauffmann, N. Stransky, M. Imielinski, Y. E. Maruvka, A. D. Cherniack, A. Tsherniak, F. Vazquez, J. D. Jaffe, A. A. Lane, D. M. Weinstock, C. M. Johannessen, M. P. Morrissey, F. Stegmeier, R. Schlegel, W. C. Hahn, G. Getz, G. B. Mills, J. S. Boehm, T. R. Golub, L. A. Garraway, W. R. Sellers, Next-generation characterization of the Cancer Cell Line Encyclopedia. Nature 569, 503-508 (2019).

45. A. G. Grandea, 3rd, T. N. Golovina, S. E. Hamilton, V. Sriram, T. Spies, R. R. Brutkiewicz, J. T. Harty, L. C. Eisenlohr, L. Van Kaer, Impaired assembly yet normal trafficking of MHC class I molecules in Tapasin mutant mice. Immunity 13, 213-222 (2000).

46. V. van den Boom, H. Maat, M. Geugien, A. Rodriguez López, A. M. Sotoca, J. Jaques, A. Z. Brouwers-Vos, F. Fusetti, R. W. Groen, H. Yuan, A. C. Martens, H. G. Stunnenberg, E. Vellenga, J. H. Martens, J. J. Schuringa, Non-canonical PRC1.1 Targets Active Genes Independent of H3K27me3 and Is Essential for Leukemogenesis. Cell Rep 14, 332-346 (2016).

47. J. M. Hübner, T. Muller, D. N. Papageorgiou, M. Mauermann, J. Krijgsveld, R. B. Russell, D. W. Ellison, S. M. Pfister, K. W. Pajtler, M. Kool, EZHIP/CXorf67 mimics K27M mutated oncohistones and functions as an intrinsic inhibitor of PRC2 function in aggressive posterior fossa ependymoma. Neuro-oncology 21, 878-889 (2019).

48. A. Basu, F. H. Wilkinson, K. Colavita, C. Fennelly, M. L. Atchison, YY1 DNA binding and interaction with YAF2 is essential for Polycomb recruitment. Nucleic acids research 42, 2208-2223 (2014).

49. E. K. Brinkman, T. Chen, M. Amendola, B. van Steensel, Easy quantitative assessment of genome editing by sequence trace decomposition. Nucleic acids research 42, e168 (2014).

50. Y. Yan, W. Zhao, Y. Huang, H. Tong, Y. Xia, Q. Jiang, J. Qin, Loss of Polycomb Group Protein Pcgf1 Severely Compromises Proper Differentiation of Embryonic Stem Cells. Scientific reports 7, 46276 (2017).

51. K. Yoshihara, M. Shahmoradgoli, E. Martinez, R. Vegesna, H. Kim, W. Torres-Garcia, V. Treviño, H. Shen, P. W. Laird, D. A. Levine, S. L. Carter, G. Getz, K. Stemke-Hale, G. B. Mills, R. G. Verhaak, Inferring tumour purity and stromal and immune cell admixture from expression data. Nat Commun 4, 2612 (2013).

52. M. Czech-Sioli, S. Siebels, S. Radau, R. P. Zahedi, C. Schmidt, T. Dobner, A. Grundhoff, N. Fischer, The Ubiquitin-Specific Protease Usp7, a Novel Merkel Cell Polyomavirus Large T-Antigen Interaction Partner, Modulates Viral DNA Replication. Journal of virology 94, (2020).

53. S. Bhattacharya, D. Chakraborty, M. Basu, M. K. Ghosh, Emerging insights into HAUSP (USP7) in physiology, cancer and other diseases. Signal Transduction and Targeted Therapy 3, 17 (2018).

54. N. J. Schauer, X. Liu, R. S. Magin, L. M. Doherty, W. C. Chan, S. B. Ficarro, W. Hu, R. M. Roberts, R. E. Iacob, B. Stolte, A. O. Giacomelli, S. Perera, K. McKay, S. A. Boswell, E. L. Weisberg, A. Ray, D. Chauhan, S. Dhe-Paganon, K. C. Anderson, J. D. Griffin, J. Li, W. C. Hahn, P. K. Sorger, J. R. Engen, K. Stegmaier, J. A. Marto, S. J. Buhrlage, Selective USP7 inhibition elicits cancer cell killing through a p53-dependent mechanism. Scientific reports 10, 5324 (2020).

55. H. Maat, J. Jaques, A. Rodriguez López, S. M. Hogeling, M. P. d. Vries, C. Gravesteijn, A. Z. Brouwers-Vos, N. van der Meer, G. Huls, E. Vellenga, V. van den Boom, J. J. Schuringa, USP7 as part of non-canonical PRC1.1 is a druggable target in leukemia. bioRxiv, 221093 (2019).

56. C. Sánchez, I. Sánchez, J. A. Demmers, P. Rodriguez, J. Strouboulis, M. Vidal, Proteomics analysis of Ring1B/Rnf2 interactors identifies a novel complex with the Fbxl10/Jhdm1B histone demethylase and the Bcl6 interacting corepressor. Molecular & cellular proteomics: MCP 6, 820-834 (2007).

57. E. Lecona, V. Narendra, D. Reinberg, USP7 cooperates with SCML2 to regulate the activity of PRC1. Molecular and cellular biology 35, 1157-1168 (2015).

58. J. M. Dempster, J. Rossen, M. Kazachkova, J. Pan, G. Kugener, D. E. Root, A. Tsherniak, Extracting Biological Insights from the Project Achilles Genome-Scale CRISPR Screens in Cancer Cell Lines. bioRxiv, 720243 (2019).

59. R. M. Meyers, J. G. Bryan, J. M. McFarland, B. A. Weir, A. E. Sizemore, H. Xu, N. V. Dharia, P. G. Montgomery, G. S. Cowley, S. Pantel, A. Goodale, Y. Lee, L. D. Ali, G. Jiang, R. Lubonja, W. F. Harrington, M. Strickland, T. Wu, D. C. Hawes, V. A. Zhivich, M. R. Wyatt, Z. Kalani, J. J. Chang, M. Okamoto, K. Stegmaier, T. R. Golub, J. S. Boehm, F. Vazquez, D. E. Root, W. C. Hahn, A. Tsherniak, Computational correction of copy number effect improves specificity of CRISPR-Cas9 essentiality screens in cancer cells. Nat Genet 49, 1779-1784 (2017).

60. Broad Institute. 2020. “DepMap 20Q2 Public.” Figshare. May 2020. https://doi.org/10.6084/m9.figshare.12280541.v3.

61. G. J. Starrett, M. Thakuria, T. Chen, C. Marcelus, J. Cheng, J. Nomburg, A. R. Thorner, M. K. Slevin, W. Powers, R. T. Burns, C. Perry, A. Piris, F. C. Kuo, G. Rabinowits, A. Giobbie-Hurder, L. E. MacConaill, J. A. DeCaprio, Clinical and molecular characterization of virus-positive and virus-negative Merkel cell carcinoma. Genome Medicine 12, 30 (2020).

62. L. T. Peltenburg, R. Dee, P. I. Schrier, Downregulation of HLA class I expression by c-myc in human melanoma is independent of enhancer A. Nucleic acids research 21, 1179-1185 (1993).

63. R. Bernards, S. K. Dessain, R. A. Weinberg, N-myc amplification causes down-modulation of MHC class I antigen expression in neuroblastoma. Cell 47, 667-674 (1986).

64. M. L. Burr, C. E. Sparbier, K. L. Chan, Y. C. Chan, A. Kersbergen, E. Y. N. Lam, E. Azidis-Yates, D. Vassiliadis, C. C. Bell, O. Gilan, S. Jackson, L. Tan, S. Q. Wong, S. Hollizeck, E. M. Michalak, H. V. Siddle, M. T. McCabe, R. K. Prinjha, G. R. Guerra, B. J. Solomon, S. Sandhu, S. J. Dawson, P. A. Beavis, R. W. Tothill, C. Cullinane, P. J. Lehner, K. D. Sutherland, M. A. Dawson, An Evolutionarily Conserved Function of Polycomb Silences the MHC Class I Antigen Presentation Pathway and Enables Immune Evasion in Cancer. Cancer cell 36, 385-401.e388 (2019).

65. D. Dersh, J. D. Phelan, M. E. Gumina, B. Wang, J. H. Arbuckle, J. Holly, R. J. Kishton, T. E. Markowitz, M. O. Seedhom, N. Fridlyand, G. W. Wright, D. W. Huang, M. Ceribelli, C. J. Thomas, J. B. Lack, N. P. Restifo, T. M. Kristie, L. M. Staudt, J. W. Yewdell, Genome-wide Screens Identify Lineage- and Tumor-Specific Genes Modulating MHC-I- and MHC-II-Restricted Immunosurveillance of Human Lymphomas. Immunity 54, 116-131.e110 (2021).

66. E. C. Townsend, M. A. Murakami, A. Christodoulou, A. L. Christie, J. Koster, T. A. DeSouza, E. A. Morgan, S. P. Kallgren, H. Liu, S.-C. Wu, O. Plana, J. Montero, K. E. Stevenson, P. Rao, R. Vadhi, M. Andreeff, P. Armand, K. K. Ballen, P. Barzaghi-Rinaudo, S. Cahill, R. A. Clark, V. G. Cooke, M. S. Davids, D. J. DeAngelo, D. M. Dorfman, H. Eaton, B. L. Ebert, J. Etchin, B. Firestone, D. C. Fisher, A. S. Freedman, I. A. Galinsky, H. Gao, J. S. Garcia, F. Garnache-Ottou, T. A. Graubert, A. Gutierrez, E. Halilovic, M. H. Harris, Z. T. Herbert, S. M. Horwitz, G. Inghirami, A. M. Intlekofer, M. Ito, S. Izraeli, E. D. Jacobsen, C. A. Jacobson, S. Jeay, I. Jeremias, M. A. Kelliher, R. Koch, M. Konopleva, N. Kopp, S. M. Komblau, A. L. Kung, T. S. Kupper, N. R. LeBoeuf, A. S. LaCasce, E. Lees, L. S. Li, A. T. Look, M. Murakami, M. Muschen, D. Neuberg, S. Y. Ng, O. O. Odejide, S. H. Orkin, R. R. Paquette, A. E. Place, J. E. Roderick, J. A. Ryan, S. E. Sallan, B. Shoji, L. B. Silverman, R. J. Soiffer, D. P. Steensma, K. Stegmaier, R. M. Stone, J. Tamburini, A. R. Thorner, P. van Hummelen, M. Wadleigh, M. Wiesmann, A. P. Weng, J. U. Wuerthner, D. A. Williams, B. M. Wollison, A. A. Lane, A. Letai, M. M. Bertagnolli, J. Ritz, M. Brown, H. Long, J. C. Aster, M. A. Shipp, J. D. Griffin, D. M. Weinstock, The Public Repository of Xenografts Enables Discovery and Randomized Phase II-like Trials in Mice. Cancer cell 29, 574-586 (2016).

67. Z. Hu, D. E. Leet, R. L. Allesøe, G. Oliveira, S. Li, A. M. Luoma, J. Liu, J. Forman, T. Huang, J. B. Iorgulescu, R. Holden, S. Sarkizova, S. H. Gohil, R. A. Redd, J. Sun, L. Elagina, A. Giobbie-Hurder, W. Zhang, L. Peter, Z. Ciantra, S. Rodig, O. Olive, K. Shetty, J. Pyrdol, M. Uduman, P. C. Lee, P. Bachireddy, E. I. Buchbinder, C. H. Yoon, D. Neuberg, B. L. Pentelute, N. Hacohen, K. J. Livak, S. A. Shukla, L. R. Olsen, D. H. Barouch, K. W. Wucherpfennig, E. F. Fritsch, D. B. Keskin, C. J. Wu, P. A. Ott, Personal neoantigen vaccines induce persistent memory T cell responses and epitope spreading in patients with melanoma. Nature medicine 27, 515-525 (2021).

68. Í. F. do Valle, E. Giampieri, G. Simonetti, A. Padella, M. Manfrini, A. Ferrari, C. Papayannidis, I. Zironi, M. Garonzi, S. Bernardi, M. Delledonne, G. Martinelli, D. Remondini, G. Castellani, Optimized pipeline of MuTect and GATK tools to improve the detection of somatic single nucleotide polymorphisms in whole-exome sequencing data. BMC Bioinformatics 17, 341 (2016).

69. D. Benjamin, T. Sato, K. Cibulskis, G. Getz, C. Stewart, L. Lichtenstein, Calling Somatic SNVs and Indels with Mutect2. bioRxiv, 861054 (2019).

70. W. McLaren, L. Gil, S. E. Hunt, H. S. Riat, G. R. S. Ritchie, A. Thormann, P. Flicek, F. Cunningham, The Ensembl Variant Effect Predictor. Genome Biology 17, 122 (2016).

71. A. Mayakonda, D. C. Lin, Y. Assenov, C. Plass, H. P. Koeffler, Maftools: efficient and comprehensive analysis of somatic variants in cancer. Genome research 28, 1747-1756 (2018).

72. Y. Zhu, R. M. Stephens, P. S. Meltzer, S. R. Davis, SRAdb: query and use public next-generation sequencing data from within R. BMC Bioinformatics 14, 19 (2013).

73. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics (Oxford, England) 29, 15-21 (2013).

74. Y. Liao, G. K. Smyth, W. Shi, featureCounts: an efficient general purpose program for assigning sequence reads to genomic features. Bioinformatics (Oxford, England) 30, 923-930 (2014).

75. M. I. Love, W. Huber, S. Anders, Moderated estimation of fold change and dispersion for RNA-seq data with DESeq2. Genome Biology 15, 550 (2014).

76. D. Risso, J. Ngai, T. P. Speed, S. Dudoit, Normalization of RNA-seq data using factor analysis of control genes or samples. Nature biotechnology 32, 896-902 (2014).

77. H. Li, B. Handsaker, A. Wysoker, T. Fennell, J. Ruan, N. Homer, G. Marth, G. Abecasis, R. Durbin, The Sequence Alignment/Map format and SAMtools. Bioinformatics (Oxford, England) 25, 2078-2079 (2009).

78. H. Li, R. Durbin, Fast and accurate short read alignment with Burrows-Wheeler transform. Bioinformatics (Oxford, England) 25, 1754-1760 (2009).

79. G. X. Y. Zheng, J. M. Terry, P. Belgrader, P. Ryvkin, Z. W. Bent, R. Wilson, S. B. Ziraldo, T. D. Wheeler, G. P. McDermott, J. Zhu, M. T. Gregory, J. Shuga, L. Montesclaros, J. G. Underwood, D. A. Masquelier, S. Y. Nishimura, M. Schnall-Levin, P. W. Wyatt, C. M. Hindson, R. Bharadwaj, A. Wong, K. D. Ness, L. W. Beppu, H. J. Deeg, C. McFarland, K. R. Loeb, W. J. Valente, N. G. Ericson, E. A. Stevens, J. P. Radich, T. S. Mikkelsen, B. J. Hindson, J. H. Bielas, Massively parallel digital transcriptional profiling of single cells. Nature Communications 8, 14049 (2017).

80. T. Stuart, A. Butler, P. Hoffman, C. Hafemeister, E. Papalexi, W. M. Mauck, 3rd, Y. Hao, M. Stoeckius, P. Smibert, R. Satija, Comprehensive Integration of Single-Cell Data. Cell 177, 1888-1902.e1821 (2019).

81. I. Korsunsky, N. Millard, J. Fan, K. Slowikowski, F. Zhang, K. Wei, Y. Baglaenko, M. Brenner, P. R. Loh, S. Raychaudhuri, Fast, sensitive and accurate integration of single-cell data with Harmony. Nature methods 16, 1289-1296 (2019).

82. J. G. Abelin, D. B. Keskin, S. Sarkizova, C. R. Hartigan, W. Zhang, J. Sidney, J. Stevens, W. Lane, G. L. Zhang, T. M. Eisenhaure, K. R. Clauser, N. Hacohen, M. S. Rooney, S. A. Carr, C. J. Wu, Mass Spectrometry Profiling of HLA-Associated Peptidomes in Mono-allelic Cells Enables More Accurate Epitope Prediction. Immunity 46, 315-326 (2017).

83. S. Klaeger, A. Apffel, K. R. Clauser, S. Sarkizova, G. Oliveira, S. Rachimi, P. M. Le, A. Tarren, V. Chea, J. G. Abelin, D. A. Braun, P. A. Ott, H. Keshishian, N. Hacohen, D. B. Keskin, C. J. Wu, S. A. Carr, Optimized liquid and gas phase fractionations increase HLA-peptidome coverage for primary cell and tissue samples. Molecular Cellular Proteomics, (Under Revision).

84. P. Mertins, L. C. Tang, K. Krug, D. J. Clark, M. A. Gritsenko, L. Chen, K. R. Clauser, T. R. Clauss, P. Shah, M. A. Gillette, V. A. Petyuk, S. N. Thomas, D. R. Mani, F. Mundt, R. J. Moore, Y. Hu, R. Zhao, M. Schnaubelt, H. Keshishian, M. E. Monroe, Z. Zhang, N. D. Udeshi, D. Mani, S. R. Davies, R. R. Townsend, D. W. Chan, R. D. Smith, H. Zhang, T. Liu, S. A. Carr, Reproducible workflow for multiplexed deep-scale proteome and phosphoproteome analysis of tumor tissues by liquid chromatography-mass spectrometry. Nature protocols 13, 1632-1661 (2018).

85. I. P. Shadforth, T. P. J. Dunkley, K. S. Lilley, C. Bessant, i-Tracker: For quantitative proteomics using iTRAQ™. BMC Genomics 6, 145 (2005).

86. D. B. Keskin, B. B. Reinhold, G. L. Zhang, A. R. Ivanov, B. L. Karger, E. L. Reinherz, Physical detection of influenza A epitopes identifies a stealth subset on human lung epithelium evading natural CD8 immunity. Proceedings of the National Academy of Sciences 112, 2151 (2015).

87. S. Anders, P. T. Pyl, W. Huber, HTSeq--a Python framework to work with high-throughput sequencing data. Bioinformatics (Oxford, England) 31, 166-169 (2015).

88. R. Patro, G. Duggal, M. I. Love, R. A. Irizarry, C. Kingsford, Salmon provides fast and bias-aware quantification of transcript expression. Nature methods 14, 417-419 (2017).

89. M. Love, C. Soneson, R. Patro, Swimming downstream: statistical analysis of differential transcript usage following Salmon quantification. F1000Research 7, 952 (2018).

90. C. Soneson, M. I. Love, M. D. Robinson, Differential analyses for RNA-seq: transcript-level estimates improve gene-level inferences. F1000Res 4, 1521 (2015).

91. M. I. Love, W. Huber, S. Anders, Moderated estimation of fold change and dispersion for RNA-seq data with DESeq2. Genome Biol 15, 550 (2014).

### Tables

TABLE 1

Summary of clinical characteristics of MCC patient samples and the methods by which their cell lines were derived

| Patient ID | Sex | Cell Line Source | MCPyV Viral Status | Prior Treatment |
|---|---|---|---|---|
| 277 | M | PDX | MCPyV+ | CE, RT; MLN0128; CAV; octreotide; imiquimod; cabozantinib |
| 282 | M | PDX | MCPyV- | RT |
| 290 | F | PDX | MCPyV- | none |
| 301 | M | PDX | MCPyV+ | CE, RT |
| 320 | M | PDX | MCPyV- | CE, RT |
| 336 | F | Tumor | MCPyV+ | CE, RT |
| 350 | M | Tumor | MCPyV- | RT |
| 358 | F | Tumor | MCPyV+ | RT |
| 367 | M | PDX | MCPyV+ | RT |
| 383 | M | Tumor | MCPyV+ | RT |
| 2314 | F | PDX | MCPyV+ | Everolimus; CE; Paclitaxel |

PDX = patient-derived xenograft;
MCPyV = Merkel cell polyomavirus;
CE = cisplatin and etoposide;
RT = radiation therapy;
MLN0128 = sapanisertib;
CAV = cyclophosphamide, doxorubicin, and vincristine

TABLE 2

HLA I and IFN Mutations

| Sample | T/CL/both | Gene Sym | Chr | Variant Classification | Ref Allele | Tumor Allele1 | Tumor Allele2 | HGVSc | HGVSp_short | Transcript_ID | PolyPhen |
|---|---|---|---|---|---|---|---|---|---|---|---|
| MCC-336 | both | EIF4E | 4 | Missense_Mutation | T | T | C | c.212A>G | p.D71G | ENST00000505992 | possibly_damaging(0.86) |
| MCC-336 | both | EIF4E | 4 | Missense_Mutation | T | T | G | c.194A>C | p.K65T | ENST00000505992 | benign(0.009) |
| MCC-336 | both | TRIM66 | 11 | Missense_Mutation | A | A | T | c.922T>A | p.F308I | ENST00000402157 | probably_damaging(0.942) |
| MCC-350 | both | HLA-DQA1 | 6 | Nonsense_Mutation | G | G | A | c.581G>A | p.W194* | ENST00000343139 | |
| MCC-350 | both | DDX58 | 9 | Missense_Mutation | T | T | A | c.1751A>T | p.Q584L | ENST00000379883 | benign(0.28) |
| MCC-350 | both | TRIM6 | 11 | Missense_Mutation | G | G | A | c.656G>A | p.R219Q | ENST00000380097 | possibly_damaging(0.535) |
| MCC-350 | both | EIF4G3 | 1 | 5′UTR | G | G | A | c.-558C>T | | ENST00000602326 | |
| MCC-350 | T | GBP6 | 1 | Missense_Mutation | A | A | T | c.628A>T | p.N210Y | ENST00000370456 | benign(0.012) |
| MCC-2314 | CL | NUP155 | 5 | Splice_Site | GCCTT | GCCTT | - | c.724-3_725del | p.X242_splice | ENST00000231498 | |
| MCC-2314 | CL | IFNA16 | 9 | Frame_Shift_Del | GCAAG | GCAAG | - | c.540_544del | p.N180Kfs*17 | ENST00000380216 | |
| MCC-2314 | T | NUP214 | 9 | Missense_Mutation | G | G | T | c.2541G>T | p.R847S | ENST00000359428 | benign(0.085) |
| MCC-2314 | T | TRIM8 | 10 | Frame_Shift_Ins | - | - | GA | c.611_612dup | p.Q205Sfs*22 | ENST00000302424 | |
| MCC-2314 | T | NCAM1 | 11 | Intron | T | T | C | c.2603+1072T>C | | ENST00000524665 | |
| MCC-2314 | T | NCAM1 | 11 | Intron | G | G | A | c.2603+1075G>A | | ENST00000524665 | |
| MCC-301 | CL | TRIM33 | 1 | Frame_Shift_Del | CGCAGCACAAG (SEQ ID NO: 24) | CGCAGCACAAG (SEQ ID NO: 25) | - | c.2697_2707del | p.L900Kfs*17 | ENST00000358465 | |
| MCC-301 | T | PDE12 | 3 | Missense_Mutation | A | A | G | c.1532A>G | p.N511S | ENST00000311180 | benign(0.005) |
| MCC-301 | T | CAMK2G | 10 | Missense_Mutation | A | A | G | c.1204T>C | P.S402P | ENST00000322680 | benign(0) |
| MCC-301 | T | CAMK2G | 10 | Missense_Mutation | G | G | A | c.1184C>T | p.S395L | ENST00000322680 | benign(0) |
| MCC-301 | T | KPNA1 | 3 | Intron | T | T | C | c.432+3458A>G | | ENST00000344337 | |
| MCC-320 | both | ADAR | 1 | Missense_Mutation | G | G | A | c.1718C>T | p.A573V | ENST00000368474 | probably_damaging(0.985) |
| MCC-320 | both | SEC13 | 3 | Missense_Mutation | C | C | T | c.260G>A | p.R87K | ENST00000350697 | benign(0) |
| MCC-320 | both | NUP210 | 3 | Missense_Mutation | G | G | A | c.4595C>T | p.S1532F | ENST00000254508 | benign(0.065) |
| MCC-320 | both | NUP210 | 3 | Splice_Site | T | T | C | c.1046-2A>G | p.X349_splice | ENST00000254508 | |
| MCC-320 | both | HLA-F | 6 | Missense_Mutation | GG | GG | AA | c.726_727delinsAA | p.E243K | ENST00000259951 | |
| MCC-320 | both | TRIM3 | 11 | Missense_Mutation | G | G | A | c.1130C>T | p.P377L | ENST00000525074 | benign(0.005) |
| MCC-320 | both | TRIM66 | 11 | Missense_Mutation | GG | GG | AA | c.3146_3147delinsTT | p.P1049L | ENST00000402157 | benign(0.283) |
| MCC-320 | both | CALR3 | 19 | Missense_Mutation | G | G | T | c.755C>A | p.P252Q | ENST00000269881 | benign(0.001) |
| MCC-320 | CL | HLA-H | 6 | RNA | AGG | AGG | - | n.620_622del | | ENST00000383326 | |
| MCC-320 | both | EIF4A1 | 17 | Intron | C | C | T | c.906+37C>T | | ENST00000293831 | |
| MCC-320 | T | TRIM29 | 11 | Frame_Shift_Del | GCCGATGCAGGAGTCGCACAGC (SEQ ID NO: 26) | GCCGATGCAGGAGTCGCACAGC (SEQ ID NO: 27) | - | c.513_534del | p.L172Tfs*80 | ENST00000341846 | |
| MCC-367 | T | PIAS1 | 15 | Missense_Mutation | A | A | C | c.830A>C | p.N277T | ENST00000249636 | benign(0.005) |

TABLE 3

| Patient | Tumor and Cell Line WES | Cell Line WGS | TumorRNA-seq | Cell Line +/- IFN: RNA-seq | Cell Line +/- IFN: Full and Phospho-Proteome | ATAC-seq | WGBS | Tumor: HLA Peptidome | Cell Line +/- IFN: HLA Peptidome |
|---|---|---|---|---|---|---|---|---|---|
| 277 | X | X | X | X | X | X | X | X | X |
| 282 | | X | X | X | | X | X | X | |
| 290 | | X | X | X | X | X | X | X | X |
| 301 | X | X | X | X | X | X | X | | X |
| 320 | X | X | | X | | X | X | X | |
| 336 | X | X | X | X | | X | X | X | |
| 350 | X | X | X | X | X | X | X | | |
| 358 | | | X | X | | | | X | |
| 367 | X | X | X | X | | X | X | | X |
| 383 | | | | X | | | | | |
| 2314 | X | X | X | X | | X | X | | |

TABLE 4

| | | | | | |
|---|---|---|---|---|---|
| Patient | HLA Allele | Tumor | | Cell Line | |
| MCC- 277 | HLA-A | HLA-A*11:01:01 | HLA-A*32:01:01 | HLA-A*11:01:01 | HLA-A*32:01:01 |
| | HLA-B | HLA-B*14:01:01 | HLA-B*51:01:01 | HLA-B*14:01:01 | HLA-B*51:01:01 |
| | HLA-C | HLA-C*15:02:01 | HLA-C*08:02:01 | HLA-C*15:02:01 | HLA-C*08:02:01 |
| MCC- 301 | HLA-A | HLA-A*24:02:01:01 | HLA-A*02:01:01:01 | HLA-A*24:02:01:01 | HLA-A*02:01:01:01 |
| | HLA-B | HLA-B*15:18:01 | HLA-B*44:02:01:01 | HLA-B*15:18:01 | HLA-B*44:02:01 :01 |
| | HLA-C | HLA-C*07:04:01 | HLA-C*05:01:01:02 | HLA-C*07:04:01 | HLA-C*05:01:01:02 |
| MCC- 320 | HLA-A | HLA-A*01:01:01:01 | HLA-A*25:01:01 | HLA-A*01:01:01:01 | HLA-A*25:01:01 |
| | HLA-B | HLA-B*14:01:01 | HLA-B*18:01:01:02 | HLA-B*14:01:01 | HLA-B*18:01:01:02 |
| | HLA-C | HLA-C*12:03:01:01 | HLA-C*08:02:01 | HLA-C*12:03:01:01 | HLA-C*08:02:01 |
| MCC- 336 | HLA-A | HLA-A*02:01:01:01 | HLA-A*02:01:01:01 | HLA-A*02:01:01:01 | HLA-A*02:01:01:01 |
| | HLA-B | HLA-B*35:02:01 | HLA-B*52:01:01:02 | HLA-B*35:02:01 | HLA-B*52:01:01:02 |
| | HLA-C | HLA-C*12:02:02 | HLA-C*04:01:01:01 | HLA-C*12:02:02 | HLA-C*04:01:01:01 |
| MCC- 350 | HLA-A | HLA-A*24:02:01:01 | HLA-A*29:02:01:01 | HLA-A*24:02:01:01 | HLA-A*29:02:01:01 |
| | HLA-B | HLA-B*07:02:01 | HLA-B*08:01:01 | HLA-B*07:02:01 | HLA-B*08:01:01 |
| | HLA-C | HLA-C*07:02:01:01 | HLA-C*07:01:01:01 | HLA-C*07:02:01:01 | HLA-C*07:01:01:01 |
| MCC- 367 | HLA-A | HLA-A*01:01:01:01 | HLA-A*31:01:02 | HLA-A*01:01:01:01 | HLA-A*31:01:02 |
| | HLA-B | HLA-B*49:01:01 | HLA-B*51:01:01 | HLA-B*49:01:01 | HLA-B*51:01:01 |
| | HLA-C | HLA-C*12:03:01:01 | HLA-C*01:02:01 | HLA-C*12:03:01:01 | HLA-C*01:02:01 |
| MCC- 2314 | HLA-A | HLA-A*24:02:01:01 | HLA-A*02:01:01:01 | HLA-A*24:02:01:01 | HLA-A*02:01:01:01 |
| | HLA-B | HLA-B*07:02:01 | HLA-B*44:02:01:01 | HLA-B*07:02:01 | HLA-B*44:02:01:01 |
| | HLA-C | HLA-C*07:02:01:03 | HLA-C*05:01:01:02 | HLA-C*07:02:01:03 | HLA-C*05:01:01:02 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An immunogenic composition for the treatment of Merkel Cell Carcinoma (MCC) comprising a peptide or polynucleotide encoding for the peptide derived from the OBD polypeptide of Merkel Cell Polyomavirus (MCPyV) large T antigen (MCPyV LT).

2. The immunogenic composition of claim 1, wherein the peptide corresponds to amino acids 341-349 of MCPyV LT.

3. The immunogenic composition of claim 1, wherein the peptide comprises TSDKAIELY (SEQ ID NO:1).

4. The immunogenic composition of any of claims 1 to 3, wherein the peptide is an HLA*A01:01-restricted class I epitope.

5. The immunogenic composition of any of claims 1 to 4, wherein the peptide is presented on an antigen presenting cell.

6. The immunogenic composition of claim 5, wherein the antigen presenting cell is a dendritic cell.

7. The immunogenic composition of any of claims 1 to 4, wherein the peptide is presented by an HLA tetramer.

8. An ex-vivo immune cell for the treatment of Merkel Cell Carcinoma (MCC) comprising a chimeric antigen receptor (CAR), endogenous T cell receptor (TCR) or exogenous T cell receptor (TCR) specific for a peptide derived from the OBD polypeptide of Merkel Cell Polyomavirus (MCPyV) large T antigen (MCPyV LT).

9. The immune cell of claim 8, wherein the peptide corresponds to amino acids 341-349 of MCPyV LT.

10. The immune cell of claim 8, wherein the peptide comprises TSDKAIELY (SEQ ID NO: 1).

11. The immune cell of any of claims 8 to 10, wherein the peptide is an HLA*A01:01-restricted class I epitope.

12. The immune cell of any of claims 8 to 11, wherein the immune cell is a T cell or NK cell.

13. The immune cell of any of claims 8 to 12, wherein the immune cell is an autologous T cell.

14. An antibody for the treatment of Merkel Cell Carcinoma (MCC) specific for a peptide derived from the OBD polypeptide of Merkel Cell Polyomavirus (MCPyV) large T antigen (MCPyV LT).

15. The antibody of claim 14, wherein the peptide corresponds to amino acids 341-349 of MCPyV LT.

16. The antibody of claim 14, wherein the peptide comprises TSDKAIELY (SEQ ID NO:1).

17. The antibody of any of claims 14 to 16, wherein the peptide is an HLA*A01:01-restricted class I epitope.

18. The antibody of any of claims 14 to 17, wherein the antibody is a bispecific antibody or antibody drug conjugate.

19. The antibody of claim 18, wherein the bi-specific antibody is a bi-specific T-cell engager (BiTE).

20. A method of treatment comprising administering the immunogenic composition, immune cell or antibody of any of claims 1 to 19 to a subject in need thereof.

21. The method of claim 20, further comprising administering a treatment that increases HLA class I expression prior or concurrently, wherein the treatment is selected from the group consisting of an interferon gamma therapy and a USP7 inhibitor.
